(19) United States
(12) Patent Application Publication
(10) Pub. No.: US 2025/0171452 A1
(43) Pub. Date: May 29, 2025

(54) TRYPTANTHRIN DERIVATIVES AND USES THEREOF

(71) Applicant: Viscient Bio, Inc., Rolling Hills, CA (US)

(72) Inventor: John Howard HUTCHINSON, San Diego, CA (US)

(21) Appl. No.: 18/922,032

(22) Filed: Oct. 21, 2024

Related U.S. Application Data
(63) Continuation of application No. PCT/US2023/019859, filed on Apr. 25, 2023.
(60) Provisional application No. 63/334,878, filed on Apr. 26, 2022.

(51) Int. Cl. C07D 487/04; A61K 31/519; A61K 31/5377; A61K 31/675; C07D 471/14; C07F 9/6512

(52) U.S. Cl. CPC C07D 487/04; A61K 31/519; A61K 31/5377; A61K 31/675; C07D 471/14; C07F 9/65128

(57) ABSTRACT

The present disclosure provides tryptanthrin derivatives that are modulators of IDO2 and pharmaceutical compositions comprising these compounds. The present disclosure further provides methods of using these compounds for the treatment of disease.

### CROSS-REFERENCE

This application is a continuation of International Application No. PCT/US2023/019859 filed Apr. 25, 2023, which claims the benefit of U.S. Provisional Patent Application No. 63/334,878, filed Apr. 26, 2022, each of which is hereby incorporated by reference in its entirety.

### BACKGROUND OF THE INVENTION

Indole-2,3-dioxygenase 1 (“IDO1”) and Indole-2,3-dioxygenase 2 (“IDO2”) are two closely related enzymes encoded by linked genes. IDO1 and IDO2 are heme-containing enzymes that constitutes the rate-limiting enzyme in the mammalian tryptophan metabolism pathway. IDO enzymes catalyze oxidation of the essential amino acid tryptophan to produce N-formylkynurenine and is responsible for the tryptophan catabolism in vivo. By degrading tryptophan, IDO enzymes produce a tryptophan-poor microenvironment in vivo, and therefore potentially contribute to various diseases, including cancer, cataracts, and neurological disorders.

The IDO pathway is also immunomodulatory, and IDO1 is a well-characterized mediator of tumor immune evasion. Due to its homology with IDO1, IDO2 has been proposed to perform a similar immunoregulatory function and it has been demonstrated that IDO2, like IDO1, is necessary for the differentiation of regulatory T cells in vitro. However, compared to IDO1, in vivo studies have demonstrated a different function for IDO2. Experiments in preclinical models of autoimmune arthritis have suggested a proinflammatory role for IDO2 in mediating B and T cell activation driving autoimmune disease. As IDO enzymes have been implicated in several different diseases, including cancer, there remains a need for highly efficient IDO inhibitors, including those with selectivity between IDO1 and IDO2.

### SUMMARY OF THE INVENTION

In one aspect, the present disclosure provides compound of Formula (I):

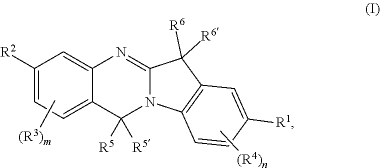

or a pharmaceutically acceptable salt thereof; wherein
R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —NO₂, —N₃, and —CN;
when R¹ is hydroxyl, —O—C₁ alkyl, or —NO₂, R² is selected from
   fluoro, bromo, iodo, —OR²², SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, and —CN;
   C₁₋₆ alkyl, optionally substituted with one or more substituent independently selected from R³⁰; and
   C₃₋₆ carbocycle and 3- to 6-membered heterocycle, any of which is optionally substituted with one or more substituent independently selected from R³⁰;
when R¹ is fluoro, R² is selected from
   bromo, OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³), —N(R²³)P(O)(OR²³)R²³, —S(O)₂R²³, —S(O)₂N(R²³)₂, —NO₂, and —CN;
   C₁₋₆ alkyl, optionally substituted with one or more substituent independently selected from R³⁰; and
   C₃₋₆ carbocycle, 3- to 4-membered heterocycle,

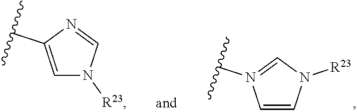

any of which is optionally substituted with one or more substituent independently selected from R³⁰;
when R¹ is C₁ alkyl, —O—C₁ haloalkyl, hydroxy, chloro, or bromo, R² is selected from:
   iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
   C₂₋₆ alkyl, optionally substituted with one or more substituent independently selected from R³⁰;
   C₃₋₆ carbocycle and 3- to 5-membered heterocycle, any of which is optionally substituted with one or more substituent independently selected from R³⁰; and
   when R¹ is selected from bromo, hydroxy, and C₁ alkyl, R² is further selected from fluoro;
when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN, R² is selected from
   halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
   C₁₋₆ alkyl, optionally substituted with one or more substituent independently selected from R³⁰; and
   C₃₋₆ carbocycle and 3- to 6-membered heterocycle, any of which is optionally substituted with one or more substituent independently selected from R³⁰;
   provided when R¹ is —C(O)OR¹¹, R² cannot be substituted 6-membered heterocycle;
R³ and R⁴ are each independently selected at each occurrence from
   halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
   C₁₋₄ alkyl, optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN; and
   C₃₋₄ carbocycle and 3- to 4-membered heterocycle, any of which is optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
R⁵ and R^{5′} are each independently selected from hydrogen and hydroxyl; or R⁵ and R^{5′} taken together are ═O;
R⁶ and R^{6′} are each independently selected from hydrogen and hydroxyl; or R⁶ and R^{6′} taken together are ═O, ═N—OR²⁶, or ═NR²⁶.
m is selected from 0, 1, 2, and 3;
n is selected from 0, 1, 2, and 3;
R¹¹ and R¹⁴ are each independently selected at each occurrence from hydrogen;
   C₁₋₆ alkyl optionally substituted with one or more substituent independently selected from R³¹; and
   C₃₋₆ carbocycle and 3- to 6-membered heterocycle, any of which is optionally substituted with one or more substituent independently selected from R³¹;
R¹² is selected at each occurrence from
   C₁₋₆ alkyl substituted with one or more substituent independently selected from R³²; and
   C₃₋₆ carbocycle and 3- to 6-membered heterocycle, any of which is optionally substituted with one or more substituent independently selected from R³³;
R¹³ is selected at each occurrence from hydrogen;
   C₁₋₆ alkyl optionally substituted with one or more substituent independently selected from R³⁴; and
   C₃₋₅ carbocycle and 3- to 6-membered heterocycle, any of which is optionally substituted with one or more substituent independently selected from R³⁴;
R²¹, R²³, R²⁴, and R²⁶ are each independently selected at each occurrence from hydrogen;
   C₁₋₆ alkyl optionally substituted with one or more substituents independently selected from R³⁴; and
   C₃₋₆ carbocycle and 3- to 6-membered heterocycle, any of which is optionally substituted with one or more substituent independently selected from C₁₋₄ alkyl, C₁₋₄ haloalkyl, and R³⁴;
R²² and R²⁵ are each independently selected at each occurrence from C₂₋₆ alkyl, C₂₋₆ haloalkyl, C₂₋₆ hydroxyalkyl; and
R³⁰, R³¹, R³³, and R³⁴ are each independently selected at each occurrence from
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle;
R³² is independently selected at each occurrence from
   —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, and —CN; and
R⁴¹ is independently selected at each occurrence from hydrogen, C₁₋₆ alkyl, C₁₋₆ haloalkyl, and C₁₋₆ hydroxyalkyl.

In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —N(R¹³)₂, —C(O)OR¹¹, —C(O)N(R¹¹)₂, —N(R¹¹)C(O)R¹¹, —N(R¹¹)S(O)₂(R¹¹), —S(O)₂R¹¹, —S(O)₂N(R¹¹)₂, —NO₂, —N₃, and —CN. In some embodiments, R¹ is selected from fluoro, —C₁₋₆ haloalkyl, —O—C₁₋₆ haloalkyl, —N(R¹³)₂, —C(O)OR¹¹, —C(O)N(R¹¹)₂, —N(R¹¹)C(O)R¹¹, —S(O)₂R¹¹, —NO₂, —N₃, and —CN. In some embodiments, R¹ is hydroxyl, —O—C₁ alkyl, or —NO₂; and R² is selected from fluoro, bromo, iodo, —OR²², SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, —CN, and optionally substituted C₁₋₆ alkyl. In some embodiments, R¹ is NO₂.

In some embodiments, R² is selected from fluoro, bromo, —N(R²¹)₂, —N(R²¹)C(O)R²¹, —N(R²¹)C(O)OR²¹, —NO₂, —CN, and optionally substituted C₁₋₆ alkyl. In some embodiments, R¹ is fluoro; and R² is selected from bromo, OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³), —N(R²³)P(O)(OR²³)R²³, —S(O)₂R²³, —S(O)₂N(R²³)₂, —NO₂, —CN,

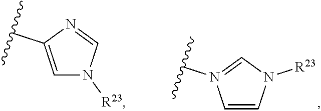

and optionally substituted C₁₋₆ alkyl. In some embodiments, R² is selected from bromo, OR²³, —N(H)R²³, —N(R²³)C(O)R²³, —N(R²³)C(O)OR²³, —NO₂, —CN, and optionally substituted C₁₋₆ alkyl. In some embodiments, R¹ is C₁ alkyl, —O—C₁ haloalkyl, hydroxyl, chloro, or bromo; and R² is selected from iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, —CN, and optionally substituted C₂₋₆ alkyl. In some embodiments, R¹ is —O—C₁ haloalkyl, chloro, or bromo; and R² is selected from —OR²⁵, —C(O)OR²⁵, —C(O)N(R²⁴)₂, —N(R²⁴)C(O)R²⁴, —N(R²⁴)C(O)OR²⁴, —NO₂, —CN, and optionally substituted C₂₋₆ alkyl.

In some embodiments, R¹ is selected from C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, and —CN; and R² is selected from halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, —CN, and optionally substituted C₁₋₆ alkyl. In some embodiments, R¹ is selected from C₂₋₆ alkyl, C₁₋₆ haloalkyl, —O—C₂₋₆ alkyl, —O—C₂₋₆ haloalkyl, —OR¹², —N(R¹³)₂, —C(O)N(R¹¹)₂, —N(R¹¹)C(O)R¹¹, —N(R¹¹)C(O)OR¹¹, —S(O)₂R¹¹, —S(O)₂N(R¹¹)₂, —N₃, and —CN. In some embodiments, R¹ is selected from —N(R¹³)₂, —C(O)N(R¹¹)₂, —N(R¹¹)C(O)R¹, —S(O)₂R¹¹, —N₃, and —CN. In some embodiments, R¹ is —CN. In some embodiments, R² is selected from halogen, —OR²⁶, —SR²⁶, —N(R²⁶)₂, —C(O)R²⁶, —C(O)OR²⁶, —C(O)N(R²⁶)₂, —N(R²⁶)C(O)R²⁶, —N(R²⁶)C(O)OR²⁶, —N(R²⁶)S(O)₂(R²⁶), —S(O)₂R²⁶, —S(O)₂N(R²⁶)₂, —NO₂, —CN, and optionally substituted C₁₋₆ alkyl

In some embodiments, R⁵ and R^{5′} taken together are ═O. In some embodiments, R⁶ and R^{6′} taken together are ═O. In some embodiments, R⁶ and R^{6′} taken together are ═N—OR²⁶. In some embodiments, m is 0 or 1; and n is 0 or 1.

In some embodiments, R¹¹ and R¹⁴ are each independently selected at each occurrence from hydrogen and optionally substituted C₁₋₆ alkyl; R¹² is independently selected at each occurrence from optionally substituted C₁₋₆ alkyl; and R¹³ is independently selected at each occurrence from hydrogen and optionally substituted C₁₋₆ alkyl. In some embodiments, R²¹, R²³, R²⁴, and R²⁶ are each independently selected at each occurrence from hydrogen, optionally substituted C₁₋₆ alkyl, and optionally substituted 3- to 6-membered heterocycle. In some embodiments, R²² and R²⁵ are each independently selected at each occurrence from C₃₋₆ alkyl and C₃₋₆ haloalkyl.

In some embodiments, R³⁰, R³¹, R³³, and R³⁴ are each independently selected at each occurrence from halogen, —OR⁴¹, —N(R⁴¹)₂, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, ═O, and —CN. In some embodiments, R³⁰, R³¹, R³³, and R³⁴ are each independently selected at each occurrence from halogen, —N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, and ═O. In some embodiments, R³² is independently selected at each occurrence from halogen, —N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, and ═O. In some embodiments, R⁴¹ is independently selected at each occurrence from hydrogen, and C₁₋₆ alkyl.

In another aspect, the present disclosure provides a compound of Formula (II):

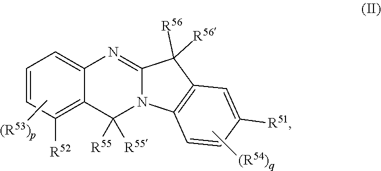

or a pharmaceutically acceptable salt thereof; wherein
R⁵¹ is selected from fluoro, chloro, bromo, iodo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, —NO₂, and —CN;
when R⁵¹ is fluoro, chloro, or iodo, R⁵² is selected from
   bromo, iodo, —N(R⁷¹)₂, —OR⁷¹, —SR⁷¹, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), N(R⁷¹)SO₂N(R⁷¹), —N(R⁷¹)P(O)(OR⁷¹)R⁷¹, —S(O)R⁷¹, —S(O)₂R⁷¹, —S(O)₂N(R⁷¹)₂, —NO₂, and —CN;
   C₁ alkyl substituted with one or more substituent independently selected from R⁸⁰, and C₂₋₆ alkyl optionally substituted with one or more substituent independently selected from R⁸⁰; and
   C₃₋₆ carbocycle and 3- to 5-membered heterocycle, any of which is optionally substituted with one or more substituent independently selected from R⁸⁰;
when R⁵¹ is C₁ alkyl or —NO₂, R⁵² is selected from
   fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, and —CN;
   C₁₋₆ alkyl, optionally substituted with one or more substituent independently selected from R⁸⁰; and
   C₃₋₆ carbocycle and 3- to 6-membered heterocycle, any of which is optionally substituted with one or more substituent independently selected from R⁸⁰;
when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN;
R⁵² is selected from
   halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN;
   C₁₋₆ alkyl, optionally substituted with one or more substituent independently selected from R⁸⁰; and
   C₃₋₆ carbocycle and 3- to 6-membered heterocycle, any of which is optionally substituted with one or more substituent independently selected from R⁸⁰;
R⁵³ and R⁵⁴ are each independently selected at each occurrence from
   halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
   C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN; and
   C₃₋₄ carbocycle and 3- to 4-membered heterocycle, any of which is optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
R⁵⁵ and R^{55′} are each independently selected from hydrogen and hydroxyl; or R⁵⁵ and R^{55′} taken together are ═O;
R⁵⁶ and R^{56′} are each independently selected from hydrogen and hydroxyl; or R⁵⁶ and R^{56′} taken together are ═O, ═N—OR⁶⁴, or ═NR⁶⁴;
p is selected from 0, 1, 2, and 3;
q is selected from 0, 1, 2, and 3;
R⁶¹ and R⁶⁴ are each independently selected at each occurrence from hydrogen;
   C₁₋₆ alkyl optionally substituted with one or more substituent independently selected from R⁸¹; and
   C₃₋₆ carbocycle and 3- to 6-membered heterocycle, any of which is optionally substituted with one or more substituent independently selected from R⁸¹;
R⁷¹, R⁷², and R⁷³ are each independently selected at each occurrence from hydrogen;
   C₁₋₆ alkyl optionally substituted with one or more substituents independently selected from R⁸²; and
   C₃₋₆ carbocycle and 3- to 6-membered heterocycle, any of which is optionally substituted with one or more substituent independently selected from C₁₋₄ alkyl, C₁₋₄ haloalkyl, and R⁸²;
R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)C(O)OR⁹¹, —N(R⁹¹)C(O)N(R⁹¹)₂, —N(R⁹¹)S(O)₂(R⁹¹), —N(R⁹¹)SO₂N(R⁹¹), —N(R⁹¹)P(O)(OR⁹¹)R⁹¹, —S(O)R⁹¹, —S(O)₂R⁹¹, —S(O)₂N(R⁹¹)₂, —NO₂, ═O, ═S, —CN, C₃₋₆ carbocycle, and 3- to 7-membered heterocycle; and
R⁹¹ is independently selected at each occurrence from hydrogen, C₁₋₆ alkyl, C₁₋₆ haloalkyl, and C₁₋₆ hydroxyalkyl.

In some embodiments, R⁵¹ is selected from fluoro, chloro, bromo, hydroxyl, —C₁₋₆ alkyl, —C₁₋₆ haloalkyl —N(R⁶¹)₂, —OR⁶¹, —C(O)R⁶¹, —C(O)OR⁶¹, —C(O)N(R⁶¹)₂, —N(R⁶¹)C(O)R⁶¹—N(R⁶¹)S(O)₂(R⁶¹), —S(O)₂R⁶¹, —S(O)₂N(R⁶¹)₂, —NO₂, and —CN. In some embodiments, R⁵¹ is selected from —C(O)R⁶¹, —C(O)OR⁶¹, —C(O)N(R⁶¹)₂, and —NO₂. In some embodiments, R⁵¹ is fluoro, chloro, or iodo. In some embodiments, C₁ alkyl or —NO₂; and R⁵² is selected from fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, —CN, and optionally substituted C₁₋₆ alkyl. In some embodiments, R⁵¹ is —NO₂. In some embodiments, R⁵² is selected from fluoro, —OR⁷², —NO₂, —CN, and optionally substituted C₁₋₆ alkyl.

In some embodiments, wherein R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN. In some embodiments, R⁵¹ is selected from —OR⁶¹, —C(O)R⁶¹, —C(O)OR⁶¹, —C(O)N(R⁶¹)₂, and —CN; and R⁵² is selected from halogen, —OR⁷³, —SR⁷³, —N(R⁷³)₂, —C(O)R⁷³, —C(O)OR⁷³, —N(R⁷³)C(O)R⁷³, —N(R⁷³)C(O)OR⁷³, —N(R⁷³)C(O)N(R⁷³)₂, —N(R⁷³)S(O)₂(R⁷³), —N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN. In some embodiments, R⁵¹ is selected from —C(O)R⁶¹, —C(O)OR⁶¹, and —C(O)N(R⁶¹)₂. In some embodiments, R⁵² is selected from halogen. In some embodiments, R⁵² is chloro.

In some embodiments, R⁵³ and R⁵⁴ are each independently selected at each occurrence from halogen. In some embodiments, R⁵³ is independently selected at each occurrence from halogen. In some embodiments, R⁵⁵ and R^{55′} taken together are ═O. In some embodiments, R⁵⁶ and R^{56′} taken together are ═O. In some embodiments, R⁵⁶ and R^{56′} taken together are ═N—OR⁶⁴. In some embodiments, p is selected from 0 and 1. In some embodiments, q is 0.

In some embodiments, R⁶¹ and R⁶⁴ are each independently selected at each occurrence from hydrogen, optionally substituted C₁₋₆ alkyl, and optionally substituted 3- to 6-membered heterocycle. In some embodiments, R⁸⁰, R⁸¹, and R⁸² are each independently selected at each occurrence from —N(R⁹¹)₂, —C(O)OR⁹¹, and —N(R⁹¹)SO₂N(R⁹¹). In some embodiments, R⁹¹ is independently selected at each occurrence from hydrogen, and C₁₋₆ alkyl.

In another aspect, the present disclosure provides compound of Formula (III):

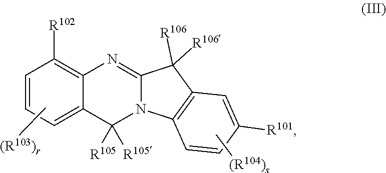

or a pharmaceutically acceptable salt thereof; wherein
R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —SR¹¹¹, —C(O)R¹¹¹, —C(O)OR¹¹¹, —OC(O)R¹¹¹, —OC(O)N(R¹¹¹)₂, —C(O)N(R¹¹¹)₂, —N(R¹¹¹)C(O)R¹¹, —N(R¹¹¹)C(O)OR¹¹, —N(R¹¹¹)C(O)N(R¹¹¹)₂, —N(R¹¹¹)S(O)₂(R¹¹¹), —S(O)₂R¹¹¹, —S(O)₂N(R¹¹¹)₂, —NO₂, and —CN;
when R¹⁰¹ is fluoro, R¹⁰² is selected from
   chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R²¹)C(O)OR¹²¹, —N(R¹²¹)C(O)N(R¹²¹)₂, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN;
   C₁ alkyl substituted with one or more substituent independently selected from R¹²³, and C₂₋₆ alkyl optionally substituted with one or more substituent independently selected from R¹³⁰; and
   C₃₋₆ carbocycle and 3- to 6-membered heterocycle, any of which is optionally substituted with one or more substituent independently selected from R¹³⁰;
when R¹⁰¹ is bromo, iodo, hydroxyl, —O—C₁ alkyl, —C(O)OR¹¹, —C(O)N(H)(C₂ alkyl), —S(O)₂R¹¹¹, or —S(O)₂N(R¹¹)₂, R¹⁰² is selected from
   fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN;
   C₁₋₆ alkyl, optionally substituted with one or more substituent independently selected from R¹³⁰; and
   C₃₋₆ carbocycle and 3- to 6-membered heterocycle, any of which is optionally substituted with one or more substituent independently selected from R¹³⁰;
when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN; R¹⁰² is selected from
   halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
   C₁₋₆ alkyl, optionally substituted with one or more substituent independently selected from R¹³⁰; and
   C₃₋₆ carbocycle and 3- to 6-membered heterocycle, any of which is optionally substituted with one or more substituent independently selected from R¹³⁰;
   provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;
R¹⁰³ and R¹⁰⁴ are each independently selected at each occurrence from
   halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
   C₁₋₄ alkyl, optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN; and
   C₃₋₄ carbocycle and 3- to 4-membered heterocycle, any of which is optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
R¹⁰⁵ and R^{105′} are each independently selected from hydrogen and hydroxyl; or R¹⁰⁵ and R^{105′} taken together are ═O;
R¹⁰⁶ and R^{106′} are each independently selected from hydrogen and hydroxyl; or R¹⁰⁶ and R^{106′} taken together are ═O, ═N—OR¹¹⁴, or ═NR¹¹⁴;
r is selected from 0, 1, 2, and 3;
s is selected from 0, 1, 2, and 3;
R¹¹¹ and R¹¹⁴ are each independently selected at each occurrence from hydrogen;
   C₁₋₆ alkyl optionally substituted with one or more substituent independently selected from R¹³¹; and
   C₃₋₆ carbocycle and 3- to 6-membered heterocycle, any of which is optionally substituted with one or more substituent independently selected from R¹³¹;
R¹¹² is selected at each occurrence from
   C₁₋₆ alkyl substituted with one or more substituent independently selected from R¹³²; and
   C₃₋₆ carbocycle and 3- to 6-membered heterocycle, any of which is optionally substituted with one or more substituent independently selected from R¹³³;
R¹¹³ is selected at each occurrence from hydrogen;
   C₁ alkyl and C₃₋₆ alkyl optionally substituted with one or more substituent independently selected from R¹³⁴, and C₂ alkyl substituted with one or more substituent independently selected from R¹³⁴; and
   C₃₋₅ carbocycle and 3- to 6-membered heterocycle, any of which is optionally substituted with one or more substituent independently selected from R¹³⁴;
R¹²¹, R¹²⁴, and R¹²⁶ are each independently selected at each occurrence from hydrogen;
   C₁₋₆ alkyl optionally substituted with one or more substituents independently selected from R¹³⁴; and
   C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²² and R¹²⁵ are each independently selected at each occurrence from
   C₂₋₆ alkyl optionally substituted with one or more substituents independently selected from R¹³⁴; and
   C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²³ is independently selected at each occurrence from hydrogen;
   C₁₋₆ alkyl optionally substituted with one or more substituents independently selected from R¹³⁴; and
   C₃₋₅ carbocycle and 3- to 6-membered heterocycle, any of which is optionally substituted with one or more substituent independently selected from C₁₋₄ alkyl, C₁₋₄ haloalkyl, and R¹³⁴;
R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —OC(O)N(R¹⁴¹)₂, —C(O)N(R¹⁴¹)₂, —N(R¹⁴¹)C(O)R¹⁴¹, —N(R¹⁴¹)C(O)OR¹⁴¹, —N(R¹⁴¹)C(O)N(R¹⁴¹)₂, —N(R¹⁴¹)S(O)₂(R¹⁴¹), —N(R¹⁴¹)SO₂N(R¹⁴¹), —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, —CN, C₃₋₆ carbocycle, and 3- to 7-membered heterocycle; and
R¹⁴¹ is independently selected at each occurrence from hydrogen, C₁₋₆ alkyl, C₁₋₆ haloalkyl, and C₁₋₆ hydroxyalkyl.

In some embodiments, R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², C(O)R¹¹¹, —C(O)OR¹¹¹, C(O)N(R¹¹¹)₂, N(R¹¹¹)S(O)₂(R¹¹¹), —S(O)₂R¹¹¹, —S(O)₂N(R¹¹¹)₂, —NO₂, and —CN. In some embodiments, R¹⁰¹ is selected from fluoro, —NO₂, and —CN. In some embodiments, R¹⁰¹ is fluoro. In some embodiments, R¹⁰² is selected from chloro, bromo, iodo, —N(R¹²¹)₂, —OR¹²², —C(O)R¹²¹, —C(O)OR¹²¹, C(O)N(R¹²¹)₂, —N(R¹²¹)C(O)R¹²¹, —N(R¹²¹)S(O)₂(R¹²¹), —S(O)₂R¹²¹, —S(O)₂N(R¹²¹)₂, —NO₂, and —CN. In some embodiments, R¹⁰² is chloro or bromo. In some embodiments, R¹⁰¹ is selected from bromo, iodo, hydroxyl, —O—C₁ alkyl, —C(O)OR¹¹¹, —C(O)N(H)(C₂ alkyl), —S(O)₂R¹¹¹, and —S(O)₂N(R¹¹¹)₂. In some embodiments, R¹⁰¹ is selected from chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —N(R¹¹¹)C(O)OR¹¹, —N(R¹¹¹)C(O)N(R¹¹¹)₂, —N(R¹¹¹)S(O)₂(R¹¹¹), —NO₂, and —CN. In some embodiments, R¹⁰¹ is selected from —NO₂ and —CN. In some embodiments, R¹⁰² is selected from halogen and optionally substituted C₁₋₆ alkyl.

In some embodiments, R¹⁰³ and R¹⁰⁴ are each independently selected at each occurrence from halogen and C₁₋₄ alkyl. In some embodiments, R¹⁰⁵ and R^{105′} taken together are ═O. In some embodiments, R¹⁰⁶ and R^{106′} taken together are ═O. In some embodiments, R¹⁰⁶ and R^{106′} taken together are ═N—OR¹¹⁴. In some embodiments, r is selected from 0 and 1. In some embodiments, s is 0.

In another aspect, the present disclosure provides compound of Formula (IV):

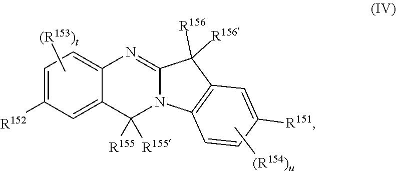

or a pharmaceutically acceptable salt thereof; wherein
R¹⁵¹ is selected from fluoro, bromo, —N(R¹⁶¹)₂, —C(O)R¹⁶¹, —C(O)OR¹⁶¹, —OC(O)R¹⁶¹, —OC(O)N(R¹⁶¹)₂, —C(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), —N(R¹⁶¹)S(O)₂N(R¹⁶¹)₂, —SR¹⁶¹, —S(O)R¹⁶¹, —S(O)₂R¹⁶¹, —S(O)₂N(R¹⁶¹)₂, —CN, —NO₂, —C₁₋₆ haloalkyl, —O—C₂₋₆ alkyl, —O—C₁₋₆ haloalkyl, —OR₃₋₆ carbocycle, and —OR₃₋₆ heterocycle; and

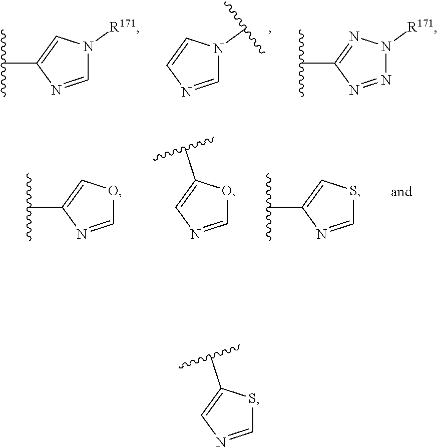

each of which is optionally substituted with one or more substituent independently selected from R¹⁷¹;
when R¹⁵¹ is fluoro, R¹⁵² is selected from
   iodo, —NHR¹⁹², —OR¹⁷¹, —SR¹⁷¹, —C(═NR¹⁹⁶)N(R¹⁹⁶)₂, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁹³)C(O)R¹⁷¹, —N(H)C(O)R¹⁹⁴, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, —S(O)₂N(R¹⁷¹)₂, —NR¹⁹⁶(C═NR¹⁹⁶)N(R¹⁹⁶)₂, —N₃, and —CN;
   C₁ alkyl substituted with one or more substituent independently selected from R¹⁹⁵, and C₂₋₆ alkyl optionally substituted with one or more substituent independently selected from R¹⁸⁰; and
   C₃₋₆ carbocycle, 3-membered heterocycle, 4-membered heterocycle,

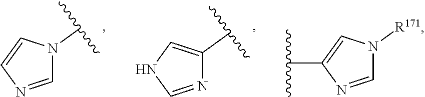

and 6-membered heterocycle, any of which is optionally substituted with one or more substituent independently selected from R¹⁸⁰;
when R¹⁵¹ is

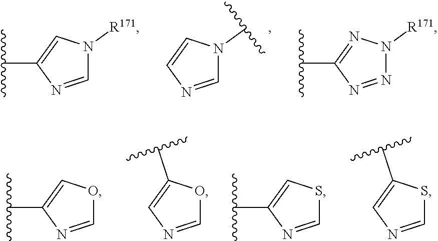

—SR¹⁶¹, —S(O)R¹⁶¹, —S(O)₂R¹⁶¹, or —S(O)₂N(R¹⁶¹)₂, or —CN, R¹⁵² is selected from
halogen, —N(R¹⁷⁴)₂, —OR¹⁷⁴, —SR¹⁷⁴, —C(O)R¹⁷⁴, —C(O)OR¹⁷⁴, —OC(O)R¹⁷⁴, —OC(O)N(R¹⁷⁴)₂, —C(O)N(R¹⁷⁴)₂, —N(R¹⁷⁴)C(O)R¹⁷⁴, —N(R¹⁷⁴)C(O)OR¹⁷⁴, —N(R¹⁷⁴)C(O)N(R¹⁷⁴)₂, —N(R¹⁷⁴)S(O)₂(R¹⁷⁴), —N(R¹²⁴)SO₂N(R¹⁷⁴)₂, —N(R¹⁷⁴)P(O)(OR¹⁷⁴)R¹⁷⁴, —S(O)R¹⁷⁴, —S(O)₂R¹⁷⁴, —S(O)₂N(R¹⁷⁴)₂, —NO₂, and —CN;
   C₁₋₆ alkyl, optionally substituted with one or more substituent independently selected from R¹⁸⁰; and
   C₃₋₆ carbocycle and 3- to 6-membered heterocycle, any of which is optionally substituted with one or more substituent independently selected from R¹⁸⁰;
when R¹⁵¹ is bromo or —NO₂, R¹⁵² is selected from
   iodo, —OR¹⁹⁷, —SR¹⁷⁶, —NHR¹⁷⁸, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
   C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰;
   C₃₋₆ carbocycle and 3- to 6-membered heterocycle, any of which is optionally substituted with one or more substituent independently selected from R¹⁸⁰; and
   provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl;
when R¹⁵¹ is —C₁₋₆ haloalkyl, —O—C₂₋₆ alkyl, —O—C₁₋₆ haloalkyl, —OR₃₋₆ carbocycle, —OR₃₋₆ heterocycle, —N(R¹⁶¹)₂, —C(O)R¹⁶¹, —OC(O)R¹⁶¹, —C(O)N(R¹⁶¹)₂, —OC(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), or —N(R¹⁶¹)S(O)₂N(R¹⁶¹)₂, R¹⁵² is selected from
   halogen, —OR¹⁷⁶, —SR¹⁷⁶, —N(R¹⁷⁶)₂, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
   C₁₋₆ alkyl, optionally substituted with one or more substituent independently selected from R¹⁷⁷; and
   C₃₋₆ carbocycle and 3- to 6-membered heterocycle, any of which is optionally substituted with one or more substituent independently selected from R¹⁷⁷;
R¹⁵³ and R¹⁵⁴ are each independently selected at each occurrence from
   halogen, —OR¹⁶⁴, —NHR¹⁷⁸, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
   C₁₋₄ alkyl, optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN; and
   C₃₋₆ carbocycle and 3- to 5-membered heterocycle, any of which is optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
R¹⁵⁵ and R^{155′} are each independently selected from hydrogen, hydroxyl, and methyl; or R¹⁵⁵ and R^{155′} taken together are ═O;
R¹⁵⁶ and R^{156′} are each independently selected from hydrogen, hydroxyl, and methyl; or R¹⁵⁶ and R^{156′} taken together are ═O, ═N—OR¹⁷¹, or ═NR¹⁷¹;
t is selected from 0, 1, 2, and 3;
u is selected from 0, 1, 2, and 3;
R¹⁶¹ and R¹⁶⁴ are each independently selected at each occurrence from hydrogen;
   C₁₋₆ alkyl optionally substituted with one or more substituent independently selected from R¹⁷⁷; and
   C₃₋₆ carbocycle and 3- to 6-membered heterocycle, any of which is optionally substituted with one or more substituent independently selected from R¹⁷⁷;
R¹⁷¹, R¹⁷⁴, and R¹⁷⁶ are each independently selected at each occurrence from hydrogen;
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁷⁷ and R¹⁸⁰ are each independently selected at each occurrence from halogen, —OR¹⁹¹, —SR¹⁹¹, —N(R¹⁹¹)₂, —C(O)R¹⁹¹, —C(O)OR¹⁹¹, —OC(O)R¹⁹¹, —OC(O)N(R¹⁹¹)₂, —C(O)N(R¹⁹¹)₂, —N(R¹⁹¹)C(O)R¹⁹¹, —N(R¹⁹¹)C(O)OR¹⁹¹, —N(R¹⁹¹)C(O)N(R¹⁹¹)₂, —N(R¹⁹¹)S(O)₂(R¹⁹¹), —N(R¹⁹¹)SO₂N(R¹⁹¹)₂, —N(R¹⁹¹)P(O)(OR¹⁹¹)R¹⁹¹, —NR¹⁹⁶(C═NR¹⁹⁶)N(R¹⁹⁶)₂, —S(O)R¹⁹¹, —S(O)₂R¹⁹¹, —S(O)₂N(R¹⁹¹)₂, —NO₂, ═O, ═S, —CN, C₁₋₆ alkyl, C₃₋₆ carbocycle, and 3- to 7-membered heterocycle;
R¹⁷⁸ is selected at each occurrence from
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from R¹⁸⁰
R¹⁹¹ is independently selected at each occurrence from hydrogen, C₁₋₆ alkyl, C₁₋₆ haloalkyl, and C₁₋₆ hydroxyalkyl.
R¹⁹² is selected at each occurrence from
   C₃₋₆ carbocycle and 3- to 6-membered heterocycle, any of which is optionally substituted with one or more substituent independently selected from R¹⁸⁰
R¹⁹³ is selected at each occurrence from
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁴ is selected at each occurrence from
   C₁₋₆ alkyl substituted with one or more substituents independently selected from R¹⁸⁰;
R¹⁹⁵ is independently selected at each occurrence from halogen, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)S(O)₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
   C₁₋₆ alkyl optionally substituted with one or more substituents independently selected from R¹⁸⁰; and
   C₅₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁶ is selected at each occurrence from
   hydrogen, —CN, and OR¹⁷¹;
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁷ is selected at each occurrence from
   hydrogen;
   C₂₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁸ is independently selected at each occurrence from fluoro, chloro, iodo, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
   C₂₋₆ alkyl optionally substituted with one or more substituents independently selected from R¹⁸⁰; and
   C₅₋₆ carbocycle and 3- to 6-membered heterocycle, any of which is optionally substituted with one or more substituent independently selected from C₁₋₄ alkyl, C₁₋₄ haloalkyl, and R¹⁸⁰;

In some embodiments, R¹⁵¹ is selected from fluoro, bromo, —C(O)R¹⁶¹, —C(O)OR¹⁶¹, —C(O)N(R¹⁶¹)₂, —N(R¹⁶¹)C(O)R¹⁶¹, —N(R¹⁶¹)S(O)₂(R¹⁶¹), —SR¹⁶¹, —S(O)R¹⁶¹, —S(O)₂R¹⁶¹, —S(O)₂N(R¹⁶¹)₂, —CN, —NO₂, —C₁₋₆ haloalkyl, —O—C₂₋₆ alkyl, —O—C₁₋₆ haloalkyl, and

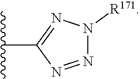

In some embodiments, R¹⁵¹ is selected from fluoro, bromo, —C(O)R¹⁶¹, —C(O)OR¹⁶¹, —C(O)N(R¹⁶¹)₂, —N(R¹⁶¹)C(O)R¹⁶¹, —SR¹⁶¹, —S(O)₂R¹⁶¹, —S(O)₂N(R¹⁶¹)₂, —CN, —NO₂, and

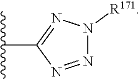

In some embodiments, R¹⁵¹ is selected from fluoro, bromo, —SR¹⁶¹, —S(O)₂R¹⁶¹, —CN, —NO₂, and

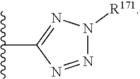

In some embodiments, R¹⁵¹ is fluoro. In some embodiments, R¹⁵² is selected from —NHR¹⁹², —OR¹⁷¹, —SR¹⁷¹, —C(═NR¹⁹⁶)N(R¹⁹⁶)₂, —C(O)R¹⁷¹, —C(O)OR¹⁷¹, —OC(O)R¹⁷¹, —C(O)N(R¹⁷¹)₂, —N(H)C(O)R¹⁹⁴, —N(R¹⁷¹)S(O)₂(R¹⁷¹), —S(O)₂R¹⁷¹, —S(O)₂N(R¹⁷¹)₂, —NR¹⁹⁶(C═NR¹⁹⁶)N(R¹⁹⁶)₂, —N₃, —CN, C₁ alkyl substituted with one or more substituent independently selected from R¹⁹⁵,

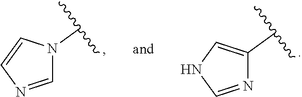 [SMILES: *n1ccnc1 ; *c1c[nH]cn1]

In some embodiments, R¹⁵² is selected from —NHR¹⁹², —C(═NR¹⁹⁶)N(R¹⁹⁶)₂, —C(O)R¹⁷¹, —C(O)OR¹⁷¹, —C(O)N(R¹⁷¹)₂, —N(H)C(O)R¹⁹⁴, —NR¹⁹⁶(C═NR¹⁹⁶)N(R¹⁹⁶)₂, —N₃, —CN, C₁ alkyl substituted with one or more substituent independently selected from R¹⁹⁵,

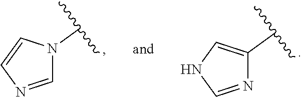 [SMILES: *n1ccnc1 ; *c1c[nH]cn1]

In some embodiments, R¹⁵¹ is selected from —SR¹⁶¹, —S(O)₂R¹⁶¹, —CN, and

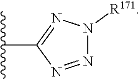

In some embodiments, R¹⁵² is selected from halogen, —N(R¹⁷⁴)₂, —OR¹⁷⁴, —SR¹⁷⁴, —C(O)R¹⁷⁴, —C(O)OR¹⁷⁴, —OC(O)R¹⁷⁴, —C(O)N(R¹⁷⁴)₂, —N(R¹⁷⁴)C(O)R¹⁷⁴, —N(R¹⁷⁴)C(O)OR¹⁷⁴, —N(R¹⁷⁴)S(O)₂(R¹⁷⁴), —S(O)₂R¹⁷⁴, —S(O)₂N(R¹⁷⁴)₂, —NO₂, —CN, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, R¹⁵² is selected from halogen, —N(R¹⁷⁴)₂, —OR¹⁷⁴, —C(O)N(R¹⁷⁴)₂, —N(R¹⁷⁴)C(O)R¹⁷⁴, —N(R¹⁷⁴)C(O)OR¹⁷⁴, —NO₂, —CN, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰.

In some embodiments, R¹⁵¹ is selected from bromo and —NO₂. In some embodiments, R¹⁵² is selected from —OR¹⁹⁷, —SR¹⁷⁶, —NHR¹⁷⁸, —C(O)R¹⁷⁶, —C(O)OR¹⁷⁶, —C(O)N(R¹⁷⁶)₂, —N(R¹⁷⁶)C(O)R¹⁷⁶, —N(R¹⁷⁶)S(O)₂(R¹⁷⁶), —S(O)₂R¹⁷⁶, —S(O)₂N(R¹⁷⁶)₂, —NO₂, —CN, C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰ provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl. In some embodiments, R¹⁵² is selected from C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰ provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl. In some embodiments, R¹⁵¹ is bromo and R¹⁵² is C₁ alkyl substituted with one or more substituent independently selected from R¹⁸⁰. In some embodiments, R¹⁵¹ is —NO₂ and R¹⁵² is C₁ alkyl substituted with one or more substituent independently selected from R¹⁹⁸.

In some embodiments, R¹⁵³ and R¹⁵⁴ are each independently selected at each occurrence from halogen and C₁₋₄ alkyl. In some embodiments, R¹⁵⁵ and R^{155′} taken together are ═O. In some embodiments, R¹⁵⁶ and R^{156′} taken together are ═O. In some embodiments, R¹⁵⁶ and R^{156′} taken together are ═N—OR¹⁷¹. In some embodiments, R¹⁵⁶ and R^{156′} are each independently selected from hydroxyl and methyl. In some embodiments, t is selected from 0 and 1. In some embodiments, u is 0.

In another aspect, the present disclosure provides a pharmaceutical composition comprising a pharmaceutically acceptable excipient and a compound of Formulas (I), (II), (III), or (IV).

In another aspect, the present disclosure provides a method of modulating indoleamine 2,3-dioxygenase 2 (IDO2) in a subject in need thereof, comprising administering to the subject a compound or salt of Formulas (I), (II), (III), or (IV), or a pharmaceutical composition comprising a compound or salt of Formulas (I), (II), (III), or (IV) and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided herein are IDO2 modulators that are useful in the treatment of several diseases or disorders, including cancer and autoimmune diseases. The present disclosure further provides tryptanthrin analogs that are potent IDO2 modulators having different selectivity profiles. In some embodiments, the present disclosure provides modulators of IDO1 and IDO2. In some embodiments, the present disclosure provides IDO2/IDO1 dual modulators. In some embodiments, the present disclosure provides selective IDO2 modulators, which preferentially modulate IDO2 activity over IDO1.

### Definitions

Unless defined otherwise, all technical and scientific terms used herein have the same meaning as is commonly understood by one of skill in the art to which this invention belongs. All patents and publications referred to herein are incorporated by reference. As used in the specification and claims, the singular form “a” “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene-refers to a substituted or unsubstituted alkylene chain with from x to y carbons in the alkylene chain. For example, —C₁₋₆ alkylene— may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene-refers to a substituted or unsubstituted alkenylene chain with from x to y carbons in the alkenylene chain. For example, —C₂₋₆ alkenylene—may be selected from ethenylene, propenylene, butenylene, pentenylene, and hexynylene, any one of which is optionally substituted. An alkenylene chain may have one double bond or more than one double bond in the alkenylene chain. The term —C_{x-y} alkynylene-refers to a substituted or unsubstituted alkynylene chain with from x to y carbons in the alkynylene chain. For example, —C₂₋₆ alkynylene—may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3-to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3-to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocyclic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo(═N—OH), hydrazine(═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine(═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In one aspect, provided herein is a compound having the structure of Formula (I):

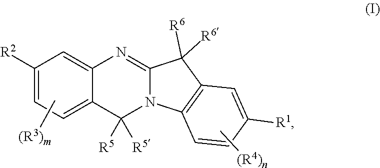

or a pharmaceutically acceptable salt thereof; wherein
R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —NO₂, —N₃, and —CN;
when R¹ is hydroxyl, —O—C₁ alkyl, or —NO₂, R² is selected from
   fluoro, bromo, iodo, —OR²², —SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, and —CN;
   C₁₋₆ alkyl, optionally substituted with one or more substituent independently selected from R³⁰; and
   C₃₋₆ carbocycle and 3- to 6-membered heterocycle, any of which is optionally substituted with one or more substituent independently selected from R³⁰;
when R¹ is fluoro, R² is selected from
   bromo, —OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³), —N(R²³)P(O)(OR²³)R²³, —S(O)₂R²³, —S(O)₂N(R²³)₂, —NO₂, and —CN; C₁₋₆ alkyl, optionally substituted with one or more substituent independently selected from R³⁰; and
C₃₋₆ carbocycle and 3- to 4-membered heterocycle,

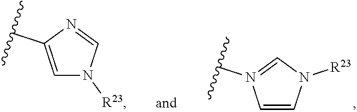

any of which is optionally substituted with one or more substituent independently selected from R³⁰;
when R¹ is C₁ alkyl, —O—C₁ haloalkyl, hydroxy, chloro, or bromo, R² is selected from:
   iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
   C₂₋₆ alkyl, optionally substituted with one or more substituent independently selected from R³⁰;
   C₃₋₆ carbocycle and 3- to 5-membered heterocycle, any of which is optionally substituted with one or more substituent independently selected from R³⁰; and when R¹ is selected from bromo, hydroxy, and C₁ alkyl, R² is further selected from fluoro;
when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN, R² is selected from
   halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
   C₁₋₆ alkyl, optionally substituted with one or more substituent independently selected from R³⁰; and
   C₃₋₆ carbocycle and 3- to 6-membered heterocycle, any of which is optionally substituted with one or more substituent independently selected from R³⁰;
      provided when R¹ is —C(O)OR¹¹, R² cannot be substituted 6-membered heterocycle;
R³ and R⁴ are each independently selected at each occurrence from
   halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
   C₁₋₄ alkyl, optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
   C₃₋₄ carbocycle and 3- to 4-membered heterocycle, any of which is optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
R⁵ and R^{5′} are each independently selected from hydrogen and hydroxyl; or R⁵ and R^{5′} taken together are ═O;
R⁶ and R^{6′} are each independently selected from hydrogen and hydroxyl; or R⁶ and R^{6′} taken together are ═O, ═N—OR²⁶, or ═NR²⁶;
m is selected from 0, 1, 2, and 3;
n is selected from 0, 1, 2, and 3;
R¹¹ and R¹⁴ are each independently selected at each occurrence from hydrogen;
   C₁₋₆ alkyl optionally substituted with one or more substituent independently selected from R³¹;
   C₃₋₆ carbocycle and 3- to 6-membered heterocycle, any of which is optionally substituted with one or more substituent independently selected from R³¹;
R¹² is selected at each occurrence from
   C₁₋₆ alkyl substituted with one or more substituent independently selected from R³²;
   C₃₋₆ carbocycle and 3- to 6-membered heterocycle, any of which is optionally substituted with one or more substituent independently selected from R³³;
R¹³ is selected at each occurrence from hydrogen;
   C₁₋₆ alkyl optionally substituted with one or more substituent independently selected from R³⁴;
   C₃₋₅ carbocycle and 3- to 6-membered heterocycle, any of which is optionally substituted with one or more substituent independently selected from R³⁴;
R²¹, R²³, R²⁴, and R²⁶ are each independently selected at each occurrence from hydrogen;
   C₁₋₆ alkyl optionally substituted with one or more substituents independently selected from R³⁴;
   C₃₋₆ carbocycle and 3- to 6-membered heterocycle, any of which is optionally substituted with one or more substituent independently selected from C₁₋₄ alkyl, C₁₋₄ haloalkyl, and R³⁴;
R²² and R²⁵ are each independently selected at each occurrence from C₂₋₆ alkyl, C₂₋₆ haloalkyl, and C₂₋₆ hydroxyalkyl;
R³⁰, R³¹, R³³, and R³⁴ are each independently selected at each occurrence from halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle;
R³² is independently selected at each occurrence from —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, and —CN; and
R⁴¹ is independently selected at each occurrence from hydrogen, C₁₋₆ alkyl, C₁₋₆ haloalkyl, and C₁₋₆ hydroxyalkyl.

In some embodiment, for the compound or salt of Formula (I),
R¹ is selected from fluoro, —C₁₋₆ haloalkyl, —O—C₁₋₆ haloalkyl, —SR¹¹, —N(R¹³)₂, —C(O)OR¹, —C(O)N(R¹¹)₂, —N(R¹¹)C(O)R¹¹, —S(O)₂R¹¹, —NO₂, —N₃, and —CN;
when R¹ is —NO₂, R² is selected from: fluoro, bromo, iodo, —OR²², —N(R²¹)₂, —C(O)R²³, —C(O)N(R²³)₂, —C(O)OR²³, —OC(O)R²³, —OC(O)N(R²³)₂, —N(R²³)C(O)R²³; —N(R²¹)C(O)OR²¹, —NO₂, —CN, and C₁₋₆ alkyl, optionally substituted with one or more substituent independently selected from R³⁰;
   when R¹ is fluoro, R² is selected from: bromo, —OR²³, —N(H)R²³, —C(O)R²³, —C(O)N(R²³)₂, —C(O)OR²³, —OC(O)R²³, —OC(O)N(R²³)₂, —N(R²³)C(O)R²³, —NO₂, —CN,

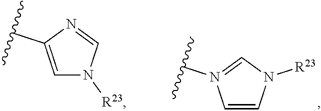

and C₁₋₆ alkyl, optionally substituted with one or more substituent independently selected from R³⁰;
when R¹ is —O—C₁ haloalkyl, R² is selected from: iodo, —OR²⁵, —C(O)R²⁴, —C(O)OR²⁵, —OC(O)R²⁴, —C(O)N(R²⁴)₂, —N(R²⁴)C(O)R²⁴, —NO₂, —CN, and C₂₋₆ alkyl optionally substituted with one or more substituent independently selected from R³⁰;
when R¹ is selected from bromo, hydroxy, and C₁ alkyl, R² is further selected from fluoro;
when R¹ is —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, —N₃, or —CN, R² is selected from halogen, —OR²⁶, —N(R²⁶)₂, —C(O)R²⁶, —C(O)OR²⁶, —OC(O)R²⁶, —C(O)N(R²⁶)₂, —N(R²⁶)C(O)R²⁶, —NO₂, —CN, C₁₋₆ alkyl, optionally substituted with one or more substituent independently selected from R³⁰;
   R³ and R⁴ are each independently selected at each occurrence from halogen, —OR¹⁴, —N(R¹⁴)₂, —NO₂, and —CN; R⁵ and R^{5′} are each independently selected from hydrogen and hydroxyl; or R⁵ and R^{5′} taken together are ═O; R⁶ and R^{6′} are each independently selected from hydrogen and hydroxyl; or R⁶ and R^{6′} taken together are ═O, ═N—OR²⁶, or ═NR²⁶; m is selected from 0 and 1; n is selected from 0 and 1;
   R¹¹ and R¹⁴ are each independently selected at each occurrence from hydrogen; C₁₋₆ alkyl optionally substituted with one or more substituent independently selected from R³¹; and 3- to 6-membered heterocycle optionally substituted with one or more substituent independently selected from R³¹; R¹² is selected at each occurrence from C₁₋₆ alkyl substituted with one or more substituent independently selected from R³²; R¹³ is selected at each occurrence from hydrogen;
   R²¹, R²³, R²⁴, and R²⁶ are each independently selected at each occurrence from hydrogen; C₁₋₆ alkyl optionally substituted with one or more substituents independently selected from R³⁴; and 3- to 6-membered heterocycle optionally substituted with one or more substituent independently selected from C₁₋₄ alkyl, and C₁₋₄ haloalkyl; R²² and R²⁵ are each independently selected at each occurrence from C₂₋₆ alkyl;
   R³⁰, R³¹, R³³, and R³⁴ are each independently selected at each occurrence from halogen, —OR⁴¹, —N(R⁴¹)₂, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, ═O, —CN, and C₃₋₆ carbocycle; R³² is independently selected at each occurrence from —OR⁴¹, —SR⁴¹, —N(R⁴¹)₂, ═O, and —CN; and
   R⁴¹ is independently selected at each occurrence from hydrogen and C₁₋₆ alkyl.

In some embodiments, for the compound or salt of Formula (I), R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹, —N(R¹³)₂, —C(O)R¹, —C(O)OR¹, —OC(O)R¹, —C(O)N(R¹¹)₂, —N(R¹¹)C(O)R¹, —N(R¹¹)S(O)₂(R¹¹), —S(O)₂R¹¹, —S(O)₂N(R¹¹)₂, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹, —C(O)OR¹, —OC(O)R¹, —C(O)N(R¹¹)₂, —N(R¹¹)C(O)R¹, —N(R¹¹)S(O)₂(R¹¹), —S(O)₂R¹¹, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)OR¹¹, —C(O)N(R¹¹)₂, —N(R¹¹)C(O)R¹¹, —S(O)₂R¹¹, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —SR¹¹, —N(R¹³)₂, —C(O)OR¹¹, —C(O)N(R¹¹)₂, —N(R¹¹)C(O)R¹¹, —S(O)₂R¹¹, —NO₂, —N₃, and —CN. In some embodiments, R¹ is selected from fluoro, chloro, bromo, —C₁₋₆ haloalkyl, —O—C₁₋₆ haloalkyl, —SR¹¹, —N(R¹³)₂, —C(O)OR¹, —C(O)N(R¹¹)₂, —N(R¹¹)C(O)R¹¹, —S(O)₂R¹¹, —NO₂, —N₃, and —CN. In some embodiments, R¹ is selected from fluoro, —C₁₋₆ haloalkyl, —O—C₁₋₆ haloalkyl, —SR¹¹, —N(R¹³)₂, —C(O)OR¹, —C(O)N(R¹¹)₂, —N(R¹¹)C(O)R¹¹, —S(O)₂R¹¹, —NO₂, —N₃, and —CN.

In some embodiments, for the compound or salt of Formula (I), R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹, —C(O)OR¹¹, —C(O)N(R¹¹)₂, —N(R¹¹)C(O)R¹¹, —N(R¹¹)C(O)OR¹¹, —N(R¹¹)S(O)₂(R¹¹), —S(O)₂R¹¹, —S(O)₂N(R¹¹)₂, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ haloalkyl, —SR¹¹, —N(R¹³)₂, —C(O)R¹¹, —C(O)OR¹¹, —C(O)N(R¹¹)₂, —N(R¹¹)C(O)R¹¹, —N(R¹¹)C(O)OR¹¹, —N(R¹¹)S(O)₂(R¹¹), —S(O)₂R¹¹, —S(O)₂N(R¹¹)₂, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ haloalkyl, —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —NO₂, —N₃, and —CN. In some embodiments, R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —NO₂, —N₃, and —CN.

In some embodiments, for the compound or salt of Formula (I), R¹ is selected from fluoro, chloro, bromo, and hydroxyl. In some embodiments, R¹ is selected from —C₁₋₆ alkyl, and —C₁₋₆ haloalkyl. In some embodiments, R¹ is selected from hydroxyl, —O—C₁₋₆ alkyl, —O—C₁₋₆ haloalkyl, —OR¹², —OC(O)R¹¹, and —OC(O)N(R¹¹)₂. In some embodiments, R¹ is selected from —NO₂, and —CN. In some embodiments, R¹ is selected from —N(R¹³)₂, —N(R¹¹)C(O)R¹¹, —N(R¹¹)C(O)OR¹¹, —N(R¹¹)S(O)₂(R¹¹), and —N(R¹¹)C(O)N(R¹¹)₂. In some embodiments, R¹ is selected from —C(O)R¹¹, —C(O)OR¹¹, and —C(O)N(R¹¹)₂. In some embodiments, R¹ is selected —SR¹¹, —S(O)₂R¹¹, and —S(O)₂N(R¹¹)₂.

In some embodiments, for the compound or salt of Formula (I), R¹ is fluoro. In some embodiments, R¹ is —C₁₋₆ haloalkyl. In some embodiments, R¹ is —O—C₁₋₆ haloalkyl. In some embodiments, R¹ is —SR¹¹. In some embodiments, R¹ is —N(R¹³)₂. In some embodiments, R¹ is —C(O)OR¹¹. In some embodiments, R¹ is —C(O)N(R¹¹)₂. In some embodiments, R¹ is —N(R¹¹)C(O)R¹¹. In some embodiments, R¹ is —S(O)₂R¹¹. In some embodiments, R¹ is —NO₂. In some embodiments, R¹ is —CN. In some embodiments, R¹ is —N₃.

In some embodiments, for the compound or salt of Formula (I), R¹ is hydroxyl, —O—C₁ alkyl, —NO₂, fluoro, C₁ alkyl, —O—C₁ haloalkyl, hydroxy, chloro, or bromo. In some embodiments, R¹ is hydroxyl, —O—C₁ alkyl, or —NO₂. In some embodiments, R¹ is fluoro. In some embodiments, R¹ is C₁ alkyl, —O—C₁ haloalkyl, hydroxy, chloro, or bromo. In some embodiments, R¹ is hydroxyl, —O—C₁ alkyl, —NO₂, or fluoro. In some embodiments, R¹ is, —NO₂ or fluoro. In some embodiments, R¹ is hydroxyl, —O—C₁ alkyl, —NO₂, C₁ alkyl, —O—C₁ haloalkyl, hydroxy, chloro, or bromo. In some embodiments, R¹ is —NO₂ or —O—C₁ haloalkyl. In some embodiments, R¹ is fluoro, C₁ alkyl, —O—C₁ haloalkyl, hydroxy, chloro, or bromo. In some embodiments, R¹ is fluoro or —O—C₁ haloalkyl.

In some embodiments, for the compound or salt of Formula (I), when R¹ is hydroxyl, —O—C₁ alkyl, or —NO₂, R² is selected from
fluoro, bromo, iodo, —OR²², SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, and —CN;
   C₁₋₆ alkyl, optionally substituted with one or more substituent independently selected from R³⁰; and
   C₃₋₆ carbocycle and 3- to 6-membered heterocycle, any of which is optionally substituted with one or more substituent independently selected from R³⁰.

In some embodiments, for the compound or salt of Formula (I), when R¹ is hydroxyl, —O—C₁ alkyl, or —NO₂; R² is selected from fluoro, bromo, iodo, —OR²², —SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, —CN, and C₁₋₆ alkyl, optionally substituted with one or more substituent independently selected from R³⁰. In some embodiments, when R¹ is hydroxyl, —O—C₁ alkyl, or —NO₂; R² is selected from fluoro, bromo, iodo, —OR²², —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)S(O)₂(R²¹), —S(O)₂R²¹, —S(O)₂N(R²¹)₂, —NO₂, —CN, and C₁₋₆ alkyl, optionally substituted with one or more substituent independently selected from R³⁰. In some embodiments, when R¹ is hydroxyl, —O—C₁ alkyl, or —NO₂; R² is selected from fluoro, bromo, iodo, —OR²², —N(R²¹)₂, —C(O)R²¹, —N(R²¹)C(O)R²¹, —N(R²¹)C(O)OR²¹, —N(R²¹)S(O)₂(R²¹), —NO₂, —CN, C₁₋₆ alkyl, and C₁₋₆ haloalkyl. In some embodiments, when R¹ is hydroxyl, —O—C₁ alkyl, or —NO₂; R² is selected from fluoro, bromo, iodo, —OR²², —N(R²¹)₂, —N(R²¹)C(O)OR²¹, —NO₂, —CN, and C₁₋₆ alkyl. In some embodiments, when R¹ is hydroxyl, —O—C₁ alkyl, or —NO₂; R² is selected from fluoro, bromo, iodo, —N(R²¹)₂, —N(R²¹)C(O)OR²¹, and C₁₋₆ alkyl. In some embodiments, when R¹ is hydroxyl, —O—C₁ alkyl, or —NO₂; R² is selected from fluoro, —N(R²¹)₂, —N(R²¹)C(O)OR²¹, and C₁₋₆ alkyl.

In some embodiments, for the compound or salt of Formula (I), when R¹ is —NO₂; R² is selected from fluoro, bromo, iodo, —OR²², —SR²¹, —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, —CN, and C₁₋₆ alkyl, optionally substituted with one or more substituent independently selected from R³⁰. In some embodiments, when R¹ is —NO₂; R² is selected from fluoro, bromo, iodo, —OR²², —N(R²¹)₂, —C(O)R²¹, —OC(O)R²¹, —OC(O)N(R²¹)₂, —N(R²¹)C(O)R²¹, —N(R²¹)C(O)OR²¹, —N(R²¹)S(O)₂(R²¹), —S(O)₂R²¹, —S(O)₂N(R²¹)₂, —NO₂, —CN, and C₁₋₆ alkyl, optionally substituted with one or more substituent independently selected from R³⁰. In some embodiments, when R¹ is —NO₂; R² is selected from fluoro, bromo, iodo, —OR²², —N(R²¹)₂, —C(O)R²¹, —N(R²¹)C(O)R²¹, —N(R²¹)C(O)OR²¹, —N(R²¹)S(O)₂(R²¹), —NO₂, —CN, C₁₋₆ alkyl, and C₁₋₆ haloalkyl. In some embodiments, when R¹ is —NO₂; R² is selected from fluoro, bromo, iodo, —OR²², —N(R²¹)₂, —N(R²¹)C(O)OR²¹, —NO₂, —CN, and C₁₋₆ alkyl. In some embodiments, when R¹ is —NO₂; R² is selected from fluoro, bromo, iodo, —N(R²¹)₂, —N(R²¹)C(O)OR²¹, and C₁₋₆ alkyl. In some embodiments, when R¹ is —NO₂; R² is selected from fluoro, —N(R²¹)₂, —N(R²¹)C(O)OR²¹, and C₁₋₆ alkyl.

In some embodiments, for the compound or salt of Formula (I), when R¹ is fluoro; R² is selected from:
bromo, —OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³), —N(R²³)P(O)(OR²³)R²³, —S(O)₂R²³, —S(O)₂N(R²³)₂, —NO₂, and —CN;
C₁₋₆ alkyl, optionally substituted with one or more substituent independently selected from R³⁰; and
C₃₋₆ carbocycle, 3- to 4-membered heterocycle,

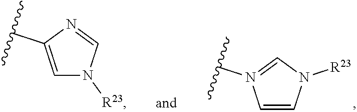

any of which is optionally substituted with one or more substituent independently selected from R³⁰.

In some embodiments, for the compound or salt of Formula (I), when R¹ is fluoro; R² is selected from bromo, —OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³), —N(R²³)P(O)(OR²³)R²³, —S(O)₂R²³, —S(O)₂N(R²³)₂, —NO₂, —CN,

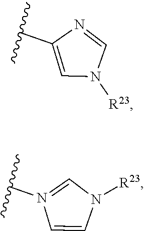

and C₁₋₆ alkyl optionally substituted with one or more substituent independently selected from R³⁰. In some embodiments, when R¹ is fluoro; R² is selected from bromo, —OR²³, —N(H)R²³, —C(O)R²³, —C(O)N(R²³)₂, —C(O)OR²³, —OC(O)R²³, —OC(O)N(R²³)₂, —N(R²³)C(O)R²³, —NO₂, —CN,

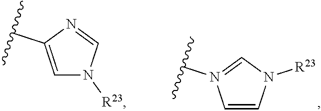

and C₁₋₆ alkyl optionally substituted with one or more substituent independently selected from R³⁰. In some embodiments, when R¹ is fluoro; R² is selected from bromo, —OR²³—N(H)R²³, —C(O)R²³, —C(O)N(R²³)₂, —C(O)OR²³, —NO₂, —CN,

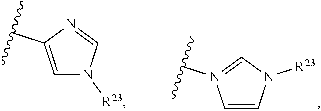

and C₁₋₆ alkyl optionally substituted with one or more substituent independently selected from R³⁰. In some embodiments, when R¹ is fluoro; R² is selected from bromo, —OR²³, —N(H)R²³, —C(O)R²³, —C(O)N(R²³)₂, —C(O)OR²³, —NO₂, —CN,

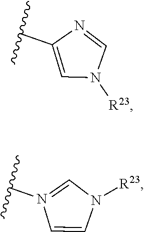

and C₁₋₆ alkyl optionally substituted with one or more substituent independently selected from R³⁰. In some embodiments, when R¹ is fluoro; R² is selected from bromo, —OR²³, —N(H)R²³, —NO₂, —CN,

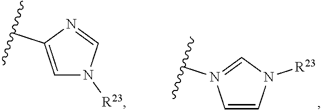

and C₁₋₆ alkyl optionally substituted with one or more substituent independently selected from R³⁰. In some embodiments, when R¹ is fluoro;
R² is selected from bromo, —OR²³, —N(H)R²³,

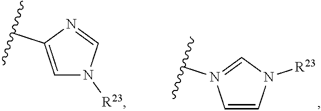

and C₁₋₆ alkyl optionally substituted with one or more substituent independently selected from R³⁰. In some embodiments, when R¹ is fluoro; R² is selected from C₁₋₆ alkyl optionally substituted with one or more substituent independently selected from R³⁰

In some embodiments, for the compound or salt of Formula (I), when R¹ is C₁ alkyl, —O—C₁ haloalkyl, hydroxy, chloro, or bromo; R² is selected from:
iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
C₂₋₆ alkyl, optionally substituted with one or more substituent independently selected from R³⁰;
C₃₋₆ carbocycle and 3- to 5-membered heterocycle, any of which is optionally substituted with one or more substituent independently selected from R³⁰; and
when R¹ is selected from bromo, hydroxy, and C₁ alkyl, R² is further selected from fluoro.

In some embodiments, for the compound or salt of Formula (I), when R¹ is C₁ alkyl, —O—C₁ haloalkyl, hydroxy, chloro, or bromo; R² is selected from:
iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
C₂₋₆ alkyl, optionally substituted with one or more substituent independently selected from R³⁰; and
when R¹ is selected from bromo, hydroxy, and C₁ alkyl, R² is further selected from fluoro.

In some embodiments, for the compound or salt of Formula (I), when R¹ is C₁ alkyl, —O—C₁ haloalkyl, hydroxy, chloro, or bromo, R² is selected from: iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)S(O)₂(R²⁴), —S(O)₂R²⁴, —NO₂, and —CN; and when R¹ is selected from bromo, hydroxy, and C₁ alkyl, R² is further selected from fluoro.

In some embodiments, when R¹ is C₁ alkyl, —O—C₁ haloalkyl, hydroxy, chloro, or bromo; R² is selected from: iodo, —OR²⁵, —C(O)R²⁴, —C(O)OR²⁵, —C(O)N(R²⁴)₂, —N(R²⁴)C(O)R²⁴, —N(R²⁴)S(O)₂(R²⁴), —S(O)₂R²⁴, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: iodo, —OR²⁵, —OC(O)R²⁴, —OC(O)N(R²⁴)₂, —C(O)N(R²⁴)₂, —N(R²⁴)C(O)R²⁴, —N(R²⁴)S(O)₂(R²⁴), —S(O)₂R²⁴, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: iodo, —OR²⁵, —C(O)R²⁴, —C(O)OR²⁵, —OC(O)R²⁴, —OC(O)N(R²⁴)₂, —C(O)N(R²⁴)₂, —S(O)₂R²⁴, —NO₂, and —CN; and when R¹ is selected from bromo, hydroxy, and C₁ alkyl, R² is further selected from fluoro.

In some embodiments, when R¹ is C₁ alkyl, —O—C₁ haloalkyl, hydroxy, chloro, or bromo; R² is selected from: iodo, —OR²⁵, —C(O)R²⁴, —S(O)₂R²⁴, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: iodo, —OR²⁵, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: iodo, —NO₂, and —CN; and when R¹ is selected from bromo, hydroxy, and C₁ alkyl, R² is further selected from fluoro. In some embodiments, when R¹ is C₁ alkyl, —O—C₁ haloalkyl, hydroxy, chloro, or bromo; R² is selected from: —NO₂, and —CN; and when R¹ is selected from bromo, hydroxy, and C₁ alkyl, R² is further selected from fluoro.

In some embodiments, for the compound or salt of Formula (I), when R¹ is —O—C₁ haloalkyl, hydroxy, or bromo; R² is selected from:
fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
C₂₋₆ alkyl, optionally substituted with one or more substituent independently selected from R³⁰;
C₃₋₆ carbocycle and 3- to 5-membered heterocycle, any of which is optionally substituted with one or more substituent independently selected from R³⁰.

In some embodiments, for the compound or salt of Formula (I), when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, —CN, and C₂₋₆ alkyl optionally substituted with one or more substituent independently selected from R³⁰.

In some embodiments, for the compound or salt of Formula (I), when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN. In some embodiments, when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —C(O)N(R²⁴)₂, —N(R²⁴)C(O)R²⁴, —N(R²⁴)S(O)₂(R²⁴), —S(O)R²⁴, —S(O)₂R²⁴, —S(O)₂N(R²⁴)₂, —NO₂, and —CN. In some embodiments, when R¹ is —O—C₁ haloalkyl, hydroxy, or bromo; R² is selected from: fluoro, iodo, —OR²⁵, —OC(O)R²⁴, —C(O)N(R²⁴)₂, —N(R²⁴)C(O)R²⁴, —N(R²⁴)S(O)₂(R²⁴)—S(O)R²⁴, —S(O)₂R²⁴, —S(O)₂N(R²⁴)₂, —NO₂, and —CN. In some embodiments, when R¹ is —O—C₁ haloalkyl, hydroxy, or bromo; R² is selected from: fluoro, iodo, —OR²⁵, —C(O)R²⁴, —C(O)OR²⁵, —C(O)N(R²⁴)₂, —N(R²⁴)C(O)R²⁴, —N(R²⁴)S(O)₂(R²⁴), —S(O)R²⁴, —S(O)₂R²⁴, —S(O)₂N(R²⁴)₂, —NO₂, and —CN. In some embodiments, when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —C(O)N(R²⁴)₂, —S(O)R²⁴, —S(O)₂R²⁴, —S(O)₂N(R²⁴)₂, —NO₂, and —CN. In some embodiments, when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)S(O)₂(R²⁴), —NO₂, and —CN.

In some embodiments, for the compound or salt of Formula (I), when R¹ is —O—C₁ haloalkyl, hydroxy, or bromo; R² is selected from: fluoro, iodo, —OR²⁵, —C(O)R²⁴, —C(O)OR²⁵, —OC(O)R²⁴, —C(O)N(R²⁴)₂, —N(R²⁴)C(O)R²⁴, —N(R²⁴)S(O)₂(R²⁴), —S(O)₂R²⁴, —NO₂, and —CN. In some embodiments, when R¹ is —O—C₁ haloalkyl, hydroxy, or bromo; R² is selected from: fluoro, iodo, —OR²⁵, —C(O)R²⁴, —S(O)₂R²⁴, —NO₂, and —CN. In some embodiments, when R¹ is —O—C₁ haloalkyl, hydroxy, or bromo; R² is selected from: fluoro, iodo, —OR²⁵, —C(O)R²⁴, —NO₂, and —CN. In some embodiments, when R¹ is —O—C₁ haloalkyl, hydroxy, or bromo; R² is selected from: fluoro, iodo, —OR²⁵, —NO₂, and —CN. In some embodiments, when R¹ is —O—C₁ haloalkyl, hydroxy, or bromo; R² is selected from: fluoro, iodo, —NO₂, and —CN. In some embodiments, when R¹ is —O—C₁ haloalkyl, hydroxy, or bromo; R² is selected from: fluoro, —OR²⁵, —NO₂, and —CN. In some embodiments, when R¹ is —O—C₁ haloalkyl, hydroxy, or bromo; R² is selected from: fluoro, —NO₂, and —CN. In some embodiments, when R¹ is —O—C₁ haloalkyl, hydroxy, or bromo; R² is selected from: fluoro and —CN. In some embodiments, for the compound or salt of Formula (I), when R¹ is —O—C₁ haloalkyl, hydroxy, or bromo; R² is selected from: fluoro and —NO₂. In some embodiments, when R¹ is —O—C₁ haloalkyl, hydroxy, or bromo; R² is fluoro.

In some embodiments, for the compound or salt of Formula (I), when R¹ is —O—C₁ haloalkyl or bromo; R² is selected from:
fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN;
C₂₋₆ alkyl, optionally substituted with one or more substituent independently selected from R³⁰;
C₃₋₆ carbocycle and 3- to 5-membered heterocycle, any of which is optionally substituted with one or more substituent independently selected from R³⁰.

In some embodiments, for the compound or salt of Formula (I), when R¹ is —O—C₁ haloalkyl or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, —CN, and C₂₋₆ alkyl optionally substituted with one or more substituent independently selected from R³⁰.

In some embodiments, for the compound or salt of Formula (I), when R¹ is —O—C₁ haloalkyl or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), —N(R²⁴)SO₂N(R²⁴), —N(R²⁴)P(O)(OR²⁴)R²⁴, —S(O)R²⁴, —S(O)₂R²⁴, —S(O)₂N(R²⁴)₂, —NO₂, and —CN. In some embodiments, when R¹ is —O—C₁ haloalkyl or bromo; R² is selected from: fluoro, iodo, —OR²⁵, —C(O)R²⁴, —C(O)OR²⁵, —OC(O)R²⁴, —C(O)N(R²⁴)₂, —N(R²⁴)C(O)R²⁴, —N(R²⁴)S(O)₂(R²⁴), —S(O)R²⁴, —S(O)₂R²⁴, —S(O)₂N(R²⁴)₂, —NO₂, and —CN. In some embodiments, when R¹ is —O—C₁ haloalkyl or bromo; R² is selected from: fluoro, iodo, —OR²⁵, —OC(O)R²⁴, —C(O)N(R²⁴)₂, —N(R²⁴)C(O)R²⁴, —N(R²⁴)S(O)₂(R²⁴), —S(O)R²⁴, —S(O)₂R²⁴, —S(O)₂N(R²⁴)₂, —NO₂, and —CN. In some embodiments, when R¹ is —O—C₁ haloalkyl or bromo; R² is selected from: fluoro, iodo, —OR²⁵, —C(O)R²⁴, —C(O)OR²⁵, —C(O)N(R²⁴)₂, —N(R²⁴)C(O)R²⁴, —N(R²⁴)S(O)₂(R²⁴), —S(O)R²⁴, —S(O)₂R²⁴, —S(O)₂N(R²⁴)₂, —NO₂, and —CN. In some embodiments, when R¹ is —O—C₁ haloalkyl or bromo; R² is selected from: fluoro, iodo, —OR²⁵, —C(O)R²⁴, —C(O)OR²⁵, —OC(O)R²⁴, —C(O)N(R²⁴)₂, —S(O)R²⁴, —S(O)₂R²⁴, —S(O)₂N(R²⁴)₂, —NO₂, and —CN. In some embodiments, when R¹ is —O—C₁ haloalkyl or bromo; R² is selected from: fluoro, iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)S(O)₂(R²⁴), —NO₂, and —CN.

In some embodiments, for the compound or salt of Formula (I), when R¹ is —O—C₁ haloalkyl or bromo; R² is selected from: fluoro, iodo, —OR²⁵, —C(O)R²⁴, —C(O)OR²⁵, —OC(O)R²⁴, —C(O)N(R²⁴)₂, —N(R²⁴)C(O)R²⁴, —N(R²⁴)S(O)₂(R²⁴), —S(O)₂R²⁴, —NO₂, and —CN. In some embodiments, when R¹ is —O—C₁ haloalkyl or bromo; R² is selected from: fluoro, iodo, —OR²⁵, —C(O)R²⁴, —S(O)₂R²⁴, —NO₂, and —CN. In some embodiments, when R¹ is —O—C₁ haloalkyl or bromo; R² is selected from: fluoro, iodo, —OR²⁵, —C(O)R²⁴, —NO₂, and —CN. In some embodiments, when R¹ is —O—C₁ haloalkyl or bromo; R² is selected from: fluoro, iodo, —OR²⁵, —NO₂, and —CN. In some embodiments, when R¹ is —O—C₁ haloalkyl or bromo; R² is selected from: fluoro, iodo, —NO₂, and —CN. In some embodiments, when R¹ is —O—C₁ haloalkyl or bromo; R² is selected from: fluoro, —OR²⁵, —NO₂, and —CN. In some embodiments, when R¹ is —O—C₁ haloalkyl or bromo; R² is selected from: fluoro, —NO₂, and —CN. In some embodiments, when R¹ is —O—C₁ haloalkyl or bromo; R² is selected from: fluoro and —CN. In some embodiments, when R¹ is —O—C₁ haloalkyl or bromo; R² is selected from: fluoro and —NO₂. In some embodiments, when R¹ is —O—C₁ haloalkyl or bromo; R² is fluoro.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from:
halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
C₁₋₆ alkyl, optionally substituted with one or more substituent independently selected from R³⁰; and
C₃₋₆ carbocycle and 3- to 6-membered heterocycle, any of which is optionally substituted with one or more substituent independently selected from R³⁰;
provided when R¹ is —C(O)OR¹¹, R² cannot be substituted 6-membered heterocycle.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from:
halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), —N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
C₁₋₆ alkyl, optionally substituted with one or more substituent independently selected from R³⁰.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), —N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, —CN, and C₁₋₆ alkyl optionally substituted with one or more substituent independently selected from R³⁰.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —OC(O)R²⁶, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —S(O)₂R²⁶, —NO₂, —CN, and C₁₋₆ alkyl.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —C(O)OR²⁶, —C(O)N(R²⁶)₂, —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —C(O)R²⁶, —S(O)₂R²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —SR²⁶, —N(R²⁶)₂, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —NO₂, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —OR²⁶, —NO₂, —CN, and C₁₋₆ alkyl.

In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹, —OC(O)R¹¹, —OC(O)N(R¹¹)₂, —C(O)N(R¹¹)₂, —N(R¹¹)C(O)R¹, —N(R¹¹)C(O)OR¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —NO₂, —CN, and C₁₋₆ alkyl optionally substituted with one or more substituent independently selected from R³⁰. In some embodiments, for the compound or salt of Formula (I), when R¹ is C₂₋₆ alkyl, C₁₋₆ haloalkyl, —O—C₂₋₆ alkyl, —O—C₂₋₆ haloalkyl, —OR¹², —SR¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —NO₂, —CN, and C₁₋₆ alkyl. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen, —CN, and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen and C₁₋₆ alkyl optionally substituted with one or more substituent independently selected from R³⁰. In some embodiments, when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN; R² is selected from: halogen and C₁₋₆ alkyl.

In some embodiments, for the compound or salt of Formula (I), when R¹ is —SR¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from:
halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
C₁₋₆ alkyl, optionally substituted with one or more substituent independently selected from R³⁰; and
C₃₋₆ carbocycle and 3- to 6-membered heterocycle, any of which is optionally substituted with one or more substituent independently selected from R³⁰;
provided when R¹ is —C(O)OR¹¹, R² cannot be substituted 6-membered heterocycle.

In some embodiments, for the compound or salt of Formula (I), when R¹ is —SR¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from:
halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), —N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, and —CN;
C₁₋₆ alkyl, optionally substituted with one or more substituent independently selected from R³⁰.

In some embodiments, for the compound or salt of Formula (I), when R¹ is —SR¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), —N(R²⁶)SO₂N(R²⁶), —N(R²⁶)P(O)(OR²⁶)R²⁶, —S(O)R²⁶, —S(O)₂R²⁶, —S(O)₂N(R²⁶)₂, —NO₂, —CN, and C₁₋₆ alkyl optionally substituted with one or more substituent independently selected from R³⁰.

In some embodiments, for the compound or salt of Formula (I), when R¹ is —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is —SR¹¹, —N(R¹³)₂, —C(O)R¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰.

In some embodiments, for the compound or salt of Formula (I), when R¹ is —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —N(R²⁶)₂, —C(O)R²⁶, —C(O)OR²⁶, —OC(O)R²⁶, —C(O)N(R²⁶)₂, —N(R²⁶)C(O)R²⁶, —N(R²⁶)S(O)₂(R²⁶), —NO₂, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —OC(O)R²⁶, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)N(R²⁶)₂, —N(R²⁶)C(O)R²⁶, —N(R²⁶)S(O)₂(R²⁶), —S(O)₂R²⁶, —NO₂, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —S(O)₂R²⁶, —NO₂, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —OC(O)R²⁶, —C(O)N(R²⁶)₂, —S(O)₂R²⁶, —NO₂, —CN, and C₁₋₆ alkyl.

In some embodiments, for the compound or salt of Formula (I), when R¹ is —SR¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —C(O)OR²⁶, —C(O)N(R²⁶)₂, —S(O)₂R²⁶, —NO₂, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —C(O)R²⁶, —S(O)₂R²⁶, —NO₂, —CN, and C₁₋₆ alkyl optionally substituted with one or more substituent independently selected from R³⁰. In some embodiments, when R¹ is —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —SR²⁶, —N(R²⁶)₂, —NO₂, —CN, and C₁₋₆ alkyl optionally substituted with one or more substituent independently selected from R³⁰. In some embodiments, when R¹ is —SR¹¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —NO₂, —CN, and C₁₋₆ alkyl optionally substituted with one or more substituent independently selected from R³⁰. In some embodiments, when R¹ is —SR¹¹, —N(R¹³)₂, —C(O)R¹¹, —C(O)OR¹, —C(O)N(R¹¹)₂, —N(R¹¹)C(O)R¹¹, —S(O)₂R¹¹, —S(O)₂N(R¹¹)₂, or —CN; R² is selected from: halogen, —OR²⁶, —NO₂, —CN, and C₁₋₆ alkyl.

In some embodiments, for the compound or salt of Formula (I), when R¹ is —SR¹, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —NO₂, —CN, and C₁₋₆ alkyl optionally substituted with one or more substituent independently selected from R³⁰. In some embodiments, for the compound or salt of Formula (I), when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen, —NO₂, —CN, and C₁₋₆ alkyl. In some embodiments, when R¹ is —SR^{1I}, —N(R¹³)₂, —C(O)R¹¹, —C(O)OR¹, —C(O)N(R¹¹)₂, —N(R¹¹)C(O)R¹¹, —S(O)₂R¹¹, —S(O)₂N(R¹¹)₂, or —CN; R² is selected from: halogen, —CN, and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹, —C(O)N(R¹¹)₂, —N(R¹¹)C(O)R¹¹, —S(O)₂R¹¹, —S(O)₂N(R¹¹)₂, or —CN; R² is selected from: halogen and C₁₋₆ alkyl optionally substituted with one or more substituent independently selected from R³⁰. In some embodiments, when R¹ is —SR^{1I}, —N(R¹³)₂, —C(O)R¹¹, —C(O)OR¹¹, —C(O)N(R¹¹)₂, —N(R¹¹)C(O)R¹¹, —S(O)₂R¹¹, —S(O)₂N(R¹¹)₂, or —CN; R² is selected from: halogen and C₁₋₆ alkyl.

In some embodiments, for the compound or salt of Formula (I), R³ and R⁴ are each independently selected at each occurrence from:
halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN; and
C₁₋₄ alkyl, optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN.

In some embodiments, for the compound or salt of Formula (I), R³ and R⁴ are each independently selected at each occurrence from:
halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN; and C₁₋₄ alkyl, optionally substituted with one or more substituents independently selected from halogen, —OR¹⁴, —N(R¹⁴)₂, —NO₂, and —CN.

In some embodiments, for the compound or salt of Formula (I), R³ and R⁴ are each independently selected at each occurrence from:
halogen, —OR¹⁴, —N(R¹⁴)₂, —NO₂, and —CN; and C₁₋₄ alkyl, optionally substituted with one or more substituents independently selected from halogen, —OR¹⁴, —N(R¹⁴)₂, —NO₂, and —CN.

In some embodiments, for the compound or salt of Formula (I), R³ and R⁴ are each independently selected at each occurrence from: halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, —CN, C₁₋₄ alkyl, and C₁₋₄ haloalkyl. In some embodiments, R³ and R⁴ are each independently selected at each occurrence from: halogen, —OR¹⁴, —N(R¹⁴)₂, —N(R¹⁴)C(O)R¹⁴, —C(O)R¹⁴, —C(O)OR¹⁴, —C(O)N(R¹⁴)₂, —NO₂, —CN, C₁₋₄ alkyl, and C₁₋₄ haloalkyl. In some embodiments, R³ and R⁴ are each independently selected at each occurrence from: halogen, —OR¹⁴, —N(R¹⁴)₂, —N(R¹⁴)C(O)R¹⁴, —NO₂, —CN, C₁₋₄ alkyl, and C₁. 4 haloalkyl. In some embodiments, R³ and R⁴ are each independently selected at each occurrence from: halogen, —OR¹⁴, —N(R¹⁴)₂, —C(O)R¹⁴, —C(O)OR¹⁴, —NO₂, —CN, C₁₋₄ alkyl, and C₁₋₄ haloalkyl. In some embodiments, R³ and R⁴ are each independently selected at each occurrence from: halogen, —OR¹⁴, —N(R¹⁴)₂, —NO₂, —CN, C₁₋₄ alkyl, and C₁₋₄ haloalkyl. In some embodiments, R³ and R⁴ are each independently selected at each occurrence from: halogen, —OR¹⁴, —N(R¹⁴)₂, —CN, C₁₋₄ alkyl, and C₁₋₄ haloalkyl. In some embodiments, R³ and R⁴ are each independently selected at each occurrence from: halogen, —OR¹⁴, —CN, C₁₋₄ alkyl, and C₁₋₄ haloalkyl. In some embodiments, R³ and R⁴ are each independently selected at each occurrence from: halogen, —CN, C₁₋₄ alkyl, and C₁₋₄ haloalkyl. In some embodiments, R³ and R⁴ are each independently selected at each occurrence from: halogen, C₁₋₄ alkyl, and C₁₋₄ haloalkyl. In some embodiments, R³ and R⁴ are each independently selected at each occurrence from halogen.

In some embodiments, for the compound or salt of Formula (I), R⁵ and R^{5′} are each independently selected from hydrogen and hydroxyl; or R⁵ and R^{5′} taken together are ═O. In some embodiments, R⁵ and R^{5′} are each independently selected from hydrogen; or R⁵ and R^{5′} taken together are ═O. In some embodiments, R⁵ and R^{5′} are each independently selected from hydrogen and hydroxyl. In some embodiments, R⁵ and R^{5′} taken together are ═O. In some embodiments, R⁵ and R^{5′} are each hydrogen.

In some embodiments, for the compound or salt of Formula (I), R⁶ and R^{6′} are each independently selected from hydrogen and hydroxyl; or R⁶ and R^{6′} taken together are ═O, N—OR²⁶, or ═NR²⁶. In some embodiments, R⁶ and R^{6′} are each independently selected from hydrogen; or R⁶ and R^{6′} taken together are ═O or ═N—OR²³. In some embodiments, R⁶ and R^{6′} are each independently selected from hydrogen and hydroxyl. In some embodiments, R⁶ and R^{6′} taken together are ═O. In some embodiments, R⁶ and R^{6′} taken together are ═N—OR²⁶. In some embodiments, R⁶ and R^{6′} are each hydrogen.

In some embodiments, for the compound or salt of Formula (I), m is selected from 0, 1, 2, and 3. In some embodiments, m is selected from 0, 1, and 2. In some embodiments, m is selected from 0 and 1. In some embodiments, m is selected from 1 and 2. In some embodiments, m is 0. In some embodiments, m is 1. In some embodiments, m is 2.

In some embodiments, for the compound or salt of Formula (I), n is selected from 0, 1, 2, and 3. In some embodiments, n is selected from 0, 1, and 2. In some embodiments, n is selected from 0 and 1. In some embodiments, n is selected from 1 and 2. In some embodiments, n is 0. In some embodiments, n is 1. In some embodiments, n is 2.

In some embodiments, for the compound or salt of Formula (I), R¹¹ and R¹⁴ are each independently selected at each occurrence from:
hydrogen;
C₁₋₆ alkyl optionally substituted with one or more substituent independently selected from R³¹;
C₃₋₆ carbocycle and 3- to 6-membered heterocycle, any of which is optionally substituted with one or more substituent independently selected from R³¹.

In some embodiments, for the compound or salt of Formula (I), R¹¹ and R¹⁴ are each independently selected at each occurrence from: hydrogen; C₁₋₆ alkyl optionally substituted with one or more substituent independently selected from R³¹; and 3- to 6-membered heterocycle, any of which is optionally substituted with one or more substituent independently selected from R³¹. In some embodiments, R¹¹ and R¹⁴ are each independently selected at each occurrence from: hydrogen, C₁₋₆ alkyl, C₃₋₆ carbocycle, and 3- to 6-membered heterocycle. In some embodiments, R¹¹ and R¹⁴ are each independently selected at each occurrence from: hydrogen, C₁₋₆ alkyl, and 3- to 6-membered heterocycle.

In some embodiments, R¹¹ and R¹⁴ are each independently selected at each occurrence from: hydrogen and C₁₋₆ alkyl optionally substituted with one or more substituent independently selected from R³¹. In some embodiments, R¹¹ and R¹⁴ are each independently selected at each occurrence from hydrogen and C₁₋₆ alkyl. In some embodiments, R¹¹ and R¹⁴ are each independently selected at each occurrence from hydrogen and 3- to 6-membered heterocycle optionally substituted with one or more substituent independently selected from R³¹. In some embodiments, R¹¹ and R¹⁴ are each independently selected at each occurrence from hydrogen and 3- to 6-membered heterocycle.

In some embodiments, for the compound or salt of Formula (I), R¹¹ and R¹⁴ are each independently selected at each occurrence from:
hydrogen;
C₁₋₆ alkyl optionally substituted with one or more substituent independently selected from:
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle;
C₃₋₆ carbocycle and 3- to 6-membered heterocycle, any of which is optionally substituted with one or more substituent independently selected from:
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle.

In some embodiments, for the compound or salt of Formula (I), R¹¹ and R¹⁴ are each independently selected at each occurrence from:
hydrogen;
C₁₋₆ alkyl optionally substituted with one or more substituent independently selected from:
   halogen, —OR⁴¹, —N(R⁴¹)₂, —C(O)OR⁴¹, —C(O)N(R⁴¹)₂, —N(R⁴¹)C(O)R⁴¹, —N(R⁴¹)SO₂N(R⁴¹), —N(R⁴¹)P(O)(OR⁴¹)R⁴¹, —S(O)₂R⁴¹, ═O, —CN, and C₃₋₆ carbocycle;
C₃₋₆ carbocycle and 3- to 6-membered heterocycle, any of which is optionally substituted with one or more substituent independently selected from:
   halogen, —OR⁴¹, —N(R⁴¹)₂, —C(O)R⁴¹, —C(O)OR⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, ═O, —CN, and C₃₋₆ carbocycle.

In some embodiments, for the compound or salt of Formula (I), R¹¹ and R¹⁴ are each independently selected at each occurrence from:
hydrogen;
C₁₋₆ alkyl optionally substituted with one or more substituent independently selected from:
   halogen, —OR⁴¹, —N(R⁴¹)₂, —C(O)OR⁴¹—N(R⁴¹)SO₂N(R⁴¹), —N(R⁴¹)P(O)(OR⁴¹)R⁴¹, ═O, —CN, and C₃₋₆ carbocycle;
C₃₋₆ carbocycle and 3- to 6-membered heterocycle, any of which is optionally substituted with one or more substituent independently selected from:
   halogen, —OR⁴¹, —N(R⁴¹)₂, —C(O)OR⁴¹—N(R⁴¹)SO₂N(R⁴¹), —N(R⁴¹)P(O)(OR⁴¹)R⁴¹, ═O, —CN, and C₃₋₆ carbocycle.

In some embodiments, for the compound or salt of Formula (I), R¹² is selected at each occurrence from
C₁₋₆ alkyl substituted with one or more substituent independently selected from R³²;
C₃₋₆ carbocycle and 3- to 6-membered heterocycle, any of which is optionally substituted with one or more substituent independently selected from R³³.

In some embodiments, for the compound or salt of Formula (I), R¹² is selected at each occurrence from C₁₋₆ alkyl substituted with one or more substituent independently selected from R³². In some embodiments, R¹² is selected at each occurrence from C₁₋₆ alkyl optionally substituted with one or more substituent independently selected from R³². In some embodiments, R¹² is selected at each occurrence from C₁₋₆ alkyl.

In some embodiments, for the compound or salt of Formula (I), R¹² is selected at each occurrence from C₁₋₆ alkyl substituted with one or more substituent independently selected from: —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, and —CN. In some embodiments, R¹² is selected at each occurrence from C₁₋₆ alkyl substituted with one or more substituent independently selected from: —OR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂N(R⁴¹)₂, —NO₂, ═O, and —CN. In some embodiments, R¹² is selected at each occurrence from C₁₋₆ alkyl substituted with one or more substituent independently selected from: —OR⁴¹, —N(R⁴¹)₂, —C(O)R⁴¹, —C(O)OR⁴¹, —C(O)N(R⁴¹)₂, —N(R⁴¹)C(O)R⁴¹, ═O, and —CN. In some embodiments, R¹² is selected at each occurrence from C₁₋₆ alkyl substituted with one or more substituent independently selected from: —OR⁴¹, —N(R⁴¹)₂, ═O, and —CN.

In some embodiments, for the compound or salt of Formula (I), R¹³ is selected at each occurrence from
hydrogen;
C₁₋₆ alkyl optionally substituted with one or more substituent independently selected
from R³⁴;
C₃₋₅ carbocycle and 3- to 6-membered heterocycle, any of which is optionally substituted with one or more substituent independently selected from R³⁴.

In some embodiments, for the compound or salt of Formula (I), R¹³ is selected at each occurrence from hydrogen, C₁₋₆ alkyl, C₃₋₅ carbocycle and 3- to 6-membered heterocycle. In some embodiments, R¹³ is selected at each occurrence from hydrogen and C₁₋₆ alkyl optionally substituted with one or more substituent independently selected from R³⁴. In some embodiments, R¹³ is selected at each occurrence from hydrogen and C₁₋₆ alkyl. In some embodiments, R¹³ is selected at each occurrence from hydrogen, C₃₋₅ carbocycle, and 3- to 6-membered heterocycle, any of which is optionally substituted with one or more substituent independently selected from R³⁴. In some embodiments, R¹³ is selected at each occurrence from hydrogen, C₃₋₅ carbocycle, and 3- to 6-membered heterocycle. In some embodiments, R¹³ is selected at each occurrence from hydrogen.

In some embodiments, for the compound or salt of Formula (I), R¹³ is independently selected at each occurrence from:
hydrogen;
C₁₋₆ alkyl optionally substituted with one or more substituent independently selected from:
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN,
   C₃₋₆ carbocycle, and 3- to 7-membered heterocycle;
C₃₋₆ carbocycle and 3- to 6-membered heterocycle, any of which is optionally substituted with one or more substituent independently selected from:
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle.

In some embodiments, for the compound or salt of Formula (I), R¹³ is independently selected at each occurrence from:
hydrogen;
C₁₋₆ alkyl optionally substituted with one or more substituent independently selected from:
   halogen, —OR⁴¹, —N(R⁴¹)₂, —C(O)OR⁴¹, —C(O)N(R⁴¹)₂, —N(R⁴¹)C(O)R⁴¹, —N(R⁴¹)SO₂N(R⁴¹), —N(R⁴¹)P(O)(OR⁴¹)R⁴¹, —S(O)₂R⁴¹, ═O, —CN, and C₃₋₆ carbocycle;
C₃₋₆ carbocycle and 3- to 6-membered heterocycle, any of which is optionally substituted with one or more substituent independently selected from:
   halogen, —OR⁴¹, —N(R⁴¹)₂, —C(O)R⁴¹, —C(O)OR⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, ═O, —CN, and C₃₋₆ carbocycle.

In some embodiments, for the compound or salt of Formula (I), R¹³ is independently selected at each occurrence from:
hydrogen;
C₁₋₆ alkyl optionally substituted with one or more substituent independently selected from:
   halogen, —OR⁴¹, —N(R⁴¹)₂, —C(O)OR⁴¹—N(R⁴¹)SO₂N(R⁴¹), —N(R⁴¹)P(O)(OR⁴¹)R⁴¹, ═O, —CN, and C₃₋₆ carbocycle;
C₃₋₆ carbocycle and 3- to 6-membered heterocycle, any of which is optionally substituted with one or more substituent independently selected from:
   halogen, —OR⁴¹, —N(R⁴¹)₂, —C(O)OR⁴¹—N(R⁴¹)SO₂N(R⁴¹), —N(R⁴¹)P(O)(OR⁴¹)R⁴¹, ═O, —CN, and C₃₋₆ carbocycle.

In some embodiments, for the compound or salt of Formula (I), R²¹, R²³, R²⁴, and R²⁶ are each independently selected at each occurrence from:
hydrogen;
C₁₋₆ alkyl optionally substituted with one or more substituents independently selected from R³⁴;
C₃₋₆ carbocycle and 3- to 6-membered heterocycle, any of which is optionally substituted with one or more substituent independently selected from C₁₋₄ alkyl, C₁₋₄ haloalkyl, and R³⁴.

In some embodiments, for the compound or salt of Formula (I), R²¹, R²³, R²⁴, and R²⁶ are each independently selected at each occurrence from:
hydrogen;
C₁₋₆ alkyl optionally substituted with one or more substituents independently selected from R³⁴;
3- to 6-membered heterocycle, any of which is optionally substituted with one or more substituent independently selected from C₁₋₄ alkyl, C₁₋₄ haloalkyl, and R³⁴.

In some embodiments, for the compound or salt of Formula (I), R²¹, R²³, R²⁴, and R²⁶ are each independently selected at each occurrence from: hydrogen and C₁₋₆ alkyl optionally substituted with one or more substituents independently selected from R³⁴. In some embodiments, for the compound or salt of Formula (I), R²¹, R²³, R²⁴, and R²⁶ are each independently selected at each occurrence from: hydrogen and 3- to 6-membered heterocycle optionally substituted with one or more substituent independently selected from C₁₋₄ alkyl, C₁₋₄ haloalkyl, and R³⁴. In some embodiments, R²¹, R²³, R²⁴, and R²⁶ are each independently selected at each occurrence from: hydrogen, C₁₋₆ alkyl, and 3- to 6-membered heterocycle.

In some embodiments, for the compound or salt of Formula (I), R²¹, R²³, R²⁴, and R²⁶ are each independently selected at each occurrence from:
hydrogen;
C₁₋₆ alkyl optionally substituted with one or more substituents independently selected from:
   halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle;
C₃₋₆ carbocycle and 3- to 6-membered heterocycle, any of which is optionally substituted with one or more substituent independently selected from
   C₁₋₄ alkyl, C₁₋₄ haloalkyl, halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle.

In some embodiments, for the compound or salt of Formula (I), R²¹, R²³, R²⁴, and R²⁶ are each independently selected at each occurrence from:
hydrogen;
C₁₋₆ alkyl optionally substituted with one or more substituents independently selected from:
   halogen, —OR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, —S(O)₂N(R⁴¹)₂, —NO₂, ═O, —CN, and C₃₋₆ carbocycle; and
C₃₋₆ carbocycle and 3- to 6-membered heterocycle, any of which is optionally substituted with one or more substituent independently selected from
   C₁₋₄ alkyl, C₁₋₄ haloalkyl, halogen, —OR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, —S(O)₂N(R⁴¹)₂, —NO₂, ═O, —CN, and C₃₋₆ carbocycle.

In some embodiments, for the compound or salt of Formula (I), R²¹, R²³, R²⁴, and R²⁶ are each independently selected at each occurrence from:
hydrogen;
C₁₋₆ alkyl optionally substituted with one or more substituents independently selected from:
   halogen, —OR⁴¹, —N(R⁴¹)₂, —C(O)R⁴¹, —C(O)OR⁴¹, —C(O)N(R⁴¹)₂, —N(R⁴¹)C(O)R⁴¹, ——N(R⁴¹)SO₂N(R⁴¹), —N(R⁴¹)P(O)(OR⁴¹)R⁴¹, ═O, —CN, and C₃₋₆ carbocycle;
C₃₋₆ carbocycle and 3- to 6-membered heterocycle, any of which is optionally substituted with one or more substituent independently selected from
   C₁₋₄ alkyl, C₁₋₄ haloalkyl, halogen, —OR⁴¹, —N(R⁴¹)₂, —C(O)R⁴¹, —C(O)OR⁴¹, —C(O)N(R⁴¹)₂, —N(R⁴¹)C(O)R⁴¹, —N(R⁴¹)SO₂N(R⁴¹), —N(R⁴¹)P(O)(OR⁴¹)R⁴¹, ═O, —CN, and C₃₋₆ carbocycle.

In some embodiments, for the compound or salt of Formula (I), R²² and R²⁵ are each independently selected at each occurrence from C₂₋₆ alkyl, C₂₋₆ haloalkyl, and C₂₋₆ hydroxyalkyl. In some embodiments, for the compound or salt of Formula (I), R²² and R²⁵ are each independently selected at each occurrence from hydrogen, C₂₋₆ alkyl, C₂₋₆ haloalkyl, and C₂₋₆ hydroxyalkyl. In some embodiments, for the compound or salt of Formula (I), R²² and R²⁵ are each independently selected at each occurrence from C₂₋₆ alkyl and C₂₋₆ haloalkyl. In some embodiments, for the compound or salt of Formula (I), R²² and R²⁵ are each independently selected at each occurrence from C₂₋₆ alkyl. In some embodiments, for the compound or salt of Formula (I), R²² and R²⁵ are each independently selected at each occurrence from C₂₋₆ haloalkyl.

In some embodiments, for the compound or salt of Formula (I), R³⁰, R³¹, R³³, and R³⁴ are each independently selected at each occurrence from: halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle. In some embodiments, R³⁰, R³¹, R³³, and R³⁴ are each independently selected at each occurrence from: halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle. In some embodiments, R³⁰, R³¹, R³³, and R³⁴ are each independently selected at each occurrence from: halogen, —OR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, —CN, C₃₋₆ carbocycle, and 3- to 7-membered heterocycle. In some embodiments, R³⁰, R³¹, R³³, and R³⁴ are each independently selected at each occurrence from: halogen, —OR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, —CN, and C₃₋₆ carbocycle.

In some embodiments, for the compound or salt of Formula (I), R³⁰, R³¹, R³³, and R³⁴ are each independently selected at each occurrence from: halogen, —OR⁴¹, —N(R⁴¹)₂, —C(O)R⁴¹, —C(O)OR⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, —S(O)₂R⁴¹, —S(O)₂N(R⁴¹)₂, —NO₂, ═O, —CN, and C₃₋₆ carbocycle. In some embodiments, R³⁰, R³¹, R³³, and R³⁴ are each independently selected at each occurrence from: halogen, —OR⁴¹, —N(R⁴¹)₂, —C(O)R⁴¹, —C(O)OR⁴¹, —C(O)N(R⁴¹)₂, —N(R⁴¹)C(O)R⁴¹, —N(R⁴¹)S(O)₂(R⁴¹), —N(R⁴¹)SO₂N(R⁴¹), —N(R⁴¹)P(O)(OR⁴¹)R⁴¹, ═O, —CN, and C₃₋₆ carbocycle. In some embodiments, R³⁰, R³¹, R³³, and R³⁴ are each independently selected at each occurrence from: halogen, —OR⁴¹, —N(R⁴¹)₂, —C(O)R⁴¹, —C(O)OR⁴¹, —C(O)N(R⁴¹)₂, —N(R⁴¹)SO₂N(R⁴¹), —N(R⁴¹)P(O)(OR⁴¹)R⁴¹, ═O, —CN, and C₃₋₆ carbocycle. In some embodiments, R³⁰, R³¹, R³³, and R³⁴ are each independently selected at each occurrence from: halogen, —OR⁴¹, —N(R⁴¹)₂, —C(O)OR⁴¹, —N(R⁴¹)SO₂N(R⁴¹), —N(R⁴¹)P(O)(OR⁴¹)R⁴¹, ═O, —CN, and C₃₋₆ carbocycle. In some embodiments, R³⁰, R³¹, R³³, and R³⁴ are each independently selected at each occurrence from: —N(R⁴¹)₂, —C(O)OR⁴¹, —N(R⁴¹)SO₂N(R⁴¹), —N(R⁴¹)P(O)(OR⁴¹)R⁴¹, and C₃₋₆ carbocycle.

In some embodiments, for the compound or salt of Formula (I), R³² is independently selected at each occurrence from: —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹), —N(R⁴¹)P(O)(OR⁴¹)R⁴¹, —S(O)R⁴¹, —S(O)₂R⁴¹, —S(O)₂N(R⁴¹)₂, —NO₂, ═O, ═S, and —CN. In some embodiments, R³² is independently selected at each occurrence from: —OR⁴¹, —SR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —S(O)₂R⁴¹, —S(O)₂N(R⁴¹)₂, —NO₂, ═O, ═S, and —CN. In some embodiments, R³² is independently selected at each occurrence from: —OR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, —N(R⁴¹)S(O)₂(R⁴¹), —S(O)₂R⁴¹, —S(O)₂N(R⁴¹)₂, ═O, and —CN. In some embodiments, R³² is independently selected at each occurrence from: —OR⁴¹, —N(R⁴¹)₂, —C(O)R⁴¹, —C(O)OR⁴¹, —OC(O)R⁴¹, —C(O)N(R⁴¹)₂, —N(R⁴¹)C(O)R⁴¹, ═O, and —CN. In some embodiments, R³² is independently selected at each occurrence from: —OR⁴¹, —N(R⁴¹)₂, ═O, and —CN.

In some embodiments, for the compound or salt of Formula (I), R⁴¹ is independently selected at each occurrence from hydrogen, C₁₋₆ alkyl, C₁₋₆ haloalkyl, and C₁₋₆ hydroxyalkyl. In some embodiments, R⁴¹ is independently selected at each occurrence from hydrogen, C₁₋₆ alkyl, and C₁₋₆ haloalkyl. In some embodiments, R⁴¹ is independently selected at each occurrence from hydrogen, C₁₋₆ alkyl, and C₁₋₆ hydroxyalkyl. In some embodiments, R⁴¹ is independently selected at each occurrence from hydrogen and C₁₋₆ alkyl. In some embodiments, R⁴¹ is independently selected at each occurrence from C₁₋₆ alkyl. In some embodiments, R⁴¹ is independently selected at each occurrence from hydrogen.

In one aspect, provided herein is a compound having the structure of Formula (II):

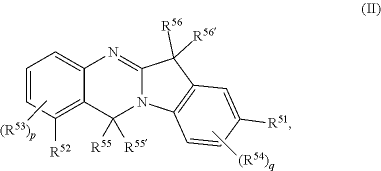

or a pharmaceutically acceptable salt or solvate thereof, wherein
R⁵¹ is selected from fluoro, chloro, bromo, iodo, hydroxyl, —C₁₋₆ alkyl, —C₁₋₆ haloalkyl —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, —NO₂, and —CN;
when R⁵¹ is fluoro, chloro, or iodo, R⁵² is selected from
   bromo, iodo, —N(R⁷¹)₂, —OR⁷¹, —SR⁷¹, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), N(R⁷¹)SO₂N(R⁷¹), —N(R⁷¹)P(O)(OR⁷¹)R⁷¹, —S(O)R⁷¹, —S(O)₂R⁷¹, —S(O)₂N(R⁷¹)₂, —NO₂, and —CN;
   C₁ alkyl substituted with one or more substituent independently selected from R⁸⁰, and C₂₋₆ alkyl optionally substituted with one or more substituent independently selected from R⁸⁰; and
   C₃₋₆ carbocycle and 3- to 5-membered heterocycle, any of which is optionally substituted with one or more substituent independently selected from R⁸⁰;
when R⁵¹ is C₁ alkyl or —NO₂, R⁵² is selected from
   fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, and —CN;
   C₁₋₆ alkyl, optionally substituted with one or more substituent independently selected from R⁸⁰; and
   C₃₋₆ carbocycle and 3- to 6-membered heterocycle, any of which is optionally substituted with one or more substituent independently selected from R⁸⁰;
when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN;
   R⁵² is selected from
   halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN;
   C₁₋₆ alkyl, optionally substituted with one or more substituent independently selected from R⁸⁰; and
   C₃₋₆ carbocycle and 3- to 6-membered heterocycle, any of which is optionally substituted with one or more substituent independently selected from R⁸⁰;
R⁵³ and R⁵⁴ are each independently selected at each occurrence from
   halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
   C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
   C₃₋₄ carbocycle and 3- to 4-membered heterocycle, any of which is optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
R⁵⁵ and R^{55′} are each independently selected from hydrogen and hydroxyl; or R⁵⁵ and R^{55′} taken together are ═O;
R⁵⁶ and R^{56′} are each independently selected from hydrogen and hydroxyl; or R⁵⁶ and R^{56′} taken together are ═O, ═N—OR⁶⁴, or ═NR⁶⁴;
p is selected from 0, 1, 2, and 3;
q is selected from 0, 1, 2, and 3;
R⁶¹ and R⁶⁴ are each independently selected at each occurrence from
   hydrogen;
   C₁₋₆ alkyl optionally substituted with one or more substituent independently selected from R⁸¹;
   C₃₋₆ carbocycle and 3- to 6-membered heterocycle, any of which is optionally substituted with one or more substituent independently selected from R⁸¹;
R⁷¹, R⁷², and R⁷³ are each independently selected at each occurrence from
   hydrogen;
   C₁₋₆ alkyl optionally substituted with one or more substituents independently selected from R⁸²;
   C₃₋₆ carbocycle and 3- to 6-membered heterocycle, any of which is optionally substituted with one or more substituent independently selected from C₁₋₄ alkyl, C₁₋₄ haloalkyl, and R⁸².
R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)C(O)OR⁹¹, —N(R⁹¹)C(O)N(R⁹¹)₂, —N(R⁹¹)S(O)₂(R⁹¹), —N(R⁹¹)SO₂N(R⁹¹), —N(R⁹¹)P(O)(OR⁹¹)R⁹¹, —S(O)R⁹¹, —S(O)₂R⁹¹, —S(O)₂N(R⁹¹)₂, —NO₂, ═O, ═S, —CN, C₃₋₆ carbocycle, and 3- to 7-membered heterocycle; and
R⁹¹ is independently selected at each occurrence from hydrogen, C₁₋₆ alkyl, C₁₋₆ haloalkyl, and C₁₋₆ hydroxyalkyl.

In some embodiments, for the compound or salt of Formula (II), R⁵¹ is selected from fluoro, chloro, bromo, iodo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, —NO₂, and —CN. In some embodiments, R⁵¹ is selected from fluoro, chloro, bromo, iodo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —C(O)N(R⁶¹)₂, —N(R⁶¹)C(O)R⁶¹, —N(R⁶¹)S(O)₂(R⁶¹), —S(O)₂R⁶¹, —S(O)₂N(R⁶¹)₂, —NO₂, and —CN. In some embodiments, R⁵¹ is selected from fluoro, chloro, bromo, iodo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —C(O)N(R⁶¹)₂, —N(R⁶¹)C(O)R⁶¹, —NO₂, and —CN. In some embodiments, R⁵¹ is selected from fluoro, chloro, bromo, iodo, hydroxyl, —C₁₋₆ alkyl, —C₁₋₆ haloalkyl, —N(R⁶¹)₂, —OR⁶¹, —C(O)R⁶¹, —C(O)OR⁶¹, —OC(O)R⁶¹, —C(O)N(R⁶¹)₂, —N(R⁶¹)C(O)R⁶¹, —NO₂, and —CN. In some embodiments, R⁵¹ is selected from fluoro, chloro, bromo, iodo, hydroxyl, —C₁₋₆ alkyl, —C₁₋₆ haloalkyl, —N(R⁶¹)₂, —OR⁶¹, —C(O)R⁶¹, —C(O)OR⁶¹, —C(O)N(R⁶¹)₂, —NO₂, and —CN. In some embodiments, R⁵¹ is selected from fluoro, chloro, bromo, iodo, hydroxyl, —N(R⁶¹)₂, —OR⁶¹, —C(O)R⁶¹, —C(O)OR⁶¹, —C(O)N(R⁶¹)₂, —NO₂, and —CN. In some embodiments, R⁵¹ is selected from fluoro, chloro, bromo, iodo, hydroxyl, —C(O)R⁶¹, —C(O)OR⁶¹, —C(O)N(R⁶¹)₂, —NO₂, and —CN. In some embodiments, R⁵¹ is selected from fluoro, chloro, bromo, —C(O)R⁶¹, —C(O)OR⁶¹, —C(O)N(R⁶¹)₂, —NO₂, and —CN. In some embodiments, R⁵¹ is selected from fluoro, —C(O)R⁶¹, —C(O)OR⁶¹, —C(O)N(R⁶¹)₂, —NO₂, and —CN. In some embodiments, R⁵¹ is selected from —C(O)R⁶¹, —C(O)OR⁶¹, —C(O)N(R⁶¹)₂, and —NO₂.

In some embodiments, for the compound or salt of Formula (II), R⁵¹ is fluoro, chloro, or iodo. In some embodiments, R⁵¹ is C₁ alkyl or —NO₂. In some embodiments, R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN. In some embodiments, for the compound or salt of Formula (II), R⁵¹ is selected from fluoro, chloro, iodo, C₁ alkyl or —NO₂.

In some embodiments, for the compound or salt of Formula (II), R⁵¹ is selected from fluoro, chloro, iodo, bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN.

In some embodiments, R⁵¹ is selected from fluoro, chloro, iodo, bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —C(O)N(R⁶¹)₂, —N(R⁶¹)S(O)₂(R⁶¹), —S(O)₂R⁶¹, —S(O)₂N(R⁶¹)₂, and —CN. In some embodiments, R⁵¹ is selected from fluoro, chloro, iodo, bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —C(O)N(R⁶¹)₂, and —CN. In some embodiments, R⁵¹ is selected from —C(O)R⁶¹, —C(O)OR⁶¹, —C(O)N(R⁶¹)₂, and —CN.

In some embodiments, for the compound or salt of Formula (II), R⁵¹ is selected from C₁ alkyl, —NO₂, bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN. In some embodiments, R⁵¹ is selected from C₁ alkyl, —NO₂, bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —C(O)N(R⁶¹)₂, —N(R⁶¹)C(O)R⁶¹, and —CN. In some embodiments, R⁵¹ is selected from —NO₂, —C(O)R⁶¹, —C(O)OR⁶¹, and —C(O)N(R⁶¹)₂.

In some embodiments, for the compound or salt of Formula (II), R⁵¹ is selected from bromo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, —NO₂, and —CN. In some embodiments, R⁵¹ is selected from fluoro, chloro, bromo, iodo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN. In some embodiments, R⁵¹ is selected from fluoro, chloro, iodo, —C₁ alkyl, and —NO₂. In some embodiments, R⁵¹ is selected from fluoro, chloro, and iodo. In some embodiments, R⁵¹ is selected from C₁ alkyl or —NO₂. In some embodiments, R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN.

In some embodiments, for the compound or salt of Formula (II), when R⁵¹ is fluoro, chloro, or iodo, R⁵² is selected from
bromo, iodo, —N(R⁷¹)₂, —OR⁷¹, —SR⁷¹, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷1, —N(R⁷¹)C(O)N(R⁷¹)₂, —N(R⁷¹)S(O)₂(R⁷¹), N(R⁷¹)SO₂N(R⁷¹), —N(R⁷¹)P(O)(OR⁷¹)R⁷¹, —S(O)R⁷¹, —S(O)₂R⁷¹, —S(O)₂N(R⁷¹)₂, —NO₂, and —CN;
C₁ alkyl substituted with one or more substituent independently selected from R⁸⁰, and C₂₋₆ alkyl optionally substituted with one or more substituent independently selected from R⁸⁰; and
C₃₋₆ carbocycle and 3- to 5-membered heterocycle, any of which is optionally substituted with one or more substituent independently selected from R⁸⁰.

In some embodiments, for the compound or salt of Formula (II), when R⁵¹ is fluoro, chloro, or iodo, R⁵² is selected from bromo, iodo, —N(R⁷¹)₂, —OR⁷¹, —SR⁷¹, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), N(R⁷¹)SO₂N(R⁷¹), —N(R⁷¹)P(O)(OR⁷¹)R⁷¹, —S(O)R⁷¹, —S(O)₂R⁷¹, —S(O)₂N(R⁷¹)₂, —NO₂, —CN, C₁ alkyl substituted with one or more substituent independently selected from R⁸⁰, and C₂₋₆ alkyl optionally substituted with one or more substituent independently selected from R⁸⁰. In some embodiments, when R⁵¹ is fluoro, chloro, or iodo, R⁵² is selected from bromo, iodo, —N(R⁷¹)₂, —OR⁷¹, —SR⁷¹, —C(O)R⁷¹, —C(O)OR⁷¹, —OC(O)R⁷¹, —C(O)N(R⁷¹)₂, —N(R⁷¹)C(O)R⁷¹, —NO₂, —CN, C₁ alkyl substituted with one or more substituent independently selected from R⁸⁰, and C₂₋₆ alkyl optionally substituted with one or more substituent independently selected from R⁸⁰. In some embodiments, when R⁵¹ is fluoro, chloro, or iodo, R⁵² is selected from bromo, iodo, —N(R⁷¹)₂, —OR⁷¹, —SR⁷¹, —C(O)R⁷¹, —C(O)OR⁷¹, —OC(O)R⁷¹, —C(O)N(R⁷¹)₂, —N(R⁷¹)C(O)R⁷¹, —NO₂, and —CN. In some embodiments, when R⁵¹ is fluoro, chloro, or iodo, R⁵² is selected from bromo, iodo, —N(R⁷¹)₂, —OR⁷¹, —NO₂, and —CN.

In some embodiments, for the compound or salt of Formula (II), when R⁵¹ is C₁ alkyl or —NO₂, R⁵² is selected from
fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, and —CN;
C₁₋₆ alkyl, optionally substituted with one or more substituent independently selected from R⁸⁰; and
C₃₋₆ carbocycle and 3- to 6-membered heterocycle, any of which is optionally substituted with one or more substituent independently selected from R⁸⁰.

In some embodiments, for the compound or salt of Formula (II), when R⁵¹ is C₁ alkyl or —NO₂, R⁵² is selected from: fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, —CN, and C₁₋₆ alkyl optionally substituted with one or more substituent independently selected from R⁸⁰. In some embodiments, when R⁵¹ is C₁ alkyl or —NO₂, R⁵² is selected from: fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —S(O)R⁷², —S(O)₂R⁷², —S(O)₂N(R⁷²)₂, —NO₂, —CN, and C₁₋₆ alkyl optionally substituted with one or more substituent independently selected from R⁸⁰. In some embodiments, when R⁵¹ is C₁ alkyl or —NO₂, R⁵² is selected from fluoro, iodo, —OR⁷², —SR⁷², —N(R⁷²)₂, —C(O)R⁷², —C(O)OR⁷², —OC(O)R⁷², —C(O)N(R⁷²)₂, —N(R⁷²)C(O)R⁷², —NO₂, —CN, and C₁₋₆ alkyl optionally substituted with one or more substituent independently selected from R⁸⁰. In some embodiments, when R⁵¹ is C₁ alkyl or —NO₂, R⁵² is selected from fluoro, iodo, —OR⁷², —SR⁷², —N(R⁷²)₂, —C(O)R⁷², —C(O)OR⁷², —OC(O)R⁷², —C(O)N(R⁷²)₂, —N(R⁷²)C(O)R⁷², —NO₂, —CN, and C₁₋₆ alkyl optionally substituted with one or more substituent independently selected from R⁸⁰. In some embodiments, when R⁵¹ is C₁ alkyl or —NO₂, R⁵² is selected from fluoro, iodo, —OR⁷², —N(R⁷²)₂, —C(O)R⁷², —C(O)OR⁷², C(O)N(R⁷²)₂, —NO₂, —CN, and C₁₋₆ alkyl optionally substituted with one or more substituent independently selected from R⁸⁰. In some embodiments, when R⁵¹ is C₁ alkyl or —NO₂, R⁵² is selected from fluoro, iodo, —OR⁷², —N(R⁷²)₂, —C(O)R⁷², —C(O)OR⁷², C(O)N(R⁷²)₂, —NO₂, —CN, and C₁₋₆ alkyl. In some embodiments, when R⁵¹ is C₁ alkyl or —NO₂, R⁵² is selected from fluoro, iodo, —OR⁷², —N(R⁷²)₂, NO₂, —CN, and C₁₋₆ alkyl optionally substituted with one or more substituent independently selected from R⁸⁰. In some embodiments, when R⁵¹ is C₁ alkyl or —NO₂, R⁵² is selected from fluoro, iodo, —OR⁷², —N(R⁷²)₂, NO₂, —CN, and C₁₋₆ alkyl. In some embodiments, when R⁵¹ is C₁ alkyl or —NO₂, R⁵² is selected from fluoro, and C₁₋₆ alkyl optionally substituted with one or more substituent independently selected from R⁸⁰. In some embodiments, when R⁵¹ is C₁ alkyl or —NO₂, R⁵² is selected from fluoro, and C₁₋₆ alkyl.

In some embodiments, for the compound or salt of Formula (II), when R⁵¹ is —NO₂, R⁵² is selected from:
fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, and —CN;
C₁₋₆ alkyl, optionally substituted with one or more substituent independently selected from R⁸⁰; and
C₃₋₆ carbocycle and 3- to 6-membered heterocycle, any of which is optionally substituted with one or more substituent independently selected from R⁸⁰.

In some embodiments, for the compound or salt of Formula (II), when R⁵¹ is —NO₂, R⁵² is selected from fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), N(R⁷²)SO₂N(R⁷²), —N(R⁷²)P(O)(OR⁷²)R⁷², —S(O)R⁷², —S(O)₂R⁷², —S(O)₂N(R⁷²)₂, —NO₂, —CN, and C₁₋₆ alkyl optionally substituted with one or more substituent independently selected from R⁸⁰. In some embodiments, when R⁵¹ is —NO₂, R⁵² is selected from fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —S(O)R⁷², —S(O)₂R⁷², —S(O)₂N(R⁷²)₂, —NO₂, —CN, and C₁₋₆ alkyl optionally substituted with one or more substituent independently selected from R⁸⁰. In some embodiments, when R⁵¹ is —NO₂, R⁵² is selected from fluoro, iodo, —OR⁷², —SR⁷², —N(R⁷²)₂, —C(O)R⁷², —C(O)OR⁷², —OC(O)R⁷², —C(O)N(R⁷²)₂, —N(R⁷²)C(O)R⁷², —NO₂, —CN, and C₁₋₆ alkyl optionally substituted with one or more substituent independently selected from R⁸⁰. In some embodiments, when R⁵¹ is —NO₂, R⁵² is selected from fluoro, iodo, —OR⁷², —SR⁷², —N(R⁷²)₂, —C(O)R⁷², —C(O)OR⁷², —OC(O)R⁷², —C(O)N(R⁷²)₂, —N(R⁷²)C(O)R⁷², —NO₂, —CN, and C₁₋₆ alkyl optionally substituted with one or more substituent independently selected from R⁸⁰. In some embodiments, when R⁵¹ is —NO₂, R⁵² is selected from fluoro, iodo, —OR⁷², —N(R⁷²)₂, —C(O)R⁷², —C(O)OR⁷², C(O)N(R⁷²)₂, —NO₂, —CN, and C₁₋₆ alkyl optionally substituted with one or more substituent independently selected from R⁸⁰. In some embodiments, when R⁵¹ is —NO₂, R⁵² is selected from fluoro, iodo, —OR⁷², —N(R⁷²)₂, NO₂, —CN, and C₁₋₆ alkyl optionally substituted with one or more substituent independently selected from R⁸⁰. In some embodiments, when R⁵¹ is —NO₂, R⁵² is selected from fluoro, and C₁₋₆ alkyl optionally substituted with one or more substituent independently selected from R⁸⁰. In some embodiments, when R⁵¹ is —NO₂, R⁵² is selected from fluoro and C₁₋₆ alkyl.

In some embodiments, for the compound or salt of Formula (II), when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN;
R⁵² is selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰.

In some embodiments, for the compound or salt of Formula (II), when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is selected from halogen, —OR⁷³, —N(R⁷³)₂, —C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is selected from halogen, —OR⁷³, —N(R⁷³)₂, —C(O)R⁷³, —NO₂, and —CN, and C₁₋₆ alkyl optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is selected from halogen, —OR⁷³, —N(R⁷³)₂, and C₁₋₆ alkyl optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is halogen.

In some embodiments, for the compound or salt of Formula (II), when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is selected from
halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN;
C₁₋₆ alkyl, optionally substituted with one or more substituent independently selected from R⁸⁰; and
C₃₋₆ carbocycle and 3- to 6-membered heterocycle, any of which is optionally substituted with one or more substituent independently selected from R⁸⁰.
   In some embodiments, for the compound or salt of Formula (II), when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is selected from halogen, —OR⁷³, —N(R⁷³)₂, —C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is selected from halogen, —OR⁷³, —N(R⁷³)₂, —C(O)R⁷³, —NO₂, and —CN, and C₁₋₆ alkyl optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is selected from halogen, —OR⁷³, —N(R⁷³)₂, and C₁₋₆ alkyl optionally substituted with one or more substituent independently selected from R⁸⁰. In some embodiments, when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, and —CN, R⁵² is halogen.

In some embodiments, for the compound or salt of Formula (II), when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is selected from
halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN; C₁₋₆ alkyl,
optionally substituted with one or more substituent independently selected from R⁸⁰; and C₃₋₆ carbocycle and 3- to 6-membered heterocycle, any of which is optionally substituted with one or more substituent independently selected from R⁸⁰.

In some embodiments, for the compound or salt of Formula (II), when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is selected from halogen, —OR⁷³, —N(R⁷³)₂, —C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is selected from halogen, —OR⁷³, —N(R⁷³)₂, —C(O)R⁷³, —NO₂, and —CN, and C₁₋₆ alkyl optionally substituted with one or more substituent independently selected from R⁸⁰. In some embodiments, when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is selected from halogen, —OR⁷³, —N(R⁷³)₂, and C₁₋₆ alkyl optionally substituted with one or more substituent independently selected from R⁸⁰. In some embodiments, when R⁵¹ is selected from bromo, hydroxyl, —C₂₋₆ alkyl, —N(R⁶¹)₂, —OR⁶¹, —SR⁶¹, —C(O)R⁶¹, —C(O)OR⁶¹, —C(O)N(R⁶¹)₂, and —CN, R⁵² is halogen.

In some embodiments, for the compound or salt of Formula (II), when R⁵¹ is selected from —C(O)R⁶¹, —C(O)OR⁶¹, and —C(O)N(R⁶¹)₂, R⁵² is selected from
halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), N(R⁷³)SO₂N(R⁷³), —N(R⁷³)P(O)(OR⁷³)R⁷³, —S(O)R⁷³, —S(O)₂R⁷³, —S(O)₂N(R⁷³)₂, —NO₂, and —CN;
C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰.

In some embodiments, for the compound or salt of Formula (II), when R⁵¹ is selected from —C(O)R⁶¹, —C(O)OR⁶¹, and —C(O)N(R⁶¹)₂, R⁵² is selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from —C(O)R⁶¹, —C(O)OR⁶¹, and —C(O)N(R⁶¹)₂, R⁵² is selected from halogen, —OR⁷³, —N(R⁷³)₂, —C(O)R⁷³, —NO₂, and —CN; C₁₋₆ alkyl, optionally substituted with one or more substituent independently selected from R⁸⁰; and C₃₋₆ carbocycle and 3- to 6-membered heterocycle, any of which is optionally substituted with one or more substituent independently selected from R⁸⁰. In some embodiments, when R⁵¹ is selected from —C(O)R⁶¹, —C(O)OR⁶¹, and —C(O)N(R⁶¹)₂, R⁵² is selected from halogen, —OR⁷³, —N(R⁷³)₂, —C(O)R⁷³, —NO₂, and —CN, and C₁₋₆ alkyl optionally substituted with one or more substituent independently selected from R⁸⁰. In some embodiments, when R⁵¹ is selected from —C(O)R⁶¹, —C(O)OR⁶¹, and —C(O)N(R⁶¹)₂, R⁵² is selected from halogen, —OR⁷³, —N(R⁷³)₂, and C₁₋₆ alkyl optionally substituted with one or more substituent independently selected from R⁸⁰. In some embodiments, when R⁵¹ is selected from —C(O)R⁶¹, —C(O)OR⁶¹, and —C(O)N(R⁶¹)₂, R⁵² is selected from halogen, —OR⁷³, —N(R⁷³)₂, and C₁₋₆ alkyl. In some embodiments, when R⁵¹ is selected from —C(O)R⁶¹, —C(O)OR⁶¹, and —C(O)N(R⁶¹)₂, R⁵² is halogen.

In some embodiments, for the compound or salt of Formula (II), R⁵³ and R⁵⁴ are each independently selected at each occurrence from
halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN; and
and C₃₋₄ carbocycle and 3- to 4-membered heterocycle, any of which is optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN.

In some embodiments, for the compound or salt of Formula (II), R⁵³ and R⁵⁴ are each independently selected at each occurrence from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, —CN, and C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN. In some embodiments, R⁵³ and R⁵⁴ are each independently selected at each occurrence from halogen, —OR⁶⁴, —SR⁶⁴, —N(R⁶⁴)₂, —C(O)R⁶⁴, —NO₂, —CN, and C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN. In some embodiments, R⁵³ and R⁵⁴ are each independently selected at each occurrence from halogen, —OR⁶⁴, —SR⁶⁴, —N(R⁶⁴)₂, —C(O)R⁶⁴, —NO₂, and —CN. In some embodiments, R⁵³ and R⁵⁴ are each independently selected at each occurrence from halogen, —OR⁶⁴, —N(R⁶⁴)₂, —NO₂, and —CN. In some embodiments, R⁵³ and R⁵⁴ are each independently selected at each occurrence from a halogen.

In some embodiments, for the compound or salt of Formula (II), R⁵⁵ and R^{55′} are each independently selected from hydrogen and hydroxyl; or R⁵⁵ and R^{55′} taken together are ═O. In some embodiments, R⁵⁵ and R^{55′} taken together are ═O.

In some embodiments, for the compound or salt of Formula (II), R⁵⁶ and R^{56′} are each independently selected from hydrogen and hydroxyl; or R⁵⁶ and R^{56′} taken together are ═O, N—OR⁶⁴, or ═NR⁶⁴. In some embodiments, R⁵⁶ and R^{56′} are each independently selected from hydrogen and hydroxyl. In some embodiments, R⁵⁶ and R^{56′} taken together are ═O. In some embodiments, R⁵⁶ and R^{56′} taken together are ═N—OR⁶⁴.

In some embodiments, for the compound or salt of Formula (II), p is selected from 0, 1, 2, and 3. In some embodiments, p is selected from 0, and 1.

In some embodiments, for the compound or salt of Formula (II), R⁶¹ and R⁶⁴ are each independently selected at each occurrence from
hydrogen;
C₁₋₆ alkyl optionally substituted with one or more substituent independently selected from R⁸¹; and
C₃₋₆ carbocycle and 3- to 6-membered heterocycle, any of which is optionally substituted with one or more substituent independently selected from R⁸¹.

In some embodiments, for the compound or salt of Formula (II), R⁶¹ and R⁶⁴ are each independently selected at each occurrence from
hydrogen;
C₁₋₆ alkyl optionally substituted with one or more substituent independently selected from R⁸¹; and
3- to 6-membered heterocycle, optionally substituted with one or more substituent independently selected from R⁸¹.

In some embodiments, for the compound or salt of Formula (II), R⁶¹ and R⁶⁴ are each independently selected at each occurrence from
hydrogen;
C₁₋₆ alkyl; and
3- to 6-membered heterocycle, optionally substituted with one or more substituent independently selected from R⁸¹.

In some embodiments, for the compound or salt of Formula (II), R⁷¹, R⁷², and R⁷³ are each independently selected at each occurrence from
hydrogen;
C₁₋₆ alkyl optionally substituted with one or more substituents independently selected from R⁸²; and
C₃₋₆ carbocycle and 3- to 6-membered heterocycle, any of which is optionally substituted with one or more substituent independently selected from C₁₋₄ alkyl, C₁₋₄ haloalkyl, and R⁸².

In some embodiments, R⁷¹, R⁷², and R⁷³ are each independently selected at each occurrence from hydrogen and C₁₋₆ alkyl optionally substituted with one or more substituents independently selected from R⁸². In some embodiments, R⁷¹, R⁷², and R⁷³ are each independently selected at each occurrence from C₁₋₆ alkyl optionally substituted with one or more substituents independently selected from R⁸². In some embodiments, R⁷¹, R⁷², and R⁷³ are each independently selected at each occurrence from hydrogen. In some embodiments, R⁷¹, R⁷², and R⁷³ are each independently selected at each occurrence from C₃₋₆ carbocycle and 3- to 6-membered heterocycle, any of which is optionally substituted with one or more substituent independently selected from C₁₋₄ alkyl, C₁₋₄ haloalkyl, and R⁸. In some embodiments, R⁷¹, R⁷², and R⁷³ are each independently selected at each occurrence from C₃₋₆ carbocycle and 3- to 6-membered heterocycle, any of which is optionally substituted with one or more substituent independently selected from C₁₋₄ alkyl, and R⁸. In some embodiments, R⁷¹, R⁷², and R⁷³ are each independently selected at each occurrence from C₃₋₆ carbocycle and 3- to 6-membered heterocycle.

In some embodiments, for the compound or salt of Formula (II), R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)C(O)OR⁹¹, —N(R⁹¹)C(O)N(R⁹¹)₂, —N(R⁹¹)S(O)₂(R⁹¹), —N(R⁹¹)SO₂N(R⁹¹), —N(R⁹¹)P(O)(OR⁹¹)R⁹¹, —S(O)R⁹¹, —S(O)₂R⁹¹, —S(O)₂N(R⁹¹)₂, —NO₂, ═O, ═S, —CN, C₃₋₆ carbocycle, and 3- to 7-membered heterocycle. In some embodiments, R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)S(O)₂(R⁹¹), —N(R⁹¹)SO₂N(R⁹¹), —S(O)R⁹¹, —S(O)₂R⁹¹, —S(O)₂N(R⁹¹)₂, —NO₂, ═O, ═S, —CN, C₃₋₆ carbocycle, and 3- to 7-membered heterocycle. In some embodiments, R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)SO₂N(R⁹¹), —NO₂, ═O, —CN, C₃₋₆ carbocycle, and 3- to 7-membered heterocycle. In some embodiments, R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —N(R⁹¹)SO₂N(R⁹¹), —NO₂, ═O, —CN, C₃₋₆ carbocycle, and 3- to 7-membered heterocycle. In some embodiments, R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —N(R⁹¹)SO₂N(R⁹¹)—NO₂, ═O, and —CN. In some embodiments, R⁸⁰, R⁸¹, and R⁸² are each independently selected at each occurrence from halogen, —OR⁹¹, —N(R⁹¹)₂, —C(O)OR⁹¹, —N(R⁹¹)SO₂N(R⁹¹), —NO₂, ═O, and —CN. In some embodiments, R⁸⁰, R⁸¹, and R⁸² are each independently selected at each occurrence from —N(R⁹¹)₂, —C(O)OR⁹¹, and —N(R⁹¹)SO₂N(R⁹¹)

In some embodiments, for the compound or salt of Formula (II), R⁹¹ is independently selected at each occurrence from hydrogen, C₁₋₆ alkyl, C₁₋₆ haloalkyl, and C₁₋₆ hydroxyalkyl. In some embodiments, R⁹¹ is independently selected at each occurrence from hydrogen, C₁₋₆ alkyl, and C₁₋₆ haloalkyl. In some embodiments, R⁹¹ is independently selected at each occurrence from hydrogen, C₁₋₆ alkyl, and C₁₋₆ hydroxyalkyl. In some embodiments, R⁹¹ is independently selected at each occurrence from hydrogen and C₁₋₆ alkyl.

In one aspect, provided herein is a compound having the structure of Formula (III):

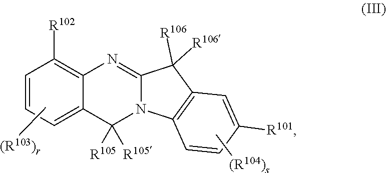

or a pharmaceutically acceptable salt thereof; wherein
R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —SR¹¹¹, —C(O)R¹¹¹, —C(O)OR¹¹¹, —OC(O)R¹¹¹, —OC(O)N(R¹¹¹)₂—C(O)N(R¹¹¹)₂, —N(R¹¹¹)C(O)R¹¹, —N(R¹¹¹)C(O)OR¹¹¹, —N(R¹¹¹)C(O)N(R¹¹)₂, —N(R¹¹¹)S(O)₂(R¹¹¹), —S(O)₂R¹¹¹, —S(O)₂N(R¹¹¹)₂, —NO₂, and —CN;
when R¹⁰¹ is fluoro, R¹⁰² is selected from
   chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R²¹)C(O)OR¹²¹, —N(R¹²¹)C(O)N(R¹²¹)₂, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN;
   C₁ alkyl substituted with one or more substituent independently selected from R¹²³, and C₂₋₆ alkyl optionally substituted with one or more substituent independently selected from R¹³⁰; and
   C₃₋₆ carbocycle and 3- to 6-membered heterocycle, any of which is optionally substituted with one or more substituent independently selected from R¹³⁰;
when R¹⁰¹ is bromo, iodo, hydroxyl, —O—C₁ alkyl, —C(O)OR¹¹, —C(O)N(H)(C₂ alkyl), —S(O)₂R¹¹, or —S(O)₂N(R¹¹¹)₂, R¹⁰² is selected from
   fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and
   C₃₋₆ carbocycle and 3- to 6-membered heterocycle, any of which is optionally substituted with one or more substituent independently selected from R¹³⁰;
when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹, —C(O)N(R¹¹³)₂, —OC(O)N(R¹¹¹)₂, —N(R¹¹¹)C(O)R¹¹¹, —N(R¹¹¹)C(O)OR¹¹¹, —N(R¹¹¹)C(O)N(R¹¹¹)₂, —N(R¹¹¹)S(O)₂(R¹¹), —NO₂, or —CN; R¹⁰² is selected from
   halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
   C₁₋₆ alkyl, optionally substituted with one or more substituent independently selected from R¹³⁰; and
   C₃₋₆ carbocycle and 3- to 6-membered heterocycle, any of which is optionally substituted with one or more substituent independently selected from R¹³⁰;
   provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;
R¹⁰³ and R¹⁰⁴ are each independently selected at each occurrence from
   halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
   C₁₋₄ alkyl, optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
   C₃₋₄ carbocycle and 3- to 4-membered heterocycle, any of which is optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
R¹⁰⁵ and R^{105′} are each independently selected from hydrogen and hydroxyl; or R¹⁰⁵ and R^{105′} taken together are ═O;
R¹⁰⁶ and R^{106′} are each independently selected from hydrogen and hydroxyl; or R¹⁰⁶ and R^{106′} taken together are ═O, ═N—OR¹¹⁴, or ═NR¹¹⁴;
r is selected from 0, 1, 2, and 3;
s is selected from 0, 1, 2, and 3;
R¹¹¹ and R¹¹⁴ are each independently selected at each occurrence from
   hydrogen;
   C₁₋₆ alkyl optionally substituted with one or more substituent independently selected from R¹³¹;
   C₃₋₆ carbocycle and 3- to 6-membered heterocycle, any of which is optionally substituted with one or more substituent independently selected from R¹³¹;
R¹¹² is selected at each occurrence from
   C₁₋₆ alkyl substituted with one or more substituent independently selected from R¹³²;
   C₃₋₆ carbocycle and 3- to 6-membered heterocycle, any of which is optionally substituted with one or more substituent independently selected from R¹³³;
R¹¹³ is selected at each occurrence from
   hydrogen;
   C₁ alkyl and C₃₋₆ alkyl optionally substituted with one or more substituent independently selected from R¹³⁴, and C₂ alkyl substituted with one or more substituent independently selected from R¹³⁴;
   C₃₋₆ carbocycle and 3- to 6-membered heterocycle, any of which is optionally substituted with one or more substituent independently selected from R¹³⁴;
R¹²¹, R¹²⁴, and R¹²⁶ are each independently selected at each occurrence from
   hydrogen;
   C₁₋₆ alkyl optionally substituted with one or more substituents independently selected from R¹³⁴;
   C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²² and R¹²⁵ are each independently selected at each occurrence from
   C₂₋₆ alkyl optionally substituted with one or more substituents independently selected from R¹³⁴;
   C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²³ is independently selected at each occurrence from
   hydrogen;
   C₁₋₆ alkyl optionally substituted with one or more substituents independently selected from R¹³⁴;
   C₃₋₅ carbocycle and 3- to 6-membered heterocycle, any of which is optionally substituted with one or more substituent independently selected from C₁₋₄ alkyl, C₁₋₄ haloalkyl, and R¹³⁴;
R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —OC(O)N(R¹⁴¹)₂, —C(O)N(R¹⁴¹)₂, —N(R¹⁴¹)C(O)R¹⁴¹, —N(R¹⁴¹)C(O)OR¹⁴¹, —N(R¹⁴¹)C(O)N(R¹⁴¹)₂, —N(R¹⁴¹)S(O)₂(R¹⁴¹), —N(R¹⁴¹)SO₂N(R¹⁴¹), —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, —CN, C₃₋₆ carbocycle, and 3- to 7-membered heterocycle; and
R¹⁴¹ is independently selected at each occurrence from hydrogen, C₁₋₆ alkyl, C₁₋₆ haloalkyl, and C₁₋₆ hydroxyalkyl.

In some embodiments, for the compound or salt of (III), R¹⁰¹ is fluoro. In some embodiments, for the compound or salt of (III), R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂. In some embodiments, for the compound or salt of (III), R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹, —N(R¹¹¹)₂, —C(O)R¹, —OC(O)R¹, —C(O)N(R¹¹³)₂, —OC(O)N(R¹¹¹)₂, —N(R¹¹¹)C(O)R¹¹, —N(R¹¹¹)C(O)OR¹¹, —N(R¹¹¹)C(O)N(R¹¹¹)₂, —N(R¹¹¹)S(O)₂(R¹¹¹), —NO₂, or —CN.

In some embodiments, for the compound or salt of (III), R¹⁰¹ is fluoro, bromo, iodo, hydroxyl, —O—C₁ alkyl, —C(O)OR¹¹¹, —C(O)N(H)(C₂ alkyl), —S(O)₂R¹¹, or —S(O)₂N(R¹¹¹)₂. In some embodiments, R¹⁰¹ is fluoro, bromo, iodo, hydroxyl, —O—C₁ alkyl, —C(O)OR¹¹, or —C(O)N(H)(C₂ alkyl). In some embodiments, R¹⁰¹ is fluoro, bromo, iodo, hydroxyl, or —O—C₁ alkyl.

In some embodiments, for the compound or salt of (III), R¹⁰¹ is fluoro, chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹¹, —C(O)N(R¹¹³)₂, —OC(O)N(R¹¹¹)₂, —N(R¹¹¹)C(O)R¹¹¹, —N(R¹¹¹)C(O)OR¹¹, —N(R¹¹¹)C(O)N(R¹¹¹)₂, —N(R¹¹¹)S(O)₂(R¹¹¹), —NO₂, or —CN. In some embodiments, R¹⁰¹ is fluoro, chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹¹¹, —C(O)N(R¹¹³)₂, —OC(O)N(R^{1I})₂, —N(R¹¹¹)C(O)R¹¹¹, —NO₂, or —CN. In some embodiments, R¹⁰¹ is fluoro, chloro, —C₁₋₆ alkyl, —C₁₋₆ haloalkyl, —O—C₂₋₆ alkyl, —OR¹¹², —SR¹¹¹, —N(R¹¹¹)₂, —C(O)R¹¹¹, —NO₂, or —CN. In some embodiments, R¹⁰¹ is fluoro, —NO₂, or —CN.

In some embodiments, for the compound or salt of (III), R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, chloro, —C₁₋₆ alkyl, —C₁₋₆ haloalkyl, —O—C₂₋₆ alkyl, —O—C₁₋₆ haloalkyl, —OR¹¹², —SR¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN. In some embodiments, R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹¹¹, —C(O)N(R¹¹³)₂, —N(R¹¹¹)C(O)R¹¹¹, —NO₂, or —CN. In some embodiments, R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, chloro, —C₁₋₆ alkyl, —C₁₋₆ haloalkyl, —O—C₂₋₆ alkyl, —OR¹¹², —SR¹¹, —N(R¹¹¹)₂, —C(O)R¹¹¹, —OC(O)R¹¹¹, —C(O)N(R¹¹³)₂, —N(R¹¹¹)C(O)R¹¹¹, —NO₂, or —CN. In some embodiments, R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, chloro, —C₁₋₆ alkyl, —C₁₋₆ haloalkyl, —O—C₂₋₆ alkyl, —OR¹¹², —SR¹¹¹, —N(R¹¹¹)₂, —NO₂, or —CN. In some embodiments, R¹⁰¹ is bromo, iodo, hydroxyl, —O—C₁ alkyl, —C(O)OR¹¹¹, —C(O)N(H)(C₂ alkyl), —S(O)₂R¹¹¹ or —S(O)₂N(R¹¹¹)₂, —NO₂, or —CN. In some embodiments, R¹⁰¹ is bromo, iodo, hydroxyl, —O—C₁ alkyl, —NO₂, or —CN.

In some embodiments, for the compound or salt of (III), R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —SR¹¹¹, —C(O)R¹¹¹, —C(O)OR¹¹¹, —OC(O)R¹¹¹, —OC(O)N(R¹¹¹)₂, —C(O)N(R¹¹¹)₂, —N(R¹¹¹)C(O)R¹¹, —N(R¹¹¹)C(O)OR¹¹, —N(R¹¹¹)C(O)N(R¹¹)₂, —N(R¹¹¹)S(O)₂(R¹¹¹), —S(O)₂R¹¹¹, —S(O)₂N(R¹¹¹)₂, —NO₂, and —CN. In some embodiments, R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹)₂, —OR¹¹², —SR¹¹¹, —C(O)R¹¹¹, —C(O)OR¹¹¹, —OC(O)R¹¹¹, —C(O)N(R¹¹¹)₂, —N(R¹¹¹)C(O)R¹¹¹, N(R¹¹¹)S(O)₂(R¹¹¹), —S(O)₂R¹¹¹, —S(O)₂N(R¹¹¹)₂, —NO₂, and —CN. In some embodiments, R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —SR¹¹¹, —C(O)R¹¹¹, —C(O)OR¹¹¹, —OC(O)R¹¹¹, —C(O)N(R¹¹¹)₂, —N(R¹¹¹)C(O)R¹¹¹, N(R¹¹¹)S(O)₂(R¹¹¹), —S(O)₂R¹¹¹, —S(O)₂N(R¹¹¹)₂, —NO₂, and —CN. In some embodiments, R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —C(O)R¹¹¹, —C(O)OR¹¹¹, —OC(O)R¹¹¹, —C(O)N(R¹¹¹)₂, —N(R¹¹¹)C(O)R¹¹¹, —S(O)₂R¹¹¹, —NO₂, and —CN. In some embodiments, R¹⁰¹ is selected from fluoro, chloro, bromo, hydroxyl, —C₁₋₆ alkyl, —C₁₋₆ haloalkyl, —O—C₁₋₆ alkyl, —N(R¹¹¹)₂, —OR¹¹², —C(O)R¹¹¹, —C(O)OR¹¹¹, —OC(O)R¹¹¹, —C(O)N(R¹¹¹)₂, —N(R¹¹¹)C(O)R¹¹¹, —NO₂, and —CN. In some embodiments, R¹⁰¹ is selected from fluoro, chloro, bromo, hydroxyl, —C₁₋₆ alkyl, —C₁₋₆ haloalkyl, —O—C₁₋₆ alkyl, —N(R¹¹¹)₂, —OR¹¹², —C(O)R¹¹, —C(O)OR¹¹, —NO₂, and —CN. In some embodiments, R¹⁰¹ is selected from fluoro, chloro, bromo, hydroxyl, —C₁₋₆ alkyl, —O—C₁₋₆ alkyl, —N(R¹¹¹)₂, —OR¹¹², —NO₂, and —CN. In some embodiments, R¹⁰¹ is selected from fluoro, —NO₂, and —CN.

In some embodiments, for the compound or salt of formula (III), when R¹⁰¹ is fluoro, R¹⁰² is selected from
chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R¹²¹)C(O)OR¹²¹, —N(R¹²¹)C(O)N(R¹²¹)₂, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN;
C₁ alkyl substituted with one or more substituent independently selected from R¹²³, and C₂₋₆ alkyl optionally substituted with one or more substituent independently selected from R¹³⁰; and
C₃₋₆ carbocycle and 3- to 6-membered heterocycle, any of which is optionally substituted with one or more substituent independently selected from R¹³⁰.

In some embodiments, for the compound or salt of formula (III), when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R²¹)C(O)OR¹²¹, —N(R¹²¹)C(O)N(R¹²¹)₂, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R¹²¹)C(O)OR¹²¹, —N(R¹²¹)C(O)N(R¹²¹)₂, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —C(O)N(R¹²¹)₂, —N(R¹²¹)C(O)R¹²¹, —N(R¹²¹)S(O)₂(R¹²¹), N(R¹²¹)SO₂N(R¹²¹), —N(R¹²¹)P(O)(OR¹²¹)R¹²¹, —S(O)R¹²¹, —S(O)₂R¹²¹, —S(O)₂N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —C(O)N(R¹²¹)₂, —N(R¹²¹)C(O)R¹²¹, —S(O)R¹²¹, —S(O)₂R¹²¹, —S(O)₂N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —C(O)N(R¹²¹)₂, —N(R¹²¹)C(O)R¹²¹, —NO₂, and —CN. In some embodiments, when R¹⁰¹ is fluoro, R¹⁰² is selected from chloro, bromo, iodo, —N(R¹²¹)₂, —OR¹²², —SR¹²¹, —C(O)R¹²¹, —C(O)OR¹²¹, —OC(O)R¹²¹, —C(O)N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —C(O)R¹²¹, —C(O)OR¹²¹, —OC(O)R¹²¹, —C(O)N(R¹²¹)₂, —NO₂, and —CN. In some embodiments, when R¹⁰¹ is fluoro, R¹⁰² is selected from chloro, bromo, iodo, —N(R¹²¹)₂, —OR¹²², —C(O)R¹²¹, —NO₂, and —CN. In some embodiments, when R¹⁰¹ is fluoro, R¹⁰² is selected from chloro, bromo, iodo, —N(R¹²¹)₂, —OR¹²², —NO₂, and —CN. In some embodiments, when R¹⁰¹ is fluoro, R¹⁰² is selected from chloro, bromo, iodo, —NO₂, and —CN. In some embodiments, when R¹⁰¹ is fluoro, R¹⁰² is selected from chloro, bromo, and iodo. In some embodiments, when R¹⁰¹ is fluoro, R¹⁰² is bromo.

In some embodiments, for the compound or salt of formula (III), when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from
fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰.

In some embodiments, for the compound or salt of formula (III), when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3-to 6-membered heterocycle, any of which is optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴) N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰.

In some embodiments, for the compound or salt of formula (III), when R¹⁰¹ is bromo, iodo, hydroxyl, —O—C₁ alkyl, —C(O)OR¹¹, —C(O)N(H)(C₂ alkyl), —S(O)₂R¹¹, or —S(O)₂N(R¹¹¹)₂, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —N(R¹²⁴)S(O)₂(R¹²⁴), N(R¹²⁴)SO₂N(R¹²⁴), —N(R¹²⁴)P(O)(OR¹²⁴)R¹²⁴, S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —C(O)R¹²⁴, —C(O)OR¹²⁴, —OC(O)R¹²⁴, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —N(R¹²⁴)₂, —OR¹²⁵, —NO₂, —CN, C₁₋₆ alkyl. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —NO₂, —CN, C₁₋₆ alkyl. In some embodiments, when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from fluoro, —NO₂, and —CN.

In some embodiments, for the compound or salt of formula (III), when R¹⁰¹ is bromo, iodo, hydroxyl, —O—C₁ alkyl, —C(O)OR¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰.

In some embodiments, for the compound or salt of formula (III), when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰.

In some embodiments, for the compound or salt of formula (III), when R¹⁰¹ is bromo, iodo, hydroxyl, —O—C₁ alkyl, —C(O)OR¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —C(O)R¹²⁴, —C(O)OR¹²⁴, —OC(O)R¹²⁴, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —NO₂, —CN, C₁₋₆ alkyl optionally substituted with one or more substituent independently selected from R¹³⁰. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —N(R¹²⁴)₂, —OR¹²⁵, —NO₂, —CN, C₁₋₆ alkyl. In some embodiments, when R¹⁰¹ is bromo, iodo, hydroxyl, —O—C₁ alkyl, —C(O)OR¹¹¹, or —C(O)N(H)(C₂ alkyl), R¹⁰² is selected from fluoro, —NO₂, —CN, C₁₋₆ alkyl. In some embodiments, when R¹⁰¹ is bromo, iodo, hydroxyl, —O—C₁ alkyl, —C(O)OR¹¹, or —C(O)N(H)(C₂ alkyl), R¹⁰² is selected from fluoro, —NO₂, and —CN.

In some embodiments, for the compound or salt of formula (III), when R¹⁰¹ is bromo, iodo, or hydroxyl, R¹⁰² is selected from
fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰.

In some embodiments, for the compound or salt of formula (III), when R¹⁰¹ is bromo, iodo, or hydroxyl, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, S(O)R¹²⁴, —S(O)₂R¹²⁴, —S(O)₂N(R¹²⁴)₂, —NO₂, and —CN; C₁₋₆ alkyl, optionally substituted with one or more substituent independently selected from R¹³⁰; and C₃₋₆ carbocycle and 3- to 6-membered heterocycle, any of which is optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), N(R¹²⁴)SO₂N(R¹²⁴), —N(R¹²⁴)P(O)(OR¹²⁴)R¹²⁴, S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —N(R¹²⁴)S(O)₂(R¹²⁴), N(R¹²⁴)SO₂N(R¹²⁴), —N(R¹²⁴)P(O)(OR¹²⁴)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —SR¹²⁴, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —S(O)₂R¹²⁴, —S(O)₂N(R¹²⁴)₂, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —C(O)R¹²⁴, —C(O)OR¹²⁴, —OC(O)R¹²⁴, —C(O)N(R¹²⁴)₂, —N(R¹²⁴)C(O)R¹²⁴, —S(O)R¹²⁴, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —C(O)R¹²⁴, —C(O)OR¹²⁴, —OC(O)R¹²⁴, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —NO₂, —CN, C₁₋₆ alkyl optionally substituted with one or more substituent independently selected from R¹³⁰. In some embodiments, when R¹⁰¹ is bromo, iodo, or hydroxyl, R¹⁰² is selected from fluoro, —N(R¹²⁴)₂, —OR¹²⁵, —NO₂, —CN, C₁₋₆ alkyl. In some embodiments, when R¹⁰¹ is bromo, iodo, or hydroxyl, R¹⁰² is selected from fluoro, —NO₂, —CN, C₁₋₆ alkyl. In some embodiments, when R¹⁰¹ is bromo, iodo, or hydroxyl, R¹⁰² is selected from fluoro, —NO₂, and —CN.

In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹²—SR¹¹, —N(R¹¹)₂—C(O)R¹¹, —OC(O)R¹, —C(O)N(R¹¹³)₂, —OC(O)N(R¹¹¹)₂, —N(R¹¹¹)C(O)R¹¹¹, —N(R¹¹¹)C(O)OR¹, —N(R¹¹¹)C(O)N(R¹¹¹)₂, —N(R¹¹¹)S(O)₂(R¹¹¹), —NO₂, or —CN; R¹⁰² is selected from
halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰;
provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;

In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹²—SR¹¹, —N(R¹¹)₂—C(O)R¹¹, —OC(O)R¹¹¹, —C(O)N(R¹¹³)₂, —OC(O)N(R¹¹¹)₂, —N(R¹¹¹)C(O)R¹¹¹, —N(R¹¹¹)C(O)OR¹, —N(R¹¹¹)C(O)N(R¹¹¹)₂, —N(R¹¹¹)S(O)₂(R¹¹¹), —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹²—SR¹¹¹, —N(R¹¹¹)₂—C(O)R¹¹¹, —OC(O)R¹¹, —C(O)N(R¹¹³)₂, —OC(O)N(R¹¹¹)₂, —N(R¹¹¹)C(O)R¹¹¹, —N(R¹¹¹)C(O)OR¹, —N(R¹¹¹)C(O)N(R¹¹¹)₂, —N(R¹¹¹)S(O)₂(R¹¹¹), —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —N(R¹²⁶)S(O)₂(R¹²⁶), N(R¹²⁶)SO₂N(R¹²⁶), —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹, —N(R¹¹¹)₂, —C(O)R¹, —OC(O)R¹, —C(O)N(R¹¹³)₂, —OC(O)N(R¹¹¹)₂, —N(R¹¹¹)C(O)R¹¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN; R¹⁰² is selected from halogen, —OR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN; R¹⁰² is selected from halogen, —OR¹²⁶, —N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN; R¹⁰² is selected from halogen and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹, —N(R¹¹¹)C(O)OR¹¹, —N(R¹¹¹)C(O)N(R¹¹¹)₂, —N(R¹¹¹)S(O)₂(R¹¹¹), —NO₂, or —CN; R¹⁰² is selected from halogen and C₁₋₆ alkyl; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo.

In some embodiments, for the compound or salt of formula (III), when R¹⁰¹ is chloro, —C₁₋₆ alkyl, —C₁₋₆ haloalkyl, —O—C₂₋₆ alkyl, —OR¹¹², —SR¹¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from
halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰;
provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;

In some embodiments, for the compound or salt of formula (III), when R¹⁰¹ is chloro, —C₁₋₆ alkyl, —C₁₋₆ haloalkyl, —O—C₂₋₆ alkyl, —OR¹¹², —SR¹¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —OR¹¹²—SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —N(R¹²⁶)S(O)₂(R¹²⁶), N(R¹²⁶)SO₂N(R¹²⁶), —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —OR¹¹²—SR¹¹¹, —N(R¹¹¹)₂, —C(O)R¹¹, —OC(O)R¹¹, —C(O)N(R¹¹³)₂, —OC(O)N(R¹¹¹)₂, —N(R¹¹¹)C(O)R¹¹¹, —NO₂, or —CN; R¹⁰² is selected from halogen, —OR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —OR¹¹², —SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from halogen, —OR¹²⁶, —N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —OR¹¹², —SR¹¹¹, —N(R¹¹¹)₂—C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from halogen and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —C₁₋₆ haloalkyl, —O—C₂₋₆ alkyl, —OR¹¹², —SR¹¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —NO₂, or —CN; R¹⁰² is selected from halogen and C₁₋₆ alkyl; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo.

In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from
halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰;
provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;

In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), N(R¹²⁶)SO₂N(R¹²⁶), —N(R¹²⁶)P(O)(OR¹²⁶)R¹²⁶, —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from halogen, —OR¹²⁶, —SR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —N(R¹²⁶)S(O)₂(R¹²⁶), N(R¹²⁶)SO₂N(R¹²⁶), —S(O)R¹²⁶, —S(O)₂R¹²⁶, —S(O)₂N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from halogen, —OR¹²⁶, —N(R¹²⁶)₂, —C(O)R¹²⁶, —C(O)OR¹²⁶, —OC(O)R¹²⁶, —C(O)N(R¹²⁶)₂, —N(R¹²⁶)C(O)R¹²⁶, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from halogen, —OR¹²⁶, —N(R¹²⁶)₂, —NO₂, —CN, and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —O—C₂₋₆ alkyl, —OR¹², —N(R¹¹¹)₂, —NO₂, or —CN; R¹⁰² is selected from halogen and C₁₋₆ alkyl, optionally substituted with one or more substituent independently selected from R¹³⁰; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo. In some embodiments, for the compound or salt of formula (III), when R¹⁰¹ is chloro, —C₁₋₆ alkyl, —O—C₂₋₆ alkyl, —OR¹¹², —N(R¹¹¹)₂, —NO₂, or —CN; R¹⁰² is selected from halogen and C₁₋₆ alkyl; provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo.

In some embodiments, for the compound or salt of formula (III), R¹⁰³ and R¹⁰⁴ are each independently selected at each occurrence from
halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
C₁₋₄ alkyl, optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN; and
C₃₋₄ carbocycle and 3- to 4-membered heterocycle, any of which is optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN.

In some embodiments, for the compound or salt of formula (III), R¹⁰³ and R¹⁰⁴ are each independently selected at each occurrence from
halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
C₁₋₄ alkyl, optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN; and
C₃₋₄ carbocycle and 3- to 4-membered heterocycle.

In some embodiments, for the compound or salt of formula (III), R¹⁰³ and R¹⁰⁴ are each independently selected at each occurrence from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, —CN, and C₁₋₄ alkyl optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN. In some embodiments, R¹⁰³ and R¹⁰⁴ are each independently selected at each occurrence from halogen, —OR¹¹⁴, —SR¹¹⁴, —N(R¹¹⁴)₂, —C(O)R¹¹⁴, —NO₂, —CN, and C₁₋₄ alkyl optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN. In some embodiments, R¹⁰³ and R¹⁰⁴ are each independently selected at each occurrence from halogen, —OR¹¹⁴, —N(R¹¹⁴)₂, —NO₂, —CN, and C₁₋₄ alkyl optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN. In some embodiments, R¹⁰³ and R¹⁰⁴ are each independently selected at each occurrence from halogen, and C₁₋₄ alkyl optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN. In some embodiments, R¹⁰³ and R¹⁰⁴ are each independently selected at each occurrence from halogen, and C₁₋₄ alkyl optionally substituted with one or more substituents independently selected from halogen, —OR¹¹⁴, —SR¹¹⁴, —N(R¹¹⁴)₂, —NO₂, and —CN. In some embodiments, R¹⁰³ and R¹⁰⁴ are each independently selected at each occurrence from halogen, and C₁₋₄ alkyl optionally substituted with one or more substituents independently selected from halogen, —OR¹¹⁴, —N(R¹¹⁴)₂, —NO₂, and —CN. In some embodiments, R¹⁰³ and R¹⁰⁴ are each independently selected at each occurrence from halogen, and C₁₋₄ alkyl.

In some embodiments, for the compound or salt of formula (III), R¹⁰⁵ and R^{105′} are each independently selected from hydrogen and hydroxyl; or R¹⁰⁵ and R^{105′} taken together are ═O. In some embodiments, for the compound or salt of formula (III), R¹⁰⁵ and R^{105′} are each independently selected from hydrogen and hydroxyl In some embodiments, R¹⁰⁵ and R^{105′} taken together are ═O.

In some embodiments, for the compound or salt of formula (III), R¹⁰⁶ and R^{106′} are each independently selected from hydrogen and hydroxyl; or R¹⁰⁶ and R^{106′} taken together are ═O, ═N—OR¹¹⁴, or ═NR¹¹⁴. In some embodiments, for the compound or salt of formula (III), R¹⁰⁶ and R^{106′} are each independently selected from hydrogen and hydroxyl. In some embodiments, R¹⁰⁶ and R^{106′} taken together are ═O. In some embodiments, R¹⁰⁶ and R^{106′} taken together are ═N—OR¹¹⁴.

In some embodiments, for the compound or salt of formula (III), r is selected from 0, 1, 2, and 3. In some embodiments, r is selected from 0 and 1.

In some embodiments, for the compound or salt of formula (III), s is selected from 0, 1, 2, and 3. In some embodiments, s is 0.

In some embodiments, for the compound or salt of formula (III), R¹¹¹ and R¹¹⁴ are each independently selected at each occurrence from
hydrogen;
C₁₋₆ alkyl optionally substituted with one or more substituent independently selected from R¹³¹; and
C₃₋₆ carbocycle and 3- to 6-membered heterocycle, any of which is optionally substituted with one or more substituent independently selected from R¹³¹.

In some embodiments, for the compound or salt of formula (III), R¹¹¹ and R¹¹⁴ are each independently selected at each occurrence from hydrogen;
C₁₋₆ alkyl optionally substituted with one or more substituent independently selected from R¹³¹; and
C₃₋₆ carbocycle and 3- to 6-membered heterocycle, any of which is optionally substituted with one or more substituent independently selected from R¹³¹.

In some embodiments, R¹¹¹ and R¹¹⁴ are each independently selected at each occurrence from hydrogen; C₁₋₆ alkyl optionally substituted with one or more substituent independently selected from R¹³¹; C₃₋₆ carbocycle and 3- to 6-membered heterocycle. In some embodiments, R¹¹¹ and R¹¹⁴ are each independently selected at each occurrence from hydrogen and C₁₋₆ alkyl optionally substituted with one or more substituent independently selected from R¹³¹. In some embodiments, R¹¹¹ and R¹¹⁴ are each independently selected at each occurrence from hydrogen and C₁₋₆ alkyl. In some embodiments, R¹¹¹ and R¹¹⁴ are each hydrogen.

In some embodiments, for the compound or salt of formula (III), R¹¹² is selected at each occurrence from
C₁₋₆ alkyl substituted with one or more substituent independently selected from R¹³²; and
C₃₋₆ carbocycle and 3- to 6-membered heterocycle, any of which is optionally substituted with one or more substituent independently selected from R¹³³.

In some embodiments, for the compound or salt of formula (III), R¹¹² is selected at each occurrence from C₁₋₆ alkyl substituted with one or more substituent independently selected from R¹³²; and C₃₋₆ carbocycle and 3- to 6-membered heterocycle, any of which is optionally substituted with one or more substituent independently selected from R¹³³. In some embodiments, R¹¹² is selected at each occurrence from C₁₋₆ alkyl substituted with one or more substituent independently selected from R¹³²; and C₃₋₆ carbocycle and 3- to 6-membered heterocycle. In some embodiments, R¹¹² is C₁₋₆ alkyl substituted with one or more substituent independently selected from R¹³². In some embodiments, R¹¹² is C₁₋₆ alkyl.

In some embodiments, for the compound or salt of formula (III), R¹¹³ is selected at each occurrence from
hydrogen;
C₁ alkyl and C₃₋₆ alkyl optionally substituted with one or more substituent independently selected from R¹³⁴, and C₂ alkyl substituted with one or more substituent independently selected from R¹³⁴; and
C₃₋₅ carbocycle and 3- to 6-membered heterocycle, any of which is optionally substituted with one or more substituent independently selected from R¹³⁴.

In some embodiments, for the compound or salt of formula (III), R¹¹³ is selected at each occurrence from
hydrogen;
C₁ alkyl and C₃₋₆ alkyl optionally substituted with one or more substituent independently selected from R¹³⁴, and C₂ alkyl substituted with one or more substituent independently selected from R¹³⁴; and
C₃₋₅ carbocycle and 3- to 6-membered heterocycle.

In some embodiments, for the compound or salt of formula (III), R¹¹³ is selected at each occurrence from hydrogen, C₁ alkyl and C₃₋₆ alkyl optionally substituted with one or more substituent independently selected from R¹³⁴, and C₂ alkyl substituted with one or more substituent independently selected from R¹³⁴. In some embodiments, R¹¹³ is selected at each occurrence from hydrogen, C₁ alkyl optionally substituted with one or more substituent independently selected from R¹³⁴, and C₂ alkyl substituted with one or more substituent independently selected from R¹³⁴. In some embodiments, R¹¹³ is selected at each occurrence from hydrogen, C₁ alkyl optionally substituted with one or more substituent independently selected from R¹³⁴. In some embodiments, R¹¹³ is selected at each occurrence from hydrogen, C₁ alkyl, and C₂ alkyl. In some embodiments, R¹¹³ is selected at each occurrence from hydrogen and C₁ alkyl. In some embodiments, R¹¹³ is hydrogen.

In some embodiments, for the compound or salt of formula (III), R¹²¹, R¹²⁴, and R¹²⁶ are each independently selected at each occurrence from
hydrogen;
C₁₋₆ alkyl optionally substituted with one or more substituents independently selected from R¹³⁴; and
C₃₋₆ carbocycle and 3- to 6-membered heterocycle, any of which is optionally substituted with one or more substituent independently selected from C₁₋₄ alkyl, C₁₋₄ haloalkyl, and R¹³⁴.

In some embodiments, for the compound or salt of formula (III), R¹²¹, R¹²⁴, and R¹²⁶ are each independently selected at each occurrence from
hydrogen;
C₁₋₆ alkyl optionally substituted with one or more substituents independently selected from R¹³⁴; and
C₃₋₆ carbocycle and 3- to 6-membered heterocycle.

In some embodiments, for the compound or salt of formula (III), R¹²¹, R¹²⁴, and R¹²⁶ are each independently selected at each occurrence from hydrogen and C₁₋₆ alkyl optionally substituted with one or more substituents independently selected from R¹³⁴. In some embodiments, R¹²¹, R¹²⁴, and R¹²⁶ are each independently selected at each occurrence from hydrogen and C₁₋₆ alkyl. In some embodiments, R¹²¹, R¹²⁴, and R¹²⁶ are each hydrogen.

In some embodiments, for the compound or salt of formula (III), R¹²² and R¹²⁵ are each independently selected at each occurrence from
C₂₋₆ alkyl optionally substituted with one or more substituents independently selected from R¹³⁴; and
C₃₋₆ carbocycle and 3- to 6-membered heterocycle, any of which is optionally substituted with one or more substituent independently selected from C₁₋₄ alkyl, C₁₋₄ haloalkyl, and R¹³.

In some embodiments, for the compound or salt of formula (III), R¹²² and R¹²⁵ are each independently selected at each occurrence from C₂₋₆ alkyl optionally substituted with one or more substituents independently selected from R¹³⁴; and C₃₋₆ carbocycle and 3- to 6-membered heterocycle. In some embodiments, R¹²² and R¹²⁵ are each C₂₋₆ alkyl optionally substituted with one or more substituents independently selected from R¹³⁴. In some embodiments, R¹²² and R¹²⁵ are each C₂₋₆ alkyl.

In some embodiments, for the compound or salt of formula (III), R¹²³ is independently selected at each occurrence from
hydrogen;
C₁₋₆ alkyl optionally substituted with one or more substituents independently selected from R¹³⁴; and
C₃₋₅ carbocycle and 3- to 6-membered heterocycle, any of which is optionally substituted with one or more substituent independently selected from C₁₋₄ alkyl, C₁₋₄ haloalkyl, and R¹³⁴;

In some embodiments, for the compound or salt of formula (III), R¹²³ is independently selected at each occurrence from
hydrogen;
C₁₋₆ alkyl optionally substituted with one or more substituents independently selected from R¹³⁴; and
C₃₋₅ carbocycle and 3- to 6-membered heterocycle;

In some embodiments, for the compound or salt of formula (III), R¹²³ is independently selected at each occurrence from hydrogen and C₁₋₆ alkyl optionally substituted with one or more substituents independently selected from R¹³⁴. In some embodiments, R¹²³ is independently selected at each occurrence from hydrogen and C₁₋₆ alkyl.

In some embodiments, for the compound or salt of formula (III), R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —OC(O)N(R¹⁴¹)₂, —C(O)N(R¹⁴¹)₂, —N(R¹⁴¹)C(O)R¹⁴¹, —N(R¹⁴¹)C(O)OR¹⁴¹, —N(R¹⁴¹)C(O)N(R¹⁴¹)₂, —N(R¹⁴¹)S(O)₂(R¹⁴¹), —N(R¹⁴¹)SO₂N(R¹⁴¹), —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, —CN, C₃₋₆ carbocycle, and 3- to 7-membered heterocycle.

In some embodiments, for the compound or salt of formula (III), R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —OC(O)N(R¹⁴¹)₂, —C(O)N(R¹⁴¹)₂, —N(R¹⁴¹)C(O)R¹⁴¹, —N(R¹⁴¹)C(O)OR¹⁴¹, —N(R¹⁴¹)C(O)N(R¹⁴¹)₂, —N(R¹⁴¹)S(O)₂(R¹⁴¹), —N(R¹⁴¹)SO₂N(R¹⁴¹), —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, and —CN. In some embodiments, R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —C(O)N(R¹⁴¹)₂, —N(R¹⁴¹)C(O)R¹⁴¹, —N(R¹⁴¹)S(O)₂(R¹⁴¹), —N(R¹⁴¹)SO₂N(R¹⁴¹), —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, and —CN. In some embodiments, R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —C(O)N(R¹⁴¹)₂, —N(R¹⁴¹)C(O)R¹⁴¹, —S(O)R¹⁴¹, —S(O)₂R¹⁴¹, —S(O)₂N(R¹⁴¹)₂, —NO₂, ═O, ═S, and —CN. In some embodiments, R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —C(O)N(R¹⁴¹)₂, —N(R¹⁴¹)C(O)R¹⁴¹, —NO₂, ═O, ═S, and —CN. In some embodiments, R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —NO₂, ═O, ═S, and —CN. In some embodiments, R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —NO₂, ═O, and —CN. In some embodiments, R¹³⁰, R¹³¹, R¹³², R¹³³, and R¹³⁴ are each independently selected at each occurrence from halogen, —OR¹⁴¹, —N(R¹⁴¹)₂, —NO₂, ═O, and —CN. In some embodiments, R¹³⁰, R¹³¹, R¹³², R¹³³, and R¹³⁴ are each independently selected at each occurrence from halogen, —OR¹⁴¹, —N(R¹⁴¹)₂, —NO₂, and —CN. In some embodiments, R¹³⁰, R¹³¹, R¹³², R¹³³, and R¹³⁴ are each independently selected at each occurrence from halogen, —NO₂, and —CN.

In some embodiments, for the compound or salt of formula (III), R¹⁴¹ is independently selected at each occurrence from hydrogen, C₁₋₆ alkyl, C₁₋₆ haloalkyl, and C₁₋₆ hydroxyalkyl. In some embodiments, R¹⁴¹ is independently selected at each occurrence from hydrogen, C₁₋₆ alkyl, and C₁₋₆ haloalkyl. In some embodiments, R¹⁴¹ is independently selected at each occurrence from hydrogen, C₁₋₆ alkyl, and C₁₋₆ hydroxyalkyl. In some embodiments, R¹⁴¹ is independently selected at each occurrence from hydrogen and C₁₋₆ alkyl. In some embodiments, R¹⁴¹ is at each occurrence hydrogen.

In one aspect, provided herein is a compound having the structure of Formula (IV):

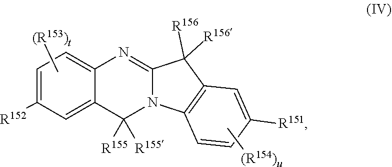

or a pharmaceutically acceptable salt thereof; wherein
R¹⁵¹ is selected from fluoro, bromo, —N(R¹⁶¹)₂, —C(O)R¹⁶¹, —C(O)OR¹⁶¹, —OC(O)R¹⁶¹, —OC(O)N(R¹⁶¹)₂, —C(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), —N(R¹⁶¹)S(O)₂N(R¹⁶¹)₂, —SR¹⁶¹, —S(O)R¹⁶¹, —S(O)₂R¹⁶¹, —S(O)₂N(R¹⁶¹)₂, —CN, —NO₂, —C₁₋₆ haloalkyl, —O—C₂₋₆ alkyl, —O—C₁₋₆ haloalkyl, —OR₃₋₆ carbocycle, and —OR₃₋₆ heterocycle; and

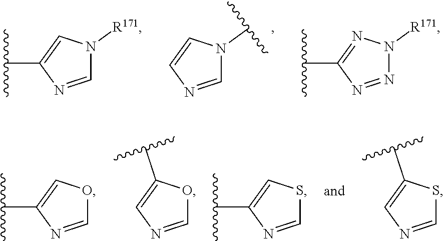

each of which is optionally substituted with one or more substituent independently selected from R¹⁷¹;
when R¹⁵¹ is fluoro, R¹⁵² is selected from
   iodo, —NHR¹⁹², —OR¹⁷¹, —SR¹⁷¹, —C(═NR¹⁹⁶)N(R¹⁹⁶)₂, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁹³)C(O)R¹⁷¹, —N(H)C(O)R¹⁹⁴, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, —S(O)₂N(R¹⁷¹)₂, —NR¹⁹⁶(C═NR¹⁹⁶)N(R¹⁹⁶)₂, —N₃, and —CN;
   C₁ alkyl substituted with one or more substituent independently selected from R¹⁹⁵, and C₂₋₆ alkyl optionally substituted with one or more substituent independently selected from R¹⁸⁰; and
C₃₋₆ carbocycle, 3-membered heterocycle, 4-membered heterocycle,

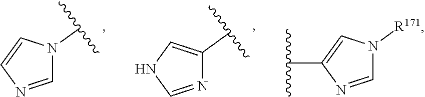

and 6-membered heterocycle, any of which is optionally substituted with one or more substituent independently selected from R¹⁸⁰
when R¹⁵¹ is

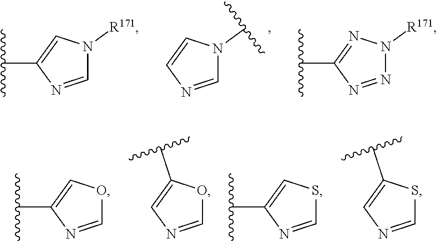

—SR¹⁶¹, —S(O)R¹⁶¹, —S(O)₂R¹⁶¹, or —S(O)₂N(R¹⁶¹)₂, or —CN, R¹⁵² is selected from
halogen, —N(R¹⁷⁴)₂, —OR¹⁷⁴, —SR¹⁷⁴, —C(O)R¹⁷⁴, —C(O)OR¹⁷⁴, —OC(O)R¹⁷⁴, —OC(O)N(R¹⁷⁴)₂, —C(O)N(R¹⁷⁴)₂, —N(R¹⁷⁴)C(O)R¹⁷⁴, —N(R¹⁷⁴)C(O)OR¹⁷⁴, —N(R¹⁷⁴)C(O)N(R¹⁷⁴)₂, —N(R¹⁷⁴)S(O)₂(R¹⁷⁴), —N(R¹²⁴)SO₂N(R¹⁷⁴)₂, —N(R¹⁷⁴)P(O)(OR¹⁷⁴)R¹⁷⁴, —S(O)R¹⁷⁴, —S(O)₂R¹⁷⁴, —S(O)₂N(R¹⁷⁴)₂, —NO₂, and —CN;
C₁₋₆ alkyl, optionally substituted with one or more substituent independently selected from R¹⁸⁰; and
C₃₋₆ carbocycle and 3- to 6-membered heterocycle, any of which is optionally substituted with one or more substituent independently selected from R¹⁸⁰
when R¹⁵¹ is bromo or —NO₂, R¹⁵² is selected from
   iodo, —OR¹⁹⁷, —SR¹⁷⁶, —NHR¹⁷⁸, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
   C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰;
   C₃₋₆ carbocycle and 3- to 6-membered heterocycle, any of which is optionally substituted with one or more substituent independently selected from R¹⁸⁰; and
   provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl;
when R¹⁵¹ is —C₁₋₆ haloalkyl, —O—C₂₋₆ alkyl, —O—C₁₋₆ haloalkyl, —OR₃₋₆ carbocycle, —OR₃₋₆ heterocycle, —N(R¹⁶¹)₂, —C(O)R¹⁶¹, —OC(O)R¹⁶¹, —C(O)N(R¹⁶¹)₂, —OC(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), or —N(R¹⁶¹)S(O)₂N(R¹⁶¹)₂, R¹⁵² is selected from
   halogen, —OR¹⁷⁶, —SR¹⁷⁶, —N(R¹⁷⁶)₂, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
   C₁₋₆ alkyl, optionally substituted with one or more substituent independently selected from R¹⁷⁷; and
   C₃₋₆ carbocycle and 3- to 6-membered heterocycle, any of which is optionally substituted with one or more substituent independently selected from R¹⁷⁷;
R¹⁵³ and R¹⁵⁴ are each independently selected at each occurrence from
   halogen, —OR¹⁶⁴, —NHR¹⁷⁸, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
   C₁₋₄ alkyl, optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN; and
   C₃₋₆ carbocycle and 3- to 5-membered heterocycle, any of which is optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
R¹⁵⁵ and R^{155′} are each independently selected from hydrogen, hydroxyl, and methyl; or R¹⁵⁵ and R^{155′} taken together are ═O;
R¹⁵⁶ and R^{156′} are each independently selected from hydrogen, hydroxyl, and methyl; or R¹⁵⁶ and R^{156′} taken together are ═O, ═N—OR¹⁷¹, or NR¹⁷¹;
t is selected from 0, 1, 2, and 3;
u is selected from 0, 1, 2, and 3;
R¹⁶¹ and R¹⁶⁴ are each independently selected at each occurrence from hydrogen;
   C₁₋₆ alkyl optionally substituted with one or more substituent independently selected from R¹⁷⁷; and
   C₃₋₆ carbocycle and 3- to 6-membered heterocycle, any of which is optionally substituted with one or more substituent independently selected from R¹⁷⁷;
R¹⁷¹, R¹⁷⁴, and R¹⁷⁶ are each independently selected at each occurrence from hydrogen;
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁷⁷ and R¹⁸⁰ are each independently selected at each occurrence from halogen, —OR¹⁹¹, —SR¹⁹¹, —N(R¹⁹¹)₂, —C(O)R¹⁹¹, —C(O)OR¹⁹¹, —OC(O)R¹⁹¹, —OC(O)N(R¹⁹¹)₂, —C(O)N(R¹⁹¹)₂, —N(R¹⁹¹)C(O)R¹⁹¹, —N(R¹⁹¹)C(O)OR¹⁹¹, —N(R¹⁹¹)C(O)N(R¹⁹¹)₂, —N(R¹⁹¹)S(O)₂(R¹⁹¹), —N(R¹⁹¹)SO₂N(R¹⁹¹)₂, —N(R¹⁹¹)P(O)(OR¹⁹¹)R¹⁹¹, —S(O)R¹⁹¹, —S(O)₂R¹⁹¹, —S(O)₂N(R¹⁹¹)₂, —NO₂, ═O, ═S, —CN, C₁₋₆ alkyl, C₃₋₆ carbocycle, and 3- to 7-membered heterocycle;
R¹⁷⁸ is selected at each occurrence from
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from R¹⁸⁰
R¹⁹¹ is independently selected at each occurrence from hydrogen, C₁₋₆ alkyl, C₁₋₆ haloalkyl, and C₁₋₆ hydroxyalkyl.
R¹⁹² is selected at each occurrence from
   C₃₋₆ carbocycle and 3- to 6-membered heterocycle, any of which is optionally substituted with one or more substituent independently selected from R¹⁸⁰
R¹⁹³ is selected at each occurrence from
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁴ is selected at each occurrence from C₁₋₆ alkyl substituted with one or more substituents independently selected from R¹⁸⁰;
R¹⁹⁵ is independently selected at each occurrence from halogen, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)S(O)₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
   C₁₋₆ alkyl optionally substituted with one or more substituents independently selected from R¹⁸⁰; and
   C₅₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁶ is selected at each occurrence from hydrogen, —CN, and OR¹⁷¹;
   C₁₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁷ is selected at each occurrence from hydrogen;
   C₂₋₆ alkyl optionally substituted with one or more substituents independently selected from R¹⁸⁰; and
   C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁸ is independently selected at each occurrence from fluoro, chloro, iodo, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
   C₂₋₆ alkyl optionally substituted with one or more substituents independently selected from R¹⁸⁰; and
   C₅₋₆ carbocycle and 3- to 6-membered heterocycle, any of which is optionally substituted with one or more substituent independently selected from C₁₋₄ alkyl, C₁₋₄ haloalkyl, and R¹⁸⁰;

In some embodiments, for the compound or salt of (IV), R¹⁵¹ is fluoro, bromo, —C(O)R¹⁶¹, —C(O)OR¹⁶¹, —C(O)N(R¹⁶¹)₂, —N(R¹⁶¹)C(O)R¹⁶¹, —N(R¹⁶¹)S(O)₂(R¹⁶¹), —SR¹⁶¹, —S(O)R¹⁶¹, —S(O)₂R¹⁶¹, —S(O)₂N(R¹⁶¹)₂, —CN, —NO₂, —C₁₋₆ haloalkyl, —O—C₂₋₆ alkyl, —O—C₁₋₆ haloalkyl, or

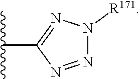

In some embodiments, for the compound or salt of (IV), R¹⁵¹ is fluoro, bromo, —C(O)R¹⁶¹, —C(O)OR¹⁶¹, —C(O)N(R¹⁶¹)₂, —N(R¹⁶¹)C(O)R¹⁶¹, —SR¹⁶¹, —S(O)₂R¹⁶¹, —S(O)₂N(R¹⁶¹)₂, —CN, —NO₂, or

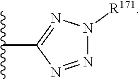

In some embodiments, for the compound or salt of (IV), R¹⁵¹ is fluoro, bromo, —SR¹⁶¹, —S(O)₂R¹⁶¹, —CN, —NO₂, or

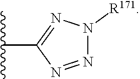

In some embodiments, for the compound or salt of (IV), R¹⁵¹ is fluoro. In some embodiments, for the compound or salt of (IV), when R¹⁵¹ is fluoro, R¹⁵² is selected from —NHR¹⁹², —OR¹⁷¹, —SR¹⁷¹, —C(═NR¹⁹⁶)N(R¹⁹⁶)₂, —C(O)R¹⁷¹, —C(O)OR¹⁷¹, —OC(O)R¹⁷¹, —C(O)N(R¹⁷¹)₂, —N(H)C(O)R¹⁹⁴, —N(R¹⁷¹)S(O)₂(R¹⁷¹), —S(O)₂R¹⁷¹, —S(O)₂N(R¹⁷¹)₂, —NR¹⁹⁶(C═NR¹⁹⁶)N(R¹⁹⁶)₂, —N₃, —CN, C₁ alkyl substituted with one or more substituent independently selected from R¹⁹⁵,

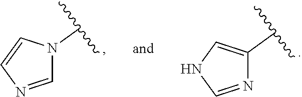 [SMILES: *n1ccnc1 ; *c1c[nH]cn1]

In some embodiments, for the compound or salt of (IV), when R¹⁵¹ is fluoro, R¹⁵² is selected from —NHR¹⁹², —C(═NR¹⁹⁶)N(R¹⁹⁶)₂, —C(O)R¹⁷¹, —C(O)OR¹⁷¹, —C(O)N(R¹⁷¹)₂, —N(H)C(O)R¹⁹⁴, —NR¹⁹⁶(C═NR¹⁹⁶)N(R¹⁹⁶)₂, —N₃, —CN, C₁ alkyl substituted with one or more substituent independently selected from R¹⁹⁵,

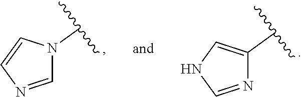 [SMILES: *n1ccnc1 ; *c1c[nH]cn1]

In some embodiments, for the compound or salt of (IV), when R¹⁵¹ is fluoro, R¹⁵² is selected from —NHR¹⁹², —C(═NR¹⁹⁶)N(R¹⁹⁶)₂, —C(O)R¹⁷¹, —C(O)OR¹⁷¹, —NR¹⁹⁶(C═NR¹⁹⁶)N(R¹⁹⁶)₂, —N₃, —CN, C₁ alkyl substituted with one or more substituent independently selected from R¹⁹⁵,

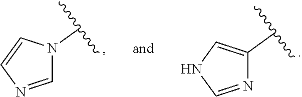 [SMILES: *n1ccnc1 ; *c1c[nH]cn1]

In some embodiments, for the compound or salt of (IV), when R¹⁵¹ is fluoro, R¹⁵² is selected from —NHR¹⁹², —C(═NR¹⁹⁶)N(R¹⁹⁶)₂, —C(O)R¹⁷¹, —C(O)OR¹⁷¹, and —NR¹⁹⁶(C═NR¹⁹⁶)N(R¹⁹⁶)₂. In some embodiments, for the compound or salt of (IV), when R¹⁵¹ is fluoro, R¹⁵² is selected from —N₃, —CN, C₁ alkyl substituted with one or more substituent independently selected from R¹⁹⁵,

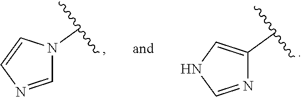 [SMILES: *n1ccnc1 ; *c1c[nH]cn1]

In some embodiments, for the compound or salt of (IV), when R¹⁵¹ is fluoro, R¹⁵² is selected from —NHR¹⁹², —C(═NR¹⁹⁶)N(R¹⁹⁶)₂, —C(O)R¹⁷¹, —C(O)OR¹⁷¹, and —NR¹⁹⁶(C═NR¹⁹⁶)N(R¹⁹⁶)₂. In some embodiments, for the compound or salt of (IV), when R¹⁵¹ is fluoro, R¹⁵² is selected from —NHR¹⁹², —C(═NR¹⁹⁶)N(R¹⁹⁶)₂, and —NR¹⁹⁶(C═NR¹⁹⁶)N(R¹⁹⁶)₂. In some embodiments, for the compound or salt of (IV), when R¹⁵¹ is fluoro, R¹⁵² is selected from —NHR¹⁹², —C(O)R¹⁷¹, and —C(O)OR¹⁷¹. In some embodiments, for the compound or salt of (IV), when R¹⁵¹ is fluoro, R¹⁵² is —NHR¹⁹². In some embodiments, for the compound or salt of (IV), when R¹⁵¹ is fluoro, R¹⁵² is —C(═NR¹⁹⁶)N(R¹⁹⁶)₂. In some embodiments, for the compound or salt of (IV), when R¹⁵¹ is fluoro, R¹⁵² is —C(O)R¹⁷¹. In some embodiments, for the compound or salt of (IV), when R¹⁵¹ is fluoro, R¹⁵² is —C(O)OR¹⁷¹. In some embodiments, for the compound or salt of (IV), when R¹⁵¹ is fluoro, R¹⁵² is —NR¹⁹⁶(C═NR¹⁹⁶)N(R¹⁹⁶)₂. In some embodiments, for the compound or salt of (IV), when R¹⁵¹ is fluoro, R¹⁵² is selected from C₁ alkyl substituted with one or more substituent independently selected from R¹⁹⁵,

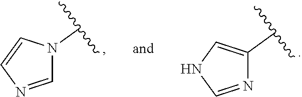 [SMILES: *n1ccnc1 ; *c1c[nH]cn1]

In some embodiments, for the compound or salt of (IV), when R¹⁵¹ is fluoro, R¹⁵² is —N₃. In some embodiments, for the compound or salt of (IV), when R¹⁵¹ is fluoro, R¹⁵² is —CN. In some embodiments, for the compound or salt of (IV), when R¹⁵¹ is fluoro, R¹⁵² is C₁ alkyl substituted with one or more substituent independently selected from R¹⁹⁵. In some embodiments, for the compound or salt of (IV), when R¹⁵¹ is fluoro, R¹⁵² is

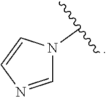 [SMILES: *n1ccnc1]

In some embodiments, for the compound or salt of (IV), when R¹⁵¹ is fluoro, R¹⁵² is

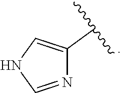 [SMILES: *c1c[nH]cn1]

In some embodiments, for the compound or salt of (IV), R¹⁵¹ is

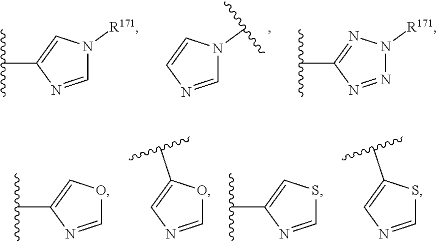

—SR¹⁶¹, —S(O)R¹⁶¹, —S(O)₂R¹⁶¹, —S(O)₂N(R¹⁶¹)₂ or —CN. In some embodiments, for the compound or salt of (IV), R¹⁵¹ is —SR¹⁶¹, —S(O)₂R¹⁶¹, —CN, or

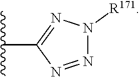

In some embodiments, for the compound or salt of (IV), R¹⁵¹ is —SR¹⁶¹. In some embodiments, for the compound or salt of (IV), R¹⁵¹ is —S(O)₂R¹⁶¹. In some embodiments, for the compound or salt of (IV), R¹⁵¹ is —CN. In some embodiments, for the compound or salt of (IV), R¹⁵¹ is

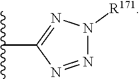

In some embodiments, for the compound or salt of (IV), when R¹⁵¹ is —SR¹⁶¹, —S(O)₂R¹⁶¹, —CN, or

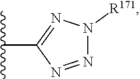

R¹⁵² is selected from halogen, —N(R¹⁷⁴)₂, —OR¹⁷⁴, —SR¹⁷⁴, —C(O)R¹⁷⁴, —C(O)OR¹⁷⁴, —OC(O)R¹⁷⁴, —C(O)N(R¹⁷⁴)₂, —N(R¹⁷⁴)C(O)R¹⁷⁴, —N(R¹⁷⁴)C(O)OR¹⁷⁴, —N(R¹⁷⁴)S(O)₂(R¹⁷⁴), —S(O)₂R¹⁷⁴, —S(O)₂N(R¹⁷⁴)₂, —NO₂, —CN, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is —SR¹⁶¹, —S(O)₂R¹⁶¹, —CN, or

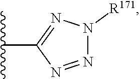

R¹⁵² is selected from halogen, —N(R¹⁷⁴)₂, —OR¹⁷⁴, —C(O)N(R¹⁷⁴)₂, —N(R¹⁷⁴)C(O)R¹⁷⁴, —N(R¹⁷⁴)C(O)OR¹⁷⁴, —NO₂, —CN, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is —SR¹⁶¹, —S(O)₂R¹⁶¹, —CN, or

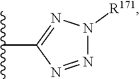

R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is —SR¹⁶¹, R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is —S(O)₂R¹⁶¹, R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is —CN, R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰. In some embodiments, for the compound or salt of (IV), when R¹⁵¹ is

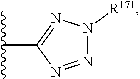

R¹⁵² is selected from —C(O)N(R¹⁷⁴)₂, —N(R¹⁷⁴)C(O)R¹⁷⁴, —N(R¹⁷⁴)C(O)OR¹⁷⁴, C₁₋₆ alkyl, optionally substituted with one or more substituent independently selected from R¹⁸⁰, and a 5-membered heterocycle which is optionally substituted with one or more substituent independently selected from R¹⁸⁰.

In some embodiments, for the compound or salt of (IV), R¹⁵¹ is bromo or —NO₂. In some embodiments, for the compound or salt of (IV), R¹⁵¹ is bromo. In some embodiments, for the compound or salt of (IV), R¹⁵¹ is —NO₂. In some embodiments, for the compound or salt of (IV), when R¹⁵¹ is bromo or —NO₂, R¹⁵² is selected from —OR¹⁹⁷, —SR¹⁷⁶, —NHR¹⁷⁸, —C(O)R¹⁷⁶, —C(O)OR¹⁷⁶, —C(O)N(R¹⁷⁶)₂, —N(R¹⁷⁶)C(O)R¹⁷⁶, —N(R¹⁷⁶)S(O)₂(R¹⁷⁶), —S(O)₂R¹⁷⁶, —S(O)₂N(R¹⁷⁶)₂, —NO₂, —CN, C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰ provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl. In some embodiments, for the compound or salt of (IV), when R¹⁵¹ is bromo or —NO₂, R¹⁵² is selected from C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰ provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl. In some embodiments, for the compound or salt of (IV), R¹⁵¹ is bromo and R¹⁵² is C₁ alkyl substituted with one or more substituent independently selected from R¹⁸⁰. In some embodiments, for the compound or salt of (IV), R¹⁵¹ is —NO₂ and R¹⁵² is C₁ alkyl substituted with one or more substituent independently selected from R¹⁹⁸. In some embodiments, for the compound or salt of (IV), when R¹⁵¹ is bromo, R¹⁵² is selected from —OR¹⁹⁷, —SR¹⁷⁶, —NHR¹⁷⁸, —C(O)R¹⁷⁶, —C(O)OR¹⁷⁶, —C(O)N(R¹⁷⁶)₂, —N(R¹⁷⁶)C(O)R¹⁷⁶, —N(R¹⁷⁶)S(O)₂(R¹⁷⁶), —S(O)₂R¹⁷⁶, —S(O)₂N(R¹⁷⁶)₂, —NO₂, —CN, C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰. In some embodiments, for the compound or salt of (IV), when R¹⁵¹ is —NO₂, R¹⁵² is selected from —OR¹⁹⁷, —SR¹⁷⁶, —NHR¹⁷⁸, —C(O)R¹⁷⁶, —C(O)OR¹⁷⁶, —C(O)N(R¹⁷⁶)₂, —N(R¹⁷⁶)C(O)R¹⁷⁶, —N(R¹⁷⁶)S(O)₂(R¹⁷⁶), —S(O)₂R¹⁷⁶, —S(O)₂N(R¹⁷⁶)₂, —NO₂, —CN, C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰ provided that (i) R¹⁵² cannot be C₁ alkyl substituted with one or more substituent independently selected from R¹⁸⁰; and (ii) R¹⁵² cannot be ethyl.

In some embodiments, for the compound or salt of (IV), R¹⁵³ and R¹⁵⁴ are each independently selected at each occurrence from halogen and C₁₋₄ alkyl. In some embodiments, for the compound or salt of (IV), R¹⁵⁵ and R^{155′} taken together are ═O. In some embodiments, for the compound or salt of (IV), R¹⁵⁶ and R^{156′} taken together are ═O. In some embodiments, for the compound or salt of (IV), R¹⁵⁶ and R^{156′} taken together are ═N—OR¹⁷¹. In some embodiments, for the compound or salt of (IV), R¹⁵⁶ and R^{156′} are each independently selected from hydroxyl and methyl. In some embodiments, for the compound or salt of (IV), t is selected from 0 and 1. In some embodiments, for the compound or salt of (IV), u is 0.

In some aspects, the compound is any of the compounds represented in Table I, or a pharmaceutically acceptable salt or solvate thereof.

TABLE 1

| Compound | |
|---|---|
| Number | Structure |
| 1 | 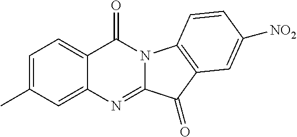 [SMILES: Cc1ccc2c(=O)n3c(nc2c1)C(=O)c1cc([N+](=O)[O-])ccc1-3] |
| 2 | 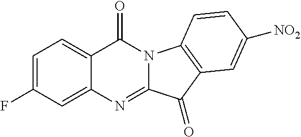 [SMILES: O=C1c2cc([N+](=O)[O-])ccc2-n2c1nc1cc(F)ccc1c2=O] |
| 3 | 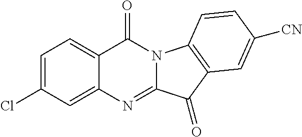 [SMILES: N#Cc1ccc2c(c1)C(=O)c1nc3cc(Cl)ccc3c(=O)n1-2] |
| 4 | 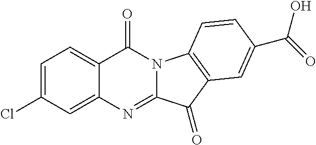 [SMILES: O=C(O)c1ccc2c(c1)C(=O)c1nc3cc(Cl)ccc3c(=O)n1-2] |
| 5 | 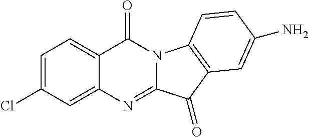 [SMILES: Nc1ccc2c(c1)C(=O)c1nc3cc(Cl)ccc3c(=O)n1-2] |
| 6 | 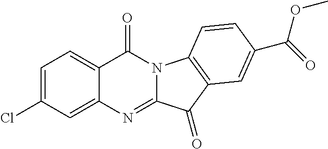 [SMILES: COC(=O)c1ccc2c(c1)C(=O)c1nc3cc(Cl)ccc3c(=O)n1-2] |
| 7 | 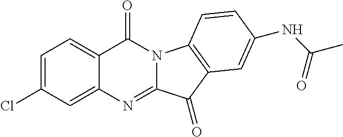 [SMILES: CC(=O)Nc1ccc2c(c1)C(=O)c1nc3cc(Cl)ccc3c(=O)n1-2] |
| 8 | 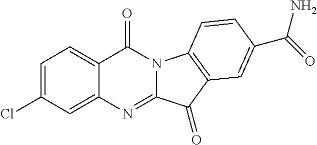 [SMILES: NC(=O)c1ccc2c(c1)C(=O)c1nc3cc(Cl)ccc3c(=O)n1-2] |
| 9 | 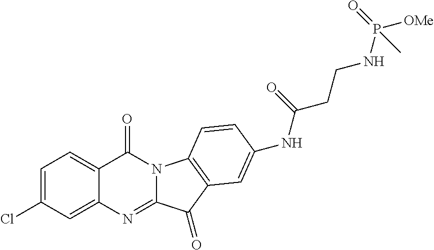 [SMILES: COP(C)(=O)NCCC(=O)Nc1ccc2c(c1)C(=O)c1nc3cc(Cl)ccc3c(=O)n1-2] |
| 10 | 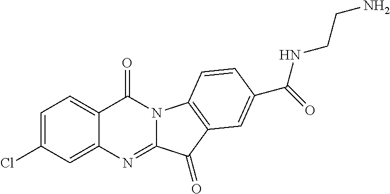 [SMILES: NCCNC(=O)c1ccc2c(c1)C(=O)c1nc3cc(Cl)ccc3c(=O)n1-2] |
| 11 | 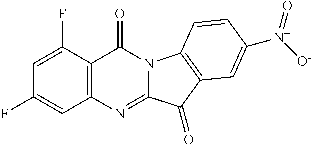 [SMILES: O=C1c2cc([N+](=O)[O-])ccc2-n2c1nc1cc(F)cc(F)c1c2=O] |
| 12 | 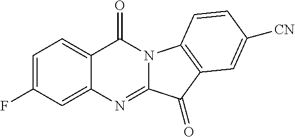 [SMILES: N#Cc1ccc2c(c1)C(=O)c1nc3cc(F)ccc3c(=O)n1-2] |
| 13 | 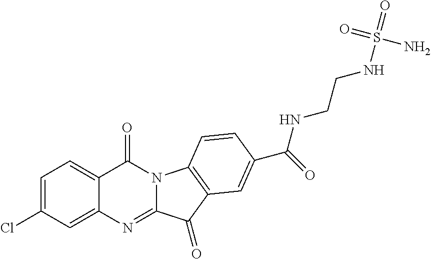 [SMILES: NS(=O)(=O)NCCNC(=O)c1ccc2c(c1)C(=O)c1nc3cc(Cl)ccc3c(=O)n1-2] |
| 14 | 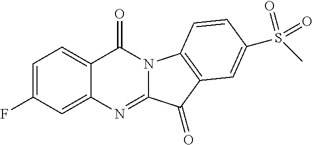 [SMILES: CS(=O)(=O)c1ccc2c(c1)C(=O)c1nc3cc(F)ccc3c(=O)n1-2] |
| 15 | 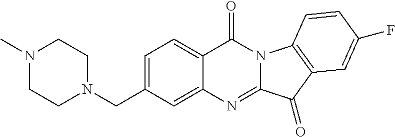 [SMILES: CN1CCN(Cc2ccc3c(=O)n4c(nc3c2)C(=O)c2cc(F)ccc2-4)CC1] |
| 16 | 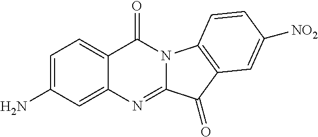 [SMILES: Nc1ccc2c(=O)n3c(nc2c1)C(=O)c1cc([N+](=O)[O-])ccc1-3] |
| 17 | 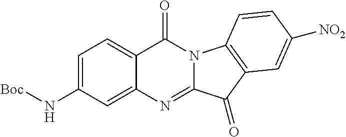 [SMILES: CC(C)(C)OC(=O)Nc1ccc2c(=O)n3c(nc2c1)C(=O)c1cc([N+](=O)[O-])ccc1-3] |
| 18 | 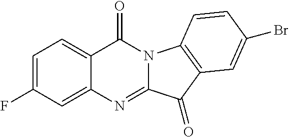 [SMILES: O=C1c2cc(Br)ccc2-n2c1nc1cc(F)ccc1c2=O] |
| 19 | 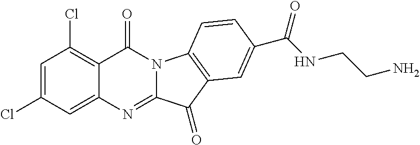 [SMILES: NCCNC(=O)c1ccc2c(c1)C(=O)c1nc3cc(Cl)cc(Cl)c3c(=O)n1-2] |
| 20 | 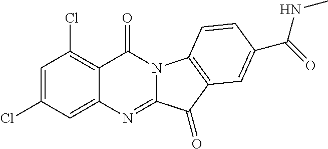 [SMILES: CNC(=O)c1ccc2c(c1)C(=O)c1nc3cc(Cl)cc(Cl)c3c(=O)n1-2] |
| 21 | 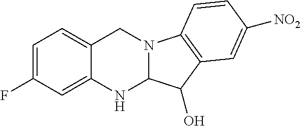 [SMILES: O=[N+]([O-])c1ccc2c(c1)C(O)C1Nc3cc(F)ccc3CN21] |
| 22 | 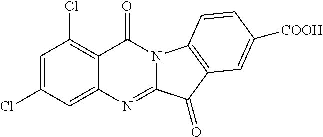 [SMILES: O=C(O)c1ccc2c(c1)C(=O)c1nc3cc(Cl)cc(Cl)c3c(=O)n1-2] |
| 23 | 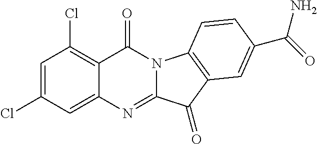 [SMILES: NC(=O)c1ccc2c(c1)C(=O)c1nc3cc(Cl)cc(Cl)c3c(=O)n1-2] |
| 24 | 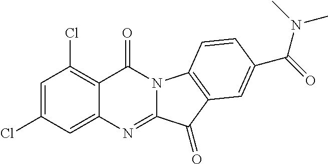 [SMILES: CN(C)C(=O)c1ccc2c(c1)C(=O)c1nc3cc(Cl)cc(Cl)c3c(=O)n1-2] |
| 25 | 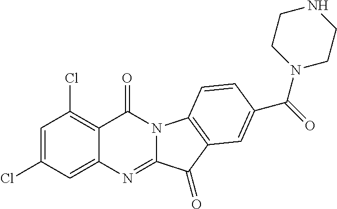 [SMILES: O=C1c2cc(C(=O)N3CCNCC3)ccc2-n2c1nc1cc(Cl)cc(Cl)c1c2=O] |
| 26 | 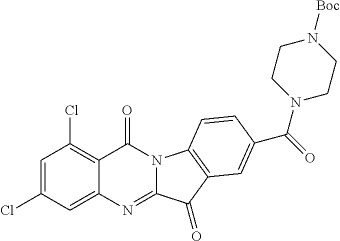 [SMILES: CC(C)(C)OC(=O)N1CCN(C(=O)c2ccc3c(c2)C(=O)c2nc4cc(Cl)cc(Cl)c4c(=O)n2-3)CC1] |
| 27 | 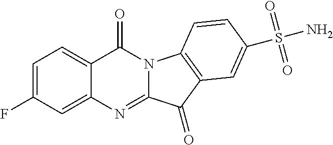 [SMILES: NS(=O)(=O)c1ccc2c(c1)C(=O)c1nc3cc(F)ccc3c(=O)n1-2] |
| 28 | 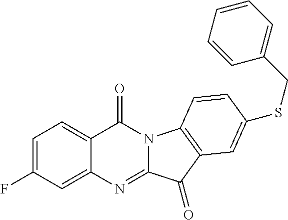 [SMILES: O=C1c2cc(SCc3ccccc3)ccc2-n2c1nc1cc(F)ccc1c2=O] |
| 29 | 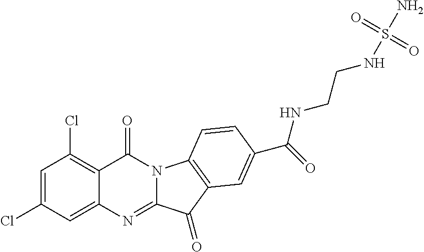 [SMILES: NS(=O)(=O)NCCNC(=O)c1ccc2c(c1)C(=O)c1nc3cc(Cl)cc(Cl)c3c(=O)n1-2] |
| 30 | 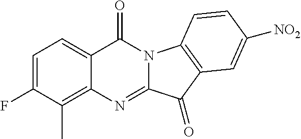 [SMILES: Cc1c(F)ccc2c(=O)n3c(nc12)C(=O)c1cc([N+](=O)[O-])ccc1-3] |
| 31 | 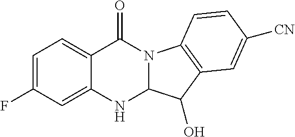 [SMILES: N#Cc1ccc2c(c1)C(O)C1Nc3cc(F)ccc3C(=O)N21] |
| 32 | 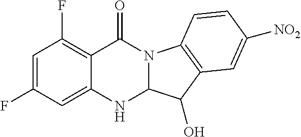 [SMILES: O=C1c2c(F)cc(F)cc2NC2C(O)c3cc([N+](=O)[O-])ccc3N12] |
| 33 | 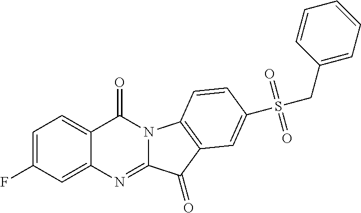 [SMILES: O=C1c2cc(S(=O)(=O)Cc3ccccc3)ccc2-n2c1nc1cc(F)ccc1c2=O] |
| 34 | 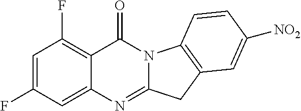 [SMILES: O=c1c2c(F)cc(F)cc2nc2n1-c1ccc([N+](=O)[O-])cc1C2] |
| 35 | 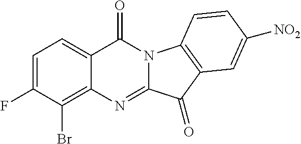 [SMILES: O=C1c2cc([N+](=O)[O-])ccc2-n2c1nc1c(Br)c(F)ccc1c2=O] |
| 36 | 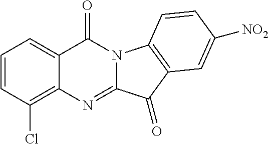 [SMILES: O=C1c2cc([N+](=O)[O-])ccc2-n2c1nc1c(Cl)cccc1c2=O] |
| 37 | 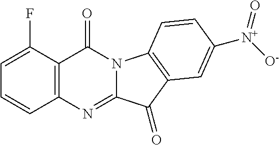 [SMILES: O=C1c2cc([N+](=O)[O-])ccc2-n2c1nc1cccc(F)c1c2=O] |
| 38 | 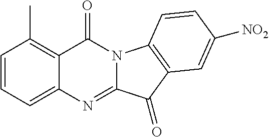 [SMILES: Cc1cccc2nc3n(c(=O)c12)-c1ccc([N+](=O)[O-])cc1C3=O] |
| 39 | 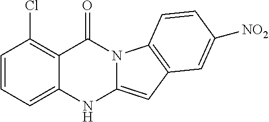 [SMILES: O=c1c2c(Cl)cccc2[nH]c2cc3cc([N+](=O)[O-])ccc3n12] |
| 40 | 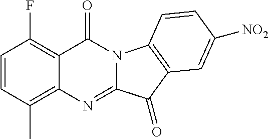 [SMILES: Cc1ccc(F)c2c(=O)n3c(nc12)C(=O)c1cc([N+](=O)[O-])ccc1-3] |
| 41 | 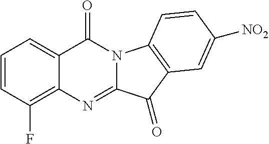 [SMILES: O=C1c2cc([N+](=O)[O-])ccc2-n2c1nc1c(F)cccc1c2=O] |
| 42 | 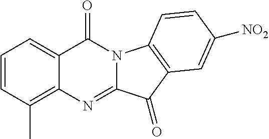 [SMILES: Cc1cccc2c(=O)n3c(nc12)C(=O)c1cc([N+](=O)[O-])ccc1-3] |
| 43 | 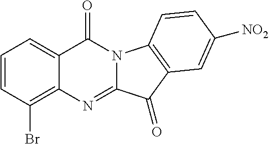 [SMILES: O=C1c2cc([N+](=O)[O-])ccc2-n2c1nc1c(Br)cccc1c2=O] |
| 44 | 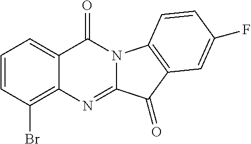 [SMILES: O=C1c2cc(F)ccc2-n2c1nc1c(Br)cccc1c2=O] |
| 45 | 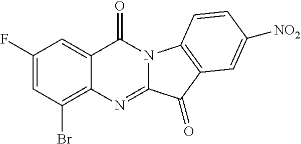 [SMILES: O=C1c2cc([N+](=O)[O-])ccc2-n2c1nc1c(Br)cc(F)cc1c2=O] |
| 46 | 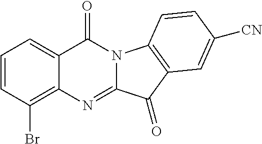 [SMILES: N#Cc1ccc2c(c1)C(=O)c1nc3c(Br)cccc3c(=O)n1-2] |
| 47 | 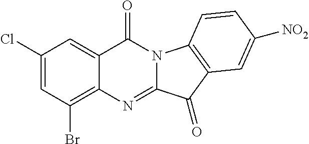 [SMILES: O=C1c2cc([N+](=O)[O-])ccc2-n2c1nc1c(Br)cc(Cl)cc1c2=O] |
| 48 | 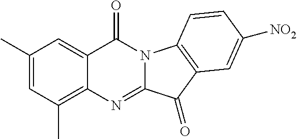 [SMILES: Cc1cc(C)c2nc3n(c(=O)c2c1)-c1ccc([N+](=O)[O-])cc1C3=O] |
| 49 | 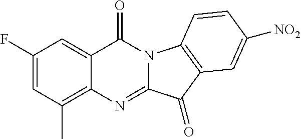 [SMILES: Cc1cc(F)cc2c(=O)n3c(nc12)C(=O)c1cc([N+](=O)[O-])ccc1-3] |
| 50 | 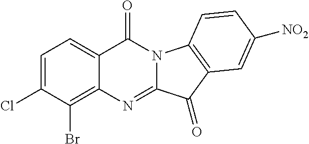 [SMILES: O=C1c2cc([N+](=O)[O-])ccc2-n2c1nc1c(Br)c(Cl)ccc1c2=O] |
| 51 | 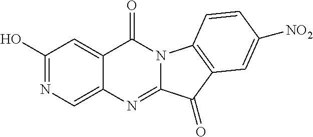 [SMILES: O=C1c2cc([N+](=O)[O-])ccc2-n2c1nc1cnc(O)cc1c2=O] |
| 52 | 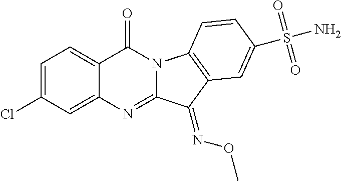 [SMILES: CO/N=C1\c2cc(S(N)(=O)=O)ccc2-n2c1nc1cc(Cl)ccc1c2=O] |
| 53 | 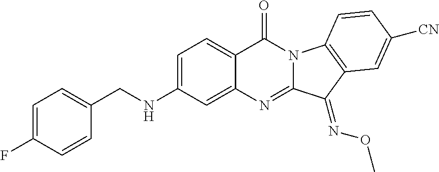 [SMILES: CO/N=C1\c2cc(C#N)ccc2-n2c1nc1cc(NCc3ccc(F)cc3)ccc1c2=O] |
| 54 | 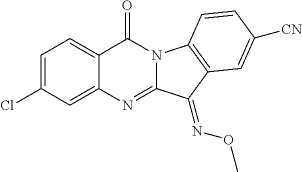 [SMILES: CO/N=C1\c2cc(C#N)ccc2-n2c1nc1cc(Cl)ccc1c2=O] |
| 55 | 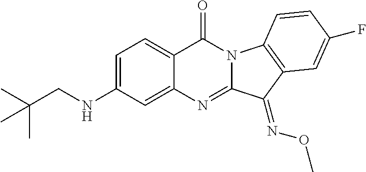 [SMILES: CO/N=C1\c2cc(F)ccc2-n2c1nc1cc(NCC(C)(C)C)ccc1c2=O] |
| 56 | 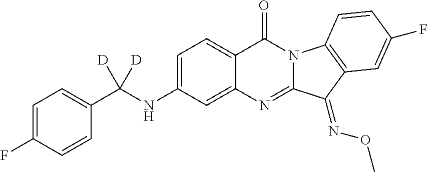 [SMILES: [2H]C([2H])(Nc1ccc2c(=O)n3c(nc2c1)/C(=N/OC)c1cc(F)ccc1-3)c1ccc(F)cc1] |
| 57 | 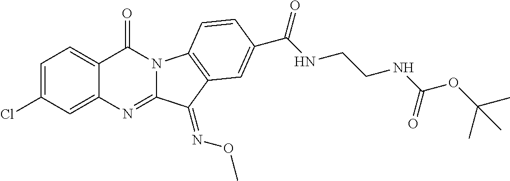 [SMILES: CO/N=C1\c2cc(C(=O)NCCNC(=O)OC(C)(C)C)ccc2-n2c1nc1cc(Cl)ccc1c2=O] |
| 58 | 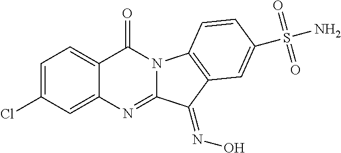 [SMILES: NS(=O)(=O)c1ccc2c(c1)/C(=N\O)c1nc3cc(Cl)ccc3c(=O)n1-2] |
| 59 | 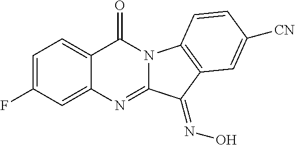 [SMILES: N#Cc1ccc2c(c1)/C(=N\O)c1nc3cc(F)ccc3c(=O)n1-2] |
| 60 | 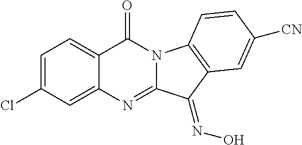 [SMILES: N#Cc1ccc2c(c1)/C(=N\O)c1nc3cc(Cl)ccc3c(=O)n1-2] |
| 61 | 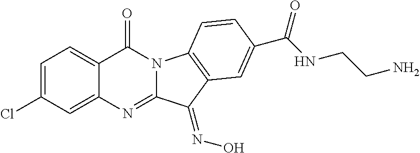 [SMILES: NCCNC(=O)c1ccc2c(c1)/C(=N\O)c1nc3cc(Cl)ccc3c(=O)n1-2] |
| 62 | 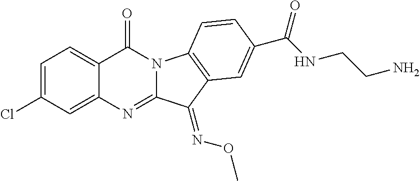 [SMILES: CO/N=C1\c2cc(C(=O)NCCN)ccc2-n2c1nc1cc(Cl)ccc1c2=O] |
| 63 | 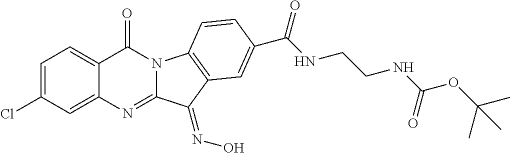 [SMILES: CC(C)(C)OC(=O)NCCNC(=O)c1ccc2c(c1)/C(=N\O)c1nc3cc(Cl)ccc3c(=O)n1-2] |
| 64 | 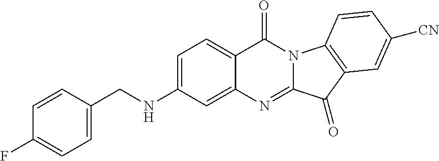 [SMILES: N#Cc1ccc2c(c1)C(=O)c1nc3cc(NCc4ccc(F)cc4)ccc3c(=O)n1-2] |
| 65 | 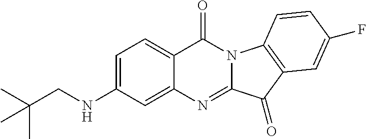 [SMILES: CC(C)(C)CNc1ccc2c(=O)n3c(nc2c1)C(=O)c1cc(F)ccc1-3] |
| 66 | 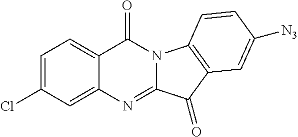 [SMILES: [N-]=[N+]=Nc1ccc2c(c1)C(=O)c1nc3cc(Cl)ccc3c(=O)n1-2] |
| 67 | 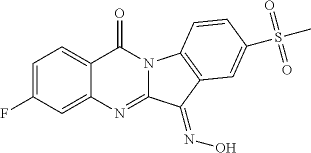 [SMILES: CS(=O)(=O)c1ccc2c(c1)/C(=N\O)c1nc3cc(F)ccc3c(=O)n1-2] |
| 68 | 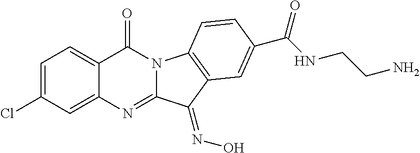 [SMILES: NCCNC(=O)c1ccc2c(c1)/C(=N\O)c1nc3cc(Cl)ccc3c(=O)n1-2] |
| 69 | 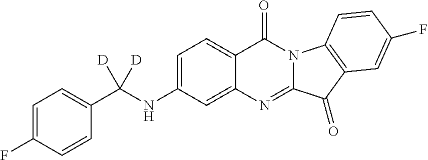 [SMILES: [2H]C([2H])(Nc1ccc2c(=O)n3c(nc2c1)C(=O)c1cc(F)ccc1-3)c1ccc(F)cc1] |
| 70 | 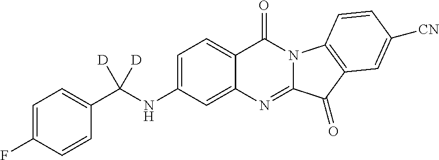 [SMILES: [2H]C([2H])(Nc1ccc2c(=O)n3c(nc2c1)C(=O)c1cc(C#N)ccc1-3)c1ccc(F)cc1] |
| 71 | 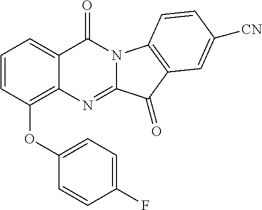 [SMILES: N#Cc1ccc2c(c1)C(=O)c1nc3c(Oc4ccc(F)cc4)cccc3c(=O)n1-2] |
| 72 | 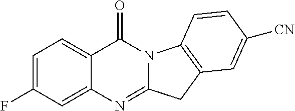 [SMILES: N#Cc1ccc2c(c1)Cc1nc3cc(F)ccc3c(=O)n1-2] |
| 73 | 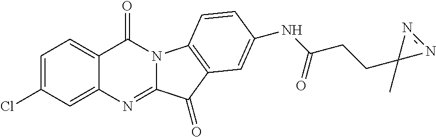 [SMILES: CC1(CCC(=O)Nc2ccc3c(c2)C(=O)c2nc4cc(Cl)ccc4c(=O)n2-3)N=N1] |
| 74 | 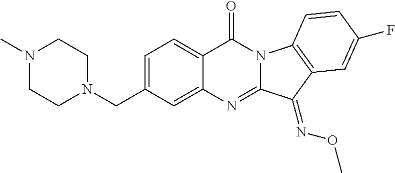 [SMILES: CO/N=C1\c2cc(F)ccc2-n2c1nc1cc(CN3CCN(C)CC3)ccc1c2=O] |
| 75 | 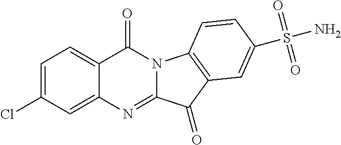 [SMILES: NS(=O)(=O)c1ccc2c(c1)C(=O)c1nc3cc(Cl)ccc3c(=O)n1-2] |
| 76 | 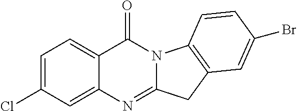 [SMILES: O=c1c2ccc(Cl)cc2nc2n1-c1ccc(Br)cc1C2] |
| 77 | 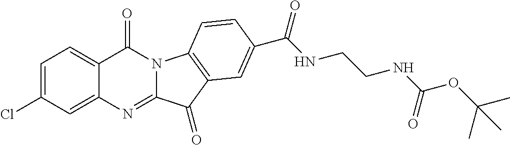 [SMILES: CC(C)(C)OC(=O)NCCNC(=O)c1ccc2c(c1)C(=O)c1nc3cc(Cl)ccc3c(=O)n1-2] |
| 78 | 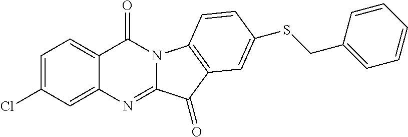 [SMILES: O=C1c2cc(SCc3ccccc3)ccc2-n2c1nc1cc(Cl)ccc1c2=O] |
| 79 | 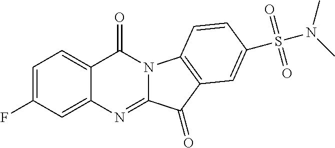 [SMILES: CN(C)S(=O)(=O)c1ccc2c(c1)C(=O)c1nc3cc(F)ccc3c(=O)n1-2] |
| 80 | 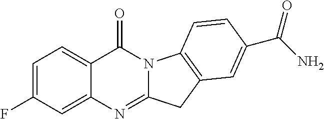 [SMILES: NC(=O)c1ccc2c(c1)Cc1nc3cc(F)ccc3c(=O)n1-2] |
| 81 | 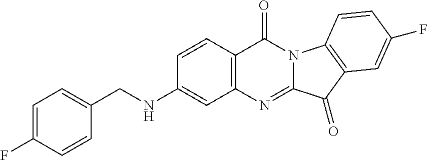 [SMILES: O=C1c2cc(F)ccc2-n2c1nc1cc(NCc3ccc(F)cc3)ccc1c2=O] |
| 82 | 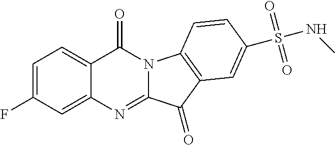 [SMILES: CNS(=O)(=O)c1ccc2c(c1)C(=O)c1nc3cc(F)ccc3c(=O)n1-2] |
| 83 | 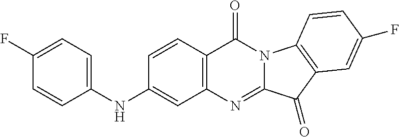 [SMILES: O=C1c2cc(F)ccc2-n2c1nc1cc(Nc3ccc(F)cc3)ccc1c2=O] |
| 84 | 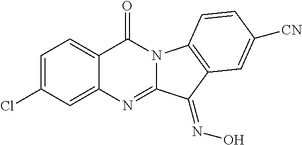 [SMILES: N#Cc1ccc2c(c1)/C(=N\O)c1nc3cc(Cl)ccc3c(=O)n1-2] |
| 85 | 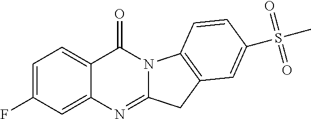 [SMILES: CS(=O)(=O)c1ccc2c(c1)Cc1nc3cc(F)ccc3c(=O)n1-2] |
| 86 | 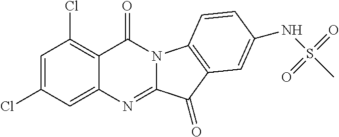 [SMILES: CS(=O)(=O)Nc1ccc2c(c1)C(=O)c1nc3cc(Cl)cc(Cl)c3c(=O)n1-2] |
| 87 | 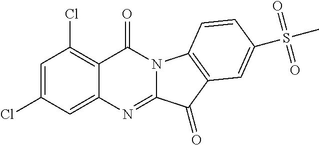 [SMILES: CS(=O)(=O)c1ccc2c(c1)C(=O)c1nc3cc(Cl)cc(Cl)c3c(=O)n1-2] |
| 88 | 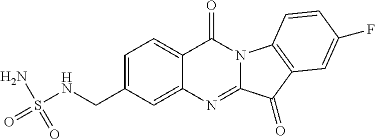 [SMILES: NS(=O)(=O)NCc1ccc2c(=O)n3c(nc2c1)C(=O)c1cc(F)ccc1-3] |
| 89 | 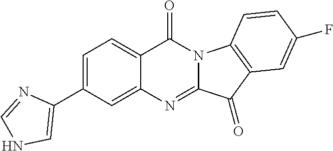 [SMILES: O=C1c2cc(F)ccc2-n2c1nc1cc(-c3c[nH]cn3)ccc1c2=O] |
| 90 | 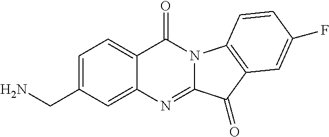 [SMILES: NCc1ccc2c(=O)n3c(nc2c1)C(=O)c1cc(F)ccc1-3] |
| 91 | 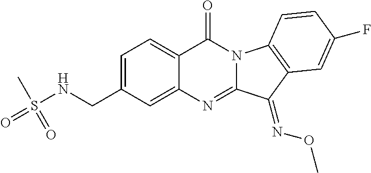 [SMILES: CO/N=C1\c2cc(F)ccc2-n2c1nc1cc(CNS(C)(=O)=O)ccc1c2=O] |
| 92 | 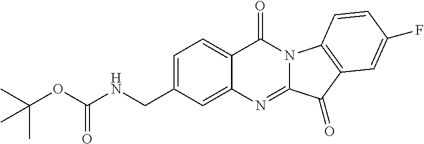 [SMILES: CC(C)(C)OC(=O)NCc1ccc2c(=O)n3c(nc2c1)C(=O)c1cc(F)ccc1-3] |
| 93 | 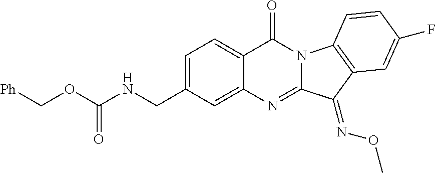 [SMILES: CO/N=C1\c2cc(F)ccc2-n2c1nc1cc(CNC(=O)OCc3ccccc3)ccc1c2=O] |
| 94 | 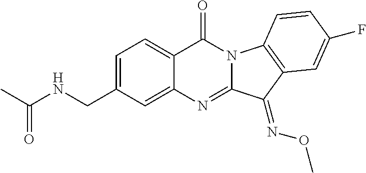 [SMILES: CO/N=C1\c2cc(F)ccc2-n2c1nc1cc(CNC(C)=O)ccc1c2=O] |
| 95 | 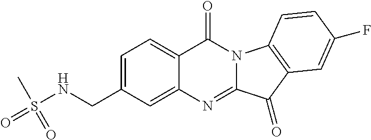 [SMILES: CS(=O)(=O)NCc1ccc2c(=O)n3c(nc2c1)C(=O)c1cc(F)ccc1-3] |
| 96 | 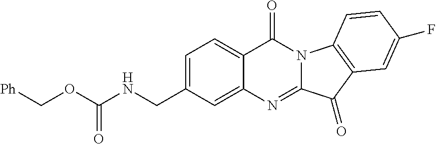 [SMILES: O=C(NCc1ccc2c(=O)n3c(nc2c1)C(=O)c1cc(F)ccc1-3)OCc1ccccc1] |
| 97 | 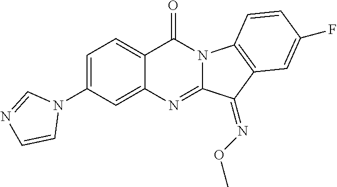 [SMILES: CO/N=C1/c2cc(F)ccc2-n2c1nc1cc(-n3ccnc3)ccc1c2=O] |
| 98 | 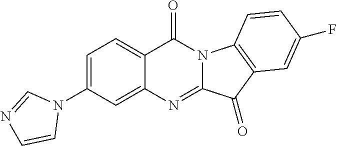 [SMILES: O=C1c2cc(F)ccc2-n2c1nc1cc(-n3ccnc3)ccc1c2=O] |
| 99 | 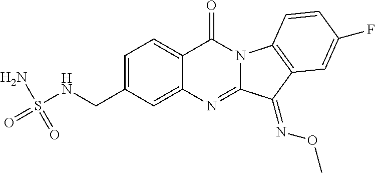 [SMILES: CO/N=C1\c2cc(F)ccc2-n2c1nc1cc(CNS(N)(=O)=O)ccc1c2=O] |
| 100 | 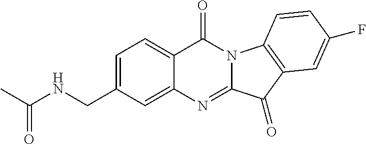 [SMILES: CC(=O)NCc1ccc2c(=O)n3c(nc2c1)C(=O)c1cc(F)ccc1-3] |
| 101 | 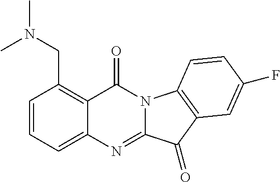 [SMILES: CN(C)Cc1cccc2nc3n(c(=O)c12)-c1ccc(F)cc1C3=O] |
| 102 | 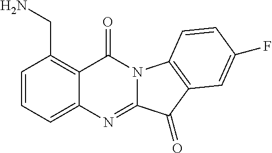 [SMILES: NCc1cccc2nc3n(c(=O)c12)-c1ccc(F)cc1C3=O] |
| 103 | 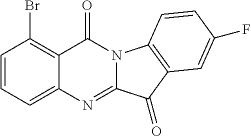 [SMILES: O=C1c2cc(F)ccc2-n2c1nc1cccc(Br)c1c2=O] |
| 104 | 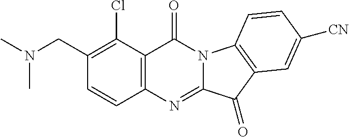 [SMILES: CN(C)Cc1ccc2nc3n(c(=O)c2c1Cl)-c1ccc(C#N)cc1C3=O] |
| 105 | 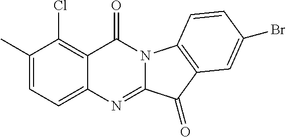 [SMILES: Cc1ccc2nc3n(c(=O)c2c1Cl)-c1ccc(Br)cc1C3=O] |
| 106 | 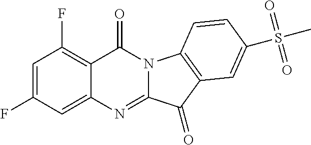 [SMILES: CS(=O)(=O)c1ccc2c(c1)C(=O)c1nc3cc(F)cc(F)c3c(=O)n1-2] |
| 107 | 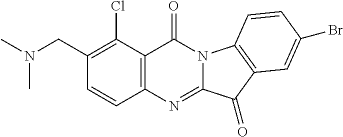 [SMILES: CN(C)Cc1ccc2nc3n(c(=O)c2c1Cl)-c1ccc(Br)cc1C3=O] |
| 108 | 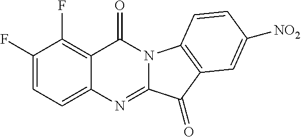 [SMILES: O=C1c2cc([N+](=O)[O-])ccc2-n2c1nc1ccc(F)c(F)c1c2=O] |
| 109 | 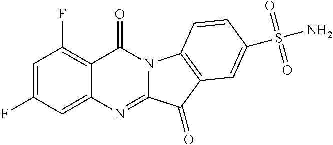 [SMILES: NS(=O)(=O)c1ccc2c(c1)C(=O)c1nc3cc(F)cc(F)c3c(=O)n1-2] |
| 110 | 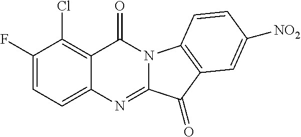 [SMILES: O=C1c2cc([N+](=O)[O-])ccc2-n2c1nc1ccc(F)c(Cl)c1c2=O] |
| 111 | 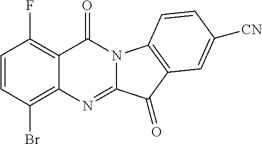 [SMILES: N#Cc1ccc2c(c1)C(=O)c1nc3c(Br)ccc(F)c3c(=O)n1-2] |
| 112 | 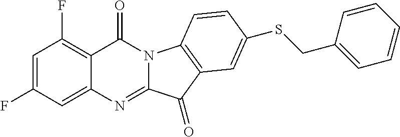 [SMILES: O=C1c2cc(SCc3ccccc3)ccc2-n2c1nc1cc(F)cc(F)c1c2=O] |
| 113 | 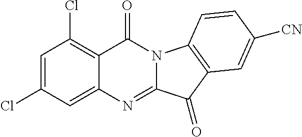 [SMILES: N#Cc1ccc2c(c1)C(=O)c1nc3cc(Cl)cc(Cl)c3c(=O)n1-2] |
| 114 | 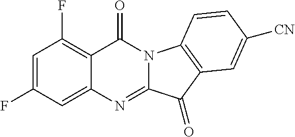 [SMILES: N#Cc1ccc2c(c1)C(=O)c1nc3cc(F)cc(F)c3c(=O)n1-2] |
| 115 | 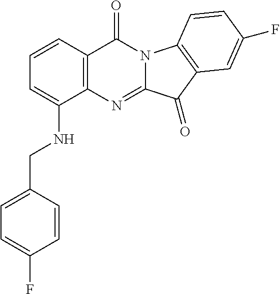 [SMILES: O=C1c2cc(F)ccc2-n2c1nc1c(NCc3ccc(F)cc3)cccc1c2=O] |
| 116 | 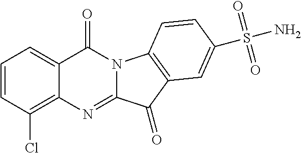 [SMILES: NS(=O)(=O)c1ccc2c(c1)C(=O)c1nc3c(Cl)cccc3c(=O)n1-2] |
| 117 | 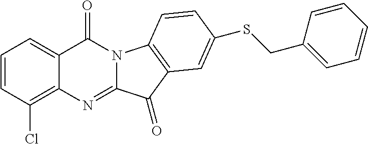 [SMILES: O=C1c2cc(SCc3ccccc3)ccc2-n2c1nc1c(Cl)cccc1c2=O] |
| 118 | 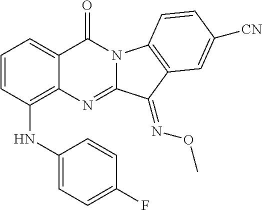 [SMILES: CO/N=C1\c2cc(C#N)ccc2-n2c1nc1c(Nc3ccc(F)cc3)cccc1c2=O] |
| 119 | 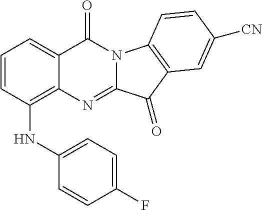 [SMILES: N#Cc1ccc2c(c1)C(=O)c1nc3c(Nc4ccc(F)cc4)cccc3c(=O)n1-2] |
| 120 | 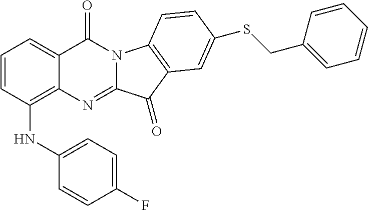 [SMILES: O=C1c2cc(SCc3ccccc3)ccc2-n2c1nc1c(Nc3ccc(F)cc3)cccc1c2=O] |
| 121 | 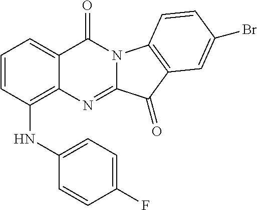 [SMILES: O=C1c2cc(Br)ccc2-n2c1nc1c(Nc3ccc(F)cc3)cccc1c2=O] |
| 122 | 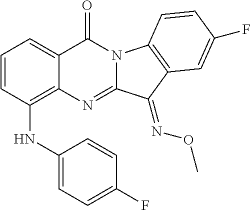 [SMILES: CO/N=C1\c2cc(F)ccc2-n2c1nc1c(Nc3ccc(F)cc3)cccc1c2=O] |
| 123 | 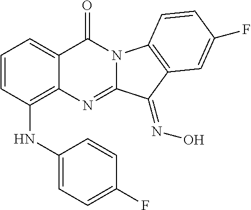 [SMILES: O=c1c2cccc(Nc3ccc(F)cc3)c2nc2n1-c1ccc(F)cc1/C2=N\O] |
| 124 | 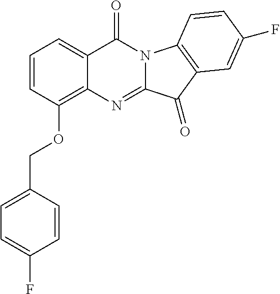 [SMILES: O=C1c2cc(F)ccc2-n2c1nc1c(OCc3ccc(F)cc3)cccc1c2=O] |
| 125 | 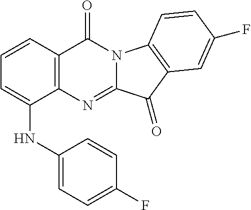 [SMILES: O=C1c2cc(F)ccc2-n2c1nc1c(Nc3ccc(F)cc3)cccc1c2=O] |
| 126 | 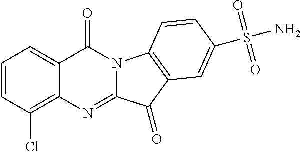 [SMILES: NS(=O)(=O)c1ccc2c(c1)C(=O)c1nc3c(Cl)cccc3c(=O)n1-2] |
| 127 | 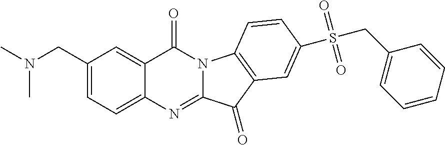 [SMILES: CN(C)Cc1ccc2nc3n(c(=O)c2c1)-c1ccc(S(=O)(=O)Cc2ccccc2)cc1C3=O] |
| 128 | 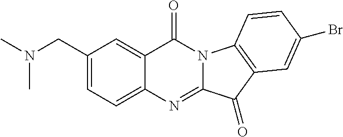 [SMILES: CN(C)Cc1ccc2nc3n(c(=O)c2c1)-c1ccc(Br)cc1C3=O] |
| 129 | 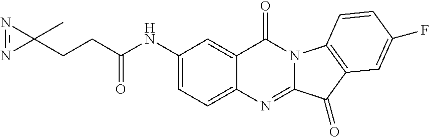 [SMILES: CC1(CCC(=O)Nc2ccc3nc4n(c(=O)c3c2)-c2ccc(F)cc2C4=O)N=N1] |
| 130 | 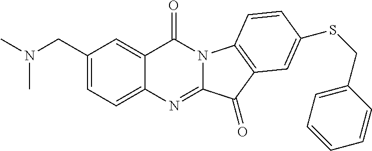 [SMILES: CN(C)Cc1ccc2nc3n(c(=O)c2c1)-c1ccc(SCc2ccccc2)cc1C3=O] |
| 131 | 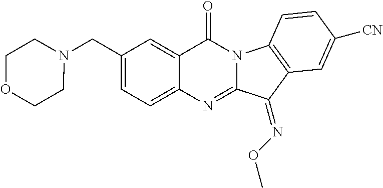 [SMILES: CO/N=C1/c2cc(C#N)ccc2-n2c1nc1ccc(CN3CCOCC3)cc1c2=O] |
| 132 | 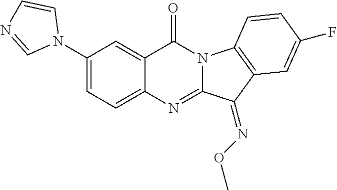 [SMILES: CO/N=C1/c2cc(F)ccc2-n2c1nc1ccc(-n3ccnc3)cc1c2=O] |
| 133 | 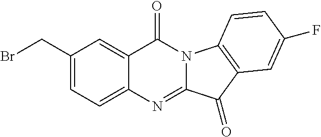 [SMILES: O=C1c2cc(F)ccc2-n2c1nc1ccc(CBr)cc1c2=O] |
| 134 | 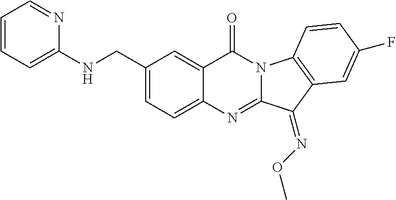 [SMILES: CO/N=C1/c2cc(F)ccc2-n2c1nc1ccc(CNc3ccccn3)cc1c2=O] |
| 135 | 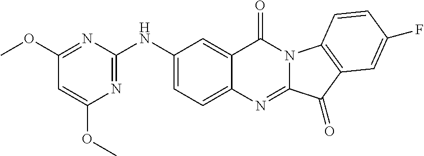 [SMILES: COc1cc(OC)nc(Nc2ccc3nc4n(c(=O)c3c2)-c2ccc(F)cc2C4=O)n1] |
| 136 | 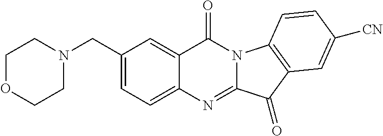 [SMILES: N#Cc1ccc2c(c1)C(=O)c1nc3ccc(CN4CCOCC4)cc3c(=O)n1-2] |
| 137 | 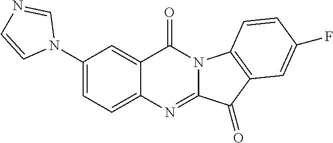 [SMILES: O=C1c2cc(F)ccc2-n2c1nc1ccc(-n3ccnc3)cc1c2=O] |
| 138 | 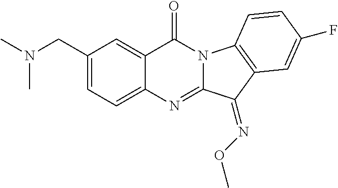 [SMILES: CO/N=C1/c2cc(F)ccc2-n2c1nc1ccc(CN(C)C)cc1c2=O] |
| 139 | 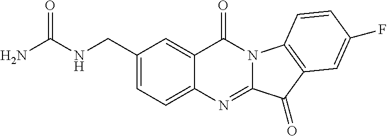 [SMILES: NC(=O)NCc1ccc2nc3n(c(=O)c2c1)-c1ccc(F)cc1C3=O] |
| 140 | 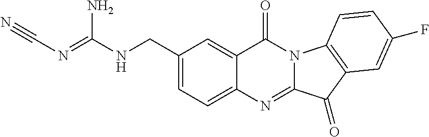 [SMILES: N#C/N=C(\N)NCc1ccc2nc3n(c(=O)c2c1)-c1ccc(F)cc1C3=O] |
| 141 | 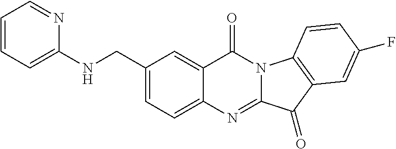 [SMILES: O=C1c2cc(F)ccc2-n2c1nc1ccc(CNc3ccccn3)cc1c2=O] |
| 142 | 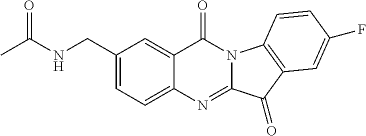 [SMILES: CC(=O)NCc1ccc2nc3n(c(=O)c2c1)-c1ccc(F)cc1C3=O] |
| 143 | 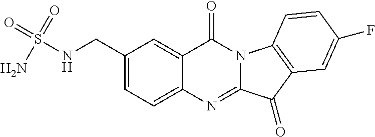 [SMILES: NS(=O)(=O)NCc1ccc2nc3n(c(=O)c2c1)-c1ccc(F)cc1C3=O] |
| 144 | 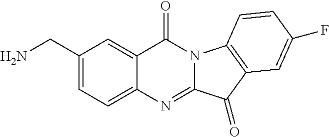 [SMILES: NCc1ccc2nc3n(c(=O)c2c1)-c1ccc(F)cc1C3=O] |
| 145 | 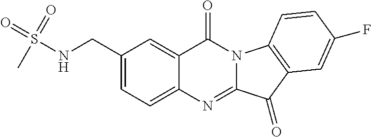 [SMILES: CS(=O)(=O)NCc1ccc2nc3n(c(=O)c2c1)-c1ccc(F)cc1C3=O] |
| 146 | 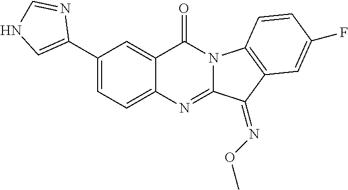 [SMILES: CO/N=C1/c2cc(F)ccc2-n2c1nc1ccc(-c3c[nH]cn3)cc1c2=O] |
| 147 | 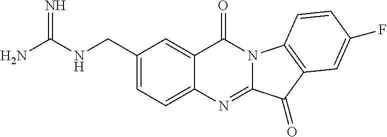 [SMILES: N=C(N)NCc1ccc2nc3n(c(=O)c2c1)-c1ccc(F)cc1C3=O] |
| 148 | 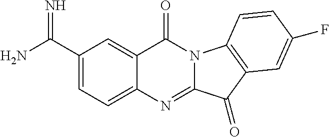 [SMILES: N=C(N)c1ccc2nc3n(c(=O)c2c1)-c1ccc(F)cc1C3=O] |
| 149 | 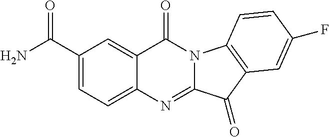 [SMILES: NC(=O)c1ccc2nc3n(c(=O)c2c1)-c1ccc(F)cc1C3=O] |
| 150 | 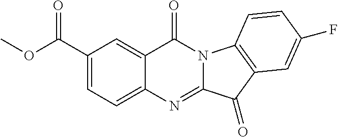 [SMILES: COC(=O)c1ccc2nc3n(c(=O)c2c1)-c1ccc(F)cc1C3=O] |
| 151 | 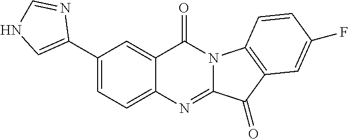 [SMILES: O=C1c2cc(F)ccc2-n2c1nc1ccc(-c3c[nH]cn3)cc1c2=O] |
| 152 | 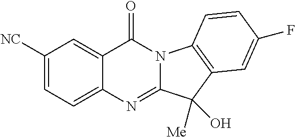 [SMILES: CC1(O)c2cc(F)ccc2-n2c1nc1ccc(C#N)cc1c2=O] |
| 153 | 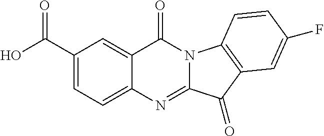 [SMILES: O=C(O)c1ccc2nc3n(c(=O)c2c1)-c1ccc(F)cc1C3=O] |
| 154 | 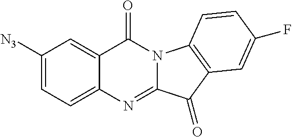 [SMILES: [N-]=[N+]=Nc1ccc2nc3n(c(=O)c2c1)-c1ccc(F)cc1C3=O] |
| 155 | 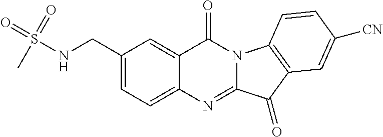 [SMILES: CS(=O)(=O)NCc1ccc2nc3n(c(=O)c2c1)-c1ccc(C#N)cc1C3=O] |
| 156 | 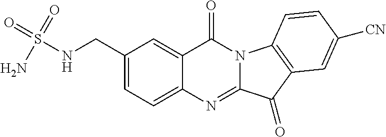 [SMILES: N#Cc1ccc2c(c1)C(=O)c1nc3ccc(CNS(N)(=O)=O)cc3c(=O)n1-2] |
| 157 | 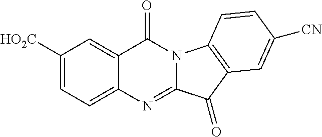 [SMILES: N#Cc1ccc2c(c1)C(=O)c1nc3ccc(C(=O)O)cc3c(=O)n1-2] |
| 158 | 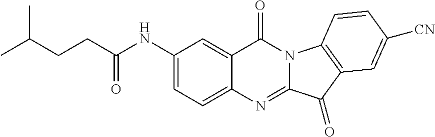 [SMILES: CC(C)CCC(=O)Nc1ccc2nc3n(c(=O)c2c1)-c1ccc(C#N)cc1C3=O] |
| 160 | 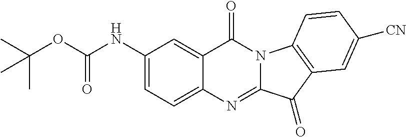 [SMILES: CC(C)(C)OC(=O)Nc1ccc2nc3n(c(=O)c2c1)-c1ccc(C#N)cc1C3=O] |
| 161 | 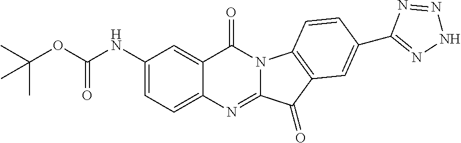 [SMILES: CC(C)(C)OC(=O)Nc1ccc2nc3n(c(=O)c2c1)-c1ccc(-c2nn[nH]n2)cc1C3=O] |
| 162 | 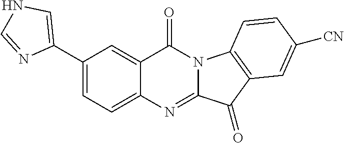 [SMILES: N#Cc1ccc2c(c1)C(=O)c1nc3ccc(-c4c[nH]cn4)cc3c(=O)n1-2] |
| 163 | 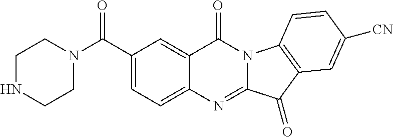 [SMILES: N#Cc1ccc2c(c1)C(=O)c1nc3ccc(C(=O)N4CCNCC4)cc3c(=O)n1-2] |
| 164 | 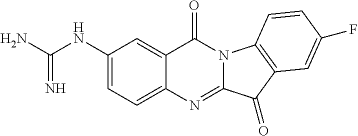 [SMILES: N=C(N)Nc1ccc2nc3n(c(=O)c2c1)-c1ccc(F)cc1C3=O] |
| 165 | 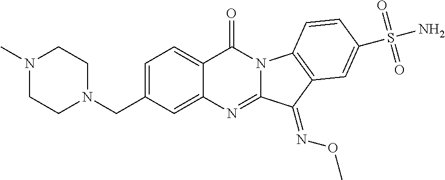 [SMILES: CO/N=C1\c2cc(S(N)(=O)=O)ccc2-n2c1nc1cc(CN3CCN(C)CC3)ccc1c2=O] |
| 166 | 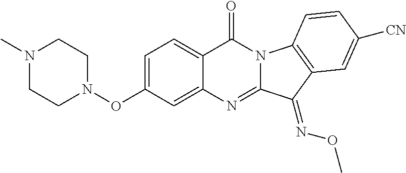 [SMILES: CO/N=C1\c2cc(C#N)ccc2-n2c1nc1cc(ON3CCN(C)CC3)ccc1c2=O] |
| 167 | 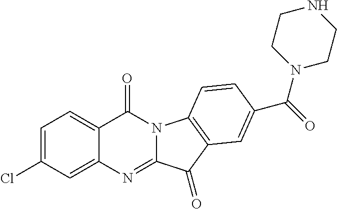 [SMILES: O=C1c2cc(C(=O)N3CCNCC3)ccc2-n2c1nc1cc(Cl)ccc1c2=O] |
| 168 | 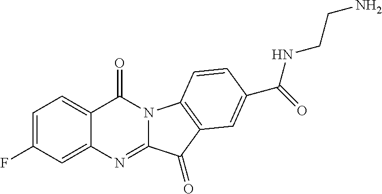 [SMILES: NCCNC(=O)c1ccc2c(c1)C(=O)c1nc3cc(F)ccc3c(=O)n1-2] |
| 169 | 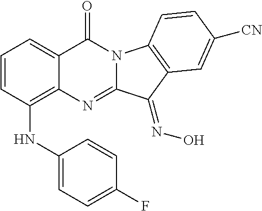 [SMILES: N#Cc1ccc2c(c1)/C(=N\O)c1nc3c(Nc4ccc(F)cc4)cccc3c(=O)n1-2] |
| 170 | 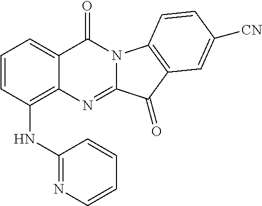 [SMILES: N#Cc1ccc2c(c1)C(=O)c1nc3c(Nc4ccccn4)cccc3c(=O)n1-2] |
| 171 | 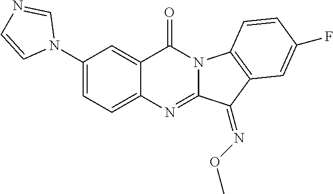 [SMILES: CO/N=C1/c2cc(F)ccc2-n2c1nc1ccc(-n3ccnc3)cc1c2=O] |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formulas (I), (II), (III), and (IV) are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as CAN- or (S)-. The present chemical entities, pharmaceutical compositions and methods are meant to include all such possible stereoisomers, including racemic mixtures, optically pure forms, mixtures of diastereomers and intermediate mixtures. Optically active CAN- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formulas (I), (II), (III), and (IV) herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can b e made by one of ordinary skill in the art by routine experimentation. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formulas (I), (II), (III), and (IV), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formulas (I), (II), (III), and (IV), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

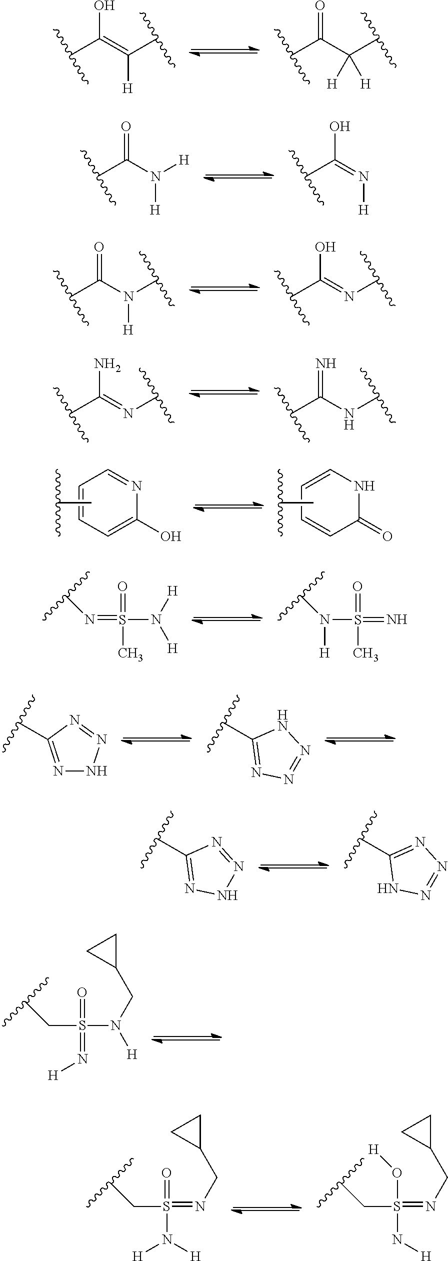

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formulas (I), (II), (III), and (IV). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formulas (I), (II), (III), and (IV), include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formulas (I), (II), (III), and (IV), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formulas (I), (II), (III), and (IV). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formulas (I), (II), (III) and (IV), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formulas (I), (II), (III), or (IV) and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Methods for formulation of the conjugates can include formulating any of the compounds, salts or conjugates with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates can be lyophilized or in powder form for re-constitution with a suitable vehicle, e.g., sterile pyrogen-free water, before use.

Pharmaceutical compositions can comprise at least one active ingredient (e.g., a compound, salt or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further can comprise more than one active compound (e.g., a compound, salt or conjugate and other agents) as necessary for the particular indication being treated. The active compounds can have complementary activities that do not adversely affect each other. For example, the composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished, for example, by filtration through sterile filtration.

The compositions can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions can be lyophilized or in powder form for reconstitution with a suitable vehicle, e.g., sterile pyrogen-free water, before use.

For parenteral administration, the compounds, salts or conjugates can be formulated in a unit dosage injectable form (e.g., solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the compound, salt or conjugate, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations can be prepared for storage by mixing a compound, salt or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

A compound or salt of any one of Formulas (I), (II), (III), and (IV) may be formulated in any suitable pharmaceutical formulation. A pharmaceutical formulation of the present disclosure typically contains an active ingredient (e.g., compound or salt of any one of Formulas (I), (II), (III) and (IV)), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

In certain embodiments, a compound or salt of Formulas (I), (II), (III), or (IV) is formulated with a chelating agent or other material capable of binding metal ions, such as ethylene diamine tetra acetic acid (EDTA) and its salts are capable of enhancing the stability of a compound or salt of Formulas (I), (II), (III), or (IV).

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of compound or salt of any one of Formulas (I), (II), (III), and (IV) will be dependent on the mammal being treated, the severity of the disorder or condition, the rate of administration, the disposition of the compound or salt of any one of Formulas (I), (II), (III), and (IV) and the discretion of the prescribing physician.

In some embodiments, the disclosure provides a pharmaceutical composition for oral administration containing at least one compound or salt of any one of Formulas (I), (II), (III), and (IV) and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formulas (I), (II), (III), and (IV) moistened with an inert liquid diluent.

In some embodiments, the disclosure provides a pharmaceutical composition for injection containing a compound or salt of any one of Formulas (I), (II), (III), and (IV) disclosed herein and a pharmaceutical excipient suitable for injection. Components and amounts of agents in the composition are as described herein.

In certain embodiments, the compound or salt of any one of Formulas (I), (II), (III), and (IV) may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of Formulas (I), (II), (III), and (IV), and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20^{th} Ed., Lippincott Williams & Wilkins, 2000; Martindale, The Extra Pharmacopoeia, Thirty-Second Edition (The Pharmaceutical Press, London, 1999).

### Methods of Treatment

In some embodiments, the compounds described herein are used in the preparation of medicaments for the prevention or treatment of diseases or conditions. In some embodiments, the present disclosure provides a method for treating any of the diseases or conditions described herein in a subject in need of such treatment, the method comprising administration of pharmaceutical compositions containing at least one compound of Formulas (I), (II), (III), or (IV), or a pharmaceutically acceptable salt thereof, in therapeutically effective amounts to said subject. In some embodiments, the disease or condition is cancer. In some embodiments, the disease or condition is a neurological disorder.

In some embodiments, the present disclosure provides a method of treating a subject in need thereof comprising administering to the subject a therapeutically effective amount of a compound of Formulas (I), (II), (III), or (IV). As used herein, the term “therapeutically effective amount” means the amount of an inhibitor that is sufficient to modulate the activity of IDO2 in a subject or in a cell.

A compound of the present disclosure may be administered to the subject using various different administration routes, including oral, rectal, transmucosal, topical, transdermal, inhalation, intravenous, subcutaneous, intradermal, intramuscular, intra-articular, intrathecal, intraventricular, intravenous, intraperitoneal, intranasal, or intraocular routes of administration.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

When used in a patient, effective amounts for this use will depend on the severity and course of the disease, disorder or condition, previous therapy, the patient's health status and response to the drugs, and the judgment of the treating physician.

In some embodiments, the present disclosure provides a method of modulating IDO2 in a subject in need thereof, comprising administering to the subject a compound of Formulas (I), (II), (III), or (IV), or pharmaceutically acceptable salt thereof. In some embodiments, the present disclosure provides a method of modulating IDO2 in a subject in need thereof, comprising administering to the subject a pharmaceutical composition comprising a compound of Formulas (I), (II), (III), or (IV), or a pharmaceutically acceptable salt thereof, and an excipient (e.g., a pharmaceutically acceptable excipient).

In some embodiments, the present disclosure provides a method of treating a disease or condition comprising administering to a subject in need thereof a compound of Formulas (I), (II), (III), or (IV), or pharmaceutically acceptable salt thereof. In some embodiments, the present disclosure provides a method of treating a disease or condition comprising administering to a subject in need thereof a pharmaceutical composition comprising a compound of Formulas (I), (II), (III), (IV), or a pharmaceutically acceptable salt thereof, and an excipient. In some embodiments, the disease or condition is osteoarthritis.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-154 show general and exemplary procedures for the preparation of the claimed compounds for Formulas (I), (II), (III), and (IV).

Abbreviations: CAN acetonitrile; AIBN azobisisobutyronitrile; BTC bis(trichloromethyl) carbonate; m-CPBA meta-chloroperoxybenzoic acid; DCM dichloromethane; DIPEA N,N-diisopropylethylamine; DMF dimethylformamide; DMSO dimethylsulfoxide; EA ethyl acetate; HATU 1-[bis(dimethylamino)methylene]-1H-1,2,3-triazolo[4,5-b]pyridinium 3-oxid hexafluorophosphate; IBX 2-iodoxybenzoic acid; NBS N-bromosuccinimide; NIS N-iodosuccinimide; PE petroleum ether; RT room temperature; TFA trifluoroacetic acid.

Reaction progress was monitored by LCMS or TLC or HPLC. Chromatography was performed by flash column chromatography using silica gel and the solvents listed.

### General Procedure 1

### Example 1: 8-Bromo-3-fluoroindolo[2,1-b]quinazoline-6,12-dione (Compound 18)

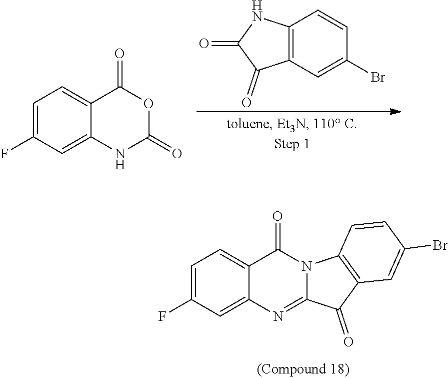 [SMILES: O=c1[nH]c2cc(F)ccc2c(=O)o1 ; O=C1Nc2ccc(Br)cc2C1=O ; O=C1c2cc(Br)ccc2-n2c1nc1cc(F)ccc1c2=O]

To a solution of 7-fluoro-1H-3,1-benzoxazine-2,4-dione (500 mg, 2.8 mmol) and 5-bromo-1H-indole-2,3-dione (624 mg, 2.8 mmol) in toluene (5.0 mL) was added Et₃N (838 mg, 8.3 mmol). The mixture was stirred at 110° C. for 12 hours and concentrated under reduced pressure. The residue was dissolved into EA and the suspension was filtered under reduced pressure. The filtered cake was washed with MeOH, DCM, and EA and dried under reduced pressure to give 8-bromo-3-fluoroindolo[2,1-b]quinazoline-6,12-dione (195 mg, 20% yield) as a yellow solid. ¹H NMR (400 MHz, DMSO-d₆) δ 8.62 (d, J=1.2 Hz, 1H), 8.39 (dd, J=8.8, 6.0 Hz, 1H), 7.91-7.79 (m, 2H), 7.73 (d, J=8.0 Hz, 1H), 7.64 (d, J=2.4 Hz, 1H). LC-MS: m/z [M+H]⁺ 344.9.

### General Procedure 2

### Example 2: 2-Methyl-8-nitroindolo[2,1-b]quinazoline-6,12-dione (Compound 52)

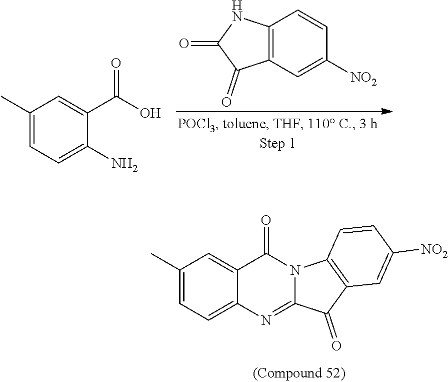 [SMILES: Cc1ccc(N)c(C(=O)O)c1 ; O=C1Nc2ccc([N+](=O)[O-])cc2C1=O ; Cc1ccc2nc3n(c(=O)c2c1)-c1ccc([N+](=O)[O-])cc1C3=O]

To a solution of 5-nitro-1H-indole-2,3-dione (635.5 mg, 3.307 mmol) in toluene (8 mL) and THF (2 mL) was added phosphoryl trichloride (2535.85 mg, 16.53 mmol) under N₂ at 70° C. The mixture was stirred at 70° C. for 30 minutes. Then a solution of 2-amino-5-methylbenzoic acid (500 mg, 3.307 mmol) in THF (2 mL) was added. The mixture was heated at 110° C. for 2 hours. The mixture was concentrated under reduced pressure. The residue was triturated with CAN. The precipitate was filtered and the filter cake was purified by prep-HPLC (Column: Xbridge prep-C18, 150*19 mm 5 μm; Mobile phase: CAN-H₂O (0.05% TFA); Gradient: 5% to 95%; Flow rate: 20 mL/min) to give 2-methyl-8-nitroindolo[2,1-b]quinazoline-6,12-dione (6 mg, 1% yield) as a brown solid. ¹H NMR (400 MHz, DMSO-d₆) δ 8.75-8.67 (m, 2H), 8.55 (d, J=2.0 Hz, 1H), 8.18 (s, 1H), 7.90 (d, J=8.4 Hz, 1H), 7.83-7.80 (m, 1H), 2.54 (s, 3H). LC-MS: m/z [M+H]⁺ 308.0.

### General Procedure 3

### Example 3: 3-Methyl-8-nitroindolo[2,1-b]quinazoline-6,12-dione (Compound 1)

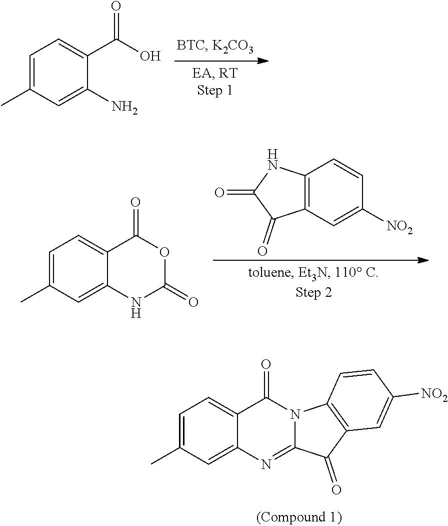 [SMILES: Cc1ccc(C(=O)O)c(N)c1 ; Cc1ccc2c(=O)oc(=O)[nH]c2c1 ; O=C1Nc2ccc([N+](=O)[O-])cc2C1=O ; Cc1ccc2c(=O)n3c(nc2c1)C(=O)c1cc([N+](=O)[O-])ccc1-3]

### Step 1: 7-methyl-2H-benzo[d][1,3]oxazine-2,4(1H)-dione

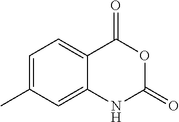

To a solution 2-amino-4-methylbenzoic acid (200 mg, 1.3 mmol) in EA (5.0 mL) was added K₂CO₃(364 mg, 2.6 mmol) and BTC (154 mg, 0.5 mmol). The mixture was stirred for 3 h at 25° C. The mixture was filtered to afford 7-methyl-2H-benzo[d][1,3]oxazine-2,4(1H)-dione (170 mg, crude) as a white solid. LC-MS: m/z [M+H]⁺ 178.1.

### Step 2: 3-methyl-8-nitroindolo[2,1-b]quinazoline-6,12-dione (Compound 1)

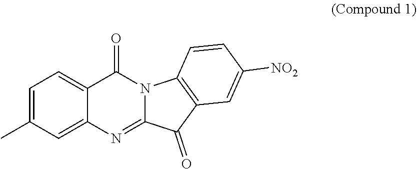 [SMILES: Cc1ccc2c(=O)n3c(nc2c1)C(=O)c1cc([N+](=O)[O-])ccc1-3]

To a solution of 7-methyl-2H-benzo[d][1,3]oxazine-2,4(1H)-dione (200 mg, 1.1 mmol) and 5-nitroindoline-2,3-dione (218 mg, 1.1 mmol) in toluene (5.0 mL) was added Me₃N (345 mg, 3.4 mmol). The mixture was stirred for 5.0 h at 110° C., and the solvent was removed under reduced pressure. The solid was washed with DCM, EA, MeOH and dried under reduced pressure to afford 3-methyl-8-nitroindolo[2,1-b]quinazoline-6,12-dione (140 mg, 40% yield) as a yellow solid. ¹H NMR (400 MHz, DMSO-d₆) δ 8.78-8.68 (m, 2H), 8.58 (d, J=2.0 Hz, 1H), 8.29 (d, J=8.0 Hz, 1H), 7.85 (s, 1H), 7.65 (d, J=8.0 Hz, 1H), 2.60-2.56 (m, 3H). LC-MS: m/z [M+H]⁺ 308.0.

### General Procedure 4

### Example 4: 8-(Trifluoromethyl)indolo[2,1-b]quinazoline-6,12-dione (Compound 53)

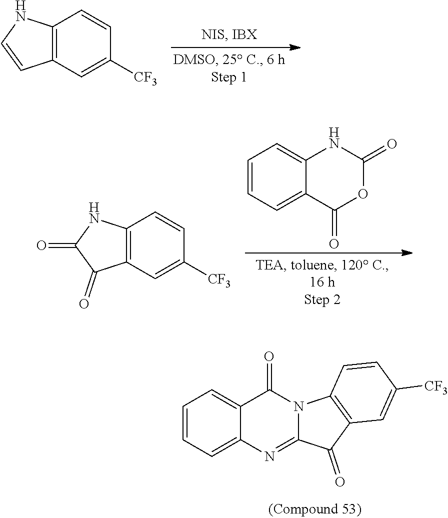 [SMILES: FC(F)(F)c1ccc2[nH]ccc2c1 ; O=C1Nc2ccc(C(F)(F)F)cc2C1=O ; O=c1[nH]c2ccccc2c(=O)o1 ; O=C1c2cc(C(F)(F)F)ccc2-n2c1nc1ccccc1c2=O]

### Step 1: 5-(trifluoromethyl)indoline-2,3-dione

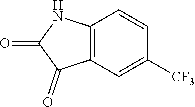 [SMILES: O=C1Nc2ccc(C(F)(F)F)cc2C1=O]

A solution of 5-(trifluoromethyl)-1H-indole (500 mg, 2.69 mmol), NIS (906 mg, 4.03 mmol) and IBX (2256 mg, 8.05 mmol) in DMSO (10 mL) was stirred under N₂ at 25° C. for 6 h. The reaction was diluted with EtOAc (50 mL). The combined organic phase was washed with saturated sodium thiosulfate solution (50 mL×3) and brine (50 mL×2), dried over Na₂SO₄, and filtered. The filtrate was concentrated under reduced pressure to give 5-(trifluoromethyl)indoline-2,3-dione (800 mg, crude) as yellow solid, which was used directly in the next step without purification. ¹H NMR (400 MHz, DMSO-d₆) δ 11.68-11.40 (m, 1H), 7.92 (d, J=8.0 Hz, 1H), 7.79 (s, 1H), 7.10 (d, J=8.0 Hz, 1H). LC-MS: m/z [M+H]⁺ 216.0.

### Step 2: 8-(trifluoromethyl)indolo[2,1-b]quinazoline-6,12-dione (Compound 53)

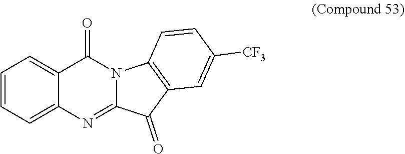 [SMILES: O=C1c2cc(C(F)(F)F)ccc2-n2c1nc1ccccc1c2=O]

To a solution of 5-(trifluoromethyl)indoline-2,3-dione (200 mg, 0.93 mmol) and 1H-3,1-benzoxazine-2,4-dione (151 mg, 0.93 mmol) in toluene (5 mL) was added Et₃N (468 mg, 4.62 mmol). Then, the mixture was stirred for 1.75 h at 110° C. under N₂ atmosphere. The mixture was concentrated, and the residue was purified by recrystallization (DMSO) to give 8-(trifluoromethyl)indolo[2,1-b]quinazoline-6,12-dione (67.8 mg, 22% yield) as a yellow solid. ¹H NMR (400 MHz, CDCl₃) δ 8.79 (d, J=8.4 Hz, 1H), 8.45 (dd, J=8.0, 1.2 Hz, 1H), 8.20-8.14 (m, 1H), 8.09-8.02 (m, 2H), 7.94-7.85 (m, 1H), 7.76-7.67 (m, 1H). LC-MS: m/z [M+H]⁺ 317.1.

### General Procedure 5

### Example 5: 3-Chloro-6,12-dioxo-6,12-dihydroindolo[2,1-b]quinazoline-8-carboxylic Acid (Compound 4)

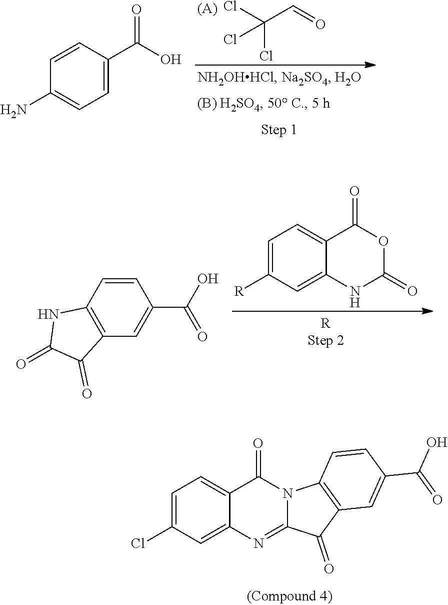

### Step 1: 2,3-dioxo-1H-indole-5-carboxylic Acid

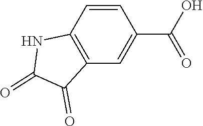 [SMILES: O=C1Nc2ccc(C(=O)O)cc2C1=O]

To a solution of hydroxylamine hydrochloride (9.3 g, 0.13 mol) and Na₂SO₄ (42 g, 0.29 mol) in H₂O (200 mL) was added 2,2,2-trichloroacetaldehyde (7.2 g, 44.4 mmol), and then 4-aminobenzoic acid (5.1 g, 37 mmol, in 100 ml of H₂O) and HCl (13 mL, 2 M) were added. The mixture was stirred for 2 h at 100° C. After cooling to 25° C., the suspension was filtered under reduced pressure. The filter cake was washed with H₂O and dried under reduced pressure to afford 4-[(2E)-2-(N-hydroxyimino)acetamido]benzoic acid (4.5 g, crude) as a white solid. This acid (4.5 g, 0.022 mol) dissolved in H₂SO₄ (15 ml) was heated to 50° C. and stirred for 5 h. The solution was poured into ice-water and extracted with EA (100 mL×2). The combined organic layers were dried over Na₂SO₄, filtered and concentrated under reduced pressure to afford 2,3-dioxo-1H-indole-5-carboxylic acid (1.5 g, crude) as a yellow solid, which would be used in the next step without purification. LC-MS: m/z [M+H]⁺ 192.1.

### Step 2: 3-chloro-6,12-dioxo-6,12-dihydroindolo[2,1-b]quinazoline-8-carboxylic Acid (Compound 4)

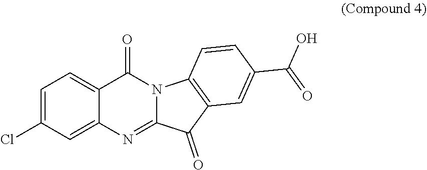 [SMILES: O=C(O)c1ccc2c(c1)C(=O)c1nc3cc(Cl)ccc3c(=O)n1-2]

To a solution of 7-chloro-2H-benzo[d][1,3]oxazine-2,4(1H)-dione (100 mg, 0.51 mmol) in toluene (5.0 mL) was added 2,3-dioxoindoline-5-carboxylic acid (117 mg, 0.61 mmol) and Me₃N (154 mg, 1.5 mmol). The mixture was stirred for 5 h at 110° C. then the solvent was removed under reduced pressure. The solid was purified by prep-HPLC (Chromatographic column:—Xbridge-C18 150×19 mm, 5 μm; Mobile Phase: CAN/H₂O (0.1% TFA)) to give 3-chloro-6,12-dioxo-6,12-dihydroindolo[2,1-b]quinazoline-8-carboxylic acid (10 mg, 6.1% yield) as a yellow solid. ¹H NMR (400 MHz, DMSO-d₆) δ 13.49 (br, 1H), 8.57 (d, J=8.4 Hz, 1H), 8.43 (dd, J=8.4, 1.6 Hz, 1H), 8.34 (d, J=8.4 Hz, 1H), 8.26 (d, J=1.6 Hz, 1H), 8.11 (d, J=2.0 Hz, 1H), 7.81 (dd, J=8.4, 2.0 Hz, 1H).

### Example 6: 4-Chloro-8-nitroindolo[2,1-b]quinazoline-6,12-dione (Compound 36)

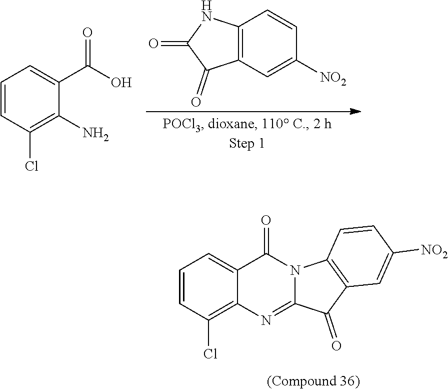 [SMILES: Nc1c(Cl)cccc1C(=O)O ; O=C1Nc2ccc([N+](=O)[O-])cc2C1=O ; O=C1c2cc([N+](=O)[O-])ccc2-n2c1nc1c(Cl)cccc1c2=O]

Following the method of General Procedure 2 but using 2-amino-3-chlorobenzoic acid as starting material. The title compound was obtained as a yellow solid. ¹H NMR (400 MHz, CDCl₃) δ 8.85 (d, J=8.0 Hz, 1H), 8.79 (d, J=4.0 Hz, 1H), 8.72-8.68 (m, 1H), 8.39 (d, J=8.0 Hz, 1H), 7.99-7.96 (m, 1H), 7.66 (t, J=8.0 Hz, 1H). LC-MS: m/z [M+H]⁺ 328.0.

### Example 7: 2-Fluoro-8-nitroindolo[2,1-b]quinazoline-6,12-dione (Compound 54)

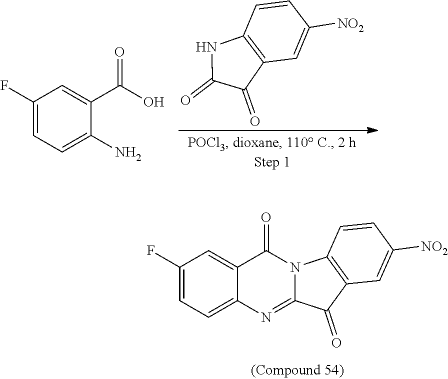

Following the method of General Procedure 2 but using 2-amino-5-fluorobenzoic acid as starting material. The title compound was obtained as a yellow solid. ¹H NMR (400 MHz, DMSO-d₆) δ 8.79-8.64 (m, 2H), 8.58 (s, 1H), 8.15-8.06 (m, 2H), 7.95-7.86 (m, 1H). ¹⁹H NMR (376.5 MHz, DMSO) δ−108.1. LC-MS: m/z [M+H]⁺ 312.0.

### Example 8: 3-Fluoro-8-nitroindolo[2,1-b]quinazoline-6,12-dione (Compound 2)

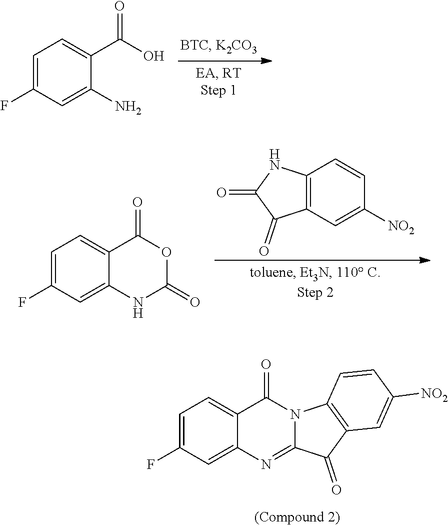 [SMILES: Nc1cc(F)ccc1C(=O)O ; O=c1[nH]c2cc(F)ccc2c(=O)o1 ; O=C1Nc2ccc([N+](=O)[O-])cc2C1=O ; O=C1c2cc([N+](=O)[O-])ccc2-n2c1nc1cc(F)ccc1c2=O]

Following the method of General Procedure 3 but using 2-amino-4-fluorobenzoic acid as starting material. The title compound was obtained as a yellow solid. ¹H NMR (400 MHz, DMSO-d₆) δ 8.73 (d, J=8.4 Hz, 1H), 8.68 (s, 1H), 8.57 (s, 1H), 8.46-8.39 (m, 1H), 7.89 (d, J=7.6 Hz, 1H), 7.66 (t, J=7.6 Hz, 1H). ¹⁹F NMR (377 MHz, DMSO-d₆) δ−102.17. LC-MS: m/z [M+H]⁺ 312.0.

### Example 9: 3-Chloro-6,12-dioxo-6,12-dihydroindolo[2,1-b]quinazoline-8-carbonitrile (Compound 3)

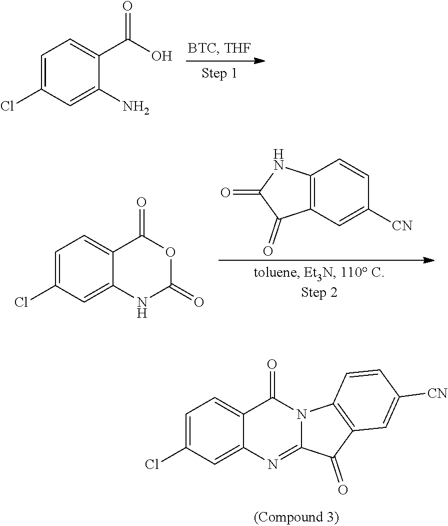 [SMILES: Nc1cc(Cl)ccc1C(=O)O ; O=c1[nH]c2cc(Cl)ccc2c(=O)o1 ; N#Cc1ccc2c(c1)C(=O)C(=O)N2 ; N#Cc1ccc2c(c1)C(=O)c1nc3cc(Cl)ccc3c(=O)n1-2]

Following the method of General Procedure 3 but using 2-amino-4-chlorobenzoic acid and 2,3-dioxo-1H-indole-5-carbonitrile as starting materials. The title compound was obtained as a yellow solid. ¹H NMR (400 MHz, DMSO-d₆) δ 8.60 (d, J=8.4 Hz, 1H), 8.47 (d, J=1.2 Hz, 1H), 8.37-8.28 (m, 2H), 8.12 (d, J=2.0 Hz, 1H), 7.82 (dd, J=8.4, 2.0 Hz, 1H).

### Example 10: Methyl 3-chloro-6,12-dioxo-6,12-dihydroindolo[2,1-b]quinazoline-8-carboxylate (Compound 6)

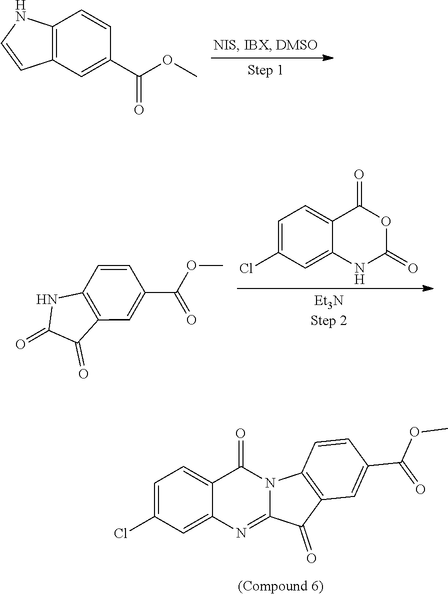

Following the method of General Procedure 4 but using 1H-indole-5-carboxylate and 7-chloro-1H-3,1-benzoxazine-2,4-dione as starting materials. The title compound was obtained as a brown solid. ¹H NMR (400 MHz, CDCl₃) δ 8.69 (d, J=8.4 Hz, 1H), 8.58 (d, J=1.6 Hz, 1H), 8.49 (dd, J=8.4, 1.6 Hz, 1H), 8.38 (d, J=8.4 Hz, 1H), 8.02 (d, J=2.0 Hz, 1H), 7.65 (dd, J=8.4, 2.0 Hz, 1H), 3.99 (s, 3H). LC-MS: m/z [M+H]⁺ 341.0.

### Example 11: 1-Fluoro-8-nitroindolo[2,1-b]quinazoline-6,12-dione (Compound 37)

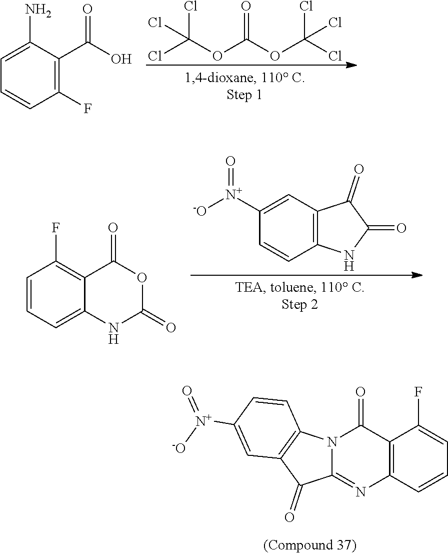

Following the method of General Procedure 3 but using 2-amino-6-fluorobenzoic acid and 5-nitro-1H-indole-2,3-dione as starting materials. The title compound was obtained as a brown solid. ¹H NMR (400 MHz, DMSO-d₆) δ 8.78-8.71 (m, 1H), 8.70-8.65 (m, 1H), 8.61-8.55 (m, 1H), 8.05-7.96 (m, 1H), 7.88-7.81 (m, 1H), 7.65-7.54 (m, 1H). ¹⁹F NMR (377 MHz, DMSO-d₆) δ−109.52. LC-MS: m/z [M+H]⁺ 312.1.

### Example 12: 1-Methyl-8-nitroindolo[2,1-b]quinazoline-6,12-dione (Compound 38)

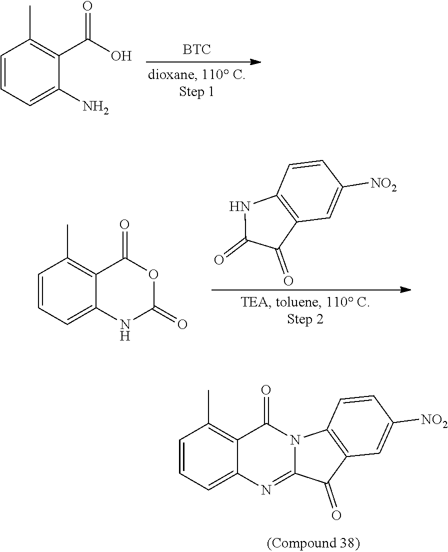

Following the method of General Procedure 3 but using 2-amino-6-methylbenzoic acid and 5-nitro-1H-indole-2,3-dione as starting materials. The title compound was obtained as a brown solid. ¹H NMR (400 MHz, DMSO-d₆) δ 8.73-8.69 (m, 2H), 8.54 (s, 1H), 7.86-7.78 (m, 2H), 7.56 (d, J=6.8 Hz, 1H), 2.90 (s, 3H). LC-MS: m/z [M+H]⁺ 330.0.

### Example 13: 1-Bromo-8-nitroindolo[2,1-b]quinazoline-6,12-dione (Compound 55)

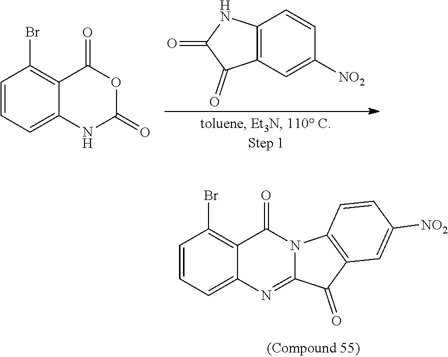 [SMILES: O=c1[nH]c2cccc(Br)c2c(=O)o1 ; O=C1Nc2ccc([N+](=O)[O-])cc2C1=O ; O=C1c2cc([N+](=O)[O-])ccc2-n2c1nc1cccc(Br)c1c2=O]

Following the method of General Procedure 2 but using 5-bromo-2H-benzo[d][1,3]oxazine-2,4(1H)-dione as starting material. The title compound was obtained as yellow solid. ¹H NMR (400 MHz, DMSO-d₆) δ 8.72-8.68 (m, 2H), 8.57 (s, 1H), 8.07-7.94 (m, 2H), 7.82 (t, J=7.6 Hz, 1H). LC-MS: m/z [M+Na]⁺394.0.

### Example 14: 1,3-Difluoro-8-nitroindolo[2,1-b]quinazoline-6,12-dione (Compound 11)

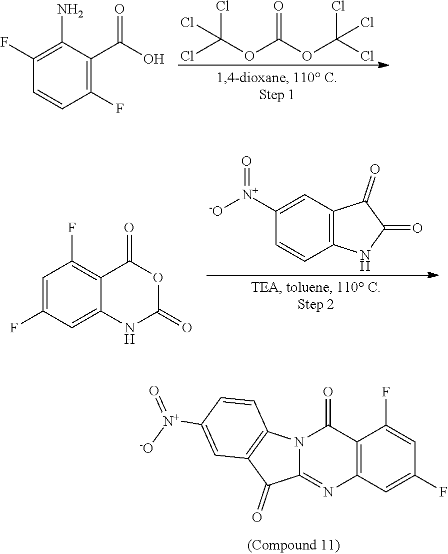

Following the method of General Procedure 3 but using 2-amino-4,6-difluorobenzoic acid and 5-nitro-1H-indole-2,3-dione as starting materials. The title compound was obtained as a brown solid. ¹H NMR (400 MHz, DMSO-d₆) δ 8.75-8.70 (m, 1H), 8.68-8.62 (m, 1H), 8.57 (d, J=2.4 Hz, 1H), 7.82-7.76 (m, 1H), 7.75-7.67 (m, 1H). ¹⁹F NMR (377 MHz, DMSO-d₆) δ−104.62, −98.81.

### Example 15: 1,3-Dichloro-8-nitroindolo[2,1-b]quinazoline-6,12-dione (Compound 56)

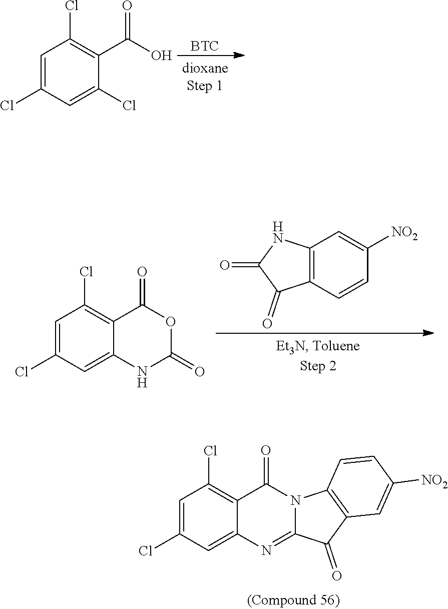

Following the method of General Procedure 3 but using 2-amino-4,6-dichlorobenzoic acid and 5-nitro-1H-indole-2,3-dione as starting materials. The title compound was obtained as a white solid. 1H NMR (400 MHz, DMSO-d₆) δ 8.72-8.68 (m, 2H), 8.58 (s, 1H), 8.11 (s, 1H), 8.00 (s, 1H). LC-MS: m/z [M+Na]⁺383.9.

### Example 16: 3-Fluoro-6,12-dioxo-6,12-dihydroindolo[2,1-b]quinazoline-8-carbonitrile (Compound 12)

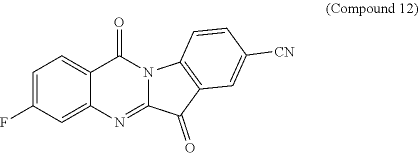 [SMILES: N#Cc1ccc2c(c1)C(=O)c1nc3cc(F)ccc3c(=O)n1-2]

Following the method of General Procedure 1 but using 7-fluoro-1H-3,1-benzoxazine-2,4-dione and 2,3-dioxo-1H-indole-5-carbonitrile as starting materials. The title compound was obtained as a brown solid. LC-MS: m/z [M+H]⁺ 292.1.

### Example 17: 3-Fluoro-8-(methylsulfonyl)indolo[2,1-b]quinazoline-6,12-dione (Compound 14)

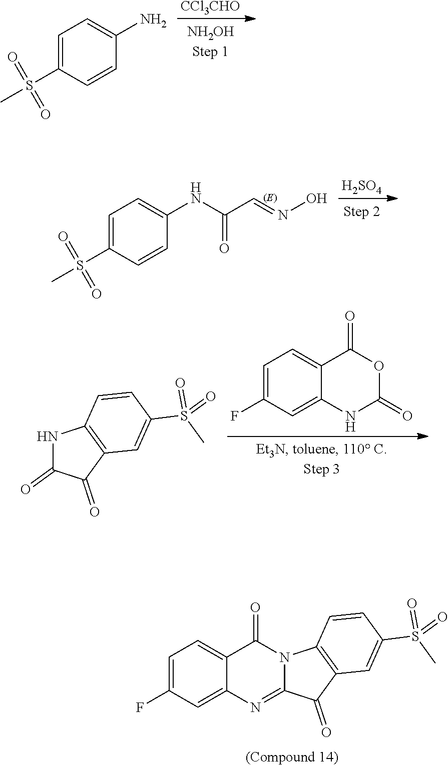

Following the method of General Procedure 5 but using 4-(methylsulfonyl)aniline and 7-fluoro-2H-benzo[d][1,3]oxazine-2,4(1H)-dione as starting materials. The title compound was obtained as a yellow solid. ¹H NMR (400 MHz, DMSO-d₆) δ 8.68 (d, J=8.4 Hz, 1H), 8.45-8.38 (m, 2H), 8.36 (d, J=1.6 Hz, 1H), 7.88 (dd, J=8.4, 1.6 Hz, 1H), 7.69-7.62 (m, 1H), 3.37 (s, 3H). LC-MS: m/z [M+H]⁺ 345.1.

### Example 18: 4-Fluoro-8-nitroindolo[2,1-b]quinazoline-6,12-dione (Compound 41)

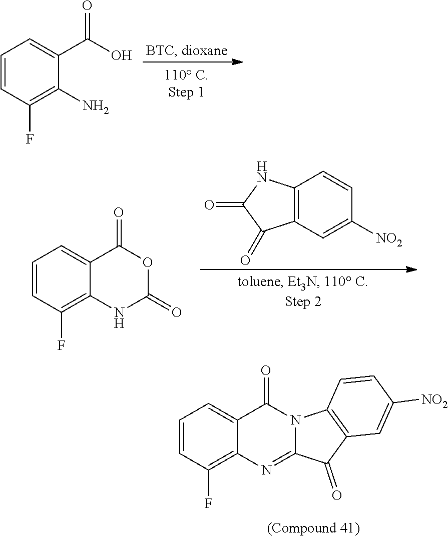

Following the method of General Procedure 3 but using 2-amino-3-fluorobenzoic acid and 5-nitroindoline-2,3-dione as starting materials. The title compound was obtained as a gray solid. ¹H NMR (400 MHz, DMSO-d₆) δ 8.75 (dd, J=8.8, 2.0 Hz, 1H), 8.70 (s, 1H), 8.60 (d, J=2.0 Hz, 1H), 8.20 (d, J=8.0 Hz, 1H), 7.95-7.89 (m, 1H), 7.84-7.77 (m, 1H). ¹⁹F NMR (376 MHz, DMSO-d₆) δ−122.06. LC-MS: m/z [M+H]⁺ 312.0

### Example 19: 4-Methyl-8-nitroindolo[2,1-b]quinazoline-6,12-dione (Compound 42)

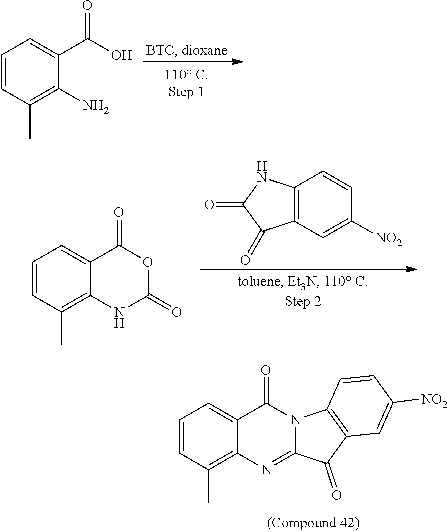

Following the method of General Procedure 3 but using 2-amino-3-methylbenzoic acid and 5-nitroindoline-2,3-dione as starting materials. The title compound was obtained as a yellow solid. ¹H NMR (400 MHz, DMSO-d₆) δ 8.70 (dd, J=15.2, 5.6 Hz, 2H), 8.57 (d, J=2.0 Hz, 1H), 8.23-8.19 (m, 1H), 7.86 (s, 1H), 7.68 (d, J=7.6 Hz, 1H), 2.65 (s, 3H). LC-MS: m/z [M+H]⁺ 308.1.

### Example 20: 4-Bromo-8-nitroindolo[2,1-b]quinazoline-6,12-dione (Compound 43)

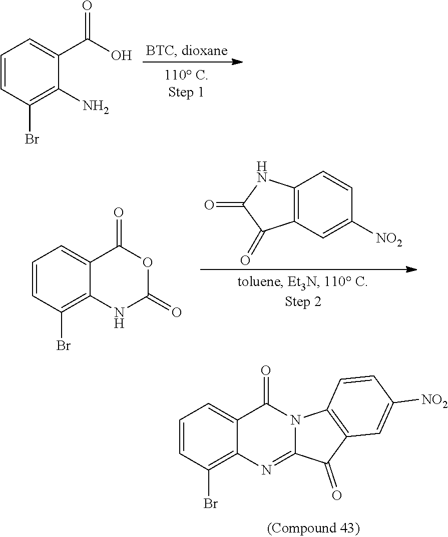

Following the method of General Procedure 3 but using 2-amino-3-bromobenzoic acid and 5-nitroindoline-2,3-dione as starting materials. The title compound was obtained as a gray solid. ¹H NMR (400 MHz, DMSO-d₆) δ 8.77-8.59 (m, 3H), 8.35 (dd, J=18.8, 7.6 Hz, 2H), 7.69 (t, J=7.6 Hz, 1H). LC-MS: m/z [M+Na]⁺393.9.

### Example 21: 3-Fluoro-4-methyl-8-nitroindolo[2,1-b]quinazoline-6,12-dione (Compound 30)

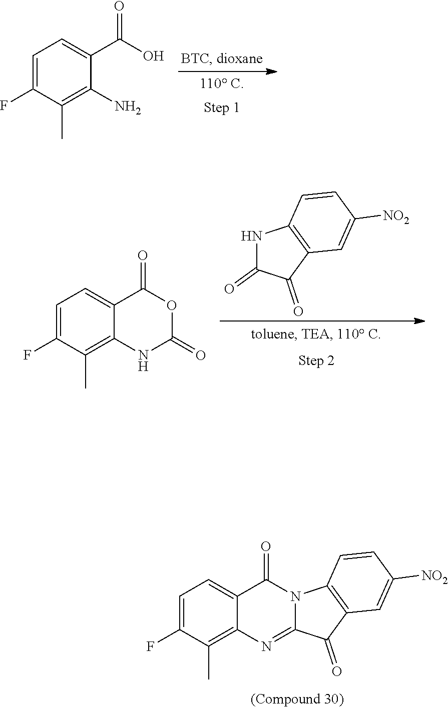 [SMILES: Cc1c(F)ccc(C(=O)O)c1N ; Cc1c(F)ccc2c(=O)oc(=O)[nH]c12 ; O=C1Nc2ccc([N+](=O)[O-])cc2C1=O ; Cc1c(F)ccc2c(=O)n3c(nc12)C(=O)c1cc([N+](=O)[O-])ccc1-3]

Following the method of General Procedure 3 but using 2-amino-4-fluoro-3-methylbenzoic acid and 5-nitroindoline-2,3-dione as starting materials. The title compound was obtained as a black solid. ¹H NMR (400 MHz, DMSO-d₆) δ 8.73 (d, J=8.8 Hz, 1H), 8.66 (d, J=8.8 Hz, 1H), 8.57 (s, 1H), 8.30-8.23 (m, 1H), 7.62 (t, J=8.8 Hz, 1H), 2.54 (s, 3H). ¹⁹F NMR (376 MHz, DMSO-d₆) δ−104.46. LC-MS: m/z [M+H]⁺ 326.1.

### Example 22: 4-Bromo-8-fluoroindolo[2,1-b]quinazoline-6,12-dione (Compound 44)

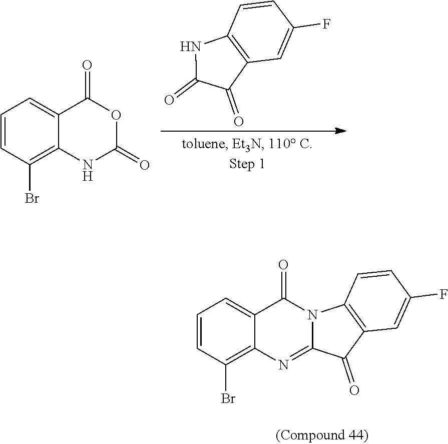 [SMILES: O=c1[nH]c2c(Br)cccc2c(=O)o1 ; O=C1Nc2ccc(F)cc2C1=O ; O=C1c2cc(F)ccc2-n2c1nc1c(Br)cccc1c2=O]

Following the method of General Procedure 1 but using 8-bromo-2H-benzo[d][1,3]oxazine-2,4(1H)-dione and 5-fluoro-1H-indole-2,3-dione as starting materials. The title compound was obtained as a yellow solid. ¹H NMR (400 MHz, DMSO-d₆) δ 8.48 (dd, J=8.8, 4.0 Hz, 1H), 8.35-8.25 (m, 2H), 7.84 (dd, J=7.2, 2.8 Hz, 1H), 7.74 (d, J=2.8 Hz, 1H), 7.64 (t, J=8.0 Hz, 1H). ¹⁹F NMR (377 MHz, DMSO-d₆) δ−114.07 (s, 1H). LC-MS: m/z [M+H]⁺ 345.0.

### Example 23: 4-Bromo-2-fluoro-8-nitroindolo[2,1-b]quinazoline-6,12-dione (Compound 45)

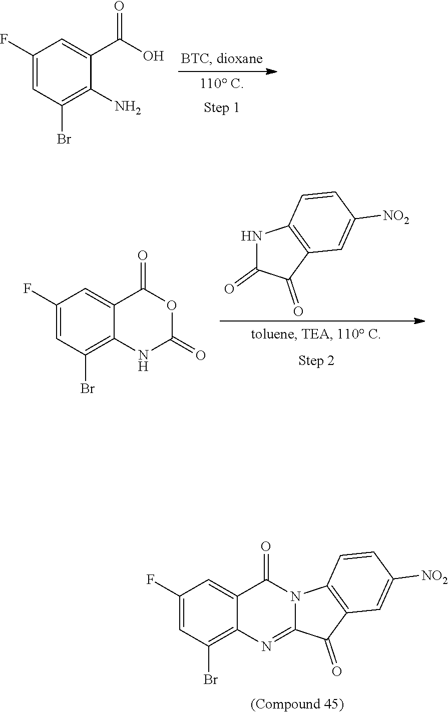 [SMILES: Nc1c(Br)cc(F)cc1C(=O)O ; O=c1[nH]c2c(Br)cc(F)cc2c(=O)o1 ; O=C1Nc2ccc([N+](=O)[O-])cc2C1=O ; O=C1c2cc([N+](=O)[O-])ccc2-n2c1nc1c(Br)cc(F)cc1c2=O]

Following the method of General Procedure 3 but using 2-amino-3-bromo-5-fluorobenzoic acid and 5-nitroindoline-2,3-dione as starting materials. The title compound was obtained as a yellow solid. ¹H NMR (400 MHz, DMSO-d₆) δ 8.75 (dd, J=8.8, 2.4 Hz, 1H), 8.67 (d, J=8.8 Hz, 1H), 8.61 (d, J=2.4 Hz, 1H), 8.41 (dd, J=8.0, 2.8 Hz, 1H), 8.16 (dd, J=8.0, 2.8 Hz, 1H). ¹⁹F NMR (400 MHz, DMSO-d₆) δ−106.80. LC-MS: m/z [M+H]⁺ 391.9.

### Example 24: 4-Bromo-6,12-dioxo-6,12-dihydroindolo[2,1-b]quinazoline-8-carbonitrile (Compound 46)

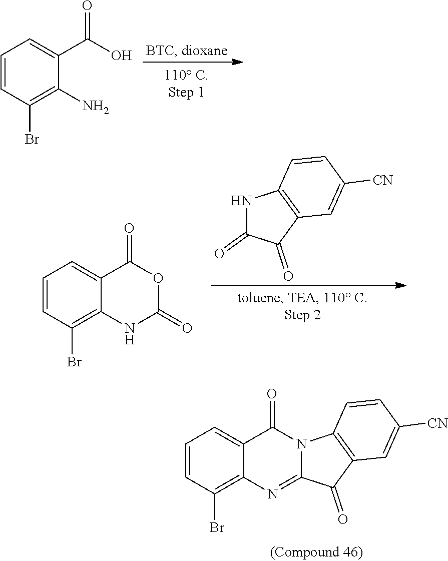

Following the method of General Procedure 3 but using 2-amino-3-bromobenzoic acid and 2,3-dioxoindoline-5-carbonitrile as starting materials. The title compound was obtained as a yellow solid. ¹H NMR (400 MHz, CDCl₃) δ 8.79 (d, J=8.4 Hz, 1H), 8.42 (d, J=8.0 Hz, 1H), 8.22 (s, 1H), 8.16 (d, J=8.0 Hz, 1H), 8.09-8.07 (m, 1H), 7.56 (t, J=8.0 Hz, 1H). LC-MS: m/z [M+H]⁺ 352.0.

### Example 25: 4-Bromo-2-chloro-8-nitroindolo[2,1-b]quinazoline-6,12-dione (Compound 47)

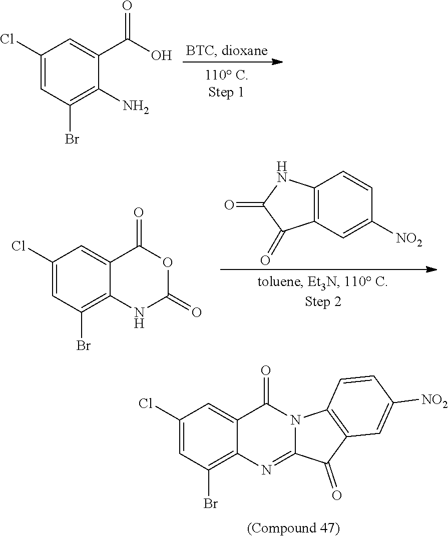

Following the method of General Procedure 3 but using 2-amino-3-bromo-5-chlorobenzoic acid and 5-nitroindoline-2,3-dione as starting materials. The title compound was obtained as a yellow solid. ¹H NMR (400 MHz, DMSO-d₆) δ 8.74 (dd, J=8.8, 2.4 Hz, 1H), 8.66 (d, J=8.8 Hz, 1H), 8.60 (d, J=2.4 Hz, 1H), 8.49 (d, J=2.4 Hz, 1H), 8.33 (d, J=2.4 Hz, 1H).

LC-MS: m/z [M+Na]⁺427.9.

### Example 26: 2,4-Dimethyl-8-nitroindolo[2,1-b]quinazoline-6,12-dione (Compound 48)

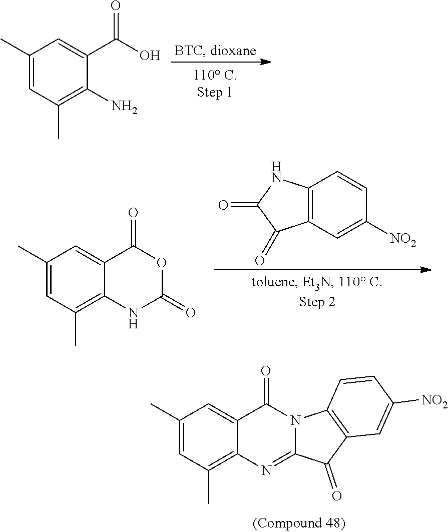

Following the method of General Procedure 3 but using 2-amino-3,5-dimethylbenzoic acid and 5-nitroindoline-2,3-dione as starting materials. The title compound was obtained as a yellow solid. 1H NMR (400 MHz, DMSO-d₆) δ 8.74-8.66 (m, 2H), 8.55 (d, J=2.0 Hz, 1H), 8.01 (s, 1H), 7.70 (s, 1H), 2.61 (s, 3H), 2.51 (s, 3H). LC-MS: m/z [M+H]⁺ 322.1.

### Example 27: 9-Nitropyrido[3′,4′:4,5]pyrimido[1,2-a]indole-5,11-dione (Compound 57)

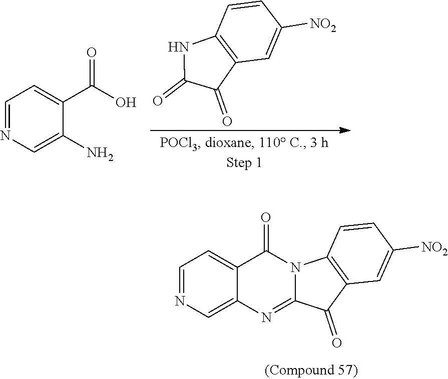

Following the method of General Procedure 2 but using 3-aminopyridine-4-carboxylic acid as starting material. The title compound was obtained as a yellow solid. ¹H NMR (400 MHz, DMSO-d₆) δ 9.36 (s, 1H), 8.94 (d, J=4.4 Hz, 1H), 8.76 (d, J=8.0 Hz, 1H), 8.69 (d, J=8.0 Hz, 1H), 8.61 (s, 1H), 8.24 (d, J=4.4 Hz, 1H). LC-MS: m/z [M+H]⁺ 295.

### Example 28: 3-Methoxy-9-nitropyrido[3′,4′:4,5]pyrimido[1,2-a]indole-5,11-dione (Compound 58)

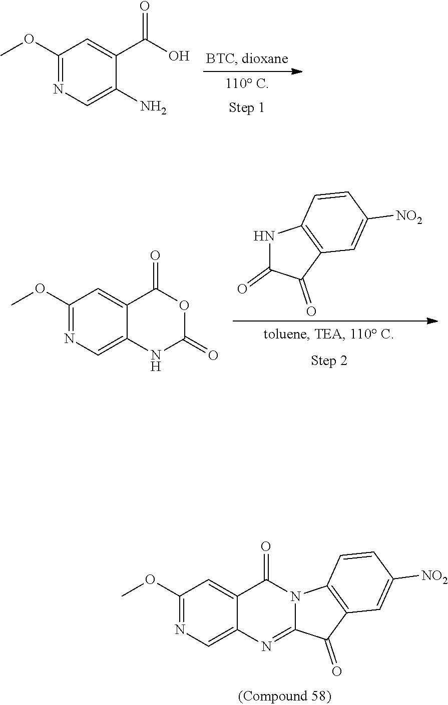 [SMILES: COc1cc(C(=O)O)c(N)cn1 ; COc1cc2c(=O)oc(=O)[nH]c2cn1 ; O=C1Nc2ccc([N+](=O)[O-])cc2C1=O ; COc1cc2c(=O)n3c(nc2cn1)C(=O)c1cc([N+](=O)[O-])ccc1-3]

Following the method of General Procedure 3 but using 5-amino-2-methoxypyridine-4-carboxylic acid and 5-nitroindoline-2,3-dione as starting materials. The title compound was obtained as a brown solid. ¹H NMR (400 MHz, DMSO-d₆) δ 9.05 (s, 1H), 8.75 (dd, J=8.8, 2.1 Hz, 1H), 8.67 (d, J=8.8 Hz, 1H), 8.58 (d, J=2.0 Hz, 1H), 7.57 (s, 1H), 4.05 (s, 3H). LC-MS: m/z [M+H]⁺ 325.1.

### Example 29: 2-Fluoro-4-methyl-8-nitroindolo[2,1-b]quinazoline-6,12-dione (Compound 49)

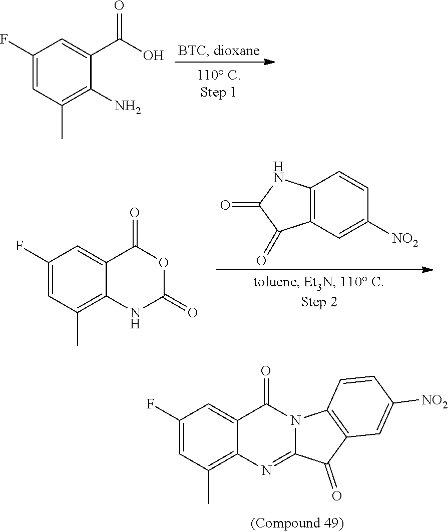

Following the method of General Procedure 3 but using 2-amino-5-fluoro-3-methylbenzoic acid and 5-nitroindoline-2,3-dione as starting materials. The title compound was obtained as a green solid. ¹H NMR (400 MHz, DMSO-d₆) δ 8.76-8.65 (m, 2H), 8.57 (d, J=2.0 Hz, 1H), 7.92 (dd, J=8.0, 2.4 Hz, 1H), 7.82 (d, J=9.2 Hz, 1H), 2.66 (s, 3H). ¹⁹F NMR (376 MHz, DMSO-d₆) δ−108.72. LC-MS: m/z [M+H]⁺ 326.1.

### Example 30: 1-Fluoro-4-methyl-8-nitroindolo[2,1-b]quinazoline-6,12-dione (Compound

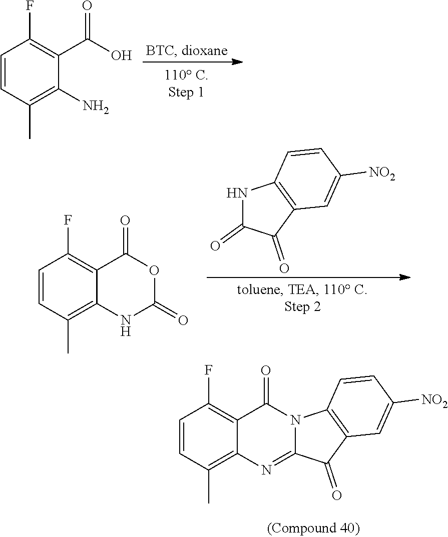

Following the method of General Procedure 3 but using 2-amino-6-fluoro-3-methylbenzoic acid and 5-nitroindoline-2,3-dione as starting materials. The title compound was obtained as a brown solid. ¹H NMR (400 MHz, DMSO-d₆) δ 8.78-8.63 (m, 2H), 8.57 (s, 1H), 7.92-7.83 (m, 1H), 7.53-7.43 (m, 1H), 2.59 (s, 3H). ¹⁹F NMR (376 MHz, DMSO-d₆) δ−112.61. LC-MS: m/z [M+H]⁺ 326.1.

### Example 31: 2-Methoxy-8-nitroindolo[2,1-b]quinazoline-6,12-dione (Compound 59)

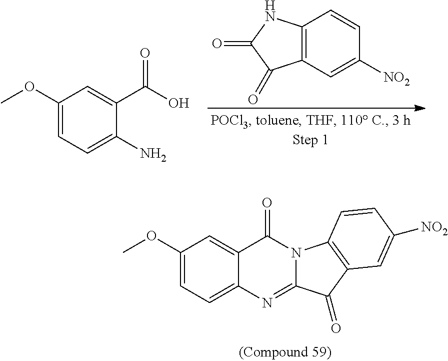 [SMILES: COc1ccc(N)c(C(=O)O)c1 ; O=C1Nc2ccc([N+](=O)[O-])cc2C1=O ; COc1ccc2nc3n(c(=O)c2c1)-c1ccc([N+](=O)[O-])cc1C3=O]

Following the method of General Procedure 2 but using 2-amino-5-methoxybenzoic acid and 5-nitroindoline-2,3-dione as starting materials. The title compound was obtained as a yellow solid. ¹H NMR (400 MHz, DMSO-d₆) δ 8.76-8.67 (m, 2H), 8.54 (d, J=2.4 Hz, 1H), 7.95 (d, J=8.0 Hz, 1H), 7.76 (d, J=3.2 Hz, 1H), 7.61-7.57 (m, 1H), 3.98 (s, 3H). LC-MS: m/z [M+H]⁺ 323.7.

### Example 32: 7,8-Difluoroindolo[2,1-b]quinazoline-6,12-dione (Compound 60)

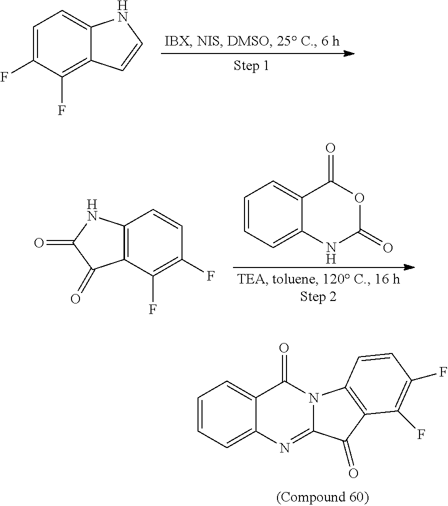 [SMILES: Fc1ccc2[nH]ccc2c1F ; O=C1Nc2ccc(F)c(F)c2C1=O ; O=c1[nH]c2ccccc2c(=O)o1 ; O=C1c2c(ccc(F)c2F)-n2c1nc1ccccc1c2=O]

Following the method of General Procedure 4 but using 4,5-difluoro-1H-indole and 1H-3,1-benzoxazine-2,4-dione as starting materials. The title compound was obtained as a yellow solid. ¹H NMR (400 MHz, DMSO-d₆) δ 8.38-8.27 (m, 2H), 8.02-7.91 (m, 3H), 7.81-7.73 (m, 1H). LC-MS: m/z [M+H]⁺ 285.0.

### Example 33: (6Z)-6-(Hydroxyimino)-8-nitroindolo[2,1-b]quinazolin-12-one (Compound

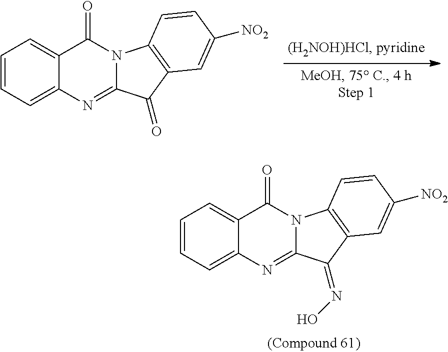 [SMILES: O=C1c2cc([N+](=O)[O-])ccc2-n2c1nc1ccccc1c2=O ; O=c1c2ccccc2nc2n1-c1ccc([N+](=O)[O-])cc1/C2=N/O]

To a solution of 8-nitroindolo[2,1-b]quinazoline-6,12-dione (commercial; 400 mg, 1.36 mmol) and pyridine (431.6 mg, 5.45 mmol) in MeOH (10 mL) was added hydroxylamine hydrochloride (190 mg, 2.73 mmol). The mixture was stirred at 75° C. for 4 h under N₂. The mixture was concentrated to give a residue which was purified by Prep-HPLC (Chromatographic column:—Xbridge-C18 150×19 mm, 5 μm; Mobile Phase: CAN/H₂O, 35-65% CAN in 0.1% TFA/water, 20 mL/min) to give (6Z)-6-(147 hydroxyamino)-8-nitroindolo[2,1-b]quinazolin-12-one (18.2 mg, 4% yield) as a yellow solid. ¹H NMR (400 MHz, DMSO-d₆) δ 9.16 (d, J=2.4 Hz, 1H), 8.74 (d, J=8.8 Hz, 1H), 8.46 (dd, J=8.8, 2.4 Hz, 1H), 8.31 (dd, J=8.0, 1.2 Hz, 1H), 7.92-7.85 (m, 1H), 7.80 (d, J=8.0 Hz, 1H), 7.57 (t, J=7.2 Hz, 1H). LC-MS: m/z [M+H]⁺ 309.1.

### Example 34: 8-Nitroindolo[2,1-b]quinazolin-12(6H)-one (Compound 62)

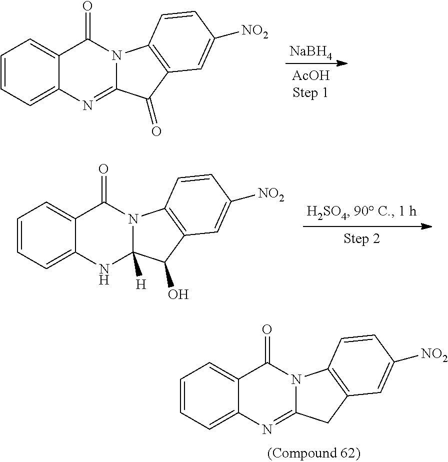 [SMILES: O=C1c2cc([N+](=O)[O-])ccc2-n2c1nc1ccccc1c2=O ; "NaBH4" ; O=C1c2ccccc2N[C@H]2[C@H](O)c3cc([N+](=O)[O-])ccc3N12 ; O=c1c2ccccc2nc2n1-c1ccc([N+](=O)[O-])cc1C2]

### Step 1: (5aR,6R)-6-hydroxy-8-nitro-5a,6-dihydroindolo[2,1-b]quinazolin-12(5H)-one

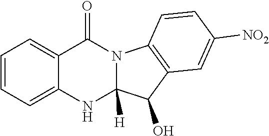 [SMILES: O=C1c2ccccc2N[C@H]2[C@H](O)c3cc([N+](=O)[O-])ccc3N12]

A solution of 8-nitroindolo[2,1-b]quinazoline-6,12-dione (commercial; 200 mg, 0.68 mmol) in AcOH (3 mL) was added NaBH₄ (78 mg, 2.0 mmol) in an ice bath. The reaction mixture was then warmed to room temperature and stirred for 1.5 h. The solution was quenched with H₂O and extracted with EA (20 mL×2). The combined organic phases were washed with NaHCO₃ (aq), dried over Na₂SO₄, filtered, and concentrated under reduced pressure. The residue was purified by chromatography eluting with EA in PE from 0% to 40% to afford (5aR,6R)-6-hydroxy-8-nitro-5a,6-dihydroindolo[2,1-b]quinazolin-12(5H)-one (98 mg, 42% yield) as a yellow solid. LC-MS: m/z [M+H]⁺ 298.1

### Step 2: 8-Nitroindolo[2,1-b]quinazolin-12(6H)-one (Compound 62)

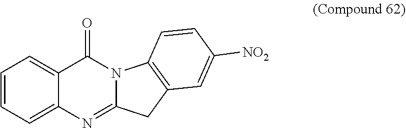 [SMILES: O=c1c2ccccc2nc2n1-c1ccc([N+](=O)[O-])cc1C2]

A solution of (5aR,6R)-6-hydroxy-8-nitro-5a,6-dihydroindolo[2,1-b]quinazolin-12(5H)-one (98 mg, 0.33 mmol) in H₂SO₄ (1.5 mL) was heated at 90° C. and stirred for 1.0 h. The solution was poured into NaHCO₃(aq) and extracted with EA (30 mL×2). The combined organic layers were dried over Na₂SO₄, filtered, and concentrated under reduced pressure. The residue was purified by chromatography eluting with EA in PE from 0% to 50% to afford 8-nitroindolo[2,1-b]quinazolin-12(6H)-one (8 mg, 9% yield) as a yellow solid. 1H NMR (400 MHz, DMSO-d₆) δ 12.02 (s, 1H), 8.74 (d, J=8.8 Hz, 1H), 8.46 (s, 1H), 8.20 (s, 1H), 8.04 (d, J=8.8 Hz, 1H), 7.75 (t, J=7.2 Hz, 1H), 7.33 (s, 1H), 7.22 (t, J=7.2 Hz, 1H), 6.26 (s, 1H). LC-MS: m/z [M+H]⁺ 280.1.

### Example 35: 1-Chloro-8-nitroindolo[2,1-b]quinazoline-6,12-dione (Compound 63)

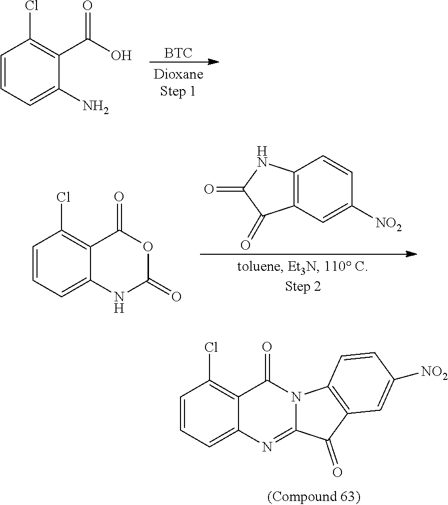

Following the method of General Procedure 2 but using 2-amino-6-chlorobenzoic acid and 5-nitroindoline-2,3-dione as starting materials. The title compound was obtained as a yellow solid. ¹H NMR (400 MHz, DMSO-d₆) δ 8.75-8.68 (m, 2H), 8.57 (d, J=2.0 Hz, 1H), 7.99-7.90 (m, 2H), 7.81 (dd, J=7.2, 1.2 Hz, 1H). LC-MS: m/z [M+H]⁺ 328.7.

### Example 36: 3-chloro-8-nitroindolo[2,1-b]quinazoline-6,12-dione (Compound 64)

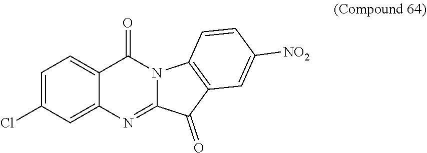 [SMILES: O=C1c2cc([N+](=O)[O-])ccc2-n2c1nc1cc(Cl)ccc1c2=O]

Following the method of General Procedure 1 but using 7-chloro-1H-3,1-benzoxazine-2,4-dione and 5-nitroindoline-2,3-dione as starting materials. The title compound was obtained as a dark yellow solid. LC-MS: m/z [M+H]⁺ 328.0.

### Example 37: 8-Amino-3-chloroindolo[2,1-b]quinazoline-6,12-dione (Compound 5)

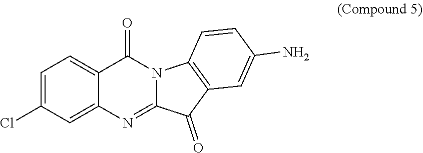 [SMILES: Nc1ccc2c(c1)C(=O)c1nc3cc(Cl)ccc3c(=O)n1-2]

To a solution of 3-chloro-8-nitroindolo[2,1-b]quinazoline-6,12-dione (from Example 36; 100 mg, 0.31 mmol) and Fe (85 mg, 1.5 mmol) in EtOH (4 mL) and H₂O (1 mL) was added NH₄Cl (163 mg, 3.1 mmol). The mixture was stirred for 1.0 h at 100° C. The solvent was removed under reduced pressure. The residue was purified by chromatography eluting with MeOH in DCM from 0% to 5% to afford 8-amino-3-chloroindolo[2,1-b]quinazoline-6,12-dione (30 mg, 33% yield) as a pink solid. ¹H NMR (400 MHz, DMSO-d₆) δ 8.25 (d, J=8.4 Hz, 1H), 8.11 (d, J=9.2 Hz, 1H), 8.01 (s, 1H), 7.74 (d, J=8.4 Hz, 1H), 7.01-6.96 (m, 2H), 5.69 (s, 2H). LC-MS: m/z [M+H]⁺ 298.0.

### Example 38: N-(3-Chloro-6,12-dioxo-6,12-dihydroindolo[2,1-b]quinazolin-8-yl)acetamide (Compound 7)

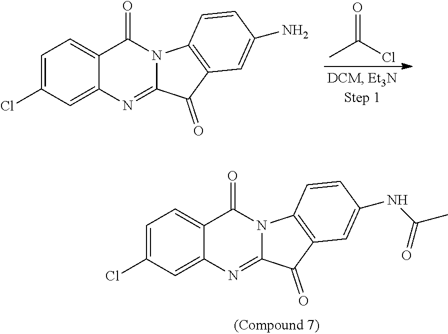

To a solution of 8-amino-3-chloroindolo[2,1-b]quinazoline-6,12-dione (from Example 37; 20 mg, 0.067 mmol) and Et₃N (20 mg, 0.20 mmol) in DCM (2.0 mL) was added acetyl chloride (8.0 mg, 0.10 mmol). The mixture was stirred for 1.0 h at 25° C. The suspension was filtered and the cake was washed with EA, DCM, and MeOH to afford N-(3-chloro-6,12-dioxo-6,12-dihydroindolo[2,1-b]quinazolin-8-yl)acetamide (11 mg, 48% yield) as a red solid. ¹H NMR (400 MHz, DMSO-d₆) δ 10.37 (s, 1H), 8.37 (d, J=8.4 Hz, 1H), 8.29 (d, J=8.4 Hz, 1H), 8.20 (s, 1H), 8.06 (s, 1H), 7.86 (d, J=8.4 Hz, 1H), 7.77 (d, J=8.4 Hz, 1H), 2.10 (s, 3H). LC-MS: m/z [M+H]⁺ 340.0.

### Example 39: Methyl N-(3-((3-chloro-6,12-dioxo-6,12-dihydroindolo[2,1-b]quinazolin-8-yl)amino)-3-oxopropyl)-P-methylphosphonamidoate (Compound 9)

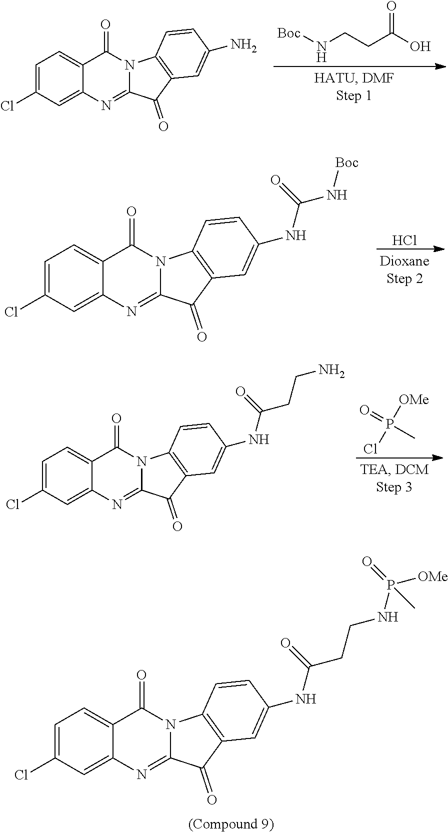

### Step 1: tert-butyl (3-((3-chloro-6,12-dioxo-6,12-dihydroindolo[2,1-b]quinazolin-8-yl)amino)-3-oxopropyl)carbamate

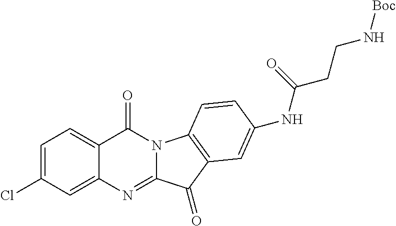 [SMILES: CC(C)(C)OC(=O)NCCC(=O)Nc1ccc2c(c1)C(=O)c1nc3cc(Cl)ccc3c(=O)n1-2]

To a solution of 8-amino-3-chloroindolo[2,1-b]quinazoline-6,12-dione (from Example 37; 0.20 g, 0.67 mmol) and 3-((tert-butoxycarbonyl)amino)propanoic acid (0.19 g, 1.0 mmol) in DMF (5.0 mL) was added HATU (0.38 g, 1.0 mmol) and DIPEA (0.26 g, 2.0 mmol). The mixture was stirred for 16 h at 25° C. quenched with H₂O, and filtered. The filter cake was dissolved into MeOH and concentrated. The residue was purified by chromatography eluting with MeOH in DCM from 0% to 5% to give the title compound (0.20 g, 63% yield) as a yellow solid. LC-MS: m/z [M+H]⁺ 469.1

### Step 2: 3-amino-N-(3-chloro-6,12-dioxo-6,12-dihydroindolo[2,1-b]quinazolin-8-yl)propanamide (Compound 9)

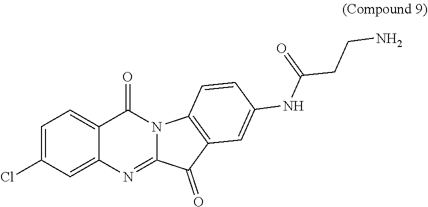 [SMILES: NCCC(=O)Nc1ccc2c(c1)C(=O)c1nc3cc(Cl)ccc3c(=O)n1-2]

To a solution of the tert-butyl carbamate from step 1 (0.20 g, 0.43 mmol) in MeOH (2.0 mL) was added HCl (4.0 M in dioxane, 2.0 mL). The mixture was stirred for 2.5 h at 50° C. The solvent was removed under reduced pressure to afford 3-amino-N-(3-chloro-6,12-dioxo-6,12-dihydroindolo[2,1-b]quinazolin-8-yl)propanamide (0.14 g, 89% yield) as a dark purple solid, which was used in the next step without purification. LC-MS: m/z [M+H]⁺ 369.0.

### Step 3: Methyl N-(3-((3-chloro-6,12-dioxo-6,12-dihydroindolo[2,1-b]quinazolin-8-yl)amino)-3-oxopropyl)-P-methylphosphonamidoate (Compound 9)

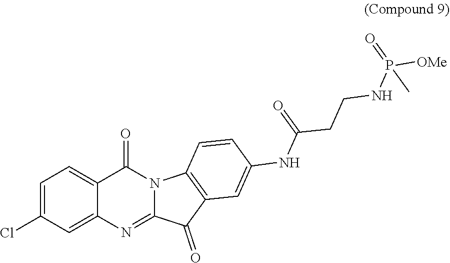 [SMILES: COP(C)(=O)NCCC(=O)Nc1ccc2c(c1)C(=O)c1nc3cc(Cl)ccc3c(=O)n1-2]

To a solution of 3-amino-N-(3-chloro-6,12-dioxo-6,12-dihydroindolo[2,1-b]quinazolin-8-yl) propanamide (0.14 g, 0.38 mmol) and Et₃N (0.12 g, 1.1 mmol) in DCM (4.0 mL) was added methyl methylphosphonochloridate (98 mg, 0.76 mmol, in 1.0 ml of DCM). The mixture was stirred for 20 min at 25° C., quenched with NaHCO₃ (aq), and extracted with DCM (20 mL×2). The combined organic layers were dried over Na₂SO₄, filtered, and concentrated under reduced pressure. The residue was purified by chromatography eluting with MeOH in DCM from 0% to 5% to afford methyl N-(3-((3-chloro-6,12-dioxo-6,12-dihydroindolo[2,1-b]quinazolin-8-yl)amino)-3-oxopropyl)-P-methylphosphonamidoate (6.0 mg, 3% yield) as a dark yellow solid. ¹H NMR (400 MHz, DMSO-d₆) δ 10.42 (s, 1H), 8.36 (d, J=8.8 Hz, 1H), 8.28 (d, J=8.4 Hz, 1H), 8.22 (t, J=3.2 Hz, 1H), 8.05 (d, J=2.0 Hz, 1H), 7.88 (dd, J=8.8, 2.0 Hz, 1H), 7.76 (dd, J=8.4, 2.0 Hz, 1H), 4.69-4.66 (m, 1H), 3.47-3.45 (m, 3H), 3.15-3.05 (m, 2H), 2.52 (d, J=6.8 Hz, 2H), 1.31-1.29 (m, 3H). LC-MS: m/z [M+H]⁺ 461.0.

### Example 40: 3-Chloro-6,12-dioxo-6,12-dihydroindolo[2,1-b]quinazoline-8-carboxamide (Compound 8)

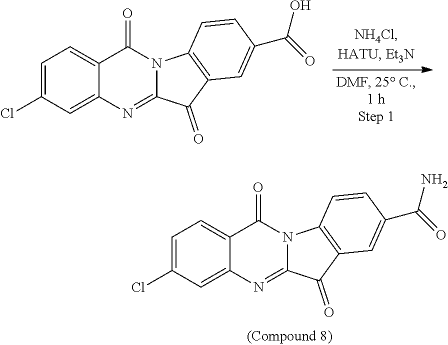 [SMILES: O=C(O)c1ccc2c(c1)C(=O)c1nc3cc(Cl)ccc3c(=O)n1-2 ; NC(=O)c1ccc2c(c1)C(=O)c1nc3cc(Cl)ccc3c(=O)n1-2]

To a solution of 3-chloro-6,12-dioxoindolo[2,1-b]quinazoline-8-carboxylic acid (from General Procedure 5, Example 5; 100 mg, 0.306 mmol) in DMF (6 mL) were added NH₄Cl (49 mg, 0.918 mmol), HATU (175 mg, 0.459 mmol), and Et₃N (93 mg, 0.918 mmol). The mixture was stirred at 25° C. for 1 hour then diluted with H₂O (50 mL). Filtration afforded 3-chloro-6,12-dioxoindolo[2,1-b]quinazoline-8-carboxamide (30 mg, 30% yield) as a green solid. ¹H NMR (400 MHz, DMSO-d₆) δ 8.51 (d, J=8.8 Hz, 1H), 8.40-8.36 (m, 2H), 8.33 (d, J=8.4 Hz, 1H), 8.25 (s, 1H), 8.09 (d, J=2.0 Hz, 1H), 7.80 (dd, J=8.4, 2.0 Hz, 1H), 7.62 (s, 1H). LC-MS: m/z [M+H]⁺ 326.0.

### Example 41: N-(2-Aminoethyl)-3-chloro-6,12-dioxo-6,12-dihydroindolo[2,1-b]quinazoline-8-carboxamide (Compound 10)

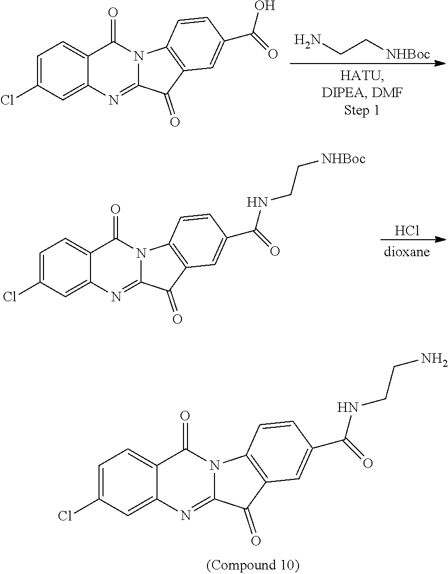 [SMILES: O=C(O)c1ccc2c(c1)C(=O)c1nc3cc(Cl)ccc3c(=O)n1-2 ; CC(C)(C)OC(=O)NCCN ; CC(C)(C)OC(=O)NCCNC(=O)c1ccc2c(c1)C(=O)c1nc3cc(Cl)ccc3c(=O)n1-2 ; Cl ; NCCNC(=O)c1ccc2c(c1)C(=O)c1nc3cc(Cl)ccc3c(=O)n1-2]

### Step 1: tert-butyl (2-(3-chloro-6,12-dioxo-6,12-dihydroindolo[2,1-b]quinazoline-8-carboxamido)ethyl)carbamate

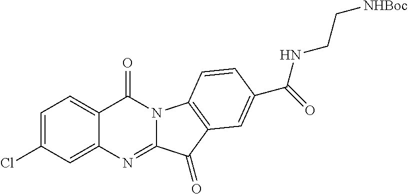 [SMILES: CC(C)(C)OC(=O)NCCNC(=O)c1ccc2c(c1)C(=O)c1nc3cc(Cl)ccc3c(=O)n1-2]

To a solution of 3-chloro-6,12-dioxo-6,12-dihydroindolo[2,1-b]quinazoline-8-carboxylic acid (from General Procedure 5, Example 5; 0.50 g, 1.5 mmol) and tert-butyl (2-aminoethyl) carbamate (0.37 g, 2.30 mmol) in DMF (20 mL) was added HATU (0.87 g, 2.30 mmol) and DIPEA (0.59 g, 4.6 mmol). The mixture was stirred for 5 h at 25° C. and quenched with water. The solids were filtered, and the filter cake was washed with DCM, MeOH and EA. The solid was dried under reduced pressure to give tert-butyl (2-(3-chloro-6,12-dioxo-6,12-dihydroindolo[2,1-b]quinazoline-8-carboxamido)ethyl)carbamate (0.50 g, 56% yield) as a yellow solid. LC-MS: m/z [M+H]⁺ 469.0.

### Step 2: N-(2-aminoethyl)-3-chloro-6,12-dioxo-6,12-dihydroindolo[2,1-b]quinazoline-8-carboxamide (Compound 10)

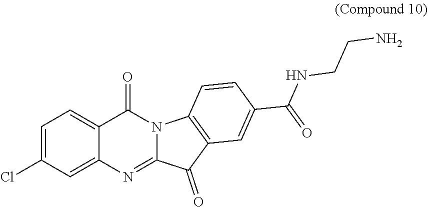 [SMILES: NCCNC(=O)c1ccc2c(c1)C(=O)c1nc3cc(Cl)ccc3c(=O)n1-2]

To a solution of tert-butyl (2-(3-chloro-6,12-dioxo-6,12-dihydroindolo[2,1-b]quinazoline-8-carboxamido)ethyl)carbamate (0.40 g, 0.85 mmol) in dioxane (5.0 mL) was added HCl (4 M in dioxane, 5 mL). The mixture was stirred for 2.0 h at 60° C. The solvent was removed under reduced pressure to afford N-(2-aminoethyl)-3-chloro-6,12-dioxoindolo[2,1-b]quinazoline-8-carboxamide (0.30 g, 95% yield) as a yellow solid. ¹H NMR (400 MHz, DMSO-d₆) δ 8.96 (s, 1H), 8.54 (d, J=8.4 Hz, 1H), 8.39-8.34 (m, 3H), 8.09 (d, J=2.0 Hz, 1H), 7.95 (s, 3H), 7.86-7.77 (m, 1H), 3.56 (d, J=8.0 Hz, 2H), 3.03 (d, J=5.2 Hz, 2H). LC-MS: m/z [M+H]⁺ 369.1.

### Example 42: 3-Chloro-6,12-dioxo-N-(2-(sulfamoylamino)ethyl)-6,12-dihydroindolo[2,1-b]quinazoline-8-carboxamide (Compound 13)

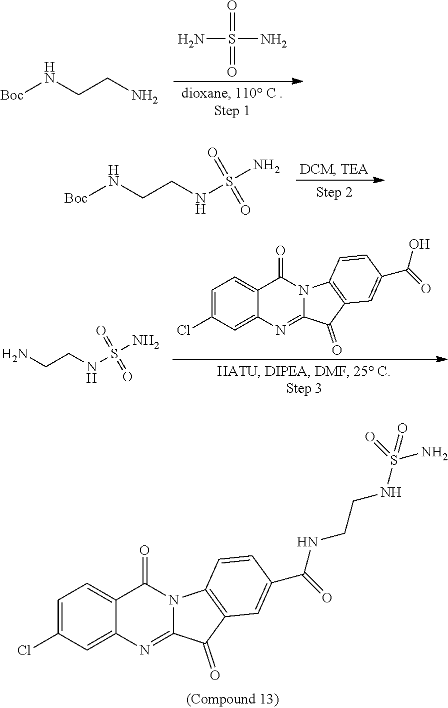

### Step 1: tert-butyl (2-(sulfamoylamino)ethyl)carbamate

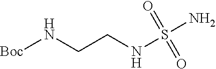 [SMILES: CC(C)(C)OC(=O)NCCNS(N)(=O)=O]

To a solution of tert-butyl (2-aminoethyl)carbamate (1.0 g, 0.0062 mol) was added sulfuric diamide (1.8 g, 0.019 mol). The reactor was evacuated and backfilled with N₂ three times. The mixture was stirred for 2.5 h at 110° C. The solvent was removed under reduced pressure, the residue was dissolved into EA (100 mL) and washed with HCl (1N) two times. The organic layer was dried over Na₂SO₄, filtered and concentrated under reduced pressure to afford tert-butyl (2-(sulfamoylamino)ethyl)carbamate (240 mg, crude) as a colorless oil. LC-MS: m/z [M+H]⁺ 240.1.

### Step 2: 2-(sulfamoylamino)ethan-1-aminium

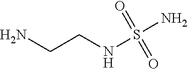

To a solution of tert-butyl[2-(sulfamoylamino)ethyl]amino formate (240 mg, 1.0 mmol) in DCM (2.0 mL) was added TFA (0.5 mL). The mixture was stirred for 6 h at 25° C. The solvent was removed under reduced pressure to afford 2-(sulfamoylamino)ethan-1-aminium (200 mg, crude) as a light yellow oil. LC-MS: m/z [M+H]⁺ 140.1.

### Step 3: 3-chloro-6,12-dioxo-N-(2-(sulfamoylamino)ethyl)-6,12-dihydroindolo[2,1-b]quinazoline-8-carboxamide (Compound 13)

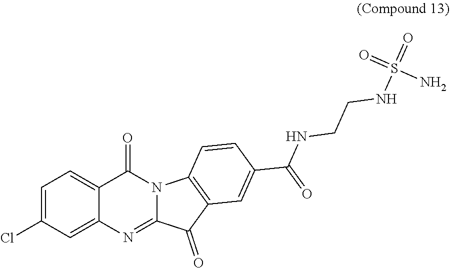 [SMILES: NS(=O)(=O)NCCNC(=O)c1ccc2c(c1)C(=O)c1nc3cc(Cl)ccc3c(=O)n1-2]

To a solution of 3-chloro-6,12-dioxo-6,12-dihydroindolo[2,1-b]quinazoline-8-carboxylic acid (from Example 5; 100 mg, 0.31 mmol) and 2-(sulfamoylamino)ethan-1-aminium (43 mg, 0.31 mmol) in DMF (5.0 mL) was added HATU (151 mg, 0.40 mmol) and DIPEA (59 mg, 0.46 mmol), the reactor was evacuated and backfilled with N₂ three times. The mixture was stirred for 3.5 h at 25° C. The solvent was removed under reduced pressure, the residue was purified by Prep-HPLC (Chromatographic column: WELCH Xtimate C18 21.2*250 mm 10 μm Mobile Phase: CAN/H₂O (0.1% TFA) to give 3-chloro-6,12-dioxo-N-(2-(sulfamoylamino)ethyl)-6,12-dihydroindolo[2,1-b]quinazoline-8-carboxamide (10 mg, 7.3% yield) as yellow solid. ¹H NMR (400 MHz, DMSO-d₆) δ 8.77 (s, 1H), 8.52 (d, J=9.2 Hz, 1H), 8.35 (d, J=6.8 Hz, 2H), 8.09 (d, J=2.0 Hz, 1H), 7.81 (dd, J=8.4, 2.0 Hz, 1H), 6.71 (t, J=6.0 Hz, 1H), 6.58 (s, 2H), 3.44 (dd, J=12.4, 6.4 Hz, 2H), 3.09 (dd, J=12.4, 6.4 Hz, 2H). LC-MS: m/z [M+H]⁺ 448.0.

### Example 43: 1-Chloro-8-nitroindolo[2,1-b]quinazolin-12(5H)-one (Compound 39)

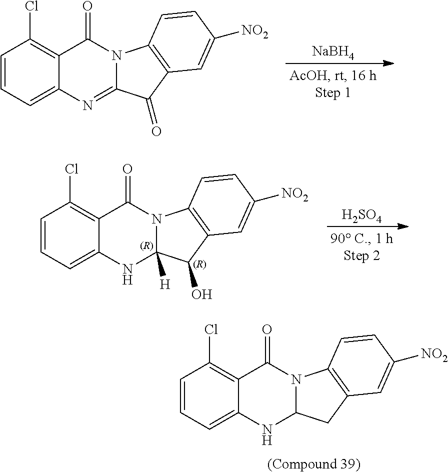

### Step 1: (5aR,6R)-1-Chloro-6-hydroxy-8-nitro-5a,6-dihydroindolo[2,1-b]quinazolin-12(5H)-one

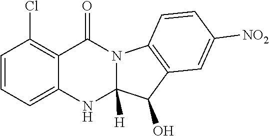 [SMILES: O=C1c2c(Cl)cccc2N[C@H]2[C@H](O)c3cc([N+](=O)[O-])ccc3N12]

To a solution of 1-chloro-8-nitroindolo[2,1-b]quinazoline-6,12-dione (from Example 35; 655 mg, 2.0 mmol) in AcOH (10 mL) at 0° C. was added NaBH₄ (378 mg, 10.0 mmol) slowly. The mixture was stirred at 25° C. for 16 h. The mixture was quenched with water (50 mL), then extracted with EA (50 mL×2). The combined organic phase was dried over Na₂SO₄, concentrated and purified by chromatography (DCM/EA=90:10) to afford product (5aR,6R)-1-chloro-6-hydroxy-8-nitro-5H,5aH,6H-indolo[2,1-b]quinazolin-12-one (40 mg) as yellow solid. LC-MS: m/z [M+H]⁺ 332.0.

### Step 2: 1-chloro-8-nitroindolo[2,1-b]quinazolin-12(5H)-one (Compound 39)

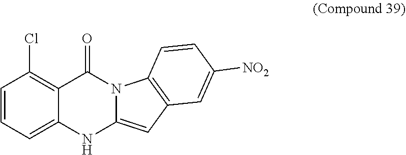 [SMILES: O=c1c2c(Cl)cccc2[nH]c2cc3cc([N+](=O)[O-])ccc3n12]

A solution of (5aR,6R)-1-chloro-6-hydroxy-8-nitro-5H,5aH,6H-indolo[2,1-b]quinazolin-12-one (40 mg, 0.12 mmol) in H₂SO₄ (2 mL) was stirred at 90° C. for 1 h. The solution was diluted with NaHCO₃ (aq, 50 mL) and extracted with EA (50 mL×2). The combined organic phase was dried over Na₂SO₄, concentrated and purified by chromatography (DCM/MeOH=80:20) to afford 1-chloro-8-nitro-6H-indolo[2,1-b]quinazolin-12-one (15 mg) as a red solid. ¹H NMR (400 MHz, DMSO-d₆): δ 12.14 (s, 1H), 8.71 (d, J=9.0 Hz, 1H), 8.42 (d, J=2.2 Hz, 1H), 8.01 (dd, J=9.0, 2.3 Hz, 1H), 7.63 (t, J=8.0 Hz, 1H), 7.23 (m, 2H), 6.21 (s, 1H). LC-MS: m/z [M+H]⁺ 314.0.

### Example 44: 8-Fluoro-3-((4-methylpiperazin-1-yl)methyl)indolo[2,1-b]quinazoline-6,12-dione (Compound 15)

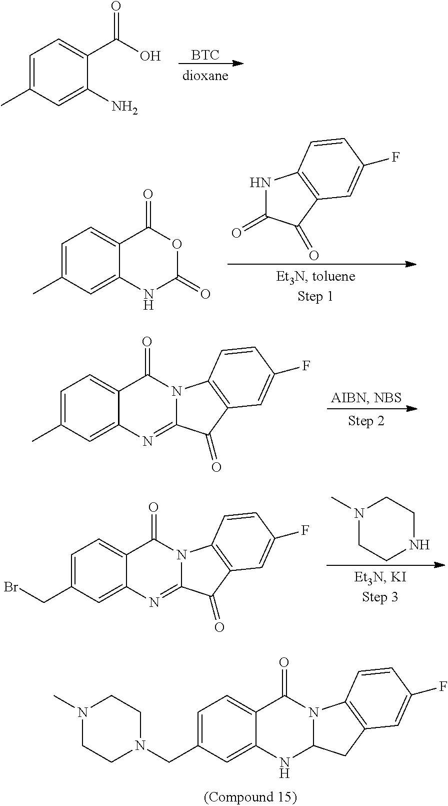

### Step 1: 8-fluoro-3-methylindolo[2,1-b]quinazoline-6,12-dione

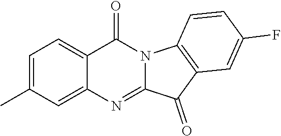 [SMILES: Cc1ccc2c(=O)n3c(nc2c1)C(=O)c1cc(F)ccc1-3]

Following the method of General Procedure 3 but using 2-amino-4-methylbenzoic acid and 5-fluoroindoline-2,3-dione as starting materials. The title compound was obtained as a yellow solid. LC-MS: m/z [M+H]⁺ 281.1.

### Step 2: 3-(bromomethyl)-8-fluoroindolo[2,1-b]quinazoline-6,12-dione

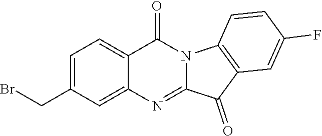 [SMILES: O=C1c2cc(F)ccc2-n2c1nc1cc(CBr)ccc1c2=O]

To a solution of 8-fluoro-3-methylindolo[2,1-b]quinazoline-6,12-dione (0.20 g, 0.71 mmol) in CCl₄ (5 mL) was added NBS (0.19 g, 1.1 mmol) and AIBN (35 mg, 0.21 mmol), and the mixture was stirred at 80° C. for 16 h. The solvent was removed under vacuum. The residue was purified by flash chromatography column eluting with MeOH in DCM from 0% to 5% to afford 3-(bromomethyl)-8-fluoroindolo[2,1-b]quinazoline-6,12-dione (110 mg, 41%) as a yellow solid. LC-MS: m/z [M+H]⁺ 360.0.

### Step 3: 8-fluoro-3-((4-methylpiperazin-1-yl)methyl)indolo[2,1-b]quinazoline-6,12-dione (Compound 15)

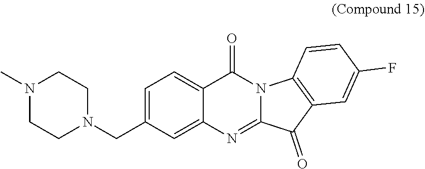 [SMILES: CN1CCN(Cc2ccc3c(=O)n4c(nc3c2)C(=O)c2cc(F)ccc2-4)CC1]

To a solution of 3-(bromomethyl)-8-fluoroindolo[2,1-b]quinazoline-6,12-dione (0.10 g, 0.28 mmol), TEA (28 mg, 0.28 mmol) in DMF (5.0 mL) was added KI (4.6 mg, 0.028 mmol) and 1-methylpiperazine (56 mg, 0.56 mmol), the reactor was evacuated and backfilled with N₂ three times. The mixture was stirred for 2.0 h at 25° C. then quenched with H₂O and extracted with the EA (50 mL×2). The combined layers were dried over Na₂SO₄, filtered, concentrated under reduced pressure. The residue was purified by chromatography eluting with MeOH in DCM from 0% to 5% to afford 8-fluoro-3-((4-methylpiperazin-1-yl)methyl)indolo[2,1-b]quinazoline-6,12-dione (35 mg, 33% yield) as a yellow solid. ¹H NMR (400 MHz, DMSO-d₆) δ 8.49 (dd, J=8.8, 4.4 Hz, 1H), 8.29 (d, J=8.0 Hz, 1H), 7.86 (s, 1H), 7.80 (dd, J=7.2, 2.8 Hz, 1H), 7.76-7.66 (m, 2H), 3.71 (s, 2H), 2.73-2.52 (m, 4H), 2.32 (s, 7H). LC-MS: m/z [M+H]⁺ 379.2.

### Example 45: 3-bromo-8-nitroindolo[2,1-b]quinazoline-6,12-dione (Compound 65)

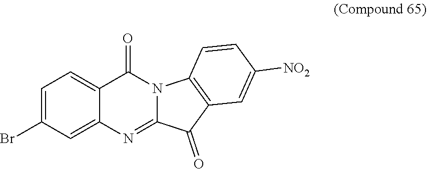 [SMILES: O=C1c2cc([N+](=O)[O-])ccc2-n2c1nc1cc(Br)ccc1c2=O]

Following the method of General Procedure 2 but using 7-bromo-2H-benzo[d][1,3]oxazine-2,4(1H)-dione and 5-nitroindoline-2,3-dione as starting materials. The title compound was obtained as a yellow solid. LC-MS: m/z [M+H]⁺ 372.1.

### Example 46: tert-Butyl (8-nitro-6,12-dioxo-6,12-dihydroindolo[2,1-b]quinazolin-3-yl)carbamate (Compound 17)

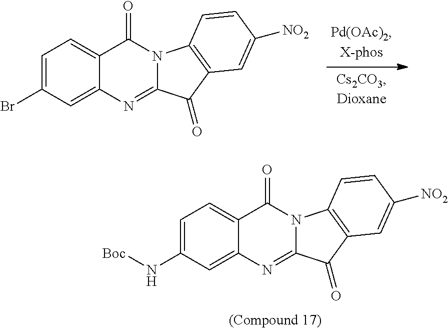 [SMILES: O=C1c2cc([N+](=O)[O-])ccc2-n2c1nc1cc(Br)ccc1c2=O ; CC(C)(C)OC(=O)Nc1ccc2c(=O)n3c(nc2c1)C(=O)c1cc([N+](=O)[O-])ccc1-3]

To a solution of 3-bromo-8-nitroindolo[2,1-b]quinazoline-6,12-dione (from Example 45; 200 mg, 0.53 mmol) and amino tert-butyl formate (0.075 g, 0.80 mmol) in dioxane (5.0 mL) was added Pd(OAc)₂ (0.018 g, 0.08 mmol), X-phos (0.088 g, 0.16 mmol) and Cs₂CO₃ (0.60 g, 1.2 mmol), the reactor was evacuated and backfilled with N₂ three times. The mixture was stirred for 2.0 h at 95° C. then the solvent was removed under reduced pressure. The residue was washed with PE, EA, and DCM. The solid was dried in vacuo to afford tert-butyl (8-nitro-6,12-dioxo-6,12-dihydroindolo[2,1-b]quinazolin-3-yl)carbamate (100 mg, 61% yield) as a gray solid. ¹H NMR (400 MHz, DMSO-d₆) δ 10.17 (s, 1H), 8.85-8.40 (m, 3H), 8.24 (s, 1H), 8.07 (s, 1H), 7.84 (s, 1H). LC-MS: m/z [M+H]⁺ 409.1.

### Example 47: 3-Amino-8-nitroindolo[2,1-b]quinazoline-6,12-dione (Compound 16)

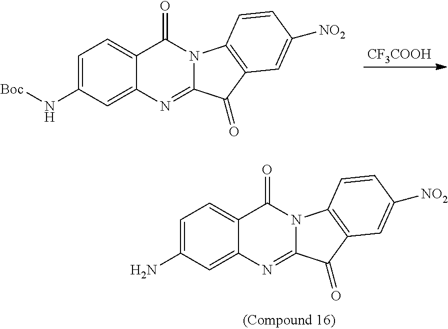 [SMILES: CC(C)(C)OC(=O)Nc1ccc2c(=O)n3c(nc2c1)C(=O)c1cc([N+](=O)[O-])ccc1-3 ; "CF3COOH" ; Nc1ccc2c(=O)n3c(nc2c1)C(=O)c1cc([N+](=O)[O-])ccc1-3]

A solution of tert-butyl (8-nitro-6,12-dioxo-6,12-dihydroindolo[2,1-b]quinazolin-3-yl) carbamate (from Example 46; 100 mg, 0.33 mmol) in TFA (5.0 mL) was stirred at 25° C. for 5 h. The solvent was removed under reduced pressure to afford 3-amino-8-nitroindolo[2,1-b]quinazoline-6,12-dione (80 mg, 60% yield) as a dark solid. ¹H NMR (400 MHz, DMSO-d₆) δ 8.69-8.66 (m, 2H), 8.62 (d, J=8.8 Hz, 1H), 8.48 (d, J=2.0 Hz, 1H), 7.98 (d, J=8.8 Hz, 1H), 6.90 (dt, J=8.8, 2.0 Hz, 2H), 6.57 (s, 2H). LC-MS: m/z [M+H]⁺ 309.0.

### Example 48: 1,3-Dichloro-6,12-dioxoindolo[2,1-b]quinazoline-8-carboxylic Acid (Compound 22)

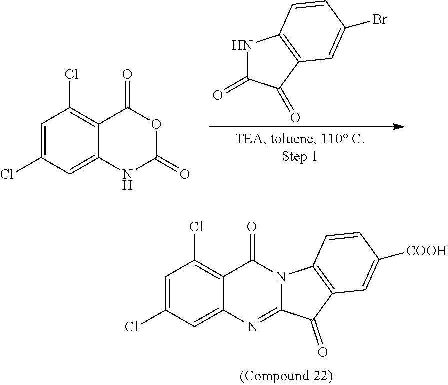 [SMILES: O=c1[nH]c2cc(Cl)cc(Cl)c2c(=O)o1 ; O=C1Nc2ccc(Br)cc2C1=O ; O=C(O)c1ccc2c(c1)C(=O)c1nc3cc(Cl)cc(Cl)c3c(=O)n1-2]

Following the method of General Procedure 1 but using 5,7-dichloro-1H-3,1-benzoxazine-2,4-dione and 2,3-dioxo-1H-indole-5-carboxylic acid as starting materials. The title compound was obtained as a green solid. ¹H NMR (400 MHz, DMSO-d₆) δ 8.57 (d, J=8.4 Hz, 1H), 8.42 (dd, J=8.4, 1.6 Hz, 1H), 8.26 (d, J=1.6 Hz, 1H), 8.08 (d, J=2.0 Hz, 1H), 7.96 (d, J=2.0 Hz, 1H). LC-MS: m/z [M+H]⁺ 361.0.

### Example 49: 1,3-Dichloro-6,12-dioxoindolo[2,1-b]quinazoline-8-carboxamide (Compound 23)

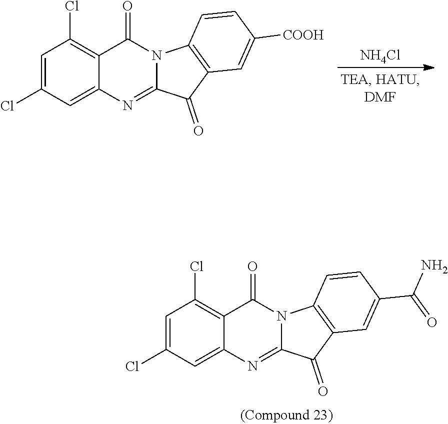 [SMILES: O=C(O)c1ccc2c(c1)C(=O)c1nc3cc(Cl)cc(Cl)c3c(=O)n1-2 ; "NH4Cl" ; NC(=O)c1ccc2c(c1)C(=O)c1nc3cc(Cl)cc(Cl)c3c(=O)n1-2]

A solution of 1,3-dichloro-6,12-dioxoindolo[2,1-b]quinazoline-8-carboxylic acid (from Example 48; 60 mg, 0.17 mmol), HATU (95 mg, 0.25 mmol), and Et₃N (50 mg, 0.5 mmol) in DMF (2 mL) was stirred at 25° C. for 10 min under N₂. Then, NH₄Cl (27 mg, 0.5 mmol) was added, and the mixture was stirred at 25° C. for 1 h under N₂. The product was recrystallized (DCM:MeOH 10:1) to give 1,3-dichloro-6,12-dioxoindolo[2,1-b]quinazoline-8-carboxamide (42.4 mg, 69% yield) as a green solid. 1H NMR (400 MHz, DMSO-d₆) δ 8.57-8.48 (m, 1H), 8.40-8.34 (m, 2H), 8.26 (s, 1H), 8.07 (d, J=2.0 Hz, 1H), 7.96 (d, J=2.0 Hz, 1H), 7.64 (s, 1H). LC-MS: m/z [M+H]⁺ 360.0.

### Example 50: 1,3-Dichloro-N-methyl-6,12-dioxo-6,12-dihydroindolo[2,1-b]quinazoline-8-carboxamide (Compound 20)

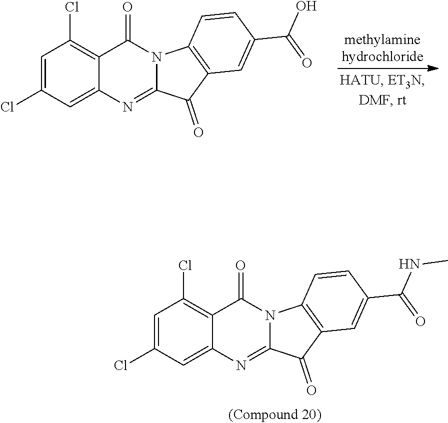 [SMILES: O=C(O)c1ccc2c(c1)C(=O)c1nc3cc(Cl)cc(Cl)c3c(=O)n1-2 ; "methylaminehydrochloride" ; CNC(=O)c1ccc2c(c1)C(=O)c1nc3cc(Cl)cc(Cl)c3c(=O)n1-2]

To a solution of 1,3-dichloro-6,12-dioxoindolo[2,1-b]quinazoline-8-carboxylic acid (from Example 48; 75 mg, 0.2 mmol) and methylamine hydrochloride (14.0 mg, 0.2 mmol) in DMF (2.0 mL) were added Et₃N (126 mg, 1.2 mmol) and HATU (237 mg, 0.6 mmol). The mixture was stirred at room temperature for one hour. The mixture was concentrated under reduced pressure. The residue was added to H₂O, and the suspension was filtered. The filtrate was washed with H₂O and CAN and dried under reduced pressure to give 1,3-dichloro-N-methyl-6,12-dioxo-6,12-dihydroindolo[2,1-b]quinazoline-8-carboxamide (35 mg, 45% yield) as a green solid. ¹H NMR (400 MHz, DMSO-d₆) δ 8.76 (d, J=4.4 Hz, 1H), 8.52 (d, J=8.4 Hz, 1H), 8.33 (s, 2H), 8.07 (d, J=2.0 Hz, 1H), 7.96 (d, J=2.0 Hz, 1H), 2.82 (d, J=4.4 Hz, 3H). LC-MS: m/z [M+H]⁺ 374.9.

### Example 51: 1,3-Dichloro-N,N-dimethyl-6,12-dioxo-6,12-dihydroindolo[2,1-b]quinazoline-8-carboxamide (Compound 24)

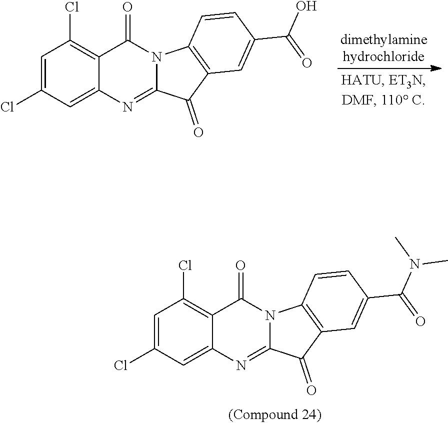 [SMILES: O=C(O)c1ccc2c(c1)C(=O)c1nc3cc(Cl)cc(Cl)c3c(=O)n1-2 ; "dimethylaminehydrochloride" ; CN(C)C(=O)c1ccc2c(c1)C(=O)c1nc3cc(Cl)cc(Cl)c3c(=O)n1-2]

To a solution of 1,3-dichloro-6,12-dioxoindolo[2,1-b]quinazoline-8-carboxylic acid (from Example 48; 100 mg, 0.3 mmol) and dimethylamine·HCl (27 mg, 0.3 mmol) in DMF (5.0 mL) were added Et₃N (168 mg, 1.7 mmol) and HATU (316 mg, 0.8 mmol). The mixture was stirred at 110° C. for 12 hours then concentrated under reduced pressure. The residue was added to H₂O, and the solids were filtered under reduced pressure. The filtered cake was washed with H₂O, MeOH and CH₃CN and dried under reduced pressure to give 1,3-dichloro-N,N-dimethyl-6,12-dioxo-6,12-dihydroindolo[2,1-b]quinazoline-8-carboxamide (65 mg, 59% yield) as a yellow solid.

¹H NMR (400 MHz, DMSO-d₆) δ 8.49 (d, J=9.2 Hz, 1H), 8.07 (d, J=2.0 Hz, 1H), 7.96 (d, J=2.2 Hz, 1H), 7.92 (d, J=7.6 Hz, 2H), 2.99 (d, J=21.6 Hz, 6H). LC-MS: m/z [M+H]⁺ 388.0.

### Example 52: tert-Butyl 4-(1,3-dichloro-6,12-dioxo-6,12-dihydroindolo[2,1-b]quinazoline-8-carbonyl)piperazine-1-carboxylate (Compound 26)

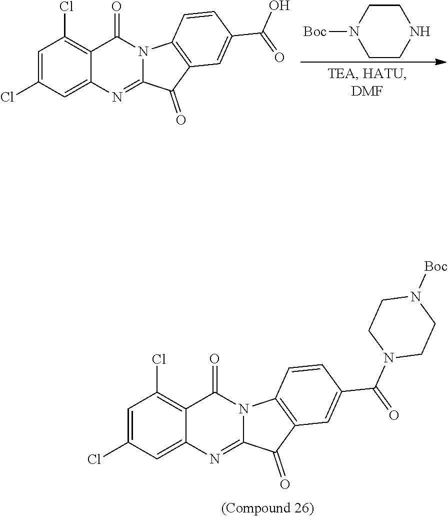 [SMILES: O=C(O)c1ccc2c(c1)C(=O)c1nc3cc(Cl)cc(Cl)c3c(=O)n1-2 ; CC(C)(C)OC(=O)N1CCNCC1 ; CC(C)(C)OC(=O)N1CCN(C(=O)c2ccc3c(c2)C(=O)c2nc4cc(Cl)cc(Cl)c4c(=O)n2-3)CC1]

To a solution of 1,3-dichloro-6,12-dioxo-6,12-dihydroindolo[2,1-b]quinazoline-8-carboxylic acid (from Example 48; 100 mg, 0.28 mmol) in DMF (5 mL) were added tert-butyl piperazine-1-carboxylate (62 mg, 0.33 mmol), HATU (158 mg, 0.42 mmol), and Et₃N (84 mg, 0.83 mmol). The mixture was stirred for 4.5 h at 25° C. Water (2 mL) was added to the solution, and the solids were filtered. The filter cake was washed with water (10 mL), EA (20 mL) and DCM (20 mL) and dried under reduced pressure to afford tert-butyl 4-(1,3-dichloro-6,12-dioxo-6,12-dihydroindolo[2,1-b]quinazoline-8-carbonyl) piperazine-1-carboxylate (160 mg) as a green solid.

¹H NMR (400 MHz, DMSO-d₆) δ 8.51 (d, J=8.8 Hz, 1H), 8.07 (s, 1H), 7.96-7.91 (m, 3H), 3.61-3.41 (m, 8H), 1.41 (s, 9H). LC-MS: m/z [M+H]⁺ 473.0.

### Example 53: 4-Bromo-6,12-dioxo-6,12-dihydroindolo[2,1-b]quinazoline-8-carbonitrile (Compound 25)

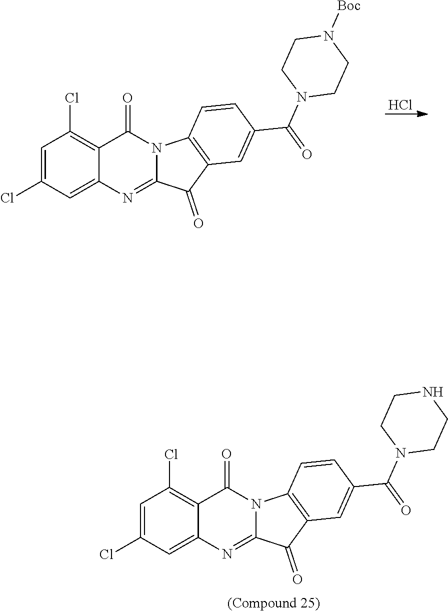 [SMILES: CC(C)(C)OC(=O)N1CCN(C(=O)c2ccc3c(c2)C(=O)c2nc4cc(Cl)cc(Cl)c4c(=O)n2-3)CC1 ; Cl ; O=C1c2cc(C(=O)N3CCNCC3)ccc2-n2c1nc1cc(Cl)cc(Cl)c1c2=O]

A solution of tert-butyl 4-(1,3-dichloro-6,12-dioxo-6,12-dihydroindolo[2,1-b]quinazoline-8-carbonyl)piperazine-1-carboxylate (from Example 52; 160 mg, 0.30 mmol) in 1,4-dioxane (4 M HCl, 5 mL) was stirred at 25° C. for 4.5 h. The solution was concentrated under reduced pressure to afford 1,3-dichloro-8-(piperazine-1-carbonyl)indolo[2,1-b]quinazoline-6,12-dione (120 mg) as a green solid. ¹H NMR (400 MHz, DMSO-d₆) δ 9.08 (s, 2H), 8.52 (d, J=8.4 Hz, 1H), 8.08 (d, J=2.0 Hz, 1H), 8.01 (s, 1H), 7.99-7.95 (m, 2H), 3.68 (s, 4H), 3.20 (s, 4H). LC-MS: m/z [M+H]⁺ 429.0.

### Example 54: N-(2-Aminoethyl)-1,3-dichloro-6,12-dioxo-6,12-dihydroindolo[2,1-b]quinazoline-8-carboxamide (Compound 19)

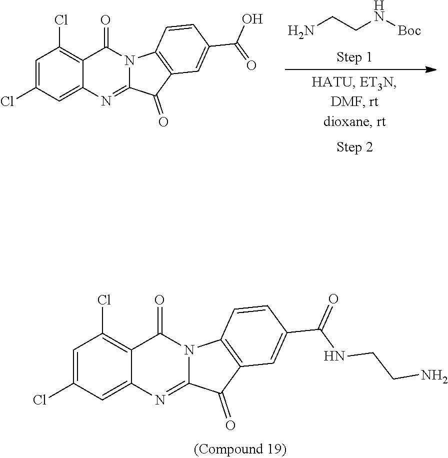 [SMILES: O=C(O)c1ccc2c(c1)C(=O)c1nc3cc(Cl)cc(Cl)c3c(=O)n1-2 ; CC(C)(C)OC(=O)NCCN ; NCCNC(=O)c1ccc2c(c1)C(=O)c1nc3cc(Cl)cc(Cl)c3c(=O)n1-2]

### Step 1: tert-butyl (2-(1,3-dichloro-6,12-dioxo-6,12-dihydroindolo[2,1-b]quinazoline-8-carboxamido)ethyl)carbamate

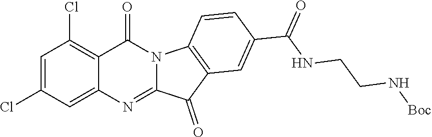 [SMILES: CC(C)(C)OC(=O)NCCNC(=O)c1ccc2c(c1)C(=O)c1nc3cc(Cl)cc(Cl)c3c(=O)n1-2]

To a solution of 1,3-dichloro-6,12-dioxoindolo[2,1-b]quinazoline-8-carboxylic acid (from Example 48; 200 mg, 0.6 mmol) and (2-aminoethyl)amino tert-butyl formate (107 mg, 0.7 mmol) in DMF (5.0 mL) were added HATU (632 mg, 1.7 mmol) and Et₃N (168 mg, 1.7 mmol). The mixture was stirred at room temperature for one hour. The mixture was concentrated under reduced pressure. The residue was added to H₂O, and the suspension was filtered. The filtrate was washed with H₂O and CAN and dried under reduced pressure to give tert-butyl (2-(1,3-dichloro-6,12-dioxo-6,12-dihydroindolo[2,1-b]quinazoline-8-carboxamido)ethyl)carbamate (240 mg, crude) as a dark green solid. LC-MS: m/z [M+H-Boc]⁺403.0.

### Step 2: N-(2-aminoethyl)-1,3-dichloro-6,12-dioxo-6,12-dihydroindolo[2,1-b]quinazoline-8-carboxamide (Compound 19)

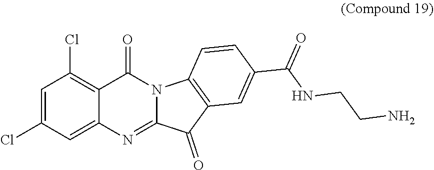 [SMILES: NCCNC(=O)c1ccc2c(c1)C(=O)c1nc3cc(Cl)cc(Cl)c3c(=O)n1-2]

A solution of tert-butyl (2-(1,3-dichloro-6,12-dioxo-6,12-dihydroindolo[2,1-b]quinazoline-8-carboxamido)ethyl)carbamate (160 mg, 0.3 mmol) in dioxane (5.0 mL) was stirred at room temperature for 1 hour. The mixture was concentrated under reduced pressure. The residue was added to H₂O and the suspension filtered under reduced pressure. The filtrate was washed with H₂O and CAN and dried under reduced pressure to give N-(2-aminoethyl)-1,3-dichloro-6,12-dioxoindolo[2,1-b]quinazoline-8-carboxamide (112 mg, 86%) as a green solid. ¹H NMR (400 MHz, DMSO-d₆) δ 8.98 (t, J=5.2 Hz, 1H), 8.54 (d, J=8.0 Hz, 1H), 8.38 (d, J=8.4 Hz, 2H), 8.02 (dd, J=40.0, 2.0 Hz, 5H), 3.56 (d, J=7.2 Hz, 2H), 3.03 (dd, J=11.4, 5.6 Hz, 2H). LC-MS: m/z [M+H]⁺ 403.0.

### Example 55: 3-Fluoro-8-nitro-5H,5aH,6H,12H-indolo[2,1-b]quinazolin-6-ol (Compound 21)

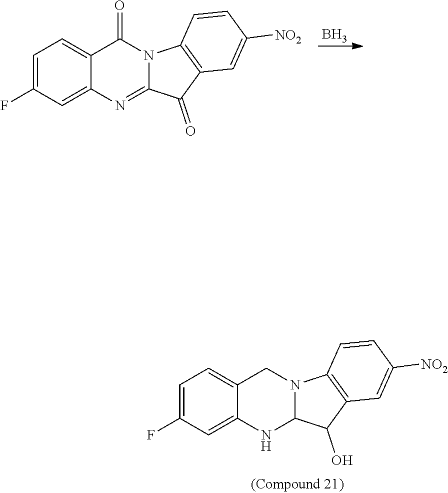

To a solution of 3-fluoro-8-nitroindolo[2,1-b]quinazoline-6,12-dione (from Example 8; 100 mg, 0.32 mmol) in THF (3 mL) was added BH₃ (1.0 M in THF) (1 mL). Then the mixture was stirred for 12 h at 70° C. under N₂. The reaction was quenched with NH₄Cl (50 mL). The solvent was extracted with EA (30 mL×3). The combined organic layers were washed with brine (50 mL×2) and dried over Na₂SO₄, filtered, and concentrated to dryness. The residue was purified by Prep-HPLC (Chromatographic column:—Xbridge-C18 150×19 mm, 5 μm Mobile Phase: CAN/H₂O, 30-60% CAN in 0.1% TFA/water, 20 mL/min) to give 3-fluoro-8-nitro-5H,5aH,6H,12H-indolo[2,1-b]quinazolin-6-ol (8.2 mg, 8% yield) as white solid. ¹H NMR (400 MHz, DMSO-d₆) δ 8.15-8.09 (m, 2H), 7.07-6.98 (m, 1H), 6.81 (d, J=8.6 Hz, 1H), 6.63 (s, 1H), 6.44-6.37 (m, 1H), 6.33-6.27 (m, 1H), 5.91 (s, 1H), 4.84 (s, 1H), 4.81 (s, 1H), 4.77 (d, J=16.6 Hz, 1H), 4.51 (d, J=16.4 Hz, 1H). ¹⁹F NMR (376 MHz, DMSO-d₆) δ−115.42. LC-MS: m/z [M+H]⁺ 320.1.

### Example 56: 3-Fluoro-6,12-dioxo-6,12-dihydroindolo[2,1-b]quinazoline-8-sulfonamide (Compound 28)

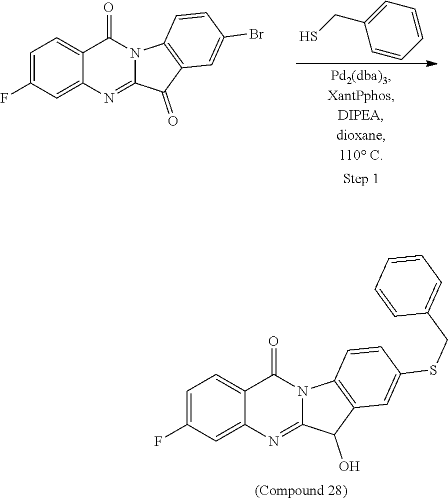 [SMILES: O=C1c2cc(Br)ccc2-n2c1nc1cc(F)ccc1c2=O ; SCc1ccccc1 ; O=c1c2ccc(F)cc2nc2n1-c1ccc(SCc3ccccc3)cc1C2O]

To a solution of 8-bromo-3-fluoroindolo[2,1-b]quinazoline-6,12-dione (from Example 1; 100 mg, 0.29 mmol), Pd₂(DBA)₃ (55 mg, 0.060 mmol) and Xantphos (65 mg, 0.15 mmol) in dioxane (2.0 mL) were added phenylmethanethiol (43 mg, 0.35 mmol) and DIPEA (75 mg, 0.60 mmol). The reactor was evacuated and backfilled with N₂ three times. The mixture was stirred for 16 h at 110° C. The solvent was removed under reduced pressure. The residue was purified by chromatography eluting with DCM in PE from 0% to 80% to afford 8-(benzylthio)-3-fluoroindolo[2,1-b]quinazoline-6,12-dione (50 mg, 32% yield) as a yellow solid. ¹H NMR (400 MHz, DMSO-d₆) δ 8.40-8.33 (m, 2H), 7.83-7.74 (m, 2H), 7.64-7.58 (m, 1H), 7.51 (d, J=7.2 Hz, 2H), 7.41-7.33 (m, 3H), 7.31-7.25 (m, 1H), 4.50 (s, 2H). LC-MS: m/z [M+H]⁺ 389.1.

### Example 57: 8-(Benzylthio)-3-fluoroindolo[2,1-b]quinazoline-6,12-dione (Compound 27)

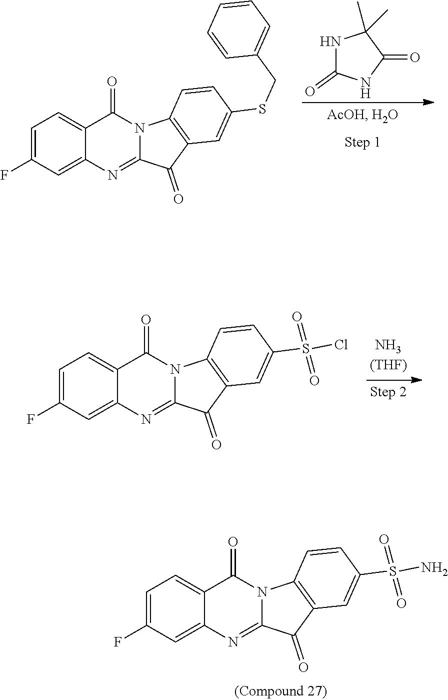

### Step 1: 3-fluoro-6,12-dioxo-6,12-dihydroindolo[2,1-b]quinazoline-8-sulfonyl Chloride

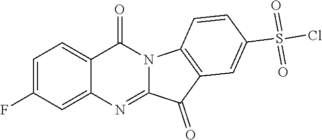 [SMILES: O=C1c2cc(S(=O)(=O)Cl)ccc2-n2c1nc1cc(F)ccc1c2=O]

To a solution of 8-(benzylthio)-3-fluoroindolo[2,1-b]quinazoline-6,12-dione (from Example 27; 50 mg, 0.13 mmol) in MeCN (4.0 ml), H₂O (0.2 mL) and AcOH (0.2 mL) was added 5,5-dimethylimidazolidine-2,4-dione (38 mg, 0.19 mol) at 0° C. The mixture was stirred for 2.0 h at 25° C. The solvent was removed under reduced pressure, and the residue was dissolved into DCM and washed with water. The organic layer was dried (Na₂SO₄), filtered, and concentrated under reduced pressure to afford the title compound (35 mg, crude) as a yellow solid. LC-MS: m/z [M+H]⁺ 347.1.

### Step 2: 3-fluoro-6,12-dioxo-6,12-dihydroindolo[2,1-b]quinazoline-8-sulfonamide (Compound 27)

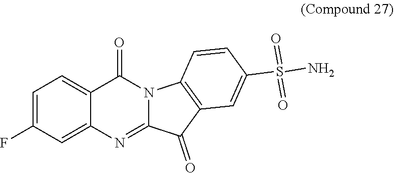 [SMILES: NS(=O)(=O)c1ccc2c(c1)C(=O)c1nc3cc(F)ccc3c(=O)n1-2]

A solution of 3-fluoro-6,12-dioxo-6,12-dihydroindolo[2,1-b]quinazoline-8-sulfonyl chloride (35 mg, 0.096 mmol) and NH₃ (1 M) in THF (2.0 mL), was stirred at 25° C. for 16 h. The solvent was removed under reduced pressure, and the residue was purified by chromatography eluting with DCM in PE from 0% to 80% to afford 3-fluoro-6,12-dioxo-6,12-dihydroindolo[2,1-b]quinazoline-8-sulfonamide (20 mg, 60% yield) as a yellow solid. ¹H NMR (400 MHz, DMSO-d₆) δ 8.89 (s, 1H), 8.42 (dd, J=8.8, 6.0 Hz, 1H), 8.08 (d, J=8.0 Hz, 1H), 7.93-7.80 (m, 4H), 7.64 (td, J=8.8, 2.4 Hz, 1H). LC-MS: m/z [M+H]⁺ 347.1.

### Example 58: 8-(Benzylsulfonyl)-3-fluoroindolo[2,1-b]quinazoline-6,12-dione (Compound 33)

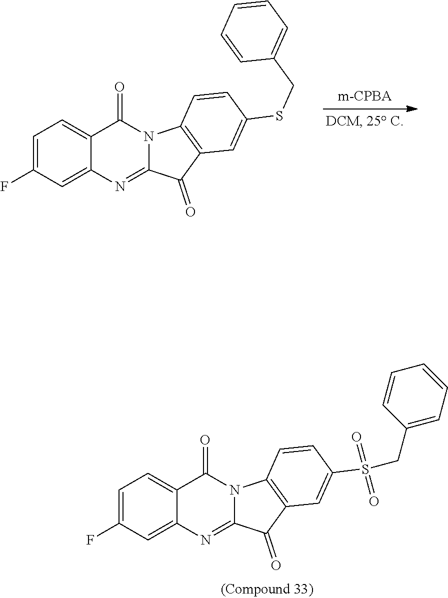 [SMILES: O=C1c2cc(SCc3ccccc3)ccc2-n2c1nc1cc(F)ccc1c2=O ; O=C1c2cc(S(=O)(=O)Cc3ccccc3)ccc2-n2c1nc1cc(F)ccc1c2=O]

To a solution of 8-(benzylthio)-3-fluoroindolo[2,1-b]quinazoline-6,12-dione (from Example 27; 0.18 g, 0.46 mmol) in DCM (5.0 mL) was added m-CPBA (0.24 g, 1.4 mmol). The reaction mixture was stirred for 16 h at 25° C. under N₂. The mixture was quenched with NaHCO₃ (aq). The organic layer was dried over Na₂SO₄, filtered, and concentrated under reduced pressure. The residue was dissolved into DMSO and water. The suspension was filtered, and the filter cake was dried under reduced pressure to afford 8-(benzylsulfonyl)-3-fluoroindolo[2,1-b]quinazoline-6,12-dione (20 mg, 10% yield) as a yellow solid. ¹H NMR (400 MHz, DMSO-d₆) δ 8.59 (d, J=8.4 Hz, 1H), 8.47-8.35 (m, 1H), 8.17-8.09 (m, 2H), 7.88 (d, J=6.8 Hz, 1H), 7.65 (s, 1H), 7.33 (s, 3H), 7.23 (s, 2H), 4.86 (s, 2H). LC-MS: m/z [M+H]421.0.

### Example 59: 3-Fluoro-6-hydroxy-12-oxo-5,5a,6,12-tetrahydroindolo[2,1-b]quinazoline-8-carbonitrile (Compound 31)

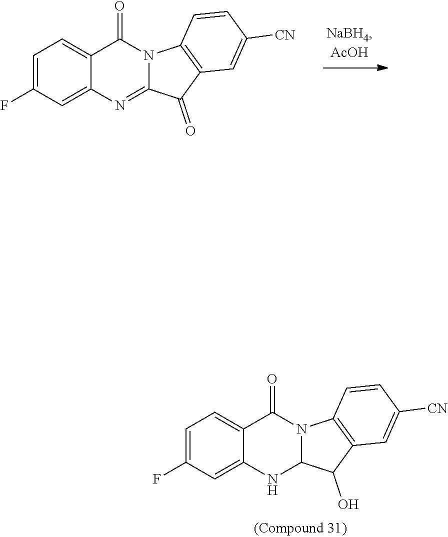 [SMILES: N#Cc1ccc2c(c1)C(=O)c1nc3cc(F)ccc3c(=O)n1-2 ; N#Cc1ccc2c(c1)C(O)C1Nc3cc(F)ccc3C(=O)N21]

A solution of 3-fluoro-6,12-dioxoindolo[2,1-b]quinazoline-8-carbonitrile (from Example 16; 250 mg, 0.86 mmol) in HOAc (3.4 mL) was stirred at 0° C. Then, NaBH₄ (325 mg, 8.6 mmol) was added to the solution at 0° C. The solution was stirred at 25° C. for 1 h under N₂. The mixture was diluted with aq. NaHCO₃ (to pH=8) and extracted with EA (50 mL×3). The combined organic layers were washed with brine (50 mL×2) and dried over Na₂SO₄, filtered, and concentrated to dryness. The residue was purified by Prep-HPLC (Chromatographic column: —Xbridge-C18 150×19 mm, 5 μm Mobile Phase: CAN/H₂O, 47-57% CAN in 0.1% TFA/water, 20 mL/min) to give 3-fluoro-6-hydroxy-12-oxo-5,5a,6,12-tetrahydroindolo[2,1-b]quinazoline-8-carbonitrile (160 mg, 63% yield) as a yellow solid. 1H NMR (400 MHz, DMSO-d₆) δ 8.20-8.11 (m, 2H), 7.91-7.77 (m, 3H), 6.81-6.64 (m, 2H), 6.60 (s, 1H), 5.32 (s, 1H), 5.27 (d, J=6.8 Hz, 1H). LC-MS: m/z [M+H]⁺ 296.1.

### Example 60: 1,3-Difluoro-6-hydroxy-8-nitro-5a,6-dihydroindolo[2,1-b]quinazolin-12(5H)-one (Compound 32)

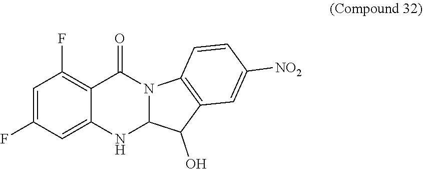 [SMILES: O=C1c2c(F)cc(F)cc2NC2C(O)c3cc([N+](=O)[O-])ccc3N12]

To a solution of 1,3-difluoro-8-nitroindolo[2,1-b]quinazoline-6,12-dione (from Example 14, 0.20 g, 0.61 mmol) in AcOH (3.0 mL) was added NaBH₄ (0.23 g, 6.1 mmol) at 0° C. The mixture was stirred for 1 h at 25° C. The mixture was quenched with ice-water and extracted with EA (30 mL×2). The combined organic layers were dried over Na₂SO₄, filtered, and concentrated under reduced pressure. The residue was purified by chromatography eluting with DCM in PE from 0% to 80% to afford 1,3-difluoro-6-hydroxy-8-nitro-5a,6-dihydroindolo[2,1-b]quinazolin-12(5H)-one (30 mg, 15% yield) as an off-white solid. 1H NMR (400 MHz, DMSO-d₆) δ 8.38 (s, 1H), 8.31 (dd, J=8.8, 2.4 Hz, 1H), 8.25 (s, 1H), 8.17 (d, J=8.8 Hz, 1H), 6.71-6.64 (m, 3H), 6.57 (dd, J=17.2, 8.4 Hz, 2H), 5.40-5.32 (m, 2H). LC-MS: m/z [M+H]⁺ 334.1.

### Example 61: 1,3-Difluoro-8-nitroindolo[2,1-b]quinazolin-12(6H)-one (Compound 34)

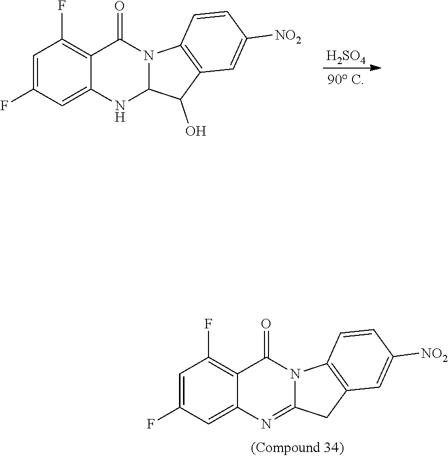

A solution of 1,3-difluoro-6-hydroxy-8-nitro-5a,6-dihydroindolo[2,1-b]quinazolin-12(5H)-one (from Example 60; 50 mg, 0.15 mmol) in H₂SO₄ (2.0 mL) was stirred for 1.0 h at 85° C. The mixture was poured into ice-water and extracted with the EA (30 mL×2). The combined organic layers were dried over Na₂SO₄, filtered, and concentrated under reduced pressure. The residue was purified by chromatography eluting with MeOH in DCM from 0% to 5% to afford 1,3-difluoro-8-nitroindolo[2,1-b]quinazolin-12(6H)-one (20 mg, 25% yield) as a dark red solid. ¹H NMR (400 MHz, DMSO-d₆) δ 12.31 (s, 1H), 8.69 (d, J=8.8 Hz, 1H), 8.45 (d, J=2.0 Hz, 1H), 8.04 (dd, J=8.8, 2.0 Hz, 1H), 7.00 (t, J=10.4 Hz, 1H), 6.87 (d, J=10.4 Hz, 1H), 6.27 (s, 1H). LC-MS: m/z [M+H]⁺ 316.0.

### Example 62: 3-Hydroxy-9-nitropyrido[3′,4′:4,5]pyrimido[1,2-a]indole-5,11-dione (Compound 51)

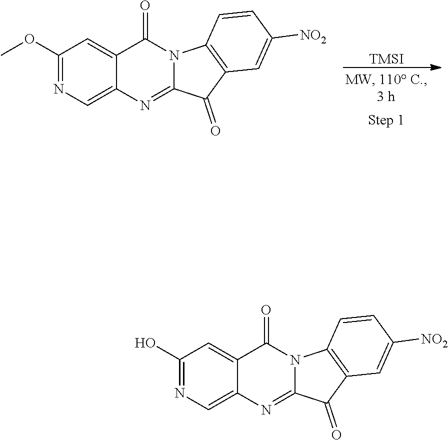 [SMILES: "TMSI" ; COc1cc2c(=O)n3c(nc2cn1)C(=O)c1cc([N+](=O)[O-])ccc1-3 ; O=C1c2cc([N+](=O)[O-])ccc2-n2c1nc1cnc(O)cc1c2=O]

To a solution of 3-methoxy-9-nitropyrido[3′,4′:4,5]pyrimido[1,2-a]indole-5,11-dione (100 mg, 0.3 mmol) in DMF (3 mL) was add TMSI (130 mg, 0.92 mmol). The mixture was stirred for 3 h at 110° C. in a microwave reactor then diluted with H₂O. The mixture was extracted with EA (50 mL×3). The combined organic layers were washed with brine (50 mL×2), dried over Na₂SO₄, filtered and concentrated to dryness. The residue was purified by prep-HPLC (Chromatographic columns: —Xbridge-C18 150×19 mm, 5 um Mobile Phase: ACN/H₂O, 10-20% ACN in 0.1% TFA/water, 20 mL/min) to give the title compound (7 mg, 7% yield) as a brown solid. ¹H NMR (400 MHz, DMSO-d₆) δ 8.71-8.62 (m, 2H), 8.57 (s, 1H), 8.49 (d, J=2.0 Hz, 1H), 8.30 (s, 1H), 6.80 (s, 1H). LC-MS: m/z [M+H]⁺ 311.1.

### Example 63: 4-Bromo-3-chloro-8-nitroindolo[2,1-b]quinazoline-6,12-dione (Compound 50)

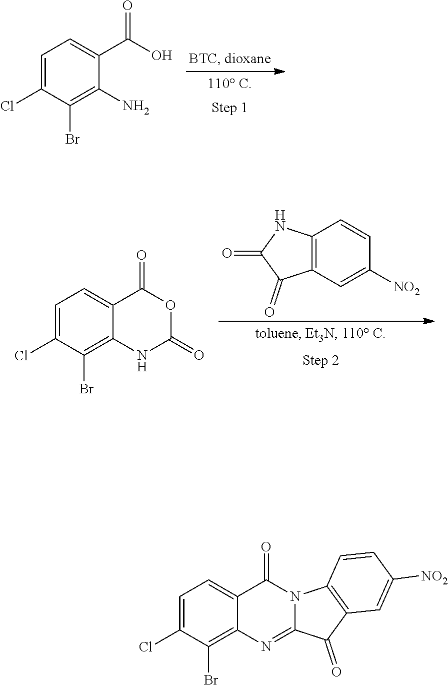 [SMILES: Nc1c(C(=O)O)ccc(Cl)c1Br ; O=c1[nH]c2c(Br)c(Cl)ccc2c(=O)o1 ; O=C1Nc2ccc([N+](=O)[O-])cc2C1=O ; O=C1c2cc([N+](=O)[O-])ccc2-n2c1nc1c(Br)c(Cl)ccc1c2=O]

Following the method of General Procedure 3 but using 2-amino-3-bromo-4-chlorobenzoic acid and 5-nitro-1H-indole-2,3-dione as starting materials the title compound was obtained as a brown solid. ¹H NMR (400 MHz, DMSO-d₆) δ 8.75 (dd, J=8.8, 2.4 Hz, 1H), 8.68-8.60 (m, 2H), 8.34 (d, J=8.4 Hz, 1H), 7.99 (d, J=8.4 Hz, 1H). LC-MS: m/z [M+H]⁺ 407.9.

### Example 64: 1,3-Dichloro-6,12-dioxo-N-(2-(sulfamoylamino)ethyl)-6,12-dihydroindolo[2,1-b]quinazoline-8-carboxamide (Compound 29)

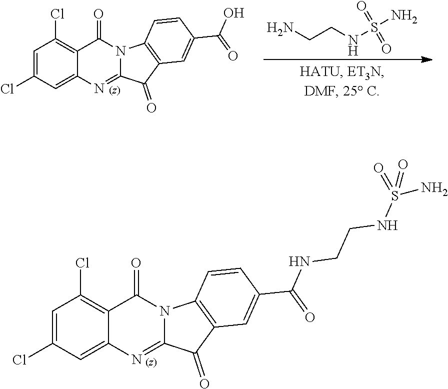 [SMILES: O=C(O)c1ccc2c(c1)C(=O)c1nc3cc(Cl)cc(Cl)c3c(=O)n1-2 ; NCCNS(N)(=O)=O ; NS(=O)(=O)NCCNC(=O)c1ccc2c(c1)C(=O)c1nc3cc(Cl)cc(Cl)c3c(=O)n1-2]

Following the procedure of Example 52 but using 1,3-dichloro-6,12-dioxo-6,12-dihydroindolo[2,1-b]quinazoline-8-carboxylic acid (Example 48); the title compound was obtained as a yellow solid. 1H NMR (400 MHz, DMSO-d₆) δ 8.78 (d, J=5.4 Hz, 1H), 8.52 (d, J=9.2 Hz, 1H), 8.35 (d, J=8.0 Hz, 2H), 8.06 (d, J=2.0 Hz, 1H), 7.95 (d, J=2.0 Hz, 1H), 6.71 (t, J=6.4 Hz, 1H), 6.58 (s, 2H), 3.44 (dd, J=12.4, 6.4 Hz, 2H), 3.09 (dd, J=12.4, 6.4 Hz, 2H). LC-MS: m/z [M+H]⁺ 484.0.

### General Procedure 5

### Example 65: Synthesis of 6-[(aminosulfoamino)methyl]-17-(methoxyimino)-9-oxo-2,10-diazatetracyclo[8.7.0.0^{3,8}.0^{11,16}]heptadeca-1,3,5,7,11(16),12,14-heptaene-14-carbonitrile (Compound 159)

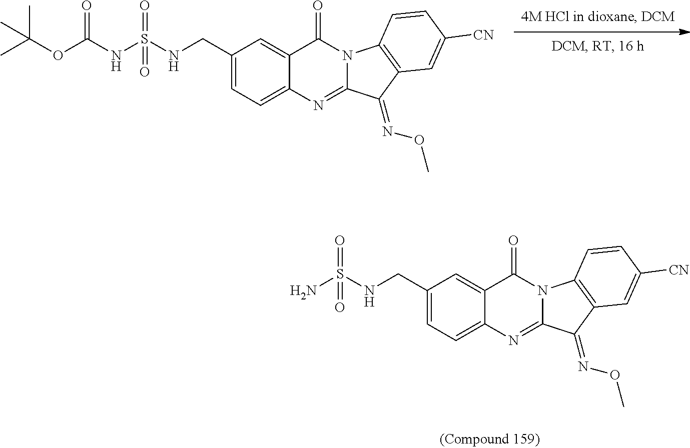 [SMILES: CO/N=C1\c2cc(C#N)ccc2-n2c1nc1ccc(CNS(=O)(=O)NC(=O)OC(C)(C)C)cc1c2=O ; CO/N=C1\c2cc(C#N)ccc2-n2c1nc1ccc(CNS(N)(=O)=O)cc1c2=O]

To the stirred solution of tert-butyl (E)-(N-((8-cyano-6-(methoxyimino)-12-oxo-6,12-dihydroindolo[2,1-b]quinazolin-2-yl)methyl)sulfamoyl)carbamate (0.3 g, 0.58 mmol, 1.0 equiv) in DCM (5 ml) at 0° C., was added 4 M HCl in dioxane (2.5 ml, 10 vol). The reaction mixture was stirred at RT for 4 hours. After complete consumption of starting material, the reaction mixture was evaporated and dried under vacuum. The solid residue was triturated with diethyl ether. The precipitates were filtered and dried to obtain 6-[(aminosulfoamino)methyl]-17-(methoxyimino)-9-oxo-2,10-diazatetracyclo[8.7.0.0^{3,8}.0^{11,16}]heptadeca-1,3,5,7,11(16),12,14-heptaene-14-carbonitrile (0.008 g, 3% yield) as a yellow solid. ¹H NMR (400 MHz, DMSO-d₆) δ 8.65 (dd, J=5.2, 3.6 Hz, 2H), 8.30 (d, J=8.0 Hz, 1H), 8.19 (dd, J=8.4 Hz, 1.6 Hz, 1H), 7.88 (dd, J=8.4, 6.8 Hz, 1H), 7.82 (d, J=8.4 Hz, 1H), 7.33-7.26 (brs, 1H), 6.75-6.66 (brs, 2H), 4.34 (s, 3H), 4.26 (s, 2H). LC-MS: m/z [M+H]⁺ 411.2.

### Example 66: Synthesis of 6-[(aminosulfoamino)methyl]-17-(oxo)-9-oxo-2,10-diazatetracyclo[8.7.0.0^{3,8}.0^{11,16}]heptadeca-1,3,5,7,11(16),12,14-heptaene-14-carbonitrile. (Compound 143)

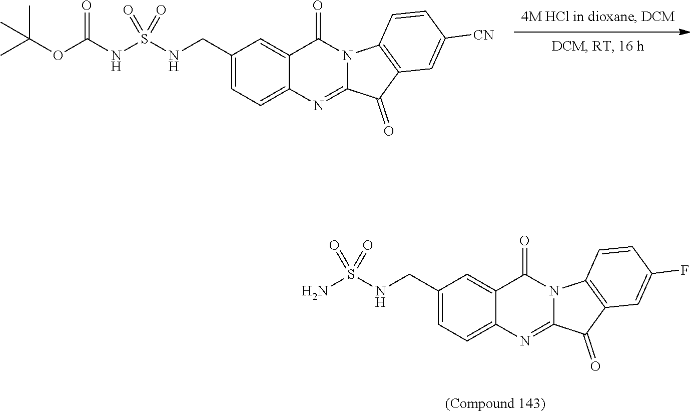 [SMILES: CC(C)(C)OC(=O)NS(=O)(=O)NCc1ccc2nc3n(c(=O)c2c1)-c1ccc(C#N)cc1C3=O ; NS(=O)(=O)NCc1ccc2nc3n(c(=O)c2c1)-c1ccc(F)cc1C3=O]

Following general procedure 5 but using tert-butyl (N-((8-cyano-6-(oxo)-12-oxo-6,12-dihydroindolo[2,1-b]quinazolin-2-yl)methyl)sulfamoyl)carbamate the title compound was obtained as a solid (7% yield). ¹H NMR (400 MHz, DMSO-d₆) δ 8.50 (dd, J=8.8, 4.0 Hz, 1H), 8.31 (s, 1H), 7.91 (s, 2H), 7.78 (dd, J=7.2, 2.8 Hz, 1H), 7.75-7.70 (m, 1H), 7.34 (brs, 1H), 6.76 (brs, 2H), 4.28 (s, 2H). LC-MS: m/z [M+H]⁺ 375.2.

### Example 67: Synthesis of N-(8-cyano-6,12-dioxo-6,12-dihydroindolo[2,1-b]quinazolin-2-yl) 4-methylpentanamide. (Compound 158)

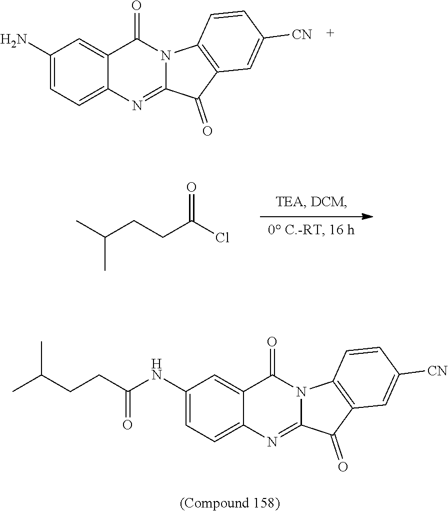

To the stirred solution of 6-amino-9,17-dioxo-2,10-diazatetracyclo[8.7.0.0^{3,8}.0^{11,16}]heptadeca-1,3,5,7,11(16),12,14-heptaene-14-carbonitrile (100 mg, 347 μmol, 1.0 equiv) in DCM (4 mL) at 0° C. under N₂ was added Et₃N (70.2 mg, 694 μmol, 2.0 equiv) and stirred for 10 min. Then, 4-methylvaleryl chloride (51.4 mg, 382 μmol, 1.1 equiv) was added and mixture was stirred at 25° C. for 16 h. After complete consumption of the starting material, the reaction mixture was quenched with H₂O (20 ml) and extracted with DCM (2×40 mL) The combined organic extracts were dried over anhydrous Na₂SO₄, filtered and concentrated under reduced pressure. The crude product purified by prep-HPLC to obtain N-{14-cyano-9,17-dioxo-2,10-diazatetracyclo[8.7.0.0^{3,8}.0^{11,16}]heptadeca-1,3,5,7,11(16),12,14-heptaen-6-yl}4-methylvaleramide (0.007 g, 5%) as a light yellow solid. ¹H NMR (400 MHz, DMSO-d₆) δ 10.5 (s, 1H), 8.71 (d, J=2.4 Hz, 1H), 8.62 (d, J=8.4 Hz, 1H), 8.42 (d, J=1.2 Hz, 1H), 8.30 (dd, J=8.4, 1.6 Hz, 1H), 8.06 (dd, J=8.4, 2.4 Hz, 1H), 7.93 (d, J=8.8 Hz, 1H), 2.42 (m, 2H), 1.55-1.63 (m, 3H), 0.92-0.89 (m, 6H). LC-MS: m/z [M+H]⁺ 387.2.

### Example 68: tert-butyl (6,12-dioxo-8-(2H-tetrazol-5-yl)-6,12-dihydroindolo[2,1-b]quinazolin-2-yl)carbamate (Compound 161)

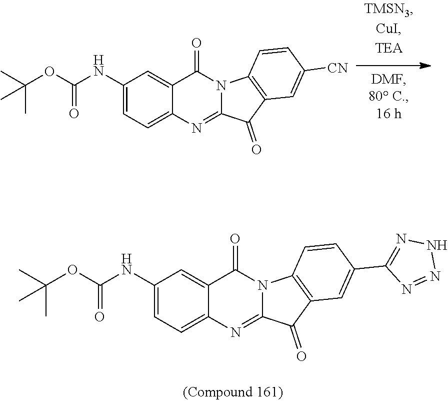 [SMILES: CC(C)(C)OC(=O)Nc1ccc2nc3n(c(=O)c2c1)-c1ccc(C#N)cc1C3=O ; CC(C)(C)OC(=O)Nc1ccc2nc3n(c(=O)c2c1)-c1ccc(-c2nn[nH]n2)cc1C3=O]

To the stirred solution of tert-butyl-14-cyano-9,17-dioxo-2,10-diazatetracyclo[8.7.0.0^{3,8}.0^{11,16}]heptadeca-1,3,5,7,11(16),12,14-heptaen-6-ylaminoformylate (0.4 g, 1.03 mmol, 1.0 equiv) in Dry DMF (12 mL) at RT under N₂ were added TMSN₃ (178 mg, 1.54 mmol, 1.5 equiv), Et₃N (0.43 ml, 3.09 mmol, 3.0 equiv) and CuI (0.019 g, 0.103 mmol, 0.1 equiv) at RT. The resulting mixture was heated at 80° C. for 16 h. After complete consumption of the starting material, the reaction mixture was cooled to RT and the mixture was quenched with H₂O (30 ml), extracted with DCM (2×50 mL). Combined organic extracts were dried over anhydrous Na₂SO₄, filtered and concentrated under reduced pressure. The crude product purified by prep-HPLC to obtain 5-{6-(tert-butoxycarbonylamino)-9,17-dioxo-2,10-diazatetracyclo[8.7.0.0^{3,8}.0^{11,16}]heptadeca-1,3,5,7,11(16),12,14-heptaen-14-yl}-2H-tetraazole (0.088 g, 20%) as a yellow solid. ¹H NMR (400 MHz, DMSO-d₆) δ 10.05 (s, 1H), 8.60-8.55 (m, 4H), 7.92-7.86 (m, 2H), 1.52 (s, 9H). Note; tetrazole NH-proton not observed. LC-MS: m/z [M+H]⁺ 432.2.

### Example 69: Synthesis of 5-(2-((tert-butoxycarbonyl)amino)-6,12-dioxo-6,12-dihydroindolo[2,1-b]quinazolin-8-yl)tetrazol-2-ide sodium salt

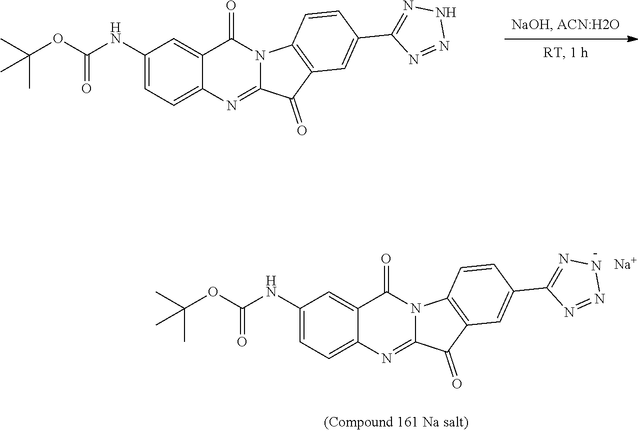 [SMILES: CC(C)(C)OC(=O)Nc1ccc2nc3n(c(=O)c2c1)-c1ccc(-c2nn[nH]n2)cc1C3=O ; CC(C)(C)OC(=O)Nc1ccc2nc3n(c(=O)c2c1)-c1ccc(-c2nn[nH]n2)cc1C3=O ; [Na+]]

To the stirred solution of 5-{6-(tert-butoxycarbonylamino)-9,17-dioxo-2,10-diazatetracyclo[8.7.0.0^{3,8}.0^{11,16}]heptadeca-1,3,5,7,11(16),12,14-heptaen-14-yl}-2H-tetraazole (18 mg, 41.7 μmol, 1.0 equiv) in acetonitrile (2.4 mL) and water (2.4 mL) (1:1) at RT under N₂ was added NaOH (16 mg, 41.7 μmol, 1 equiv). Further, the reaction mixture was stirred at 25° C. for 1 h. After 1 h, the reaction mixture was as such lyophilized to obtain 5-(2-((tert-butoxycarbonyl)amino)-6,12-dioxo-6,12-dihydroindolo[2,1-b]quinazolin-8-yl)tetrazol-2-ide sodium salt (18 mg, 95%) as a yellow solid. ¹H NMR (400 MHz, DMSO-d₆) δ 10.03 (s, 1H), 8.54 (d, J=1.6 Hz, 1H), 8.50-8.48 (m, 1H), 8.42 (dd, J=8.4, 1.6 Hz, 1H), 8.30 (brs, 1H), 7.92-7.84 (m, 2H), 1.52 (s, 9H). Note; two NH-proton not observed. LC-MS: m/z [M+H]⁺ 432.2.

### Example 70: 8-Fluoro-4-((4-fluorobenzyl)amino)indolo[2,1-b]quinazoline-6,12-dione (Compound 115)

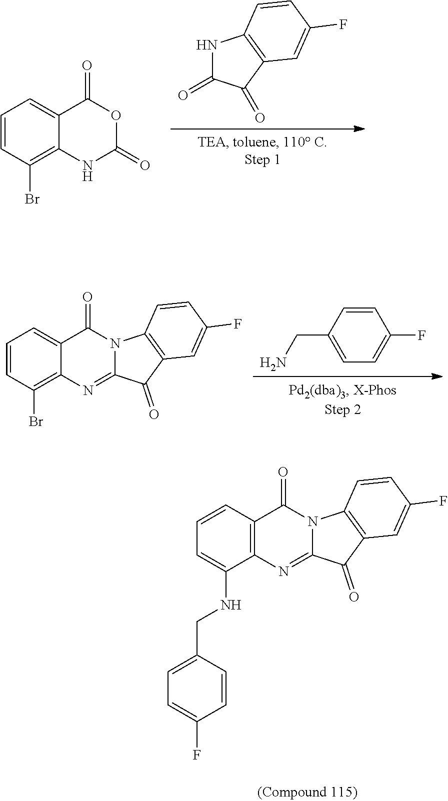 [SMILES: O=c1[nH]c2c(Br)cccc2c(=O)o1 ; O=C1Nc2ccc(F)cc2C1=O ; O=C1c2cc(F)ccc2-n2c1nc1c(Br)cccc1c2=O ; NCc1ccc(F)cc1 ; O=C1c2cc(F)ccc2-n2c1nc1c(NCc3ccc(F)cc3)cccc1c2=O]

### Step 1: 4-bromo-8-fluoroindolo[2,1-b]quinazoline-6,12-dione

Following the method of General Procedure 1 the title compound was obtained as a brown solid. LC-MS: m/z [M+H]⁺ 345.0.

### Step 2: 8-fluoro-4-((4-fluorobenzyl)amino)indolo[2,1-b]quinazoline-6,12-dione (Compound 115)

To a solution of 4-bromo-8-fluoroindolo[2,1-b]quinazoline-6,12-dione (100 mg, 0.29 mmol) and (4-fluorophenyl)methanamine (55 mg, 0.43 mmol) in dioxane (5 mL) were added Pd₂(dba)₂ (27 mg, 0.029 mmol), tripotassium phosphate (185 mg, 0.87 mmol) and X-Phos (21 mg, 0.043 mmol). Then the mixture was stirred for 12 h at 100° C. under N₂. The solvent was removed and the residue purified by Prep-HPLC (Xbridge-C18 150×19 mm, Sum; CH₃CN—H₂O, 65-95% ACN in 0.1% TFA/water) to give the title product (14.4 mg, 13% yield) as a brown solid. ¹H NMR (400 MHz, DMSO) δ 8.49 (dd, 1H), 7.78 (dd, 1H), 7.75-7.67 (m, 1H), 7.48-7.35 (m, 4H), 7.22 (t, 1H), 7.15 (t, 2H), 6.90-6.83 (m, 1H), 4.53 (d, 2H). LC-MS: m/z [M+H]⁺ 390.1.

### Example 71: Synthesis of (Z)-8-Fluoro-4-((4-fluorophenyl)amino)-6-(hydroxyimino)indolo[2,1-b]quinazolin-12(6H)-one (Compound 123)

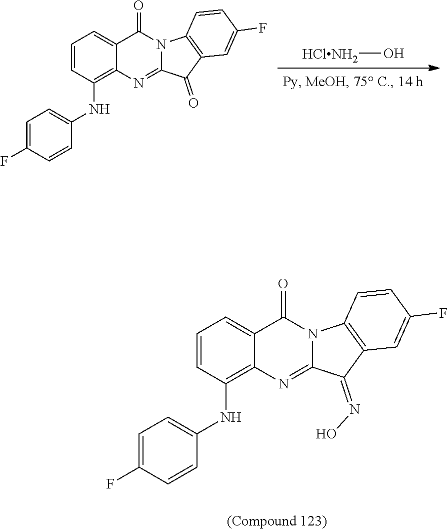

To a solution of 8-fluoro-4-[(4-fluorophenyl)amino]indolo[2,1-b]quinazoline-6,12-dione (110 mg, 0.29 mmol), NH₂OH·HCl (61 mg, 0.87 mmol) in MeOH (4 ml) was added pyridine (139 mg, 1.7 mmol). The mixture was stirred at 75° C. for 16 h then filtered and rinsed with MeOH (1 ml), water (1 ml). The residue was slurried with CH₂Cl₂ (4 ml) and filtered to provide the title compound (11 mg, 8%) as a red solid. ¹H NMR (400 MHz, DMSO) δ 13.86 (s, 1H), 8.58-8.55 (m, 1H), 8.13 (dd, J=8.4, 2.8 Hz, 1H), 8.05 (s, 1H), 7.65 (dd, J=1.6, 1.2 Hz, 1H), 7.57-7.52 (m, 1H), 7.47-7.36 (m, 4H), 7.21-7.17 (m, 2H). LC-MS: m/z [M+H]⁺ 391.1.

### Example 72: Synthesis of (Z)-8-Fluoro-4-((4-fluorophenyl)amino)-6-(methoxyimino)indolo[2,1-b]quinazolin-12(6H)-one (Compound 122)

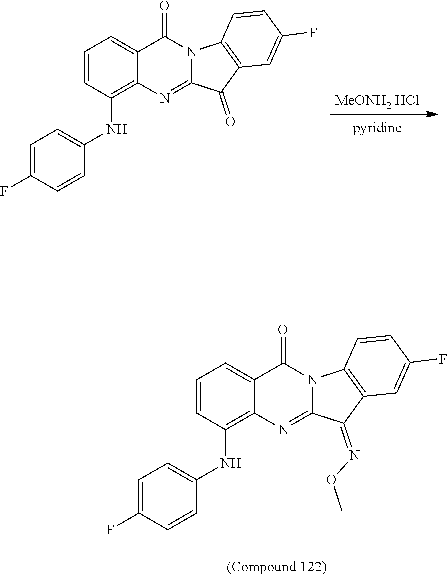 [SMILES: "MeONH2 HCl" ; O=C1c2cc(F)ccc2-n2c1nc1c(Nc3ccc(F)cc3)cccc1c2=O ; CO/N=C1/c2cc(F)ccc2-n2c1nc1c(Nc3ccc(F)cc3)cccc1c2=O]

To a solution of 8-fluoro-4-[(4-fluorophenyl)amino]indolo[2,1-b]quinazoline-6,12-dione (100 mg, 0.27 mmol), NH₂OMe·HCl (67 mg, 0.80 mmol) in MeOH (6 mL) was added pyridine (126 mg, 1.6 mmol). The mixture was stirred at 75° C. for 16 h. The mixture was filtered and rinsed with MeOH (1 mL), water (1 mL). The residue was slurried with CH₂Cl₂ (4 mL×2) and filtered to provide the title compound (14.5 mg, 13%) as a red solid. 1H NMR (400 MHz, CDCl₃) δ 8.68-8.65 (m, 1H), 8.00 (dd, 1H), 7.76-7.74 (m, 1H), 7.36 (s, 1H), 7.35 (d, 1H), 7.34-7.27 (m, 4H), 7.10-7.06 (m, 2H), 4.41 (s, 3H). LC-MS: m/z [M+H]⁺ 405.1.

### Example 73: 8-Fluoro-4-((4-fluorobenzyl)amino)indolo[2,1-b]quinazoline-6,12-dione (Compound 121)

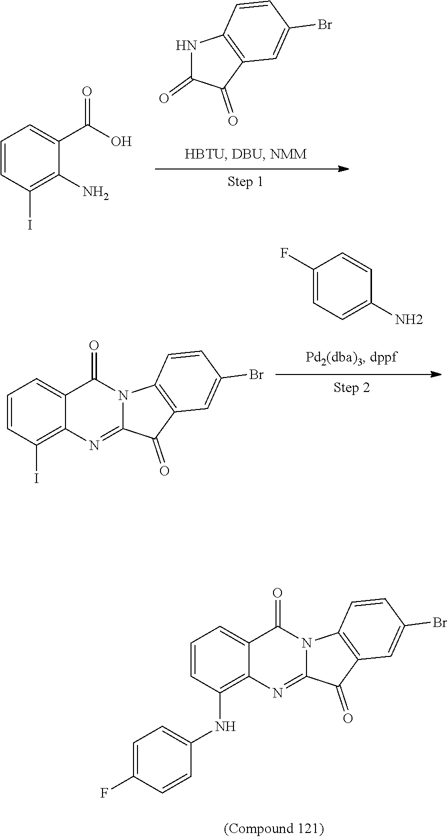 [SMILES: Nc1c(I)cccc1C(=O)O ; O=C1Nc2ccc(Br)cc2C1=O ; Nc1ccc(F)cc1 ; O=C1c2cc(Br)ccc2-n2c1nc1c(I)cccc1c2=O ; O=C1c2cc(Br)ccc2-n2c1nc1c(Nc3ccc(F)cc3)cccc1c2=O]

### Step 1: 8-bromo-4-iodoindolo[2,1-b]quinazoline-6,12-dione

Following the method of General Procedure 2 the title compound was obtained as green solid. LC-MS: m/z [M+H]⁺ 452.9.

### Step 2: 8-bromo-4-((4-fluorophenyl)amino)indolo[2,1-b]quinazoline-6,12-dione (Compound 121)

To a solution of 8-bromo-4-iodoindolo[2,1-b]quinazoline-6,12-dione (500 mg, 1.1 mmol) in toluene (6 mL) and were added 4-fluoroaniline (184 mg, 1.66 mmol), Pd₂(dba)₃ (101 mg, 0.11 mmol), dppf (92 mg, 0.16 mmol) and Cs₂CO₃ (1078 mg, 3.3 mmol) at 25° C. The mixture was stirred at 60° C. for 4 h. then purified by chromatography (DCM:MeOH=5:1) to give the title compound (400 mg, 79% yield) as yellow solid. ¹H NMR (400 MHz, DMSO) δ 8.43 (d, 2H), 8.10-8.02 (m, 2H), 7.64 (dd, 1H), 7.50 (t, 1H), 7.45-7.35 (m, 3H), 7.24-7.17 (m, 2H).

### Example 74: 8-(Benzylthio)-4-((4-fluorophenyl)amino)indolo[2,1-b]quinazoline-6,12-dione (Compound 120)

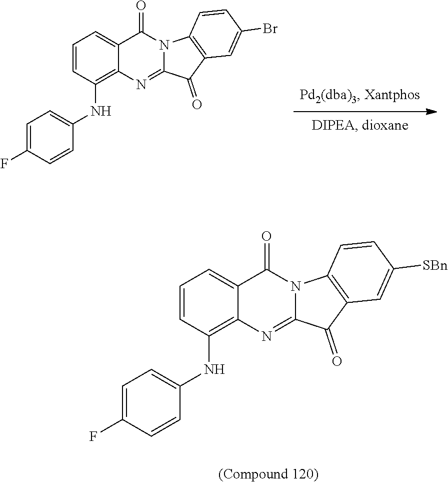

To a solution of 8-bromo-4-((4-fluorophenyl)amino)indolo[2,1-b]quinazoline-6,12-dione (250 mg, 0.57 mmol) in dioxane (10 mL) and were added phenylmethanethiol (107 mg, 0.86 mmol), Pd₂(dba)₃ (52 mg, 0.057 mmol), Xantphos (50 mg, 0.086 mmol) and N,N-Diisopropylethylamine (222 mg, 1.7 mmol) at 25° C. The reaction was stirred at 110° C. for 12 h. then concentrated to dryness. The mixture was diluted with CH₃CN (20 mL), and filtered to yield the title product (65 mg, 0.13 mmol, 24% yield) as a brown solid. ¹H NMR (400 MHz, DMSO) δ 8.45-8.32 (m, 2H), 7.84-7.74 (m, 2H), 7.63 (dd1H), 7.49 (t, 1H), 7.45-7.37 (m, 5H), 7.35-7.28 (m, 2H), 7.27-7.17 (m, 3H), 4.38 (s, 2H). LC-MS: m/z [M+H]⁺ 480.0.

### Example 75: 4-((4-Fluorophenyl)amino)-6,12-dioxo-6,12-dihydroindolo[2,1-b]quinazoline-8-carbonitrile (Compound 119)

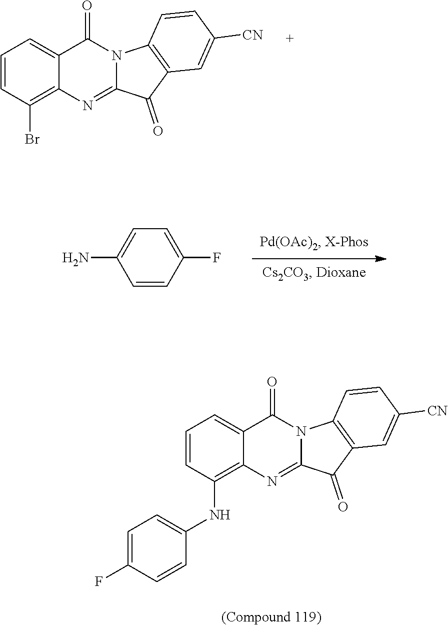 [SMILES: N#Cc1ccc2c(c1)C(=O)c1nc3c(Br)cccc3c(=O)n1-2 ; Nc1ccc(F)cc1 ; N#Cc1ccc2c(c1)C(=O)c1nc3c(Nc4ccc(F)cc4)cccc3c(=O)n1-2]

To a solution of 4-bromo-6,12-dioxoindolo[2,1-b]quinazoline-8-carbonitrile (150 mg 0.4 mmol) in dioxane (5 mL) and were added 4-fluoroaniline (71 mg, 0.6 mmol), Pd(OAc)₂ (10 mg 0.04 mmol), X-Phos (41 mg, 0.085 mmol) and Cs₂CO₃ (416 mg, 1.28 mmol) at 25° C. The mixture was stirred at 110° C. for 6 h, cooled and purified by column (SiO₂, DCM:MeOH=5:1) to give the title compound (15 mg, 9% yield) as purple solid. 1H NMR (400 MHz, CDCl₃) δ 8.64 (d, 1H), 8.51-8.43 (m, 2H), 8.32 (dd, 1H), 7.71-7.62 (m, 1H), 7.52 (t, 1H), 7.45 (dd, 1H), 7.43-7.35 (m, 2H), 7.27-7.16 (m, 2H). ¹⁹F NMR (376 MHz, CDCl₃) δ−114.99. LC-MS: m/z [M+H]⁺ 383.1.

### Example 76: (Z)-4-((4-Fluorophenyl)amino)-6-(methoxyimino)-12-oxo-6,12-dihydroindolo[2,1-b]quinazoline-8-carbonitrile (Compound 118)

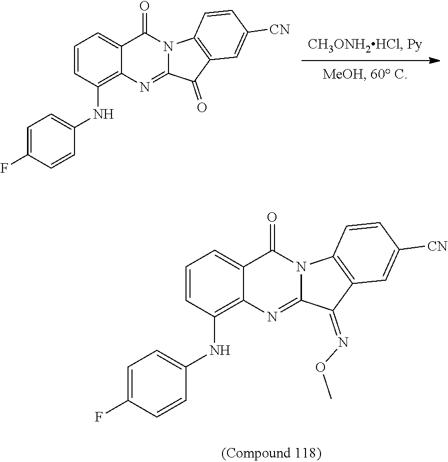 [SMILES: N#Cc1ccc2c(c1)C(=O)c1nc3c(Nc4ccc(F)cc4)cccc3c(=O)n1-2 ; CO/N=C1/c2cc(C#N)ccc2-n2c1nc1c(Nc3ccc(F)cc3)cccc1c2=O]

To a solution of 4-[(4-fluorophenyl)amino]-6,12-dioxoindolo[2,1-b]quinazoline-8-carbonitrile (50 mg, 0.13 mmol) and pyridine (41 mg, 0.52 mmol) in MeOH (5 mL) was added MeONH₂·HCl (22 mg, 0.26 mmol). The mixture was stirred at 75° C. for 12 h under N₂, then concentrated to give a residue which was recrystallized from MeOH and DCM to give the title compound (15.8 mg, 28% yield) as a purple solid. ¹H NMR (400 MHz, DMSO) δ 8.67 (d, 2H), 8.19 (dd, 1H), 8.11 (s, 1H), 7.66 (dd, 1H), 7.48-7.36 (m, 4H), 7.20 (t, 2H), 4.36 (s, 3H). LC-MS: m/z [M+H]⁺ 412.1.

### General Procedure 6

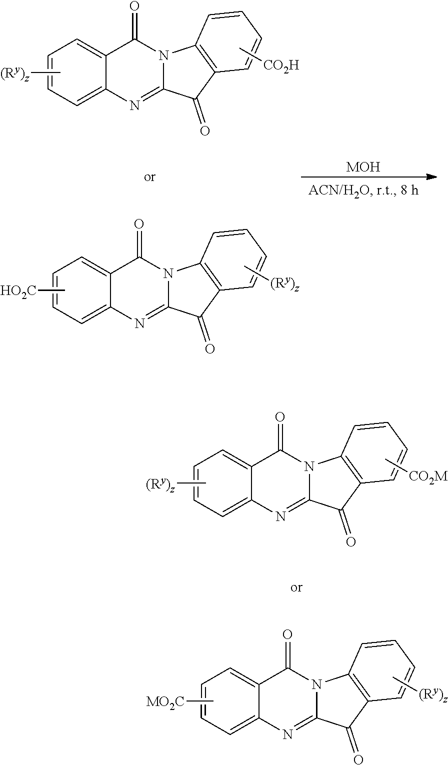

To a solution of carboxylic acid (0.25 mmol) in H₂O (10 mL)/ACN (10 mL) was added MOH (0.25 mmol), the reaction mixture was stirred for 8 h at 25° C. The mixture was concentrated under reduced pressure. The residue was triturated in hexanes/DCM, filtered, and dried under vacuum to provide metal carboxylate as a solid.

### General Procedure 7

### Example 77: Synthesis of 2-bromo-8-fluoroindolo[2,1-b]quinazoline-6,12

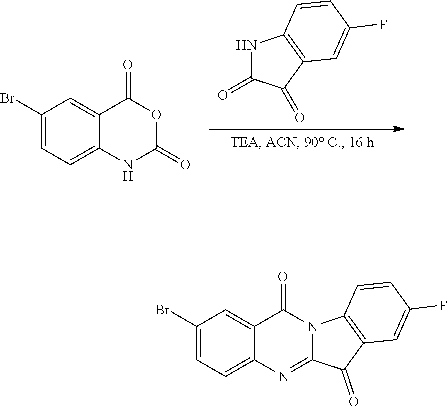 [SMILES: O=c1[nH]c2ccc(Br)cc2c(=O)o1 ; O=C1Nc2ccc(F)cc2C1=O ; O=C1c2cc(F)ccc2-n2c1nc1ccc(Br)cc1c2=O]

To the stirred solution of 5-fluoro-2,3-dihydro-1H-indole-2,3-dione (5 g, 30.3 mmol, 1.0 equiv) and 6-bromo-2,4-dihydro-1H-3,1-benzoxazine-2,4-dione (7.33 g, 30.3 mmol, 1.0 equiv) in acetonitrile (100 mL) was added Et₃N (8.43 mL, 60.6 mmol, 2.0 equiv). The mixture was stirred at 90° C. for 4 hours. After complete consumption of starting material, the reaction was cooled to RT and precipitated solid was filtered, washed with diethyl ether and dried under vacuum to obtain 2-bromo-8-fluoroindolo[2,1-b]quinazoline-6,12-dione (7.0 g, 67% yield) as a yellow solid. ¹H NMR (400 MHz, DMSO-d₆) δ 8.48 (dd, J=8.8, 4.0 Hz, 1H), 8.41 (d, J=2.0 Hz, 1H), 8.13 (dd, J=8.4, 2.0 Hz, 1H), 7.91 (d, J=8.4 Hz, 1H), 7.81 (dd, J=7.2, 2.8 Hz, 1H), 7.76-7.71 (m, 1H). LC-MS: m/z [M+H]⁺ 344.9.

### Example 78: Synthesis of 1-bromo-8-fluoroindolo[2,1-b]quinazoline-6,12-dione (Compound 103)

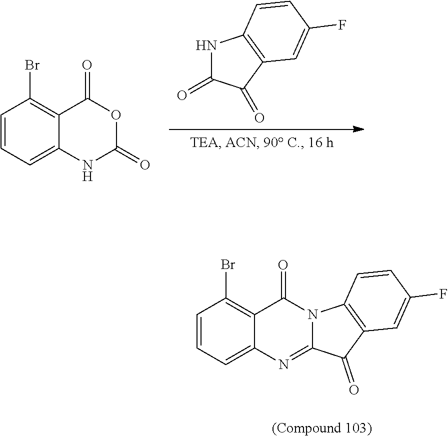 [SMILES: O=c1[nH]c2cccc(Br)c2c(=O)o1 ; O=C1Nc2ccc(F)cc2C1=O ; O=C1c2cc(F)ccc2-n2c1nc1cccc(Br)c1c2=O]

Following general procedure 7 but using 5-bromo-2H-benzo[d][1,3]oxazine-2,4(1H)-dione the title compound was obtained as a solid (35% yield). ¹H NMR (400 MHz, DMSO-d₆) δ 8.50 (dd, J=8.8, 4.0 Hz, 1H), 7.98-7.93 (m, 2H), 7.81-7.70 (m, 3H). LC-MS: m/z [M+H]⁺ 345.0.

### Example 79: Synthesis of methyl 8-fluoro-6,12-dioxo-6,12-dihydroindolo[2,1-b]quinazoline-2-carboxylate (Compound 150)

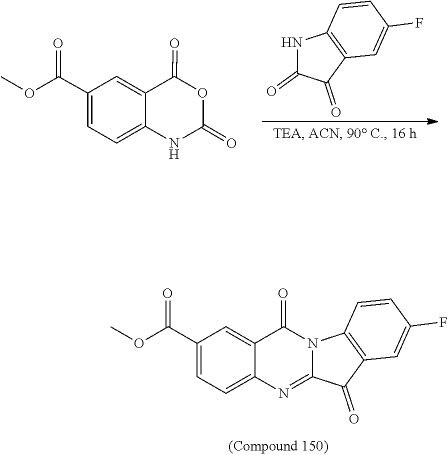 [SMILES: COC(=O)c1ccc2[nH]c(=O)oc(=O)c2c1 ; O=C1Nc2ccc(F)cc2C1=O ; COC(=O)c1ccc2nc3n(c(=O)c2c1)-c1ccc(F)cc1C3=O]

Following general procedure 7 but using methyl 2,4-dioxo-1,4-dihydro-2H-benzo[d][1,3]oxazine-6-carboxylate the title compound was obtained as a solid (6% yield). ¹H NMR (400 MHz, DMSO-d₆) δ 8.81 (d, J=1.6 Hz, 1H), 8.50 (dd, J=8.8, 4.4 Hz, 1H), 8.41 (dd, J=8.4, 1.6 Hz, 1H), 8.07 (d, J=8.4 Hz, 1H), 7.83 (dd, J=6.8, 2.4 Hz, 1H), 7.71-7.72 (m, 1H), 3.95 (s, 3H). LC-MS: m/z [M+H]⁺ 325.1.

### Example 80: Synthesis of Synthesis of 2-(bromomethyl)-8-fluoroindolo[2,1-b]quinazoline-6,12-dione (Compound 133)

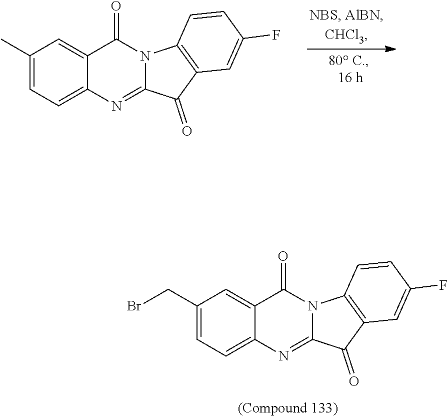 [SMILES: Cc1ccc2nc3n(c(=O)c2c1)-c1ccc(F)cc1C3=O ; O=C1c2cc(F)ccc2-n2c1nc1ccc(CBr)cc1c2=O]

To the stirred solution of 8-fluoro-2-methylindolo[2,1-b]quinazoline-6,12-dione (1.0 g 30.3 mmol, 1.0 equiv) in chloroform (50 mL), were added N-bromosuccinimide (8.43 mL, 60.6 mmol, 2.0 equiv) and benzoyl peroxide (8.43 mL, 60.6 mmol, 2.0 equiv) at RT. The mixture was further stirred at 60° C. for 16 hours. After complete consumption of starting material, the reaction was cooled to RT The precipitated solid was filtered, washed with diethyl ether and dried under vacuum to obtain 2-(bromomethyl)-8-fluoroindolo[2,1-b]quinazoline-6,12-dione (1.5 g, 67% yield) as a yellow solid. ¹H NMR (400 MHz, DMSO-d₆) δ 8.49 (dd, J=8.8, 4.0 Hz, 1H), 8.40 (d, J=2.0 Hz, 1H), 8.00 (dd, J=8.4, 2.4 Hz, 1H), 7.94 (d, J=8.0 Hz, 1H), 7.80 (dd, J=6.8, 2.4 Hz, 1H), 7.73 (td, J=6.0, 3.6 Hz, 1H), 4.95 (s, 2H). LC-MS: m/z [M+H]⁺ 359.0.

### Example 81: Synthesis of Synthesis of 2-(bromomethyl)-8-fluoroindolo[2,1-b]quinazoline-6,12-dione

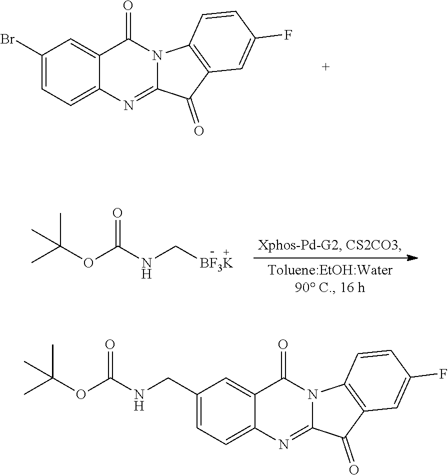

To the stirred solution of 2-bromo-8-fluoroindolo[2,1-b]quinazoline-6,12-dione (1.0 g 2.89 mmol, 1.0 equiv) in 35 mL of toluene:ethanol:water (8:2:1) ratio under N₂ at RT were added potassium tert-butyl N-[(trifluoroboranuidyl)methyl]carbamate (1.03 g, 4.35 mmol, 1.5 equiv) and Cs₂CO₃ (2.83 g, 8.69 mmol, 3 equiv) in 100 mL seal-tube. The reaction mixture was degassed with N₂ for 10 minutes, then added Xphos-Pd-G2 (0.288 g, 0.29 mmol, 0.1 equiv.). The resulting mixture was further heated at 90° C. for 16 h. After complete consumption of starting material, the mixture was cooled to RT, filtered through a celite bed and solvents evaporated under reduced pressure. The residue was dissolved in EA (50 mL) and washed with water (2×50 mL). The combined organic extracts were dried over anhydrous Na₂SO₄, filtered and concentrated under reduced pressure to obtain tert-butyl N-({8-fluoro-6,12-dioxo-6H,12H-indolo[2,1-b]quinazolin-2-yl}methyl)carbamate (1.0 g, 43% yield) as a brown solid, which was used directly in the next step without further purification. ¹H NMR (400 MHz, DMSO-d₆) δ 8.50 (dd, J=8.8, 4.0 Hz, 1H), 8.18 (br s, 1H), 7.91 (d, J=8.0 Hz, 1H), 7.80-7.76 (m, 2H), 7.72 (td, J=9.2, 2.8 Hz, 1H), 7.63 (t, J=6.0 Hz, 1H), 4.32 (d, J=6.0 Hz, 2H), 1.42 (s, 9H). LC-MS: m/z [M+H]⁺ 396.2.

### General Procedure 8

### Example 82: Synthesis of 2-(aminomethyl)-8-fluoroindolo[2,1-b]quinazoline-6,12-dione hydrochloride Salt (Compound 144)

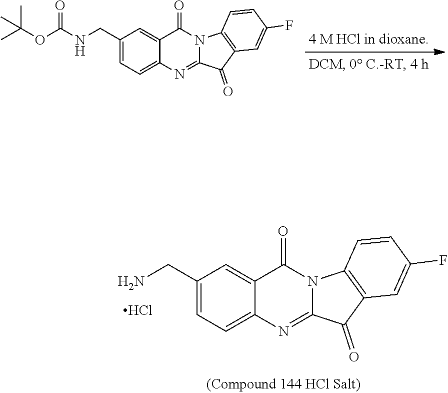 [SMILES: CC(C)(C)OC(=O)NCc1ccc2nc3n(c(=O)c2c1)-c1ccc(F)cc1C3=O ; Cl ; NCc1ccc2nc3n(c(=O)c2c1)-c1ccc(F)cc1C3=O]

To the stirred solution of tert-butyl N-({8-fluoro-6,12-dioxo-6H,12H-indolo[2,1-b]quinazolin-2-yl}methyl)carbamate (0.1 g, 0.25 mmol, 1.0 equiv) in DCM, was added 4 M HCl in dioxane (5 ml, 10 vol) at 0° C. The reaction mixture was stirred at RT for 4 hours. After complete consumption of starting material, the mixture was evaporated and dried under vacuum. The solid residue was triturated with diethyl ether, filtered and dried to obtain 2-(aminomethyl)-8-fluoroindolo[2,1-b]quinazoline-6,12-dione hydrochloride salt (0.08 g, 71% yield) as a yellow solid. ¹H NMR (400 MHz, DMSO-d₆) δ 8.51 (dd, J=8.8, 4.0 Hz, 1H), 8.47 (br s, 1H), 8.41 (br s, 2H), 8.03-7.99 (m, 2H), 7.81 (dd, J=7.2, 2.4 Hz, 1H), 7.74 (td, J=8.8, 2.8 Hz, 1H), 4.27 (br s, 2H). LC-MS: m/z [M+H]⁺ 296.1.

### Example 83: Synthesis of 3-(aminomethyl)-8-fluoroindolo[2,1-b]quinazoline-6,12-dione Hydrochloride Salt (Compound 90)

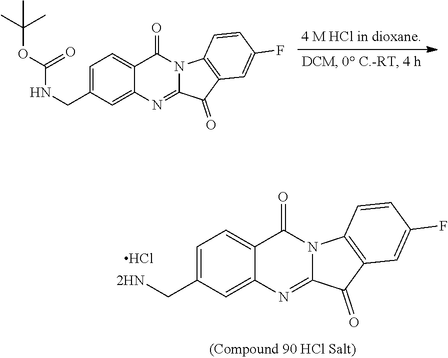

Following general procedure 8 but using tert-butyl ((8-fluoro-6,12-dioxo-6,12-dihydroindolo[2,1-b]quinazolin-3-yl)methyl)carbamate the title compound was obtained as a solid (65% yield). ¹H NMR (400 MHz, DMSO-d₆) δ 8.48 (dd, J=8.8, 4.0 Hz, 1H), 8.24 (d, J=8.4 Hz, 1H), 7.90 (br s, 1H), 7.78 (dd, J=7.2, 2.8 Hz, 1H), 7.74-7.60 (m, 2H), 3.88 (s, 2H). LC-MS: m/z [M+H]⁺ 396.1.

### General Procedure 9

### Example 84: Synthesis benzyl((8-fluoro-6,12-dioxo-6,12-dihydroindolo[2,1-b]quinazolin-2-yl)methyl)carbamate

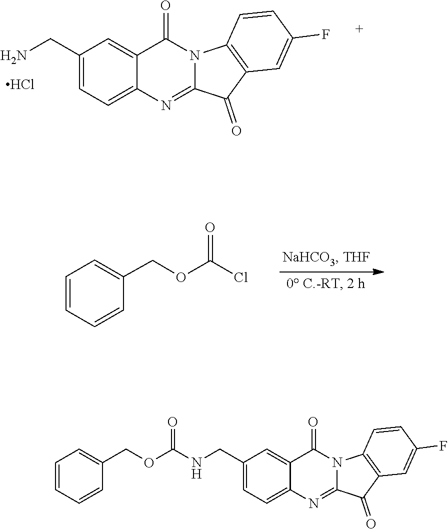 [SMILES: Cl ; NCc1ccc2nc3n(c(=O)c2c1)-c1ccc(F)cc1C3=O ; O=C(Cl)OCc1ccccc1 ; O=C(NCc1ccc2nc3n(c(=O)c2c1)-c1ccc(F)cc1C3=O)OCc1ccccc1]

To the stirred solution of 2-(aminomethyl)-8-fluoroindolo[2,1-b]quinazoline-6,12-dione hydrochloride salt (0.2 g, 0.68 mmol, 1.0 equiv) in THF (10 mL), was added sodium hydrogen carbonate (171 mg, 2.03 mmol, 3.0 equiv) followed by the addition of benzyl chloroformate (173 mg, 1.02 mmol, 1.5 equiv) at 0° C. The reaction mixture was stirred at RT for 2 hours. After complete consumption of starting material, the reaction mixture was diluted in EA (15 mL) and washed with water (2×25 mL). The combined organic extracts were dried over anhydrous Na₂SO₄, filtered and concentrated under reduced pressure. The crude compound was purified by prep-HPLC to obtain benzyl ((8-fluoro-6,12-dioxo-6,12-dihydroindolo[2,1-b]quinazolin-2-yl)methyl)carbamate (0.06 g, 23% yield) as a yellow solid. ¹H NMR (400 MHz, DMSO-d₆) δ 8.50 (dd, J=8.8, 4.0 Hz, 1H), 8.22 (d, J=2.0 Hz, 1H), 8.06 (t, J=6.0 Hz, 1H), 7.92 (d, J=8.0 Hz, 1H), 7.83-7.77 (m, 2H), 7.73 (td, J=8.8, 2.8 Hz, 1H), 7.38-7.18 (m, 5H), 5.07 (s, 2H), 4.41 (d, J=6.0 Hz, 2H). LC-MS: m/z [M+H]⁺ 430.1.

### Example 85: Synthesis of N-((8-fluoro-6,12-dioxo-6,12-dihydroindolo[2,1-b]quinazolin-2-yl)methyl)acetamide (Compound 142)

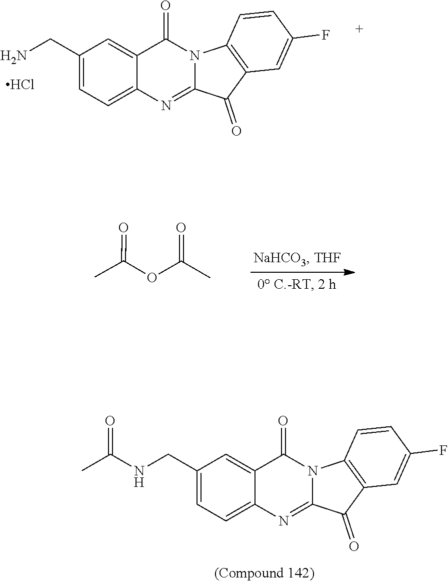

Following general procedure 9 but using acetic anhydride the title compound was obtained as a solid (3% yield). ¹H NMR (400 MHz, DMSO-d₆) δ 8.57 (t, J=6.0 Hz, 1H), 8.50 (dd, J=8.8, 4.0 Hz, 1H), 8.18 (m, 1H), 7.91 (d, J=8.4 Hz, 1H), 7.82-7.77 (m, 2H), 7.73 (td, J=9.2, 2.8 Hz, 1H), 4.44 (d, J=6.0 Hz, 2H), 1.92 (s, 3H). LC-MS: m/z [M+H]⁺ 338.1.

### Example 86: Synthesis of N-((8-fluoro-6,12-dioxo-6,12-dihydroindolo[2,1-b]quinazolin-2-yl)methyl)methanesulfonamide (Compound 145)

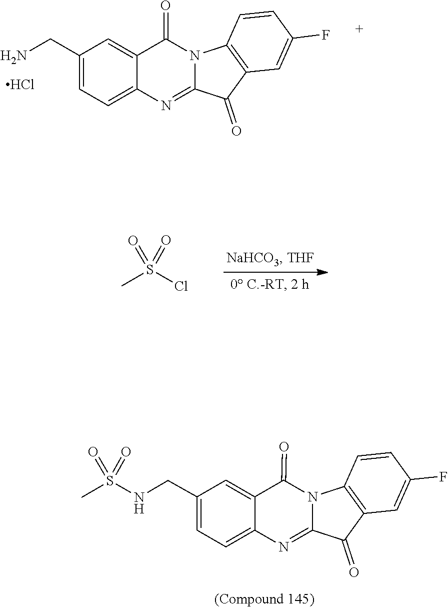

Following general procedure 9 but using MeS(O)₂C1 the title compound was obtained as a solid (3% yield). ¹H NMR (400 MHz, DMSO-d₆) δ 8.51 (dd, J=8.8, 4.4 Hz, 1H), 8.31 (d, J=1.2 Hz, 1H), 7.94 (d, J=8.0 Hz, 1H), 7.90 (dd, J=8.0, 1.6 Hz, 1H), 7.81-7.80 (m, 2H), 7.72 (td, J=8.8, 2.8 Hz, 1H), 4.39 (d, J=2.4 Hz, 2H), 2.95 (s, 3H). LC-MS: m/z [M+H]⁺ 374.0.

### Example 87: Synthesis of benzyl ((8-fluoro-6,12-dioxo-6,12-dihydroindolo[2,1-b]quinazolin-3-yl)methyl)carbamate (Compound 96)

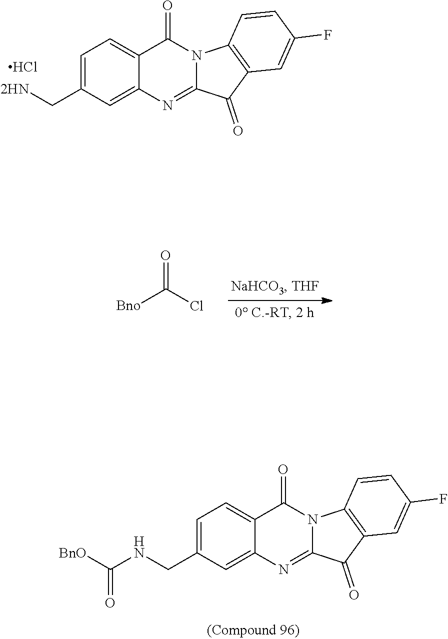

Following general procedure 9 but using 3-(aminomethyl)-8-fluoroindolo[2,1-b]quinazoline-6,12-dione hydrochloride salt and benzyl chloroformate the title compound was obtained as a solid (20% yield). ¹H NMR (400 MHz, DMSO-d₆) δ 8.50 (dd, J=8.8, 4.0 Hz, 1H), 8.28 (d, J=8.0 Hz, 1H), 8.07 (t, J=6.4 Hz, 1H), 7.81-7.78 (m, 2H), 7.73 (td, J=8.8, 2.8 Hz, 1H), 7.61 (d, J=8.4 Hz, 1H), 7.39-7.19 (m, 5H), 5.07 (s, 2H), 4.41 (d, J=6.4 Hz, 2H). LC-MS: m/z [M+H]⁺ 430.3.

### Example 88: N-((8-fluoro-6,12-dioxo-6,12-dihydroindolo[2,1-b]quinazolin-3-yl)methyl)acetamide (Compound 100)

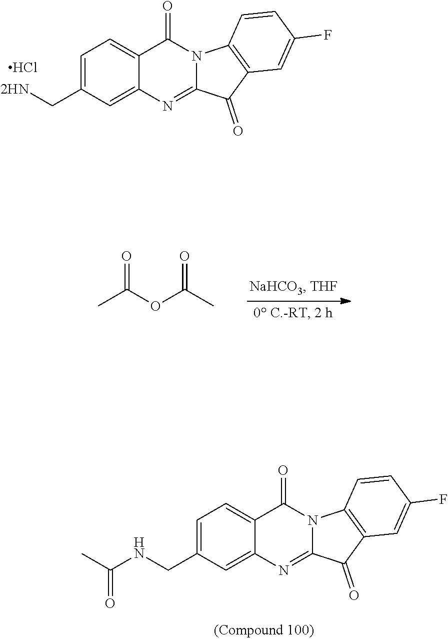

Following general procedure 9 but using 3-(aminomethyl)-8-fluoroindolo[2,1-b]quinazoline-6,12-dione hydrochloride salt and acetic anhydride the title compound was obtained as a solid (9% yield). ¹H NMR (400 MHz, DMSO-d₆) δ 8.57 (t, J=6.0 Hz, 1H), 8.50 (dd, J=8.8, 4.4 Hz, 1H), 8.26 (d, J=8.4 Hz, 1H), 7.80-7.70 (m, 3H), 7.60 (d, J=7.6 Hz, 1H), 4.45 (d, J=6.0 Hz, 2H), 1.94 (s, 3H). LC-MS: m/z [M+H]⁺ 338.3.

### Example 89: Synthesis of 8-fluoro-2-(morpholinomethyl)indolo[2,1-b]quinazoline-6,12-dione

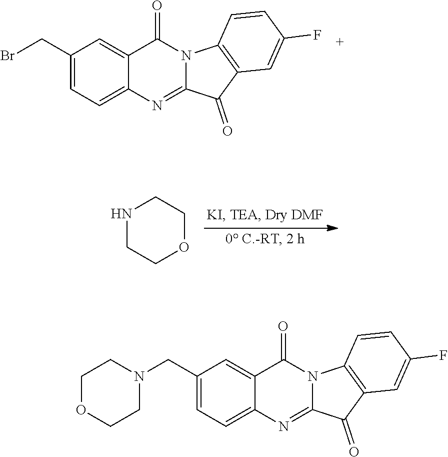 [SMILES: O=C1c2cc(F)ccc2-n2c1nc1ccc(CBr)cc1c2=O ; C1COCCN1 ; O=C1c2cc(F)ccc2-n2c1nc1ccc(CN3CCOCC3)cc1c2=O]

To the stirred solution of 2-(bromomethyl)-8-fluoroindolo[2,1-b]quinazoline-6,12-dione (0.3 g, 0.83 mmol, 1.0 equiv) and morpholine (0.14 g, 1.67 mmol, 2.0 equiv) in dry DMF (15 mL), was added Et₃N (0.35 ml, 2.50 mmol, 3.0 equiv) followed by the addition of KI (7.0 mg, 0.041 mmol 0.05 equiv) at 0° C. The reaction mixture was stirred at room temperature for 2 hours. After complete consumption of starting material, the reaction mixture was diluted with EA (15 mL) and washed with water (2×25 mL). The combined organic extracts were dried over anhydrous Na₂SO₄, filtered and concentrated under reduced pressure. The crude product was purified by prep-HPLC to obtain 8-fluoro-2-(morpholinomethyl)indolo[2,1-b]quinazoline-6,12-dione (0.07 g, 23% yield) as a green solid. ¹H NMR (400 MHz, DMSO-d₆) δ 8.50 (dd, J=8.8, 4.4 Hz, 1H), 8.25 (br s, 1H), 7.93-7.87 (m, 2H), 7.79 (dd, J=7.2, 2.8 Hz, 1H), 7.73 (td, J=9.2, 2.8 Hz, 1H), 3.68 (s, 2H), 3.60 (m, 4H), 2.42 (m, 4H). LC-MS: m/z [M+H]⁺ 366.1.

### Example 90: Synthesis of 2,3-dioxoindoline-5-carbonitrile

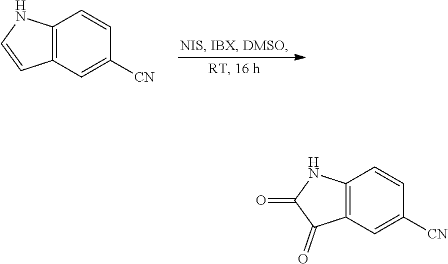 [SMILES: N#Cc1ccc2[nH]ccc2c1 ; N#Cc1ccc2c(c1)C(=O)C(=O)N2]

To the stirred solution of 5-indolecarbonitrile (15 g, 106 mmol, 1.0 equiv) in DMSO (300 ml) under N₂ at RT. was added NIS (47.5 g, 211 mmol, 2.0 equiv) and stirred for 15 min, then followed by portion wise addition of IBX (118 g, 422 mmol, 4.0 equiv) The mixture was stirred at RT for 16 hours. After complete consumption of starting material, the reaction mass was quenched with saturated solution of Na₂SO₄ (300 ml) and extracted with EA (2×500 mL). The combined organic extracts washed with saturated solution of NaHCO₃ (300 ml) and dried over anhydrous Na₂SO₄, filtered and concentrated under reduced pressure. The solid residue was triturated with diethyl ether, the precipitate was filtered and dried under vacuum to obtain 2,3-dioxo-5-indolinecarbonitrile (8.0 g, 44% yield) as an orange solid. ¹H NMR (400 MHz, DMSO-d₆) δ 11.46 (brs, 1H), 8.06-7.96 (m, 2H), 7.03 (d, J=8.0 Hz, 1H). LC-MS: m/z [M−H]⁺170.9.

### General Procedure 10

### Example 91: Synthesis of 2-bromo-6,12-dioxo-6,12-dihydroindolo[2,1-b]quinazoline-8-carbonitrile (Compound 172)

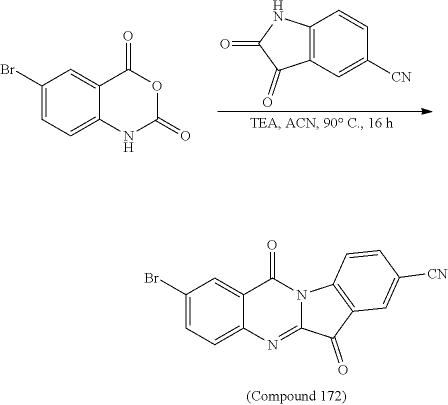 [SMILES: O=c1[nH]c2ccc(Br)cc2c(=O)o1 ; N#Cc1ccc2c(c1)C(=O)C(=O)N2 ; N#Cc1ccc2c(c1)C(=O)c1nc3ccc(Br)cc3c(=O)n1-2]

To the stirred solution of bromo-2,4-dihydro-1H-3,1-benzoxazine-2,4-dione (5 g, 20.7 mmol, 1.0 equiv) and 6-bromo-2,4-dihydro-1H-3,1-benzoxazine-2,4-dione (3.56 g, 20.7 mmol, 1.0 equiv) in acetonitrile (60 mL) was added Et₃N (8.64 mL, 62.0 mmol, 3.0 equiv). The mixture was stirred at 90° C. for 16 hours. After complete consumption of starting material, the reaction was cooled to RT and precipitated solid was filtered, washed with diethyl ether and dried under vacuum to obtain 2-bromo-6,12-dioxo-6,12-dihydroindolo[2,1-b]quinazoline-8-carbonitrile (2.0 g, 28% yield) as a yellow solid. ¹H NMR (400 MHz, DMSO-d₆) δ 8.60 (dd, J=8.8, 4.0 Hz, 1H), 8.49-8.46 (m, 2H), 8.32 (dd, J=8.4, 2.0 Hz, 1H), 8.16 (d, J=8.4 Hz, 1H), 7.93 (dd, J=7.2, 2.8 Hz, 1H). LC-MS: m/z [M+H]⁺ 351.9.

### Example 92: Synthesis of 8-cyano-6,12-dioxo-6,12-dihydroindolo[2,1-b]quinazoline-2-carboxylic Acid (Compound 157)

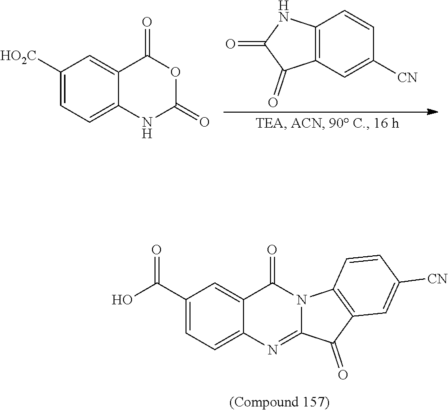 [SMILES: O=C(O)c1ccc2[nH]c(=O)oc(=O)c2c1 ; N#Cc1ccc2c(c1)C(=O)C(=O)N2 ; N#Cc1ccc2c(c1)C(=O)c1nc3ccc(C(=O)O)cc3c(=O)n1-2]

Following general procedure 10 but using 2,4-dioxo-1,4-dihydro-2H-benzo[d][1,3]oxazine-6-carboxylic acid and 2,3-dioxoindoline-5-carbonitrile the title compound was obtained as a solid (26% yield). ¹H NMR (400 MHz, DMSO-d₆) δ 8.77 (d, J=1.2 Hz, 1H), 8.64 (d, J=8.4 Hz, 1H), 8.45 (s, 1H), 8.36 (dd, J=12, 1.6 Hz, 1H), 8.30 (dd, J=8.4, 1.6 Hz, 1H), 7.91 (d, J=8.0 Hz, 1H), 7.08 (br, s, 2H). LC-MS: m/z [M+H]⁺ 317.04.

### Example 93: Synthesis of 8-fluoro-2-(1H-imidazol-1-yl)indolo[2,1-b]quinazoline-6,12-dione (Compound 137)

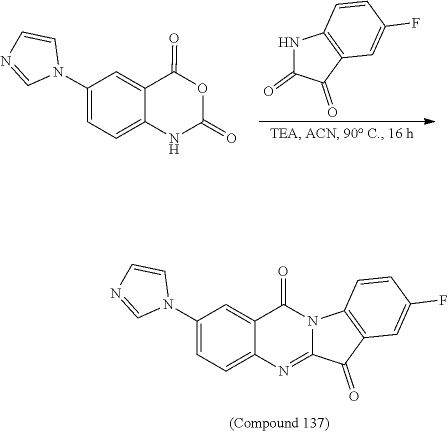 [SMILES: O=c1[nH]c2ccc(-n3ccnc3)cc2c(=O)o1 ; O=C1Nc2ccc(F)cc2C1=O ; O=C1c2cc(F)ccc2-n2c1nc1ccc(-n3ccnc3)cc1c2=O]

Following general procedure 10 but using 6-(1H-imidazol-1-yl)-2H-benzo[d][1,3]oxazine-2,4(1H)-dione and 5-fluoroindoline-2,3-dione the title compound was obtained as a solid (17% yield). ¹H NMR (400 MHz, DMSO-d₆) δ 8.56-8.50 (m, 3H), 8.28 (d, J=6.8 Hz, 1H), 8.10 (d, J=8.4 Hz, 1H), 8.04 (s, 1H), 7.82 (d, J=4.8 Hz, 1H), 7.76 (t, J=8.8 Hz, 1H), 7.19 (s, 1H). LC-MS: m/z [M+H]⁺ 332.07.

### Example 94: Synthesis of 8-fluoro-3-(1H-imidazol-1-yl)indolo[2,1-b]quinazoline-6,12-dione (Compound 98)

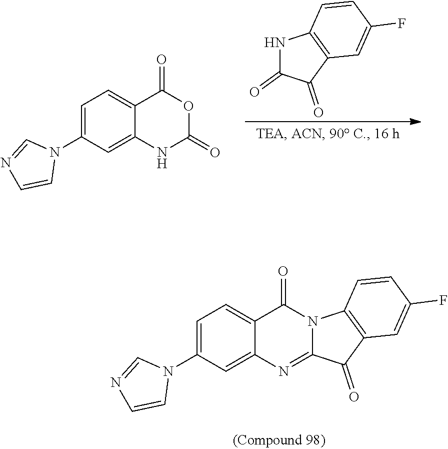 [SMILES: O=c1[nH]c2cc(-n3ccnc3)ccc2c(=O)o1 ; O=C1Nc2ccc(F)cc2C1=O ; O=C1c2cc(F)ccc2-n2c1nc1cc(-n3ccnc3)ccc1c2=O]

Following general procedure 10 but using 7-(1H-imidazol-1-yl)-2H-benzo[d][1,3]oxazine-2,4(1H)-dione and 5-fluoroindoline-2,3-dione the title compound was obtained as a solid (55% yield). ¹H NMR (400 MHz, DMSO-d₆) δ 8.62 (s, 1H), 8.49 (dd, J=8.8, 4.0 Hz, 1H), 8.41 (d, J=8.4 Hz, 1H), 8.37 (d, J=2.0 Hz, 1H), 8.09 (d, J=13.2 Hz, 2H), 7.81 (dd, J=7.2, 2.4 Hz, 1H), 7.75 (td, J=6.8, 2.4 Hz, 1H), 7.21 (s, 1H). LC-MS: m/z [M+H]⁺ 332.07.

### Example 95: Synthesis of 4-bromo-6,12-dioxo-6,12-dihydroindolo[2,1-b]quinazoline-8-carbonitrile (Compound 46)

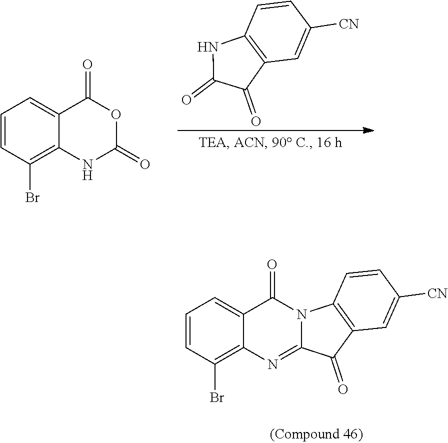 [SMILES: O=c1[nH]c2c(Br)cccc2c(=O)o1 ; N#Cc1ccc2c(c1)C(=O)C(=O)N2 ; N#Cc1ccc2c(c1)C(=O)c1nc3c(Br)cccc3c(=O)n1-2]

Following general procedure 10 but using 8-bromo-2H-benzo[d][1,3]oxazine-2,4(1H)-dione the title compound was obtained as a solid (27% yield). ¹H NMR (400 MHz, DMSO-d₆) δ 8.61 (d, J=8.4 Hz, 1H), 8.50 (s, 1H), 8.36-8.30 (m, 3H), 7.97 (t, J=8.0 Hz, 1H). LC-MS: m/z [M+H]⁺ 351.9.

### Example 96: Synthesis of tert-butyl (8-cyano-6,12-dioxo-6,12-dihydroindolo[2,1-b]quinazolin-2-yl)carbamate (Compound 160)

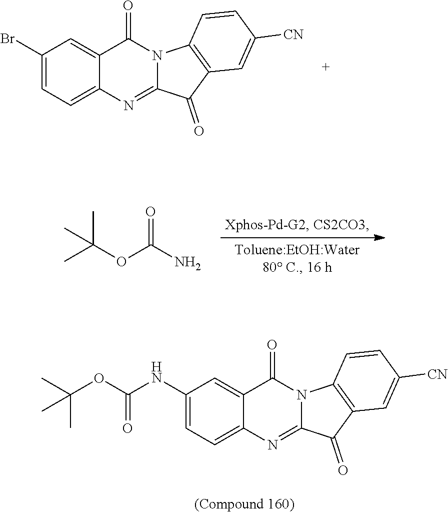

To the stirred solution of 2-bromo-6,12-dioxo-6,12-dihydroindolo[2,1-b]quinazoline-8-carbonitrile (0.25 g, 0.71 mmol, 1.0 equiv) in 5.5 mL of toluene:ethanol:water (8:2:1) ratio under nitrogen atmosphere at room temperature were added tert-butyl carbamate (0.09 g, 0.85 mmol, 1.2 equiv) and Cs₂CO₃ (0.69 g, 2.13 mmol, 3.0 equiv) in 30 mL seal-tube. The reaction mixture was degassed with N₂ for 10 minutes, then added Xphos-Pd-G2 (0.056 g, 0.07 mmol, 0.1 equiv.). The resulting mixture was further heated at 80° C. f or 16 h. After complete consumption of starting material, the reaction mixture was cooled to RT, filtered through a celite bed and solvents evaporated under reduced pressure. The residue was dissolved in EA (10 mL) and washed with water (2×20 mL). The combined organic extracts were dried over anhydrous Na₂SO₄, filtered and concentrated under reduced pressure to obtain crude product. This was purified by Prep-HPLC to obtain tert-butyl (8-cyano-6,12-dioxo-6,12-dihydroindolo[2,1-b]quinazolin-2-yl)carbamate (0.014 g, 5% yield) as a yellow solid. ¹H NMR (400 MHz, DMSO-d₆) δ 10.09 (s, 1H), 8.61 (d, J=8.4 Hz, 1H), 8.57 (s, 1H), 8.42 (s, 1H), 8.30 (d, J=8.8 Hz, 1H), 7.88-7.94 (m, 2H), 1.48 (s, 9H). LC-MS: m/z [M+H]⁺ 389.2

### General Procedure 11

### Example 97: Synthesis of 6-[(aminosulfoamino)methyl]-9,17-dioxo-2,10-diazatetracyclo[8.7.0.0^{3,8}.0^{11,16}]heptadeca-1,3,5,7,11(16),12,14-heptaene-14-carbonitrile (Compound 156)

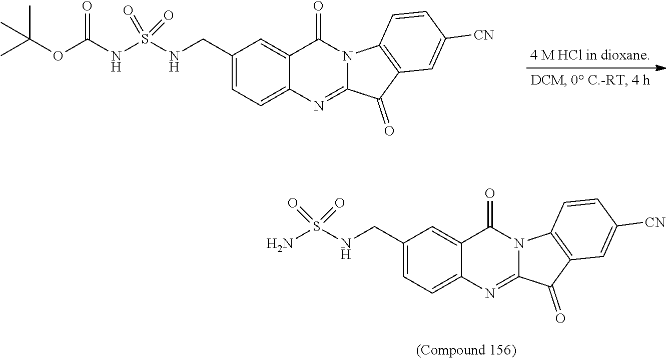 [SMILES: CC(C)(C)OC(=O)NS(=O)(=O)NCc1ccc2nc3n(c(=O)c2c1)-c1ccc(C#N)cc1C3=O ; N#Cc1ccc2c(c1)C(=O)c1nc3ccc(CNS(N)(=O)=O)cc3c(=O)n1-2]

To the stirred solution of tert-butyl-({14-cyano-9,17-dioxo-2,10-diazatetracyclo[8.7.0.0^{3,8}.0^{11,16}]heptadeca-1,3,5,7,11(16),12,14-heptaen-6-yl}methyl)aminosulfonylaminoformylate (0.2 g, 0.41 mmol, 1.0 equiv) in DCM (5 ml), was added 4 M HCl in dioxane (2.5 ml, 10 vol) at 0° C. The mixture was stirred at RT for 4 hours. After complete consumption of starting material, the mixture was evaporated and dried under vacuum. The solid residue was triturated with diethyl ether. The precipitates were filtered and dried to obtain 6-[(aminosulfoamino)methyl]-9,17-dioxo-2,10-diazatetracyclo[8.7.0.0^{3,8}.0^{11,16}]heptadeca-1,3,5,7,11(16),12,14-heptaene-14-carbonitrile (0.018 g, 11% yield) as a yellow solid. ¹H NMR (400 MHz, DMSO-d₆) δ 8.62 (d, J=8.8 Hz, 1H), 8.45 (s, 1H), 8.32 (d, J=9.6 Hz, 2H), 7.94 (s, 2H), 7.37 (br, s, 1H), 6.76 (s, 2H), 4.29 (s, 2H). LC-MS: m/z [M+H]⁺ 382.2.

### Example 98: Synthesis of N-(2-aminoethyl)-3-fluoro-6,12-dioxo-6,12-dihydroindolo[2,1-b]quinazoline-8-carboxamide hydrochloride salt (Compound 168)

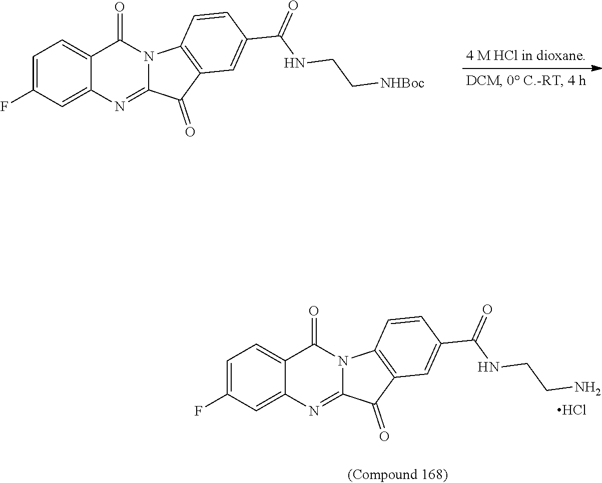 [SMILES: CC(C)(C)OC(=O)NCCNC(=O)c1ccc2c(c1)C(=O)c1nc3cc(F)ccc3c(=O)n1-2 ; NCCNC(=O)c1ccc2c(c1)C(=O)c1nc3cc(F)ccc3c(=O)n1-2 ; Cl]

Following general procedure 11 but using tert-butyl (2-(3-fluoro-6,12-dioxo-6,12-dihydroindolo[2,1-b]quinazoline-8-carboxamido)ethyl)carbamate the title compound was obtained as a solid (54% yield). ¹H NMR (400 MHz, DMSO-d₆) δ 8.95 (t, J=5.2 Hz, 1H), 8.53 (d, J=8.4 Hz, 1H), 8.36-8.42 (m, 3H), 7.92 (br, s, 2H), 7.85 (dd, J=9.2, 2.4 Hz, 1H), 6.65 (td, J=9.2, 2.4 Hz, 1H), 5.54 (q, J=11.6, 5.6 Hz, 2H), 3.02 (q, J=11.6, 5.6 Hz, 2H).). LC-MS: m/z [M+H]⁺ 352.1.

### Example 99: Synthesis of 6,12-dioxo-4-(pyridin-2-ylamino)-6,12-dihydroindolo[2,1-b]quinazoline-8-carbonitrile hydrochloride salt (Compound 170)

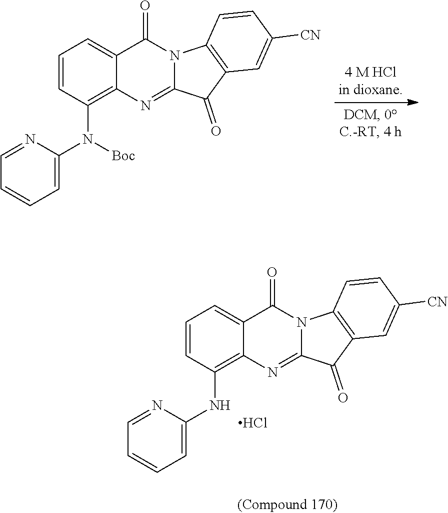 [SMILES: CC(C)(C)OC(=O)N(c1ccccn1)c1cccc2c(=O)n3c(nc12)C(=O)c1cc(C#N)ccc1-3 ; N#Cc1ccc2c(c1)C(=O)c1nc3c(Nc4ccccn4)cccc3c(=O)n1-2 ; Cl]

Following general procedure 11 but using tert-butyl (8-cyano-6,12-dioxo-6,12-dihydroindolo[2,1-b]quinazolin-4-yl)(pyridin-2-yl)carbamate the title compound was obtained as a solid (98% yield). ¹H NMR (400 MHz, DMSO-d₆) δ 9.32 (brs, 1H), 8.84 (brd, J=7.2 Hz, 1H), 8.64 (d, J=8.4 Hz, 1H), 8.48 (d, J=1.6 Hz, 1H), 8.33 (dd, J=8.0, 1.2 Hz, 1H), 8.24 (d, J=4.4 Hz, 1H), 7.90 (d, J=7.6 Hz, 1H), 7.78 (t, J=7.6 Hz, 1H), 7.71 (t, J=8.0 Hz, 1H), 7.40 (d, J=8.4 Hz, 1H), 6.98 (t, J=6.0 Hz, 1H); HCl peak merged with D2O. LC-MS: m/z [M+H]⁺ 366.2.

### Example 100: Synthesis of 6,12-dioxo-2-(piperazine-1-carbonyl)-6,12-dihydroindolo[2,1-b]quinazoline-8-carbonitrile hydrochloride salt (Compound 163)

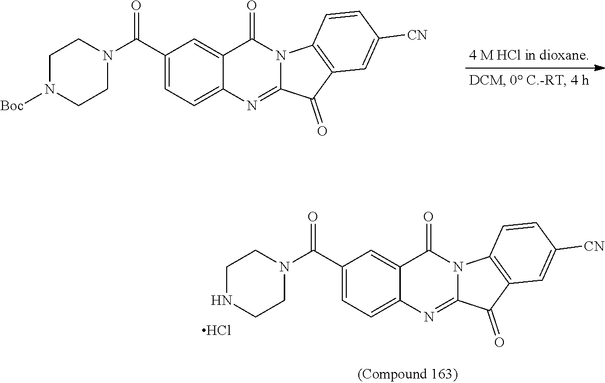 [SMILES: CC(C)(C)OC(=O)N1CCN(C(=O)c2ccc3nc4n(c(=O)c3c2)-c2ccc(C#N)cc2C4=O)CC1 ; Cl ; N#Cc1ccc2c(c1)C(=O)c1nc3ccc(C(=O)N4CCNCC4)cc3c(=O)n1-2]

Following general procedure 11 but using tert-butyl 4-(8-cyano-6,12-dioxo-6,12-dihydroindolo[2,1-b]quinazoline-2-carbonyl)piperazine-1-carboxylate the title compound was obtained as a solid (90% yield). ¹H NMR (400 MHz, DMSO-d₆) δ 9.04 (br, s, 2H), 8.61 (d, J=8.4 Hz, 1H), 8.50 (s, 1H), 8.41 (s, 1H), 8.34 (d, J=7.2 Hz, 1H), 8.05 (q, J=8.4 Hz, 2H), 3.86 (br, s, 2H), 3.60 (br, s, 2H), 3.20 (br, s, 4H). LC-MS: m/z [M+H]⁺ 386.2.

### Example 101: Synthesis of 3-chloro-8-(piperazine-1-carbonyl)indolo[2,1-b]quinazoline-6,12-dione (Compound 167)

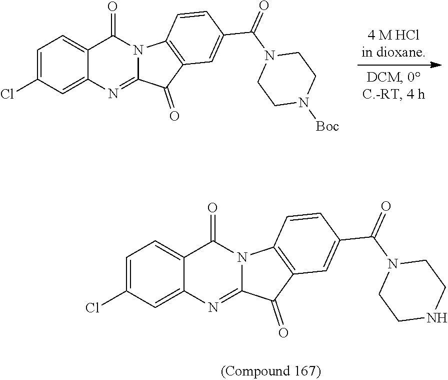 [SMILES: CC(C)(C)OC(=O)N1CCN(C(=O)c2ccc3c(c2)C(=O)c2nc4cc(Cl)ccc4c(=O)n2-3)CC1 ; O=C1c2cc(C(=O)N3CCNCC3)ccc2-n2c1nc1cc(Cl)ccc1c2=O]

Following general procedure 11 but using tert-butyl 4-(3-chloro-6,12-dioxo-6,12-dihydroindolo[2,1-b]quinazoline-8-carbonyl)piperazine-1-carboxylate the title compound was obtained as a solid (78% yield). ¹H NMR (400 MHz, DMSO-d₆) δ 8.90 (br, s, 2H), 8.52 (d, J=8.4 Hz, 1H), 8.33 (d, J=8.4 Hz, 1H), 8.10 (d, J=1.6 Hz, 1H), 8.00 (s, 1H), 7.96 (d, J=8.4 Hz, 1H), 7.81 (d, J=8.4, 1.6 Hz, 1H), 3.71 (br, s, 4H), 3.18 (br, s, 4H). LC-MS: m/z [M+H]⁺ 395.2.

### Example 102: Synthesis of Compound 88

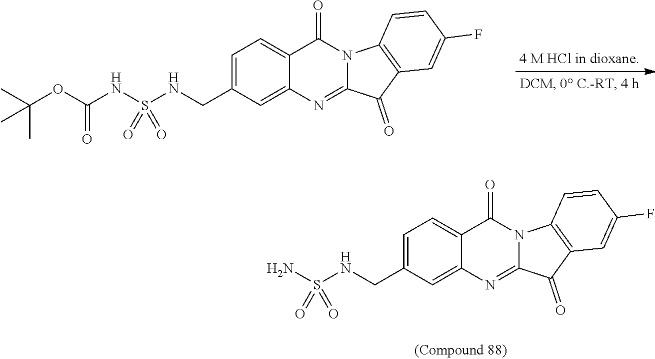 [SMILES: CC(C)(C)OC(=O)NS(=O)(=O)NCc1ccc2c(=O)n3c(nc2c1)C(=O)c1cc(F)ccc1-3 ; NS(=O)(=O)NCc1ccc2c(=O)n3c(nc2c1)C(=O)c1cc(F)ccc1-3]

Following general procedure 11 but using tert-butyl (N-((8-fluoro-6,12-dioxo-6,12-dihydroindolo[2,1-b]quinazolin-3-yl)methyl)sulfamoyl)carbamate the title compound was obtained as a solid (17% yield). ¹H NMR (400 MHz, DMSO-d₆) δ 8.49 (dd, J=8.8, 4.4 Hz, 1H), 8.25 (d, J=8.0 Hz, 1H), 7.92 (s, 1H), 7.79 (dd, J=7.2, 2.8 Hz, 1H), 7.75-7.70 (m, 2H), 7.37 (t, J=6.8 Hz, 1H), 6.76 (s, 2H), 4.29 (d, J=6.8 Hz, 2H). LC-MS: m/z [M+H]⁺ 373.2.

### Example 103: Synthesis of Compound 99

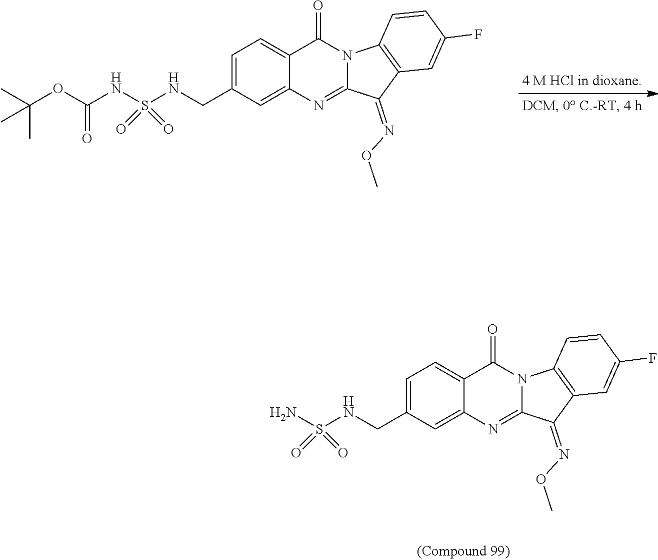 [SMILES: CO/N=C1/c2cc(F)ccc2-n2c1nc1cc(CNS(=O)(=O)NC(=O)OC(C)(C)C)ccc1c2=O ; CO/N=C1/c2cc(F)ccc2-n2c1nc1cc(CNS(N)(=O)=O)ccc1c2=O]

Following general procedure 11 but using tert-butyl (Z)—(N-((8-fluoro-6-(methoxyimino)-12-oxo-6,12-dihydroindolo[2,1-b]quinazolin-3-yl)methyl)sulfamoyl)carbamate the title compound was obtained as a solid (6% yield). ¹H NMR (400 MHz, DMSO-d₆) δ 8.52 (dd, J=9.2, 4.8 Hz, 1H), 8.23 (d, J=8.4 Hz, 1H), 8.02 (dd, J=8.0, 2.8 Hz, 1H), 7.82 (s, 1H), 7.62 (dd, J=4.0, 1.2 Hz, 1H), 7.60-7.54 (m, 1H), 7.34 (brm, 1H), 6.75 (s, 2H), 4.32 (s, 3H), 4.28 (d, J=4.4 Hz, 2H). LC-MS: m/z [M+H]⁺ 404.1.

### Example 104: Synthesis of 2-cyano-1-((8-fluoro-6,12-dioxo-6,12-dihydroindolo[2,1-b]quinazolin-2-yl)methyl)guanidine (Compound 140)

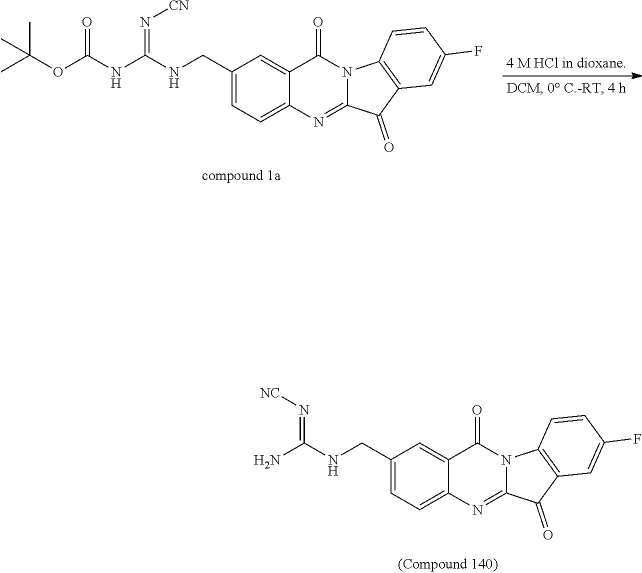 [SMILES: CC(C)(C)OC(=O)N/C(=N/C#N)NCc1ccc2nc3n(c(=O)c2c1)-c1ccc(F)cc1C3=O ; N#C/N=C(\N)NCc1ccc2nc3n(c(=O)c2c1)-c1ccc(F)cc1C3=O]

Following general procedure 11 but using compound 1a the title compound was obtained as a solid (30% yield). ¹H NMR (400 MHz, DMSO-d₆) δ 8.50 (m, 1H), 8.19 (brs, 1H), 7.92 (d, J=8.4 Hz, 1H), 7.82-7.80 (m, 2H), 7.78-7.71 (m, 1H), 7.56 (brs, 1H), 6.98 (brs, 2H), 4.47 (d, J=6.0 Hz, 2H). LC-MS: m/z [M+H]⁺ 363.2.

### Example 105: Synthesis of 1-(aminomethyl)-8-fluoroindolo[2,1-b]quinazoline-6,12-dione (Compound 101)

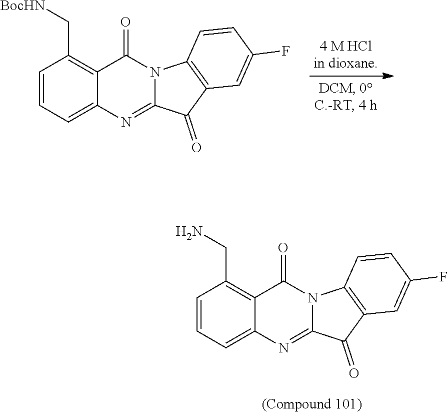 [SMILES: CC(C)(C)OC(=O)NCc1cccc2nc3n(c(=O)c12)-c1ccc(F)cc1C3=O ; NCc1cccc2nc3n(c(=O)c12)-c1ccc(F)cc1C3=O]

Following general procedure 11 but using tert-butyl ((8-fluoro-6,12-dioxo-6,12-dihydroindolo[2,1-b]quinazolin-1-yl)methyl)carbamate the title compound was obtained as a solid (85% yield). ¹H NMR (400 MHz, DMSO-d₆) δ 8.52 (dd, J=8.8, 4.0 Hz, 1H), 8.31 (brs, 3H), 8.04-7.98 (m, 2H), 7.84 (dd, J=7.2, 2.8 Hz, 1H), 7.78-7.73 (m, 2H), 4.66 (s, 2H). LC-MS: m/z [M+H]⁺ 296.1.

### Example 106: Synthesis of 8-fluoro-2-((pyridin-2-ylamino)methyl)indolo[2,1-b]quinazoline-6,12-dione (Compound 141)

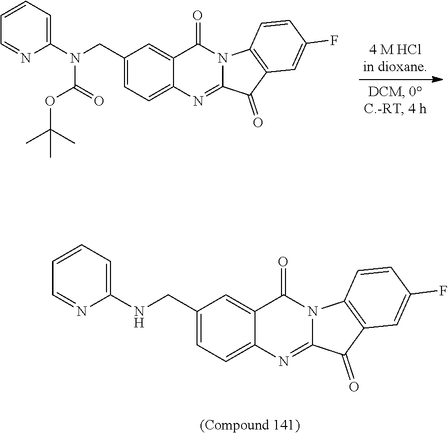 [SMILES: CC(C)(C)OC(=O)N(Cc1ccc2nc3n(c(=O)c2c1)-c1ccc(F)cc1C3=O)c1ccccn1 ; O=C1c2cc(F)ccc2-n2c1nc1ccc(CNc3ccccn3)cc1c2=O]

Following general procedure 11 but using tert-butyl ((8-fluoro-6,12-dioxo-6,12-dihydroindolo[2,1-b]quinazolin-2-yl)methyl)(pyridin-2-yl)carbamate the title compound was obtained as a solid (6% yield). ¹H NMR (400 MHz, DMSO-d₆) δ 8.60 (brs, 2H), 8.46 (dd, J=8.0, 4.0 Hz, 1H), 8.30 (d, J=5.6 Hz, 1H), 8.11 (d, J=1.6 Hz, 1H), 8.02-7.97 (m, 2H), 7.83-7.72 (m, 3H), 7.14 (d, J=8.8 Hz, 1H), 7.07 (t, J=6.0 Hz, 1H), 5.67 (s, 2H). LC-MS: m/z [M+H]⁺ 373.3.

### Example 107: Synthesis of 1-((8-fluoro-6,12-dioxo-6,12-dihydroindolo[2,1-b]quinazolin-2-yl)methyl)guanidine (Compound 147)

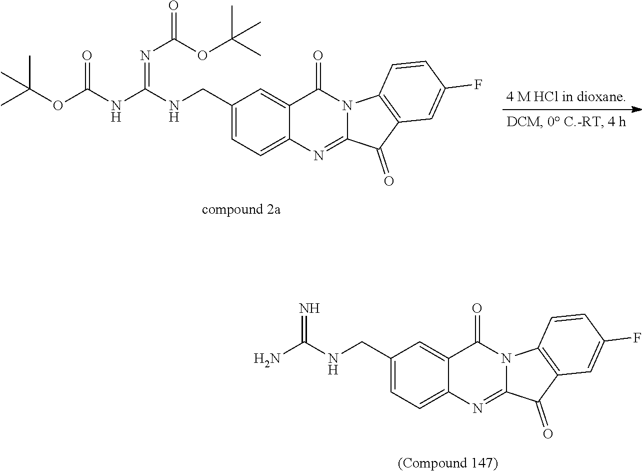 [SMILES: CC(C)(C)OC(=O)/N=C(\NCc1ccc2nc3n(c(=O)c2c1)-c1ccc(F)cc1C3=O)NC(=O)OC(C)(C)C ; N=C(N)NCc1ccc2nc3n(c(=O)c2c1)-c1ccc(F)cc1C3=O]

Following general procedure 11 but using compound 2a the title compound was obtained as a solid (84% yield). ¹H NMR (400 MHz, DMSO-d₆) δ 8.92-8.86 (m, 1H), 8.72 (s, 1H), 8.41 (d, J=8.8 Hz, 1H), 8.36 (d, J=7.6 Hz, 1H), 8.14-8.09 (m, 2H), 5.13 (s, 2H); NMR at elevated temperature in D2O. LC-MS: m/z [M+H]⁺ 338.1.

### General Procedure 12

### Example 108: Synthesis of N-((8-cyano-6,12-dioxo-6,12-dihydroindolo[2,1-b]quinazolin-2-yl)methyl)methanesulfonamide (Compound 155)

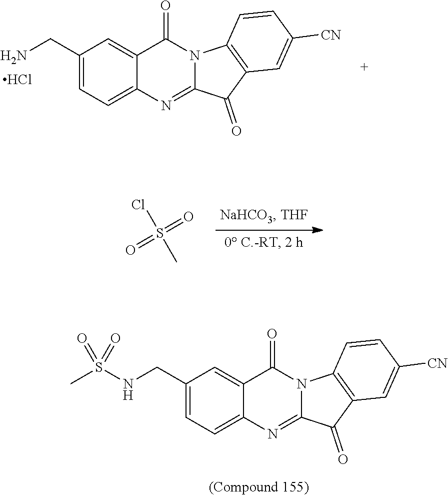

To the stirred solution of 2-(aminomethyl)-6,12-dioxo-6,12-dihydroindolo[2,1-b]quinazoline-8-carbonitrile hydrochloride salt (0.2 g, 0.59 mmol, 1.0 equiv) in THF (10 mL), was added NaHCO₃ (0.248 mg, 2.95 mmol, 5.0 equiv) followed by the addition of Mesyl chloride (135 mg, 1.18 mmol, 2.0 equiv) at 0° C. The mixture was stirred at RT for 2 hours. After complete consumption of starting material, the mixture was diluted in EA (10 mL) and washed with water (2×20 mL). The combined organic extracts were dried over anhydrous Na₂SO₄, filtered and concentrated under reduced pressure. The crude compound was purified by prep-HPLC to obtain N-((8-cyano-6,12-dioxo-6,12-dihydroindolo[2,1-b]quinazolin-2-yl)methyl)methanesulfonamide (0.015 g, 7% yield) as a yellow solid. ¹H NMR (400 MHz, DMSO-d₆) δ 8.60 (d, J=8.4 Hz, 1H), 8.28 (d, J=11.2 Hz, 2H), 8.20 (d, J=8.4 Hz, 1H), 7.89 (q, J=16.4, 8.4 Hz, 2H), 2.91 (s, 3H). LC-MS: m/z [M+H]⁺ 381.1

### Example 109: Synthesis of N-((8-fluoro-6,12-dioxo-6,12-dihydroindolo[2,1-b]quinazolin-3-yl)methyl)methanesulfonamide (Compound 95)

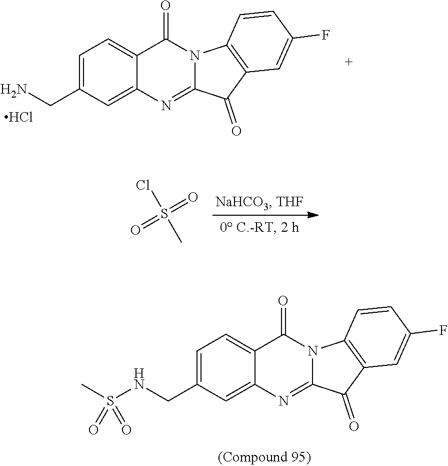

Following general procedure 12 but using 3-(aminomethyl)-8-fluoroindolo[2,1-b]quinazoline-6,12-dione hydrochloride the title compound was obtained as a solid (8% yield). ¹H NMR (400 MHz, DMSO-d₆) δ 8.49 (brm, 1H), 8.30 (d, J=8.0 Hz, 1H), 7.89-7.70 (brm, 5H), 4.39 (s, 2H), 2.95 (s, 3H). LC-MS: m/z [M+H]⁺ 374.1.

### General Procedure 13

### Example 110: Synthesis of 8-fluoro-2-(morpholinomethyl)indolo[2,1-b]quinazoline-6,12-dione

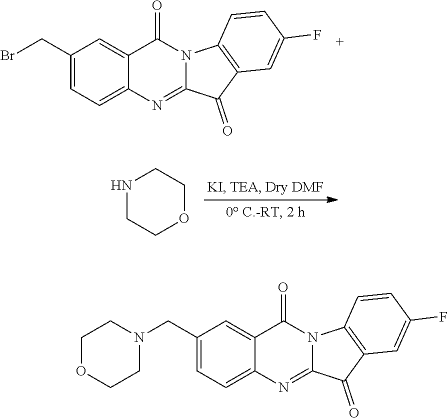 [SMILES: O=C1c2cc(F)ccc2-n2c1nc1ccc(CBr)cc1c2=O ; C1COCCN1 ; O=C1c2cc(F)ccc2-n2c1nc1ccc(CN3CCOCC3)cc1c2=O]

To the stirred solution of 2-(bromomethyl)-8-fluoroindolo[2,1-b]quinazoline-6,12-dione (0.3 g, 0.83 mmol, 1.0 equiv) and morpholine (0.14 g, 1.67 mmol, 2.0 equiv) in dry DMF (15 mL), was added Et₃N (0.35 ml, 2.50 mmol, 3.0 equiv) followed by the addition of KI (7.0 mg, 0.041 mmol) at 0° C. The reaction mixture was stirred at RT for 2 hours. After complete consumption of starting material, the mixture was diluted with EA (15 mL) and washed with water (2×25 mL). The combined organic extracts were dried over anhydrous Na₂SO₄, filtered and concentrated under reduced pressure. The crude product was purified by prep-HPLC to obtain 8-fluoro-2-(morpholinomethyl)indolo[2,1-b]quinazoline-6,12-dione (0.07 g, 23% yield) as a green solid. ¹H NMR (400 MHz, DMSO-d₆) δ 8.50 (dd, J=8.8, 4.4 Hz, 1H), 8.25 (br s, 1H), 7.93-7.87 (m, 2H), 7.79 (dd, J=7.2, 2.8 Hz, 1H), 7.73 (td, J=9.2, 2.8 Hz, 1H), 3.68 (s, 2H), 3.60 (m, 4H), 2.42 (m, 4H). LC-MS: m/z [M+H]⁺ 366.1.

### Example 111: Synthesis of 2-(morpholinomethyl)-6,12-dioxo-6,12-dihydroindolo[2,1-b]quinazoline-8-carbonitrile (Compound 136)

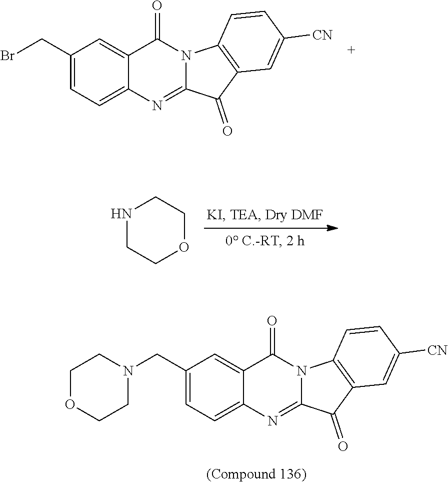 [SMILES: N#Cc1ccc2c(c1)C(=O)c1nc3ccc(CBr)cc3c(=O)n1-2 ; C1COCCN1 ; N#Cc1ccc2c(c1)C(=O)c1nc3ccc(CN4CCOCC4)cc3c(=O)n1-2]

Following general procedure 13 but using 2-(bromomethyl)-6,12-dioxo-6,12-dihydroindolo[2,1-b]quinazoline-8-carbonitrile the title compound was obtained as a solid (6% yield). ¹H NMR (400 MHz, DMSO-d₆) δ 8.59 (d, J=8.4 Hz, 1H), 8.36 (s, 1H), 8.27 (d, J=9.2 Hz, 1H), 8.24 (s, 1H), 8.91 (s, 2H), 3.57 (t, J=4.0 Hz, 4H), 2.40 (br, s, 4H). LC-MS: m/z [M+H]⁺ 373.1.

### General Procedure 14

### Example 112: Synthesis of (Z)—N-((8-fluoro-6-(methoxyimino)-12-oxo-6,12-dihydroindolo[2,1-b]quinazolin-3-yl)methyl)methanesulfonamide (Compound 91)

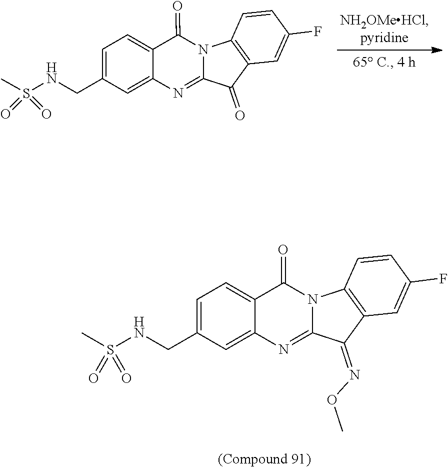 [SMILES: CS(=O)(=O)NCc1ccc2c(=O)n3c(nc2c1)C(=O)c1cc(F)ccc1-3 ; CO/N=C1/c2cc(F)ccc2-n2c1nc1cc(CNS(C)(=O)=O)ccc1c2=O]

To a stirred solution of N-((8-fluoro-6,12-dioxo-6,12-dihydroindolo[2,1-b]quinazolin-3-yl)methyl)methanesulfonamide (0.3 g, 0.80 mmol, 1.0 equiv) in pyridine (3 mL) was added NH₂OMe·HCl (0.2 mg, 2.41 mmol, 3.0 equiv) at RT. The resulting mixture was heated at 60° C. for 4 h. The mixture was cooled to RT and quench with ice cold water and stirred for 10 min, the solids were filtered, and washed with water and dried under vacuum. The solid residue was triturated with diethyl ether. The precipitate was filtered and the filter cake was purified by prep-HPLC to obtain (Z)—N-((8-fluoro-6-(methoxyimino)-12-oxo-6,12-dihydroindolo[2,1-b]quinazolin-3-yl)methyl) methanesulfonamide (0.016 g, 5% yield) as a yellow solid. ¹H NMR (400 MHz, DMSO-d₆) δ 8.51 (q, J=4.4 Hz, 1H), 8.27 (d, J=8.4 Hz, 1H), 8.02 (dd, J=8.4 Hz, 2.8 Hz, 1H), 7.81 (t, J=12.8 Hz, 6.0 Hz, 1H), 7.78 (s, 1H), 7.62-7.54 (m, 2H), 4.37 (s, 2H), 4.33 (s, 3H), 2.94 (s, 3H). LC-MS: m/z [M+H]⁺ 403.1

### Example 113: Synthesis of (Z)-6-(methoxyimino)-3-((4-methylpiperazin-1-yl)methyl)-12-oxo-6,12-dihydroindolo[2,1-b]quinazoline-8-sulfonamide (Compound 165)

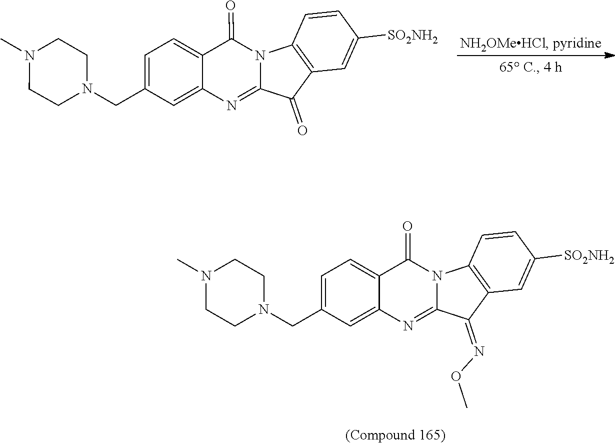 [SMILES: CN1CCN(Cc2ccc3c(=O)n4c(nc3c2)C(=O)c2cc(S(N)(=O)=O)ccc2-4)CC1 ; CO/N=C1/c2cc(S(N)(=O)=O)ccc2-n2c1nc1cc(CN3CCN(C)CC3)ccc1c2=O]

Following general procedure 14 but using 3-((4-methylpiperazin-1-yl)methyl)-6,12-dioxo-6,12-dihydroindolo[2,1-b]quinazoline-8-sulfonamide the title compound was obtained as a solid (6% yield). ¹H NMR (400 MHz, DMSO-d₆) δ 8.65-8.62 (m, 2H), 8.25 (d, J=8.4 Hz, 1H), 8.17 (dd, J=7.6, 2.0 Hz, 1H), 7.75 (s, 1H), 7.59 (d, J=7.6 Hz, 1H), 4.34 (s, 3H), 3.67 (s, 2H), 2.49-2.35 (m, 8H), 2.16 (s, 3H). LC-MS: m/z [M+H]⁺ 415.2.

### Example 114: Synthesis of (Z)-6-(methoxyimino)-2-(morpholinomethyl)-12-oxo-6,12-dihydroindolo[2,1-b]quinazoline-8-carbonitrile (Compound 131)

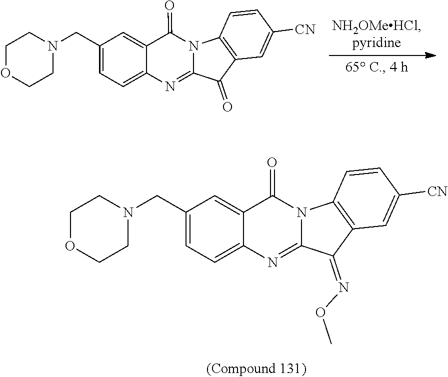 [SMILES: N#Cc1ccc2c(c1)C(=O)c1nc3ccc(CN4CCOCC4)cc3c(=O)n1-2 ; CO/N=C1/c2cc(C#N)ccc2-n2c1nc1ccc(CN3CCOCC3)cc1c2=O]

Following general procedure 14 but using 2-(morpholinomethyl)-6,12-dioxo-6,12-dihydroindolo[2,1-b]quinazoline-8-carbonitrile the title compound was obtained as a solid (16% yield). ¹H NMR (400 MHz, DMSO-d₆) δ 8.64-8.63 (m, 2H), 8.22 (s, 1H), 8.17 (d, J=8.4 Hz, 1H), 7.86 (d, J=8.4 Hz, 1H), 7.81 (d, J=8.0 Hz, 1H), 4.34 (s, 3H), 3.66 (s, 2H), 3.61-3.59 (m, 4H), 2.42-2.40 (m, 4H). LC-MS: m/z [M+H]⁺ 402.2.

### Example 115: Synthesis of (Z)-8-fluoro-6-(methoxyimino)-2-((pyridin-2-ylamino)methyl)indolo[2,1-b]quinazolin-12(6H)-one (Compound 134)

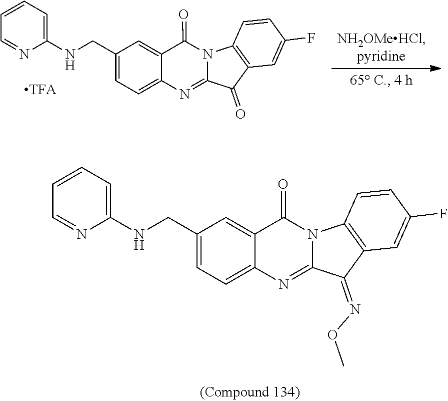 [SMILES: O=C1c2cc(F)ccc2-n2c1nc1ccc(CNc3ccccn3)cc1c2=O ; CO/N=C1/c2cc(F)ccc2-n2c1nc1ccc(CNc3ccccn3)cc1c2=O]

Following general procedure 14 but using 8-fluoro-2-((pyridin-2-ylamino)methyl)indolo[2,1-b]quinazoline-6,12-dione trifluoroacetate salt the title compound was obtained as a solid (29% yield). 1H NMR (400 MHz, DMSO-d₆) Major Isomer: δ 8.47 (dd, J=12.8, 4.0 Hz, 1H), 8.22 (s, 1H), 8.02 (dd, J=8.0, 2.4 Hz, 1H), 7.94 (d, J=4.0 Hz, 1H), 7.85-7.82 (m, 1H), 7.78 (d, J=8.4 Hz, 1H), 7.60-7.54 (m, 1H), 7.47-7.39 (m, 1H), 7.34 (brm, 1H), 6.57 (d, J=8.4 Hz, 1H), 6.50 (t, J=6.4 Hz, 1H), 4.65 (d, J=6.0 Hz, 2H), 4.31 (s, 3H); Minor Isomer: δ 8.46-8.43 (m, 1H), 4.67 (m, 2H), 2.29 (s, 3H), other peaks merged with major isomer. LC-MS: m/z [M+H]⁺ 402.25.

### Example 116: Synthesis of (Z)-2-((dimethylamino)methyl)-8-fluoro-6-(methoxyimino)indolo[2,1-b]quinazolin-12(6H)-one Trifluoroacetate Salt (Compound 138)

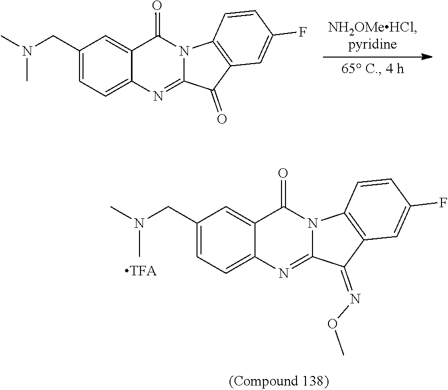 [SMILES: CN(C)Cc1ccc2nc3n(c(=O)c2c1)-c1ccc(F)cc1C3=O ; CO/N=C1/c2cc(F)ccc2-n2c1nc1ccc(CN(C)C)cc1c2=O]

Following general procedure 14 but using 2-((dimethylamino)methyl)-8-fluoroindolo[2,1-b]quinazoline-6,12-dione the title compound was obtained as a solid (19% yield). ¹H NMR (400 MHz, DMSO-d₆) δ 9.83 (s, 1H), 8.55-8.50 (i, 2H), 8.05 (dd, J=8.0 Hz, 2.4 Hz, 1H), 7.98-7.93 (m, 2H), 7.65-7.57 (i, 1H), 4.48 (s, 2H), 4.34 (s, 3H), 2.77 (s, 6H). LC-MS: m/z [M+H]⁺ 353.2.

### Example 117: Synthesis of (Z)—N-((8-fluoro-6-(methoxyimino)-12-oxo-6,12-dihydroindolo[2,1-b]quinazolin-3-yl)methyl)acetamide (Compound 94)

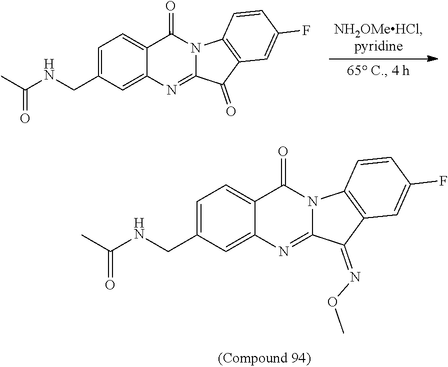 [SMILES: CC(=O)NCc1ccc2c(=O)n3c(nc2c1)C(=O)c1cc(F)ccc1-3 ; CO/N=C1/c2cc(F)ccc2-n2c1nc1cc(CNC(C)=O)ccc1c2=O]

Following general procedure 14 but using N-((8-fluoro-6,12-dioxo-6,12-dihydroindolo[2,1-b]quinazolin-3-yl)methyl)acetamide the title compound was obtained as a solid (6% yield). ¹H NMR (400 MHz, DMSO-d₆) δ 8.56 (t, J=6.0 Hz, 1H), 8.51 (q, J=4.8 Hz, 1H), 8.24 (d, J=8.4 Hz, 1H), 8.03 (dd, J=8.4 Hz, 2.8 Hz, 1H), 7.65 (s, 1H), 7.60-7.54 (m, 1H), 7.51 (dd, J=8.0 Hz, 1.6 Hz, 1H), 4.43 (d, J=5.6 Hz, 2H), 4.32 (s, 3H), 1.94 (s, 3H). LC-MS: m/z [M+H]⁺ 367.2.

### Example 118: Synthesis of (Z)-8-fluoro-3-(1H-imidazol-1-yl)-6-(methoxyimino)indolo[2,1-b]quinazolin-12(6H)-one (Compound 97)

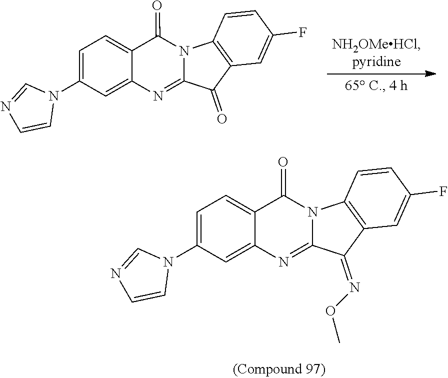 [SMILES: O=C1c2cc(F)ccc2-n2c1nc1cc(-n3ccnc3)ccc1c2=O ; CO/N=C1/c2cc(F)ccc2-n2c1nc1cc(-n3ccnc3)ccc1c2=O]

Following general procedure 14 but using 8-fluoro-3-(1H-imidazol-1-yl)indolo[2,1-b]quinazoline-6,12-dione the title compound was obtained as a solid (64% yield). ¹H NMR (400 MHz, DMSO-d₆) Major Isomer: δ 8.59 (s, 1H), 8.51 (dd, J=8.8, 4.8 Hz, 1H), 8.36 (d, J=8.8 Hz, 1H), 8.26 (d, J=2.0 Hz, 1H), 8.05-7.98 (m, 3H), 7.63-7.56 (m, 1H), 7.19 (s, 1H), 4.33 (s, 3H); Minor Isomer: δ 8.63 (s, 1H), 8.47 (dd, J=8.0, 4.4 Hz, 1H), 8.18 (d, J=2.0 Hz, 1H), 8.10 (s, 1H), 7.51-7.46 (m, 1H), 4.32 (s, 3H), other peaks are merged with major isomer. LC-MS: m/z [M+H]⁺ 362.1.

### Example 119: Synthesis of benzyl (Z)-((8-fluoro-6-(methoxyimino)-12-oxo-6,12-dihydroindolo[2,1-b]quinazolin-3-yl)methyl)carbamate (Compound 93)

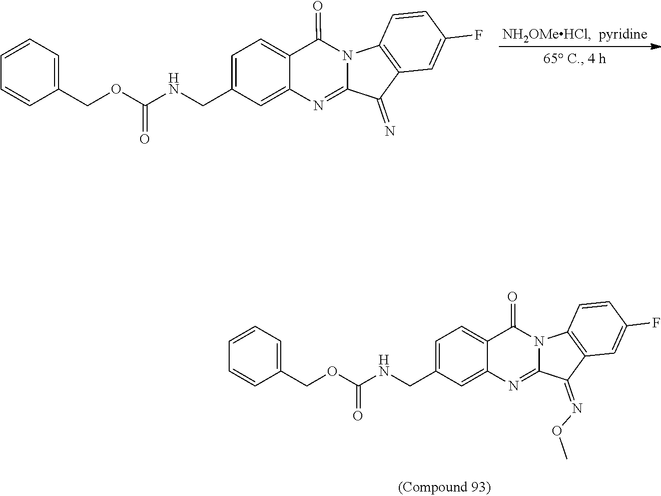 [SMILES: N=C1c2cc(F)ccc2-n2c1nc1cc(CNC(=O)OCc3ccccc3)ccc1c2=O ; CO/N=C1/c2cc(F)ccc2-n2c1nc1cc(CNC(=O)OCc3ccccc3)ccc1c2=O]

Following general procedure 14 but using benzyl ((8-fluoro-6,12-dioxo-6,12-dihydroindolo[2,1-b]quinazolin-3-yl)methyl)carbamate the title compound was obtained (13% yield). ¹H NMR (400 MHz, DMSO-d₆) δ 8.50 (brs, 1H), 8.23 (d, J=6.4, 1H), 8.03 (t, J=16 Hz, 8.0 Hz, 2H), 7.66 (s, 1H), 7.56-7.50 (m, 2H), 7.38-7.19 (m, 5H), 5.08 (s, 2H), 4.40 (s, 2H), 4.32 (s, 3H). LC-MS: m/z [M+H]⁺ 459.2.

### Example 120: Synthesis of (Z)-8-fluoro-2-(1H-imidazol-1-yl)-6-(methoxyimino)indolo[2,1-b]quinazolin-12(6H)-one (Compound 171)

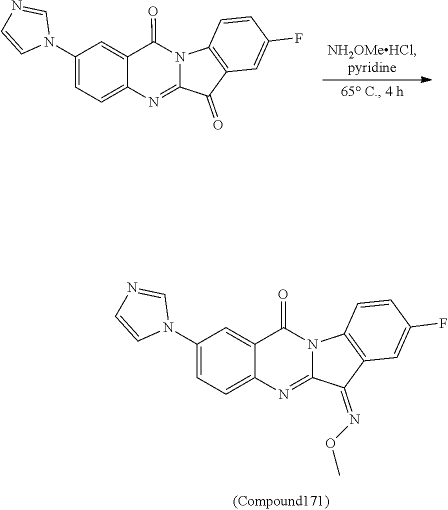 [SMILES: O=C1c2cc(F)ccc2-n2c1nc1ccc(-n3ccnc3)cc1c2=O ; CO/N=C1/c2cc(F)ccc2-n2c1nc1ccc(-n3ccnc3)cc1c2=O]

Following general procedure 14 but using 8-fluoro-2-(1H-imidazol-1-yl)indolo[2,1-b]quinazoline-6,12-dione the title compound was obtained (82% yield). ¹H NMR (400 MHz, DMSO-d₆) NMR at elevated temperature-Major Isomer: δ 8.54 (dd, J=8.0, 4.0 Hz, 1H), 8.44-8.41 (m, 2H), 8.20-8.16 (m, 1H), 8.03 (dd, J=8.4, 2.8 Hz, 1H), 7.98 (d, J=8.4 Hz, 1H), 7.88 (s, 1H), 7.59-7.53 (m, 1H), 7.18 (s, 1H), 4.36 (s, 3H); Minor Isomer: δ 8.48 (dd, J=9.2, 4.8 Hz, 1H), 7.90 (s, 1H), 7.49-7.45 (m, 1H), 4.34 (s, 3H). LC-MS: m/z [M+H]⁺ 362.2.

### Example 121: Synthesis of (Z)-6-(methoxyimino)-3-((4-methylpiperazin-1-yl)methyl)-12-oxo-6,12-dihydroindolo[2,1-b]quinazoline-8-sulfonamide (Compound 165)

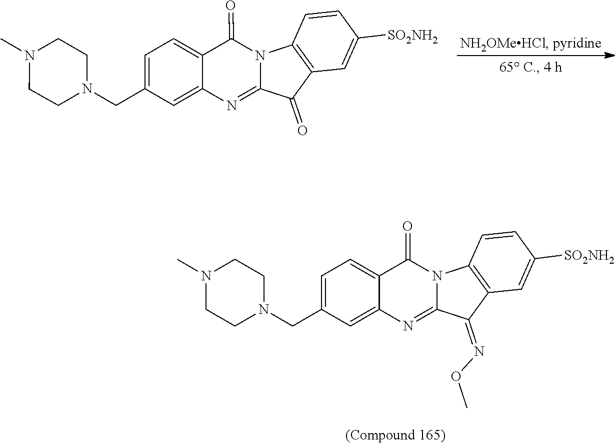 [SMILES: CN1CCN(Cc2ccc3c(=O)n4c(nc3c2)C(=O)c2cc(S(N)(=O)=O)ccc2-4)CC1 ; CO/N=C1/c2cc(S(N)(=O)=O)ccc2-n2c1nc1cc(CN3CCN(C)CC3)ccc1c2=O]

Following general procedure 14 but using 3-((4-methylpiperazin-1-yl)methyl)-6,12-dioxo-6,12-dihydroindolo[2,1-b]quinazoline-8-sulfonamide the title compound was obtained (8% yield). 1H NMR (400 MHz, DMSO-d₆) δ 8.66-8.64 (m, 2H), 8.26 (d, J=8.4 Hz, 1H), 8.13 (dd, J=8.4, 2.0 Hz, 1H), 7.75 (s, 1H), 7.61-7.59 (m, 3H), 4.36 (s, 3H), 3.67 (s, 2H), 2.50-2.35 (m, 6H), 2.16 (s, 3H); 2H merged with solvent. LC-MS: m/z [M+H]⁺ 469.3.

### Example 122: Synthesis of (Z)-6-(methoxyimino)-3-((4-methylpiperazin-1-yl)methyl)-12-oxo-6,12-dihydroindolo[2,1-b]quinazoline-8-sulfonamide (Compound 169)

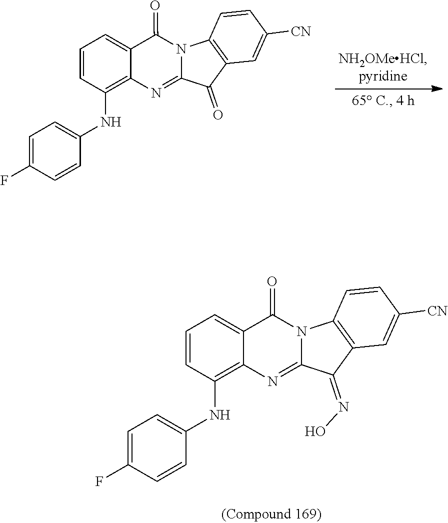 [SMILES: N#Cc1ccc2c(c1)C(=O)c1nc3c(Nc4ccc(F)cc4)cccc3c(=O)n1-2 ; N#Cc1ccc2c(c1)/C(=N/O)c1nc3c(Nc4ccc(F)cc4)cccc3c(=O)n1-2]

Following general procedure 14 but using 4-((4-fluorophenyl)amino)-6,12-dioxo-6,12-dihydroindolo[2,1-b]quinazoline-8-carbonitrile and hydroxylamine hydrochloride the title compound was obtained (400% yield). ¹H NMR (400 MHz, DMSO-d₆) δ 14.02 (S, 1H), 8.72 (d, J=1.2 Hz, 1H), 8.69 (d, J=8.4 Hz, 1H), 8.16 (dd, J=8.4, 1.6 Hz, 1H), 8.10 (s, 1H), 7.66 (dd, J=7.6, 1.6 Hz, 1H), 7.36-7.50 (m, 4H), 7.20 (t, J=8.8 Hz, 2H). LC-MS: m/z [M+H]⁺ 398.1.

### General Procedure 15

### Example 123: Synthesis of (Z)-6-(methoxyimino)-3-((4-methylpiperazin-1-yl)methyl)-12-oxo-6,12-dihydroindolo[2,1-b]quinazoline-8-sulfonamide

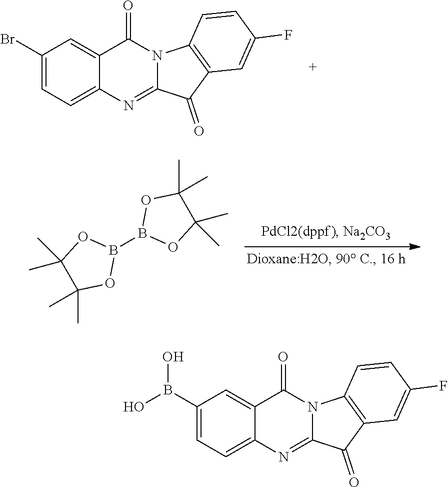

To the stirred solution of 2-bromo-8-fluoro-6H,12H-indolo[2,1-b]quinazoline-6,12-dione (1 g, 2.9 mmol, 1.0 equiv) in 1,4-dioxane (30 mL) were added 4,4,5,5-tetramethyl-2-(4,4,5,5-tetramethyl-1,3,2-dioxaborolan-2-yl)-1,3,2-dioxaborolane (1.47 g, 5.79 mmol, 2 equiv), and KOAc (853 mg, 8.69 mmol, 3 equiv) at RT. Then the reaction mixture was purged with N₂ for 15 min. After that, Pd(dppf)Cl₂ (0.107 g, 0.145 mmol, 0.05 equiv) was added and the
mixture was continuously stirred for 80° C. for 16 h. The mixture turned green black after several minutes of reaction. After complete consumption of the starting material, the mixture was cooled to RT then diluted with DCM (100 mL) and washed with brine (3×150 mL). The organic layer was dried with anhydrous Na₂SO₄ and filtered. The filtrate was concentrated to obtained {8-fluoro-6,12-dioxo-6H,12H-indolo[2,1-b]quinazolin-2-yl}boronic acid (0.9 g, 43% yield) as a brown solid with 58% LCMS Purity which was used directly in the next step without purification. ¹H NMR (400 MHz, DMSO-d₆) δ 8.53-8.49 (m, 1H), 8.05-8.03 (m, 1H), 7.75-7.69 (m, 2H), 7.56-7.48 (m, 3H). LC-MS: m/z [M+H]⁺ 311.0.

### Example 124: Synthesis of (8-fluoro-6,12-dioxo-6,12-dihydroindolo[2,1-b]quinazolin-3-yl)boronic Acid

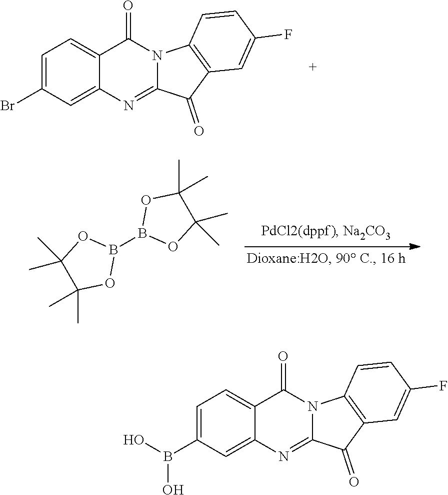

Following general procedure 15 but using 3-bromo-8-fluoroindolo[2,1-b]quinazoline-6,12-dione the title compound was obtained (42.00% yield). LC-MS: m/z [M+H]⁺ 311.0.

### Example 125: Synthesis of (8-cyano-6,12-dioxo-6,12-dihydroindolo[2,1-b]quinazolin-2-yl)boronic Acid

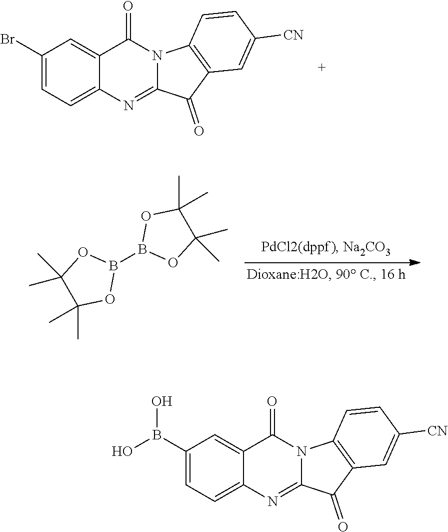

Following general procedure 15 but using 2-bromo-6,12-dioxo-6,12-dihydroindolo[2,1-b]quinazoline-8-carbonitrile the title compound was obtained (40.00% yield). LC-MS: m/z [M+H]⁺ 318.0.

### General Procedure 16

### Example 126: Synthesis of 8-fluoro-2-(1-((2-(trimethylsilyl)ethoxy)methyl)-1H-imidazol-4-yl)indolo[2,1-b]quinazoline-6,12-dione

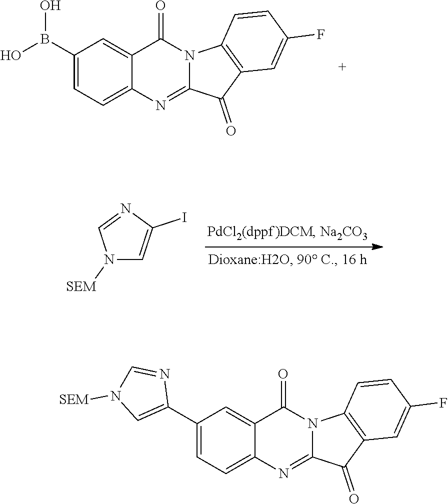 [SMILES: O=C1c2cc(F)ccc2-n2c1nc1ccc(B(O)O)cc1c2=O ; C[Si](C)(C)CCOCn1cnc(I)c1 ; C[Si](C)(C)CCOCn1cnc(-c2ccc3nc4n(c(=O)c3c2)-c2ccc(F)cc2C4=O)c1]

To the stirred solution of {8-fluoro-6,12-dioxo-6H,12H-indolo[2,1-b]quinazolin-2-yl}boronic acid (0.2 g, 0.631 mmol, 1.0 equiv) in 1,4-dioxane:water (10 mL, 9:1 ratio) under N₂ at RT were added tert-butyl 4-iodo-1H-imidazole-1-carboxylate (0.186 g, 0.631 mmol, 1.0 equiv) and Na₂CO₃ (0.167 g, 1.58 mmol, 2.5 equiv) in (100 mL) seal-tube. The reaction mixture was degassed with N₂ for 10 minutes, then added (1,1′-Bis(diphenylphosphino)ferrocene)palladium(II) dichloride DCM complex (0.025 mg, 0.031 mmol, 0.05 equiv). The resulting mixture was heated at 90° C. for 16 h. After complete consumption of starting material, the mixture was cooled to RT, filtered through a celite bed and solvents evaporated under reduced pressure. The residue was dissolved in EA (30 mL) and washed with water (2×50 mL). The combined organic extracts were dried over anhydrous Na₂SO₄, filtered and concentrated under reduced pressure to obtain solid residue. The solid residue was triturated with diethyl ether. The precipitate was filtered and the filter cake was purified by prep-HPLC to obtain 8-fluoro-2-(1-((2-(trimethylsilyl)ethoxy)methyl)-1H-imidazol-4-yl)indolo[2,1-b]quinazoline-6,12-dione (0.12 g, 43% yield) as an orange solid with LCMS purity 60% which was used directly in the next step without purification. 1H NMR not recorded, LC-MS: m/z [M+H]⁺ 462.1

### Example 127: Synthesis of 8-fluoro-3-(1-((2-(trimethylsilyl)ethoxy)methyl)-1H-imidazol-4-yl)indolo[2,1-b]quinazoline-6,12-dione

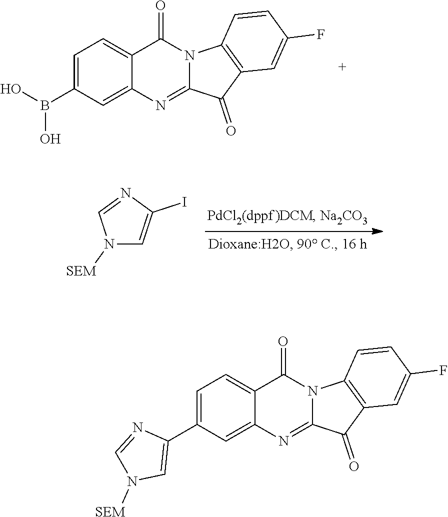 [SMILES: O=C1c2cc(F)ccc2-n2c1nc1cc(B(O)O)ccc1c2=O ; C[Si](C)(C)CCOCn1cnc(I)c1 ; C[Si](C)(C)CCOCn1cnc(-c2ccc3c(=O)n4c(nc3c2)C(=O)c2cc(F)ccc2-4)c1]

Following general procedure 16 but using (8-fluoro-6,12-dioxo-6,12-dihydroindolo[2,1-b]quinazolin-3-yl)boronic acid the title compound was obtained (20% yield). LC-MS: m/z [M+H]⁺ 462.15.

### Example 128: Synthesis of tert-butyl 4-(8-cyano-6,12-dioxo-6,12-dihydroindolo[2,1-b]quinazolin-2-yl)-1H-imidazole-1-carboxylate

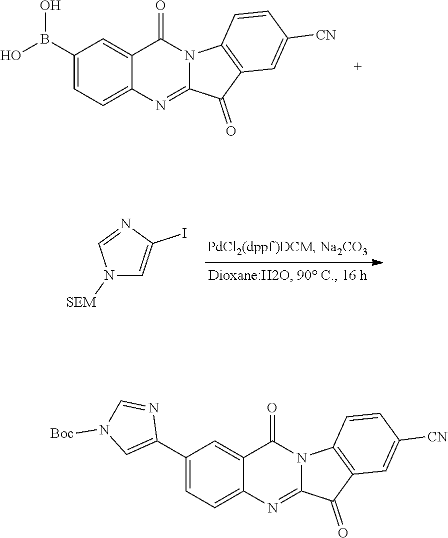 [SMILES: N#Cc1ccc2c(c1)C(=O)c1nc3ccc(B(O)O)cc3c(=O)n1-2 ; C[Si](C)(C)CCOCn1cnc(I)c1 ; CC(C)(C)OC(=O)n1cnc(-c2ccc3nc4n(c(=O)c3c2)-c2ccc(C#N)cc2C4=O)c1]

Following general procedure 16 but using (8-cyano-6,12-dioxo-6,12-dihydroindolo[2,1-b]quinazolin-2-yl)boronic acid and tert-butyl 4-iodo-1H-imidazole-1-carboxylate the title compound was obtained (20% yield). LC-MS: m/z [M+H]⁺ 462.15.

### General Procedure 17

### Example 129: Synthesis of 8-fluoro-2-(1H-imidazol-4-yl)indolo[2,1-b]quinazoline-6,12-dione (Compound 151)

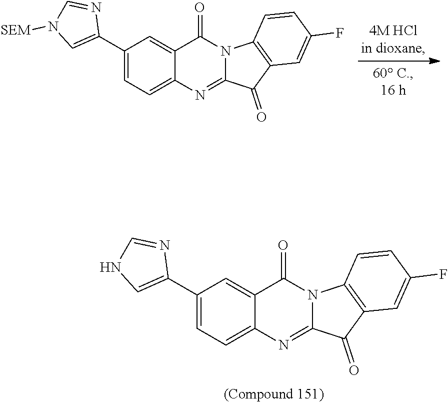 [SMILES: C[Si](C)(C)CCOCn1cnc(-c2ccc3nc4n(c(=O)c3c2)-c2ccc(F)cc2C4=O)c1 ; O=C1c2cc(F)ccc2-n2c1nc1ccc(-c3c[nH]cn3)cc1c2=O]

To the stirred solution of 8-fluoro-3-(1-{[2-(trimethylsilyl)ethoxy]methyl}-1H-imidazol-4-yl)-6H,12H-indolo[2,1-b]quinazoline-6,12-dione (0.12 mg, 0.259 mmol) in dichloromethane at RT under N₂. was added 4N HCl in dioxane (0.378 mg, 104 mmol) The resulting mixture was continuously stirred at 60° C. for 16 h. After complete consumption of starting material, the mixture was concentrated under reduce pressure. The solid residue was triturated with diethyl ether. The precipitate was filtered and the filter cake was purified by prep-HPLC to obtain 8-fluoro-3-(1H-imidazol-4-yl)-6H,12H-indolo[2,1-b]quinazoline-6,12-dione (0.019 g, 22% yield) as a greenish solid. 1H NMR (400 MHz, DMSO-d₆)−δ: 14.14 (brs, 1H), 8.75 (s, 1H), 8.67 (s, 1H), 8.51 (dd, J=8.8, 4.0 Hz, 1H), 8.34 (d, J=7.6 Hz, 1H), 8.26 (s, 1H), 8.03 (d, J=8.4 Hz, 1H), 7.82-7.79 (m, 1H), 7.77-7.73 (m, 1H). LC-MS: m/z [M+H]⁺ 333.1.

### Example 130: Synthesis of 2-(1H-imidazol-4-yl)-6,12-dioxo-6,12-dihydroindolo[2,1-b]quinazoline-8-carbonitrile (Compound 162)

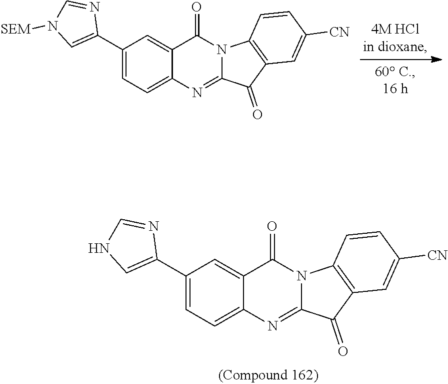 [SMILES: C[Si](C)(C)CCOCn1cnc(-c2ccc3nc4n(c(=O)c3c2)-c2ccc(C#N)cc2C4=O)c1 ; N#Cc1ccc2c(c1)C(=O)c1nc3ccc(-c4c[nH]cn4)cc3c(=O)n1-2]

Following general procedure 17 but using 6,12-dioxo-2-(1-((2-(trimethylsilyl)ethoxy)methyl)-1H-imidazol-4-yl)-6,12-dihydroindolo[2,1-b]quinazoline-8-carbonitrile the title compound was obtained (10% yield). ¹H NMR (400 MHz, DMSO-d₆)−δ 12.33 (s, 1H), 8.67 (s, 1H), 8.58 (q, J=4.8 Hz, 1H), 8.26 (d, J=7.6 Hz, 1H), 8.16 (d, J=8.4 Hz, 1.6 Hz, 1H), 7.91-7.75 (m, 3H), 7.42 (m, 1H), 1.89 (s, 1H). LC-MS: m/z [M+H]⁺ 340.3.

### Example 131: Synthesis of 8-fluoro-3-(1H-imidazol-4-yl)indolo[2,1-b]quinazoline-6,12-dione (Compound 89)

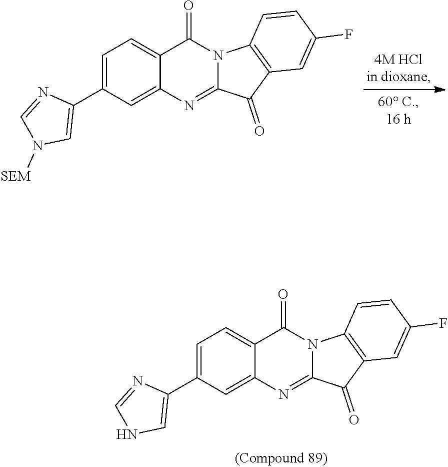 [SMILES: C[Si](C)(C)CCOCn1cnc(-c2ccc3c(=O)n4c(nc3c2)C(=O)c2cc(F)ccc2-4)c1 ; O=C1c2cc(F)ccc2-n2c1nc1cc(-c3c[nH]cn3)ccc1c2=O]

Following general procedure 17 but using 8-fluoro-3-(1-((2-(trimethylsilyl)ethoxy)methyl)-1H-imidazol-4-yl)indolo[2,1-b]quinazoline-6,12-dione the title compound was obtained (22% yield). ¹H NMR (400 MHz, DMSO-d₆)−δ: 9.13 (brs, 1H), 8.49 (q, J=4.4 Hz, 1H), 8.43 (dd, J=6.8 Hz, 2.8 Hz, 2H), 8.40 (s, 1H), 8.18 (d, J=8.4 Hz, 1.6 Hz, 1H), 7.96-7.51 (m, 3H). LC-MS: m/z [M+H]⁺ 332.07.

### Example 132: Synthesis of 2-((4,6-dimethoxypyrimidin-2-yl)amino)-8-fluoroindolo[2,1-b]quinazoline-6,12-dione (Compound 135)

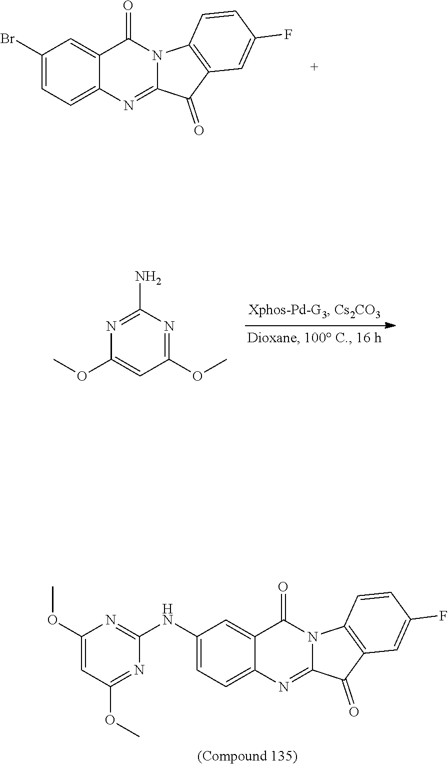

To the stirred solution of 2-bromo-8-fluoro-6H,12H-indolo[2,1-b]quinazoline-6,12-dione (0.1 g, 0.290 mmol, 1.0 equiv) and 4,6-dimethoxypyrimidin-2-amine (0.067 g, 0.435 mmol) in dioxane (3 mL) at room temperature. was added sodium tert-butoxide (0.167 g, 0.87 mmol) and the reaction mixture was purged with nitrogen gas for 10 min. then added Pd₂(dba)₃ (0.053 mg 0.015 mmol, 0.05 equiv) and Xantphos (33.5 mg, 0.015 mmol, 0.05 equiv) The resulting mixture was heated at 100° C. for 16 h. After complete consumption of starting material, the mixture was cooled to RT, filtered through a celite bed and solvents evaporated from the filtrate under reduced pressure. The residue was dissolved in EA (10 mL) and washed with water (2×20 mL). The combined organic extracts were dried over anhydrous Na₂SO₄, filtered and concentrated under reduced pressure. The solid residue was triturated with diethyl ether. The precipitate was filtered and the filter cake was purified by prep-HPLC to obtain 2-((4,6-dimethoxypyrimidin-2-yl)amino)-8-fluoroindolo[2,1-b]quinazoline-6,12-dione (0.05 g, 41%) as a yellow solid. ¹H NMR (400 MHz, DMSO-d₆)−δ: 10.26 (s, 1H), 9.15 (s, 1H), 8.51-8.50 (m, 1H), 8.12-8.10 (m, 1H), 7.87 (d, J=8.8 Hz, 1H), 7.75-7.65 (m, 2H), 5.79 (s, 1H), 3.98 (s, 6H). LC-MS: m/z [M+H]⁺ 420.3.

### Example 133: Synthesis of 1-(8-fluoro-6,12-dioxo-6,12-dihydroindolo[2,1-b]quinazolin-2-yl)guanidine Hydrochloride (Compound 164)

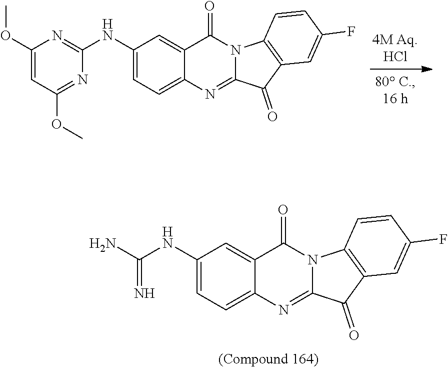 [SMILES: COc1cc(OC)nc(Nc2ccc3nc4n(c(=O)c3c2)-c2ccc(F)cc2C4=O)n1 ; N=C(N)Nc1ccc2nc3n(c(=O)c2c1)-c1ccc(F)cc1C3=O]

To the stirred solution of 2-[(4,6-dimethoxypyrimidin-2-yl)amino]-8-fluoro-6H,12H-indolo[2,1-b]quinazoline-6,12-dione (0.5 g, 1.19 mmol) in 4N HCL in water (6 mL) at RT. Then resulting mixture was heated at 80° C. for 16 hrs. After complete consumption of starting material, the reaction was cooled to RT. The precipitated solid was filtered and washed with diethyl ether and dried under vacuum. The crude material was purified by prep-HPLC to yield N-{8-fluoro-6,12-dioxo-6H,12H-indolo[2,1-b]quinazolin-2-yl}guanidine hydrochloride (7 mg, 2%) as an off-white solid. 1H NMR (400 MHz, DMSO-d₆)−δ: 10.16 (s, 1H), 8.49 (dd, J=8.8, 4.4 Hz, 1H), 8.10 (d, J=2.4 Hz, 1H), 8.01 (d, J=8.8 Hz, 1H), 7.77-7.84 (m, 7H). LC-MS: m/z [M+H]⁺ 324.1.

### Example 134: Synthesis of 1-((8-fluoro-6,12-dioxo-6,12-dihydroindolo[2,1-b]quinazolin-2-yl)methyl)urea (Compound 139)

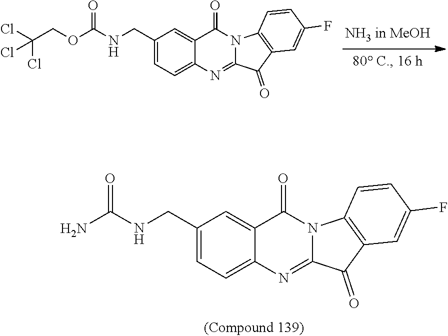 [SMILES: O=C(NCc1ccc2nc3n(c(=O)c2c1)-c1ccc(F)cc1C3=O)OCC(Cl)(Cl)Cl ; NC(=O)NCc1ccc2nc3n(c(=O)c2c1)-c1ccc(F)cc1C3=O]

To the stirred solution 2,2,2-trichloroethyl ((8-fluoro-6,12-dioxo-6,12-dihydroindolo[2,1-b]quinazolin-2-yl)methyl)carbamate (0.22 g, 0.467 mmol) in methanolic ammonia solution (4 mL) nitrogen atmosphere at rt in seal tube. The resulting mixture was heated at 80° C. for 16 h. After complete consumption of starting material, the mixture was cooled to RT and concentrated under reduced pressure. The solid residue was triturated with diethyl ether. The precipitate was filtered and the filter cake was purified by prep-HPLC to obtain 1-((8-fluoro-6,12-dioxo-6,12-dihydroindolo[2,1-b]quinazolin-2-yl)methyl)urea (0.13 g, 85%) as yellow solid. ¹H NMR (400 MHz, DMSO-d₆)−δ: 12.43 (s, 1H), 8.52-8.47 (m, 1H), 8.22 (s, 1H), 7.53-8.02 (m, 4H), 5.43 (d, J=4.4 Hz, 2H), 4.39 (d, J=6.0 Hz, 2H). LC-MS: m/z [M+H]⁺ 339.1.

### Example 135: Synthesis of 8-fluoro-6,12-dioxo-6,12-dihydroindolo[2,1-b]quinazoline-2-carboximidamide (Compound 148)

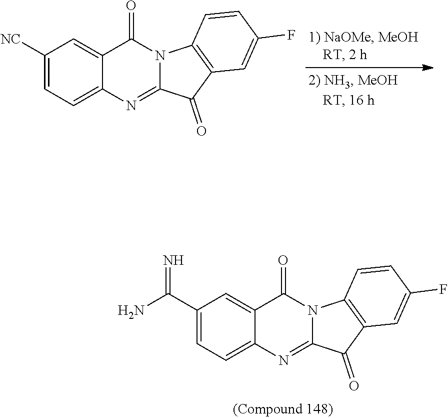 [SMILES: N#Cc1ccc2nc3n(c(=O)c2c1)-c1ccc(F)cc1C3=O ; N=C(N)c1ccc2nc3n(c(=O)c2c1)-c1ccc(F)cc1C3=O]

To the stirred solution of 8-fluoro-6,12-dioxo-6H,12H-indolo[2,1-b]quinazoline-2-carbonitrile (0.2 g, 0.687 mmol, 1.0 equiv) in MeOH (8 mL) at RT under N₂. was added 33% NaOMe in MeOH solution (1.48 g, 27.5 mmol), the mixture was stirred at RT for 2 h. After 2 h purge ammonia gas and then continuously stirred at RT for 16 h. during stirring a precipitate formed. After complete consumption of starting material, the solid precipitate was filtered and triturated with MeOH and dried under vacuum to obtain 8-fluoro-6,12-dioxo-6H,12H-indolo[2,1-b]quinazoline-2-carboximidamide (135 mg, 40%) as a brown solid. ¹H NMR (400 MHz, DMSO-d₆)−δ: 15.24 (s, 1H), 12.44 (s, 1H), 9.01 (d, J=8.8 Hz, 1H), 8.27 (d, J=1.6 Hz, 1H), 7.79 (d, J=7.2 Hz, 1H), 7.56 (d, J=6.0 Hz, 1H), 7.21-7.14 (m, 2H). LC-MS: m/z [M+H]⁺ 309.1.

### Example 136: Synthesis of 8-fluoro-6,12-dioxo-6,12-dihydroindolo[2,1-b]quinazoline-2-carboxamide (Compound 149)

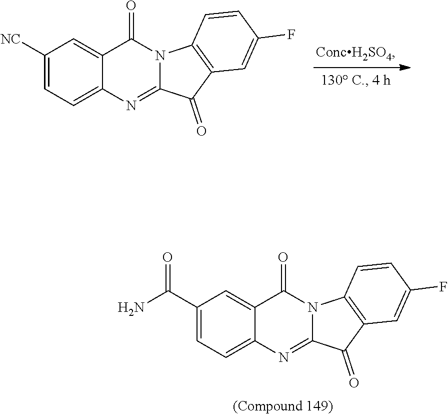 [SMILES: N#Cc1ccc2nc3n(c(=O)c2c1)-c1ccc(F)cc1C3=O ; NC(=O)c1ccc2nc3n(c(=O)c2c1)-c1ccc(F)cc1C3=O]

To the stirred solution of 8-fluoro-6,12-dioxo-6H,12H-indolo[2,1-b]quinazoline-2-carbonitrile (0.2 g, 687 μmol, 1 equiv) in sulfuric acid (2.02 mL, 37.8 mmol) was stirred at 130° C. for 4 h. After complete consumption of starting material, the reaction mixture was cooled to RT and poured into ice-cold water. The precipitated solid was filtered, washed thoroughly with MeOH and diethyl ether to afford 8-fluoro-6,12-dioxo-6H,12H-indolo[2,1-b]quinazoline-2-carboxamide (90 mg, Yield: 42%) as a yellow solid. ¹H NMR (400 MHz, DMSO-d₆)−δ: 8.81 (d, J=1.6 Hz, 1H), 8.51 (dd, J=8.8, 4.0 Hz, 1H), 8.40-8.35 (m, 2H), 8.01 (d, J=8.4 Hz, 1H), 7.83-7.80 (m, 1H), 7.77-7.71 (m, 1H), 7.68 (s, 1H). LC-MS: m/z [M+H]⁺ 310.1.

### Example 137: Synthesis of 8-fluoro-6-hydroxy-6-methyl-12-oxo-6,12-dihydroindolo[2,1-b]quinazoline-2-carbonitrile (Compound 152)

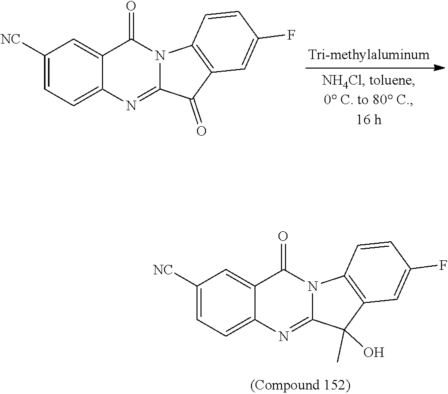 [SMILES: N#Cc1ccc2nc3n(c(=O)c2c1)-c1ccc(F)cc1C3=O ; CC1(O)c2cc(F)ccc2-n2c1nc1ccc(C#N)cc1c2=O]

To the stirred solution of ammonium chloride (36.7 mg, 687 μmol, 1 equiv) in toluene (0.4 mL) was added 2 M solution of Tri-methyl aluminum in Toluene (99 mg, 1.37 mmol, 2 equiv) at 0° C. Then, mixture was warmed to RT before addition of 8-fluoro-6,12-dioxo-6H,12H-indolo[2,1-b]quinazoline-2-carbonitrile (0.2 g, 687 μmol, 1 equiv). The mixture was heated at 80° C. for 16 h. After complete consumption of the starting material, the mixture was cooled to RT and quenched with water. Aqueous layer was extracted with EA (2×5 mL) and combined organic layers were dried over anhydrous Na₂SO₄, concentrate under vacuum. The crude product was purified via prep-HPLC to obtain 8-fluoro-6-hydroxy-6-methyl-12-oxo-6H,12H-indolo[2,1-b]quinazoline-2-carbonitrile (39 mg, Yield: 19%) obtained as an off white solid. ¹H NMR (400 MHz, DMSO-d₆)−δ: 8.68 (d, J=1.6 Hz, 1H), 8.40 (dd, J=8.8, 4.4 Hz, 1H), 8.26 (dd, J=8.4, 2.0 Hz, 1H), 7.96 (d, J=8.4 Hz, 1H), 7.58 (dd, J=8.0, 2.4 Hz, 1H), 7.41-7.36 (m, 1H), 6.50 (s, 1H), 1.72 (s, 3H). LC-MS: m/z [M+H]⁺ 308.1.

### Example 138: Synthesis of 8-fluoro-6,12-dioxo-6,12-dihydroindolo[2,1-b]quinazoline-2-carboxylic Acid (Compound 153)

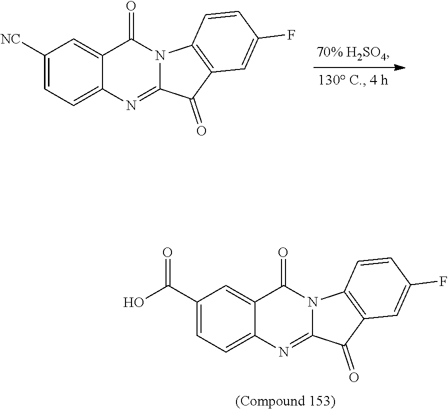 [SMILES: N#Cc1ccc2nc3n(c(=O)c2c1)-c1ccc(F)cc1C3=O ; O=C(O)c1ccc2nc3n(c(=O)c2c1)-c1ccc(F)cc1C3=O]

To the stirred solution of 8-fluoro-6,12-dioxo-6H,12H-indolo[2,1-b]quinazoline-2-carbonitrile (150 mg, 515 μmol, 1 equiv) in sulfuric acid (3 mL) and water (0.5 mL, 27.8 mmol), was heated at 130° C. for 3 h. The mixture was poured into ice water, the solid filtered and was washed thoroughly with water. The obtained material was further triturated with methanol and dried to afford 8-fluoro-6,12-dioxo-6H,12H-indolo[2,1-b]quinazoline-2-carboxylic acid (29 mg Yield: 18%) as yellow solid. ¹H NMR (400 MHz, DMSO-d₆)−δ: 8.78 (s, 1H), 8.51 (dd, J=8.8, 4.0 Hz, 1H), 8.35 (d, J=8.0 Hz, 1H), 7.90 (d, J=8.4 Hz, 1H), 7.78 (dd, J=7.2, 2.8 Hz, 1H), 7.74-7.69 (m, 1H), 7.24 (brs, 1H). LC-MS: m/z [M+H]⁺ 311.1.

### Example 139: Synthesis of 8-fluoro-2-(1H-imidazol-4-yl)indolo[2,1-b]quinazoline-6,12-dione (Compound 151)

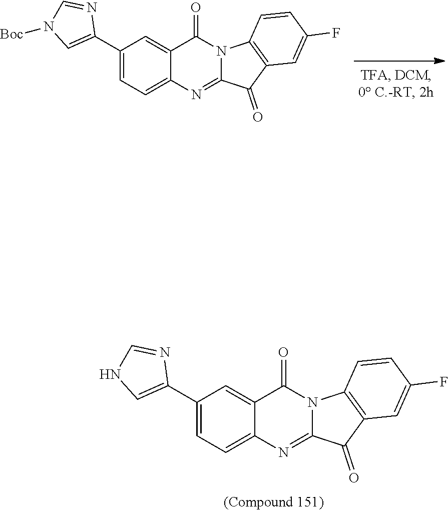 [SMILES: CC(C)(C)OC(=O)n1cnc(-c2ccc3nc4n(c(=O)c3c2)-c2ccc(F)cc2C4=O)c1 ; O=C1c2cc(F)ccc2-n2c1nc1ccc(-c3c[nH]cn3)cc1c2=O]

To the stirred solution of tert-butyl 4-{8-fluoro-6,12-dioxo-6H,12H-indolo[2,1-b]quinazolin-2-yl}-1H-imidazole-1-carboxylate (0.5 g, 1.16 mmol, 1 equiv) in DCM (10 mL), was added TFA (659 mg, 5.78 mmol, 5 equiv) at 0° C. under N₂. Then, resulting mixture was stirred at RT for 2 h. After complete consumption of starting material, saturated solution of NaHCO₃ was added (pH-7) and, aqueous layer was extracted with 10% MeOH:DCM (2×20 mL). Combined organic layers were dried over anhydrous Na₂SO₄, filtered and concentrated to give the crude product. This was purified by prep-HPLC to obtain 8-fluoro-2-(1H-imidazol-4-yl)-6H,12H-indolo[2,1-b]quinazoline-6,12-dione (18 mg, Yield: 5%) as a red colored solid. ¹H NMR (400 MHz, DMSO-d₆)−δ: 14.14 (brs, 1H), 8.75 (s, 1H), 8.67 (s, 1H), 8.51 (dd, J=8.8, 4.0 Hz, 1H), 8.34 (d, J=7.6 Hz, 1H), 8.26 (s, 1H), 8.03 (d, J=8.4 Hz, 1H), 7.82-7.79 (m, 1H), 7.77-7.73 (m, 1H). LC-MS: m/z [M+H]⁺ 333.1.

### Example 140: Synthesis of tert-butyl 4-iodo-1H-imidazole-1-carboxylate

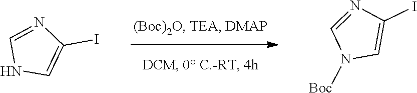 [SMILES: Ic1c[nH]cn1 ; CC(C)(C)OC(=O)n1cnc(I)c1]

To the stirred solution of 4-iodo-1H-imidazole (0.5 g, 2.58 mmol, 1 equiv) in dichloromethane (10 mL) under nitrogen atmosphere was added di-tert-butyl dicarbonate (1.13 g 5.16 mmol, 2 equiv), triethylamine (1.3 g, 12.9 mmol, 5 equiv) and DMAP (31.8 mg, 258 μmol, 0.1 equiv) at room temperature. Reaction was allowed to stir at RT for 4 h. After complete consumption of the starting material, the reaction mixture quenched with water and extracted with EA. The combined organic layers were washed with Sat. NaHCO₃ solution, dried with anhydrous Na₂SO₄ and filtered. The filtrate was concentrated to get tert-butyl 4-iodo-1H-imidazole-1-carboxylate (0.55 g, 1.87 mmol, 72%) as a light green liquid. ¹H NMR (400 MHz, DMSO-d₆)−δ: 8.15 (s, 1H), 7.74 (s, 1H), 1.38 (s, 9H).

### Example 141: Synthesis of 4-iodo-1-((2-(trimethylsilyl)ethoxy)methyl)-1H-imidazole

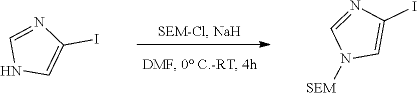 [SMILES: Ic1c[nH]cn1 ; C[Si](C)(C)CCOCn1cnc(I)c1]

To the suspension of NaH (0.148 g, 6.19 mmol, 1.2 equiv) in dry DMF (10 mL) at 0° C. under N₂ was added 4-iodo-1H-imidazole (1.0 g, 5.16 mmol, 1.0 equiv) portion wise and stirred for 1 h at the same temperature followed by addition of SEM-Cl (1.0 mL, 5.67 mmol) at 0° C. The mixture was stirred at RT for 12 h. After complete consumption of the starting material, the mixture was poured into ice-water (50 mL) and extracted with EtOAc (40 mL×3). Combined organic layers were washed with water (3×100 mL), dried over Na₂SO₄, filtered and concentrated under reduced pressure to get 4-iodo-1-{[2-(trimethylsilyl)ethoxy]methyl}-1H-imidazole (1.14 g 68%) as a liquid as crude; LCMS data shows 80% desired product and used as such for the next step. ¹H NMR (400 MHz, DMSO-d₆)−δ: 7.77 (s, 1H), 7.49 (s, 1H), 5.30 (s, 2H), 3.47-3.39 (m, 2H), 0.82 (t, J=8.0 Hz, 2H), −0.025 (s, 9H).

### General Procedure 18

### Example 142: Synthesis of methyl 4-(1H-imidazol-1-yl)-2-nitrobenzoate

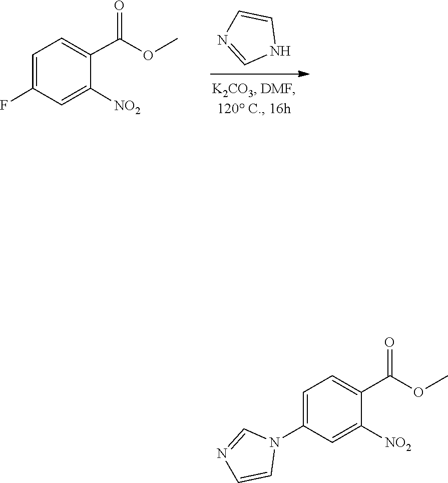

To the mixture of methyl 5-fluoro-2-nitrobenzoate (3 g, 15.1 mmol, 1.0 equiv) and 1H-imidazole (1.13 g, 16.6 mmol, 1.1 equiv) in DMF (50 mL) at RT was added K₂CO₃ (3.12 g, 22.6 mmol, 1.5 equiv). The mixture was heated at 120° C. for 16 h. After complete consumption of starting material, mixture was poured into ice water and extracted with EA. The organic layer was separated, dried over Na₂SO₄ and concentrated in vacuo to get methyl 4-(1H-imidazol-1-yl)-2-nitrobenzoate (0.23 g, 6%) as an off white solid. 1H NMR (400 MHz, DMSO-d₆)−δ: 8.53 (s, 1H), 8.43 (d, J=2.4 Hz, 1H), 8.16 (dd, J=8.4, 2.4 Hz, 1H), 8.05 (d, J=8.4 Hz, 1H), 7.99 (s, 1H), 7.18 (s, 1H), 3.87 (s, 3H).

### Example 143: Synthesis of methyl 5-(1H-imidazol-1-yl)-2-nitrobenzoate

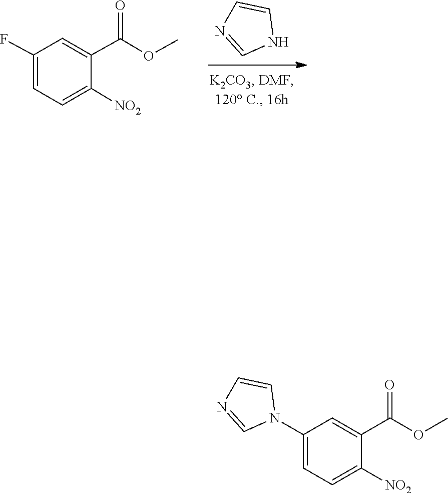

Following general procedure 18 but using methyl 5-fluoro-2-nitrobenzoate the title compound was obtained as a solid (40% yield). ¹H NMR (400 MHz, DMSO-d₆)—δ 8.54 (s, 1H), 8.29 (d, J=8.0 Hz, 1H), 8.19 (s, 1H), 8.12 (d, J=7.6 Hz, 1H), 8.00 (s, 1H), 7.19 (s, 1H), 3.90 (s, 3H). LC-MS: m/z [M+H]⁺ 247.06.

### General Procedure 19

### Example 144: Synthesis of methyl 2-amino-4-(1H-imidazol-1-yl)benzoate

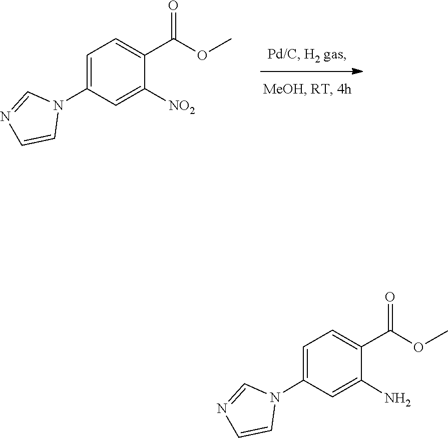 [SMILES: COC(=O)c1ccc(-n2ccnc2)cc1[N+](=O)[O-] ; COC(=O)c1ccc(-n2ccnc2)cc1N]

To the stirred solution of methyl 5-(1H-imidazol-1-yl)-2-nitrobenzoate (1.5 g, 6.07 mmol, 1.0 equiv) in methanol (40 mL) was added Pd/C (10%, 50% Wet) (1.29 g, 12.1 mmol) at RT under N₂. then stirred at 25° C. for 4 h under H₂ atmosphere. After complete consumption of starting material and was filtered through short celite bed on sintered funnel. Filtrate was concentrated under reduced pressure to get methyl 2-amino-5-(1H-imidazol-1-yl)benzoate (1.2 g, 5.52 mmol, 91%) as colorless liquid.

### Example 145: Synthesis of methyl 2-amino-5-(1H-imidazol-1-yl)benzoate

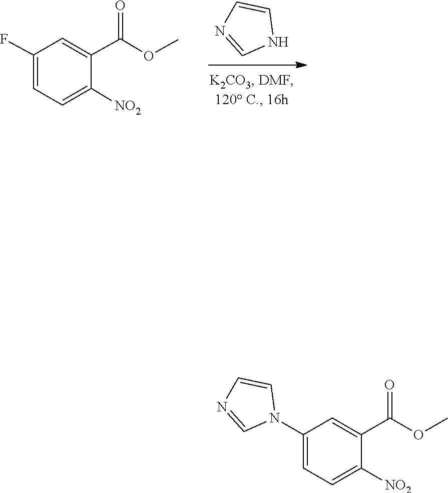

Following general procedure 19 but using methyl 5-(1H-imidazol-1-yl)-2-nitrobenzoate the title compound was obtained (91% yield). LC-MS: m/z [M+H]⁺ 217.09.

### General Procedure 20

### Example 146: Synthesis of 2-amino-4-(1H-imidazol-1-yl)benzoic Acid

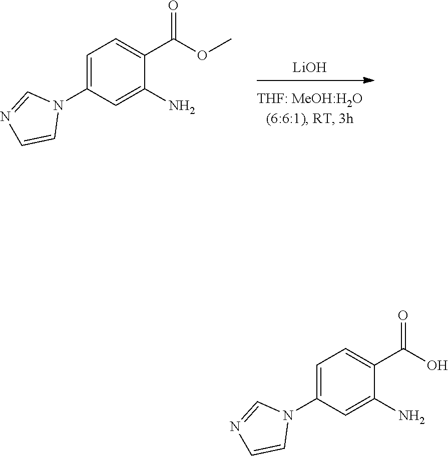 [SMILES: COC(=O)c1ccc(-n2ccnc2)cc1N ; "LiOH" ; Nc1cc(-n2ccnc2)ccc1C(=O)O]

Methyl 2-amino-4-(1H-imidazol-1-yl)benzoate (1.2 g, 5.52 mmol, 1.0 equiv) was dissolved in the mixture of THF (25 mL), water (25 mL) and MeOH at RT. To the above mixture was added LiOH (0.662 g, 27.6 mmol) and was allowed to stir at RT for 3 h. After complete consumption of SM, the solvent was removed to get the crude residue, which was diluted with water and acidified with 1N HCl up to pH-2 and extracted with EA. The organic layer was separated, dried over Na₂SO₄ and concentrated under reduced pressure to get 2-amino-4-(1H-imidazol-1-yl)benzoic acid (1.1 g, 97%) as a white solid.

### Example 147: Synthesis of 2-amino-5-(1H-imidazol-1-yl)benzoic Acid

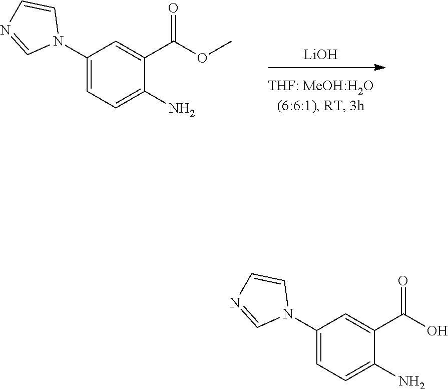 [SMILES: COC(=O)c1cc(-n2ccnc2)ccc1N ; "LiOH" ; Nc1ccc(-n2ccnc2)cc1C(=O)O]

Following general procedure 20 but using methyl 2-amino-5-(1H-imidazol-1-yl)benzoate the title compound was obtained (97% yield). LC-MS: m/z [M+H]⁺ 203.07.

### General Procedure 21

### Example 148: Synthesis of 7-(1H-imidazol-1-yl)-2H-benzo[d][1,3]oxazine-2,4(1H)-dione

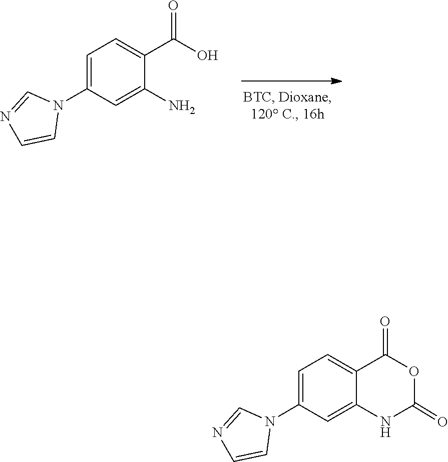 [SMILES: Nc1cc(-n2ccnc2)ccc1C(=O)O ; O=c1[nH]c2cc(-n3ccnc3)ccc2c(=O)o1]

To the stirred solution of 2-amino-4-(1H-imidazol-1-yl)benzoic acid (1 g, 4.92 mmol, 1 equiv) in 1,4-dioxane (20 mL), was added triphosgene (4.38 g, 14.8 mmol, 3 equiv) at RT. The mixture was heated at 120° C. for 16 hours. The solid formed was filtered and dried to afford 7-(1H-imidazol-1-yl)-2,4-dihydro-1H-3,1-benzoxazine-2,4-dione (1 g, 89%) as white solid.

### Example 149: Synthesis of 6-(1H-imidazol-1-yl)-2H-benzo[d][1,3]oxazine-2,4(1H)-dione

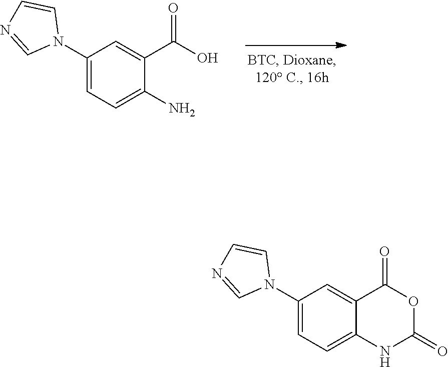 [SMILES: Nc1ccc(-n2ccnc2)cc1C(=O)O ; O=c1[nH]c2ccc(-n3ccnc3)cc2c(=O)o1]

Following general procedure 21 but using 2-amino-5-(1H-imidazol-1-yl)benzoic acid the title compound was obtained (88% yield). LC-MS: m/z [M+H]⁺ 229.05.

### General Procedure 22

### Example 150: Synthesis of tert-butyl (N-((8-fluoro-6,12-dioxo-6,12-dihydroindolo[2,1-b]quinazolin-3-yl)methyl)sulfamoyl)carbamate

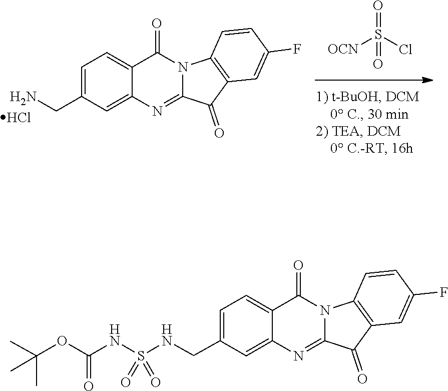

To the stirred solution of N-carbothioylsulfamoyl chloride (88.1 mg, 559 μmol, 1.1 equiv) in DCM (5 mL) was added t-butanol (53 μL, 559 μmol, 1.1 equiv) at 0° C. Then the mixture was stirred for 30 min at 0° C. Further, above solution was slowly added into the pre-dissolved solution of 2-(aminomethyl)-8-fluoro-6H,12H-indolo[2,1-b]quinazoline-6,12-dione (150 mg, 508 μmol), Et₃N (145 μL, 1.07 mmol) and DCM (6 mL, 93.7 mmol) at 0° C. under N₂. The resulting mixture was allowed to warm to RT and stirred for 16 h. The mixture was diluted with DCM (10 mL) and washed by 0.1N HCl solution and water. The organic layer was dried over anhydrous Na₂SO₄, filtered and concentrated to obtain a crude product. LCMS showed 46% desired product, which was used for next step without further purification.

### Example 151: Synthesis of tert-butyl (N-((8-fluoro-6,12-dioxo-6,12-dihydroindolo[2,1-b]quinazolin-2-yl)methyl)sulfamoyl)carbamate

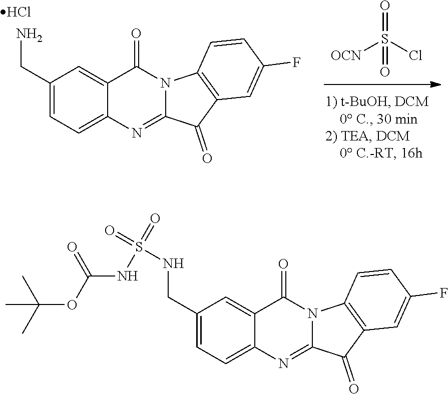

Following general procedure 22 but using 2-(aminomethyl)-8-fluoroindolo[2,1-b]quinazoline-6,12-dione hydrochloride the title compound was obtained (25% yield). LC-MS: m/z [M+H]⁺ 474.10.

### Example 152: Synthesis of tert-butyl (N-((8-cyano-6,12-dioxo-6,12-dihydroindolo[2,1-b]quinazolin-2-yl)methyl)sulfamoyl)carbamate

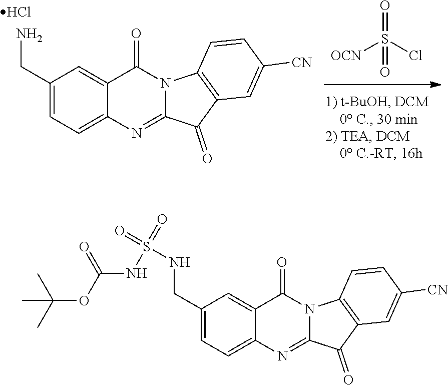

Following general procedure 22 but using 2-(aminomethyl)-6,12-dioxo-6,12-dihydroindolo[2,1-b]quinazoline-8-carbonitrile hydrochloride the title compound was obtained (27% yield). LC-MS: m/z [M+H]⁺ 481.11.

### Example 153: Cellular IDO1 Protocol

Indoleamine 2,3-dioxygenase 1 (IDO) is a heme-containing protein that catalyzes the oxidative cleavage of the C2-C3 double bond of the indole in tryptophan to provide N-formylkynurenine. This reaction is known as the initial and rate limiting step in the kynurenine pathway of tryptophan catabolism in mammals. The generated N-formyl-kynurenine is further metabolized to bioactive metabolites, including kynurenine, kynurenic acid, 3-hydroxy-kynurenine, and quinolinic acid, which are known to be involved in a number of neurological disorders, such as Alzheimer's disease. Both HeLa human cervical carcinoma cells and primary human DCs can be induced by proinflammatory cytokines to express endogenous IDO1, consequently resulting in kyn generation in the cell culture supernatant. The ability of INCB024360 to inhibit kyn production after treatment of these cells with human recombinant IFN-gamma and/or bacterial LPS was therefore determined. In the HeLa cell-based assay, only IFN-gamma was used as the stimulus because the expression of IDO1 cannot be induced by LPS. INF—r was used to stimulate HeLa cells to express IDOI and we provide the colorimetric method to determine the inhibition level of compounds against IDO.

### Cell Plating

HeLa cells were grown to the desired confluency. Cell dissociation reagent (0.25% trypsin/EDTA) was added to cells and allowed to incubate for several minutes, or until cells have detached. Cells were washed with cell culture medium, centrifuged, and the pellet resuspended in cell culture medium. Cells were then seeded (5000 cells/well, 180 μl/well) on a 96-well plate (Corning 3599) and the plate was incubated at 37° C./5% CO₂.

### Compounds Dosage Gradient Solution Preparation

On Day 2, 10 μL IFN-γ (2000 ng/mL working conc.) was added to the assay plate and incubated for 15 min in the incubator. Compounds were solubilized in 100% DMSO to a concentration of 10 mM and stored at nitrogen cabinet (RT). The compound concentration was reduced if needed to solubilize the compound. Compounds were diluted from stock to working concentration (2 mM) by DMSO. Compounds were serially diluted and added to assay plate and incubated for 48 hours at 37° C./5% CO₂.

### IDO1 Assay

After equilibrating the plates and reagents, 140 μl of the supernatant was transferred to a new plate using multichannel pipette. 10 μl of 6.1N trichloroacetic acid was added into each well, and the plate was mixed using a plate shaker at 300-500 rpm for 30 seconds. The plate was then incubated at 60° C. for 30 minutes. The plates were then centrifuged for 10 minutes at 2000 rpm. 100 μl of the supernatant was then transferred to another 96-well plate (corning-3599). To each well of the plate was added 100 μl of 2% (w/v) 4-dimethylaminobenzaldehyde in acetic acid. Optical density values were then measured by Envision at 480 nm.

### Data Analysis

Inhibition rate of the compound was calculated according to the formula: [% inhibition=100-100×(sample signal−positive control)/(negative control−positive control)]. The IC50 values for each compound was performed in a CDD system. Table 2 provides IDO1 inhibition data obtained from the procedure set forth above.

### Example 154: Cellular IDO2 Protocol

IDO2 inhibition at the cellular level was evaluated in vitro using the following procedure.

### Cell Plating

Cell dissociation medium (PBS, 0.25% trypsin, cell media) was added to cells and allowed to incubate for several minutes, or until cells have detached. Using cell culture medium, the volume of the suspension was adjusted to achieve a cell concentration of 20,000 cells/well (90 uL/well). 90 μL of the cell suspension was added to each well of assay plate (Corning 3599) according to the plate map, and the assay plate was incubated at 37° C./5% CO₂.

### Assay Medium Preparation (IDO2)

5-Methoxy-L-tryptophan powder was dissolved in the culture medium to a concentration of 10 mM, then filtered and sterilized with a 0.22 um filter. Doxycycline powder was dissolved in ddH2O to a concentration of 100 ug/ml, then filtered and sterilized with a 0.22 um filter. 5-aminolevulinic acid powder was dissolved in ddH2O to a concentration of 400 mM, then filtered and sterilized with a 0.22 um filter. A 2× Assay medium was then by diluting the doxycycline (from 100 ug/ml to 2 ug/ml) and 5-Methoxy-L-tryptophan (from 10 mM to 5 mM) in culture medium. The 5-aminolevulinic acid solution was also added to the solution (diluting the concentration from 400 mM to 2 mM) to get a 2× working solution.

Once completed, 100 μL of the 2× working solution was added to each assay plate, and the assay plate was incubated at 37° C./5% CO₂ and before adding the test compound.

### IDO2 Assay

The reference compound INCB024360 (a potent and selective indoleamine 2,3-dioxygenase (IDO1) inhibitor) and test compounds were diluted from source stock solutions, and 20 μL of diluted compound was added to each plate, which contained 180 uL culture medium. Serial dilution was performed to obtain different concentrations of compound according to the plate the plate map, and the assay plate was incubated at 37° C./5% CO₂ for 72 h.

After incubation, plates and reagents were allows to equilibrate to room temperature. From the assay plate, 140 μL of the supernatant was transferred to a new plate using multichannel pipette. Trichloroacetic acid powder was dissolved in ddH2O to prepare 6.1N trichloroacetic acid. 10 μL of 6.1N trichloroacetic acid was added into each well, mix the content using a plate shaker at 300-500 rpm for 30 seconds. The plates were incubated at 55° C. for 40 minutes, and then centrifuged for 10 min at 2500 rpm. Following centrifugation, 100 μL of the supernatant was transferred to another 96 plate, and 100 μL of 2% (w/v) 4-dimethylaminobenzaldehyde in acetic acid was added into each well. Measurement of the signal was performed using Envision at 480 nm. Throughout the foregoing process, care was taken to prevent exposure to light.

### Data Analysis

Inhibition rate of the compound was calculated according to the formula: [% inhibition=100-100×(sample signal−positive control)/(negative control−positive control)]. The IC50 values for each compound was performed in a CDD system. Table 2 provides IDO2 inhibition data obtained from the procedure set forth above.

TABLE 2

| Compound | IDO1 IC50: Average | IDO2 IC50: Average |
|---|---|---|
| Number | IC50 (nM)** | IC50 (nM)** |
| 1 | A | B |
| 2 | A | A |
| 3 | A | A |
| 4 | C | C |
| 5 | C | B |
| 6 | B | B |
| 7 | B | B |
| 8 | B | B |
| 9 | C | B |
| 10 | C | B |
| 11 | B | A |
| 12 | A | A |
| 13 | C | C |
| 14 | B | A |
| 15 | B | B |
| 16 | B | A |
| 17 | B | A |
| 18 | C | B |
| 19 | C | C |
| 20 | B | B |
| 21 | A | A |
| 22 | C | C |
| 23 | B | B |
| 24 | B | B |
| 25 | B | B |
| 26 | C | B |
| 27 | B | A |
| 28 | B | A |
| 29 | C | C |
| 30 | B | A |
| 31 | B | A |
| 32 | B | B |
| 33 | A | A |
| 34 | C | B |
| 35 | B | A |
| 36 | B | A |
| 37 | A | A |
| 38 | A | A |
| 39 | B | A |
| 40 | B | B |
| 41 | B | A |
| 42 | A | A |
| 43 | B | A |
| 44 | B | A |
| 45 | B | B |
| 46 | B | A |
| 47 | C | B |
| 48 | A | A |
| 49 | B | A |
| 50 | C | B |
| 51 | C | B |
| 52 | — | A |
| 53 | — | A |
| 54 | — | A |
| 55 | — | C |
| 56 | — | B |
| 57 | — | A |
| 58 | — | B |
| 59 | — | A |
| 60 | — | A |
| 61 | — | C |
| 62 | — | B |
| 63 | — | A |
| 64 | — | A |
| 65 | — | B |
| 66 | — | A |
| 67 | — | B |
| 68 | — | B |
| 69 | — | A |
| 70 | — | A |
| 71 | — | A |
| 72 | — | A |
| 73 | — | B |
| 74 | — | C |
| 75 | — | B |
| 76 | — | A |
| 77 | — | B |
| 78 | — | B |
| 79 | — | A |
| 80 | — | B |
| 81 | — | A |
| 82 | — | A |
| 83 | — | A |
| 84 | — | A |
| 85 | — | B |
| 86 | — | B |
| 87 | — | A |
| 88 | — | A |
| 89 | — | B |
| 90 | — | B |
| 91 | — | A |
| 92 | — | A |
| 93 | — | B |
| 94 | — | B |
| 95 | — | A |
| 96 | — | A |
| 97 | — | A |
| 98 | — | A |
| 99 | — | A |
| 100 | — | A |
| 101 | — | B |
| 102 | — | A |
| 103 | — | A |
| 104 | — | A |
| 105 | — | A |
| 106 | — | B |
| 107 | — | A |
| 108 | — | B |
| 109 | — | B |
| 110 | — | A |
| 111 | — | B |
| 112 | — | A |
| 113 | — | A |
| 114 | — | B |
| 115 | — | B |
| 116 | — | B |
| 117 | — | A |
| 118 | — | A |
| 119 | — | A |
| 120 | — | C |
| 121 | — | B |
| 122 | — | C |
| 123 | — | B |
| 124 | — | B |
| 125 | — | B |
| 126 | — | B |
| 127 | — | A |
| 128 | — | A |
| 129 | — | A |
| 130 | — | C |
| 131 | — | A |
| 132 | — | A |
| 133 | — | A |
| 134 | — | B |
| 135 | — | A |
| 136 | — | A |
| 137 | — | A |
| 138 | — | B |
| 139 | — | A |
| 140 | — | A |
| 141 | — | B |
| 142 | — | A |
| 143 | — | A |
| 144 | — | A |
| 145 | — | A |
| 146 | — | A |
| 147 | — | B |
| 148 | — | A |
| 149 | — | A |
| 150 | — | A |
| 151 | — | A |
| 152 | — | C |
| 153 | — | C |
| 171 | — | A |

**A: x < 50 nM IC₅₀; B: 50 < x < 500 nM IC₅₀; C: x > 500 nM IC₅₀; —: not determined

1. A compound of Formula (I): 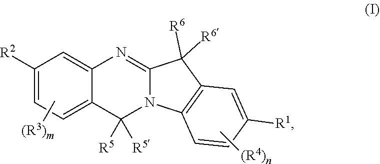
or a pharmaceutically acceptable salt thereof; wherein
R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —NO₂, —N₃, and —CN;
wherein when R¹ is hydroxyl, —O—C₁ alkyl, or —NO₂; then R² is selected from
fluoro, bromo, iodo, —OR²², SR²¹, —N(R²¹)₂, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹), —N(R²¹)P(O)(OR²¹)R²¹, —S(O)₂R²¹, —S(O)₂N(R²¹)₂, —NO₂, and —CN;
C₁₋₆ alkyl, optionally substituted with one or more substituent independently selected from R³⁰; and
C₃₋₆ carbocycle and 3- to 6-membered heterocycle, any of which is optionally substituted with one or more substituent independently selected from R³⁰;
wherein when R¹ is fluoro; then R² is selected from
bromo, OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³)₂, —N(R²³)P(O)(OR²³)R²³, —S(O)₂R²³, —S(O)₂N(R²³)₂, —NO₂, and —CN;
C₁₋₆ alkyl, optionally substituted with one or more substituent independently selected from R³⁰; and
C₃₋₆ carbocycle, 3- to 4-membered heterocycle, 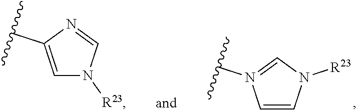
any of which is optionally substituted with one or more substituent independently selected from R³⁰;
wherein when R¹ is C₁ alkyl, —O—C₁ haloalkyl, hydroxy, chloro, or bromo; then R² is selected from:
iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴)₂, —N(R²⁴)P(O)(OR²⁴)R²⁴, —S(O)R²⁴, —S(O)₂R²⁴, —S(O)₂N(R²⁴)₂, —NO₂, and —CN;
C₂₋₆ alkyl, optionally substituted with one or more substituent independently selected from R³⁰;
C₃₋₆ carbocycle and 3- to 5-membered heterocycle, any of which is optionally substituted with one or more substituent independently selected from R³⁰; and
when R¹ is selected from bromo, hydroxy, and C₁ alkyl, R² is further selected from fluoro;
wherein when R¹ is C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)₂R¹¹, —S(O)₂N(R¹¹)₂, —N₃, or —CN;
then R² is selected from
halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), N(R²⁶)SO₂N(R²⁶)₂, —N(R²⁶)P(O)(OR²⁶)R²⁶, —S(O)R²⁶, —S(O)₂R²⁶, —S(O)₂N(R²⁶)₂, —NO₂, and —CN;
C₁₋₆ alkyl, optionally substituted with one or more substituent independently selected from R³⁰; and
C₃₋₆ carbocycle and 3- to 6-membered heterocycle, any of which is optionally substituted with one or more substituent independently selected from R³⁰;
provided when R¹ is —C(O)OR¹¹, then R² cannot be substituted 6-membered heterocycle;
R³ and R⁴ are each independently selected at each occurrence from
halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
C₁₋₄ alkyl, optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN; and
C₃₋₄ carbocycle and 3- to 4-membered heterocycle, any of which is optionally substituted with one or more substituents independently selected from halogen, —OR¹⁴, —SR¹⁴, —N(R¹⁴)₂, —N(R¹⁴)C(O)R¹⁴, —C(O)R¹⁴, —C(O)OR¹⁴, —C(O)N(R¹⁴)₂, —NO₂, and —CN;
R⁵ and R^{5′} are each independently selected from hydrogen and hydroxyl; or R⁵ and R^{5′} taken together are ═O;
R⁶ and R^{6′} are each independently selected from hydrogen and hydroxyl; or R⁶ and R^{6′} taken together are ═O, ═N—OR²⁶, or ═NR²⁶;
m is selected from 0, 1, 2, and 3;
n is selected from 0, 1, 2, and 3;
R¹¹ and R¹⁴ are each independently selected at each occurrence from hydrogen;
C₁₋₆ alkyl optionally substituted with one or more substituent independently selected from R³¹; and
C₃₋₆ carbocycle and 3- to 6-membered heterocycle, any of which is optionally substituted with one or more substituent independently selected from R³¹;
R¹² is selected at each occurrence from
C₁₋₆ alkyl substituted with one or more substituent independently selected from R³²; and
C₃₋₆ carbocycle and 3- to 6-membered heterocycle, any of which is optionally substituted with one or more substituent independently selected from R³³;
R¹³ is selected at each occurrence from hydrogen;
C₁₋₆ alkyl optionally substituted with one or more substituent independently selected from R³⁴; and
C₃₋₅ carbocycle and 3- to 6-membered heterocycle, any of which is optionally substituted with one or more substituent independently selected from R³⁴;
R²¹, R²³, R²⁴, and R²⁶ are each independently selected at each occurrence from hydrogen;
C₁₋₆ alkyl optionally substituted with one or more substituents independently selected from R³⁴; and
C₃₋₆ carbocycle and 3- to 6-membered heterocycle, any of which is optionally substituted with one or more substituent independently selected from C₁₋₄ alkyl, C₁₋₄ haloalkyl, and R³⁴;
R²² and R²⁵ are each independently selected at each occurrence from C₂₋₆ alkyl, C₂₋₆ haloalkyl, C₂₋₆ hydroxyalkyl; and
R³⁰, R³¹, R³³, and R³⁴ are each independently selected at each occurrence from
halogen, —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹)₂, —N(R⁴¹)P(O)(OR⁴¹)R⁴¹, —S(O)R⁴¹, —S(O)₂R⁴¹, —S(O)₂N(R⁴¹)₂, —NO₂, ═O, ═S, —CN, C₃₋₆ carbocycle, and 3- to 7-membered heterocycle;
R³² is independently selected at each occurrence from —OR⁴¹, —SR⁴¹, —N(R⁴¹)₂, —C(O)R⁴¹, —C(O)OR⁴¹, —OC(O)R⁴¹, —OC(O)N(R⁴¹)₂, —C(O)N(R⁴¹)₂, —N(R⁴¹)C(O)R⁴¹, —N(R⁴¹)C(O)OR⁴¹, —N(R⁴¹)C(O)N(R⁴¹)₂, —N(R⁴¹)S(O)₂(R⁴¹), —N(R⁴¹)SO₂N(R⁴¹)₂, —N(R⁴¹)P(O)(OR⁴¹)R⁴¹, —S(O)R⁴¹, —S(O)₂R⁴¹, —S(O)₂N(R⁴¹)₂, —NO₂, ═O, ═S, and —CN; and
R⁴¹ is independently selected at each occurrence from hydrogen, C₁₋₆ alkyl, C₁₋₆ haloalkyl, and C₁₋₆ hydroxyalkyl.

2. The compound or salt of claim 1, wherein R¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —OR¹², —N(R¹³)₂, —C(O)OR¹¹, —C(O)N(R¹¹)₂, —N(R¹¹)C(O)R¹¹, —N(R¹¹)S(O)₂(R¹¹), —S(O)₂R¹¹, —S(O)₂N(R¹¹)₂, —NO₂, —N₃, and —CN.

3. The compound or salt of claim 2, wherein R¹ is selected from fluoro, —C₁₋₆ haloalkyl, —O—C₁₋₆ haloalkyl, —N(R¹³)₂, —C(O)OR¹¹, —C(O)N(R¹¹)₂, —N(R¹¹)C(O)R¹¹, —S(O)₂R¹¹, —NO₂, —N₃, and —CN.

4. The compound or salt of claim 1, wherein R¹ is hydroxyl, —O—C₁ alkyl, or —NO₂; and R² is selected from fluoro, bromo, iodo, —OR²², SR²¹, —N(R²¹)₂, —OC(O)R²¹, —OC(O)N(R²¹)₂, —N(R²¹)C(O)R²¹, —N(R²¹)C(O)OR²¹, —N(R²¹)C(O)N(R²¹)₂, —N(R²¹)S(O)₂(R²¹), —N(R²¹)SO₂N(R²¹)₂, —N(R²¹)P(O)(OR²¹)R²¹, —S(O)₂R²¹, —S(O)₂N(R²¹)₂, —NO₂, —CN, and optionally substituted C₁₋₆ alkyl.

5. (canceled)

6. (canceled)

7. The compound or salt of claim 1, wherein R¹ is fluoro; and R² is selected from bromo, OR²³, —N(H)R²³, —C(O)R²³, —C(O)N(R²³)₂, —C(O)OR²³, —OC(O)R²³, —OC(O)N(R²³)₂, —N(R²³)C(O)R²³, —N(R²³)C(O)OR²³, —N(R²³)C(O)N(R²³)₂, —N(R²³)S(O)₂R²³, —N(R²³)SO₂N(R²³)₂, —N(R²³)P(O)(OR²³)R²³, —S(O)₂R²³, —S(O)₂N(R²³)₂, —NO₂, —CN, 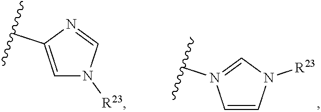
and optionally substituted C₁₋₆ alkyl.

8. (canceled)

9. The compound or salt of claim 1, wherein R¹ is C₁ alkyl, —O—C₁ haloalkyl, hydroxyl, chloro, or bromo; and R² is selected from iodo, —OR²⁵, —C(O)R²⁴, —C(O)OR²⁵, —OC(O)R²⁴, —OC(O)N(R²⁴)₂, —C(O)N(R²⁴)₂, —N(R²⁴)C(O)R²⁴, —N(R²⁴)C(O)OR²⁴, —N(R²⁴)C(O)N(R²⁴)₂, —N(R²⁴)S(O)₂(R²⁴), N(R²⁴)SO₂N(R²⁴)₂, —N(R²⁴)P(O)(OR²⁴)R²⁴, —S(O)R²⁴, —S(O)₂R²⁴, —S(O)₂N(R²⁴)₂, —NO₂, —CN, and optionally substituted C₂₋₆ alkyl.

10. (canceled)

11. The compound or salt of claim 1, wherein
R¹ is selected from C₂₋₆ alkyl, C₁₋₆ haloalkyl, —O—C₂₋₆ alkyl, —O—C₂₋₆ haloalkyl, —OR¹², —SR¹¹, —N(R¹³)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹¹, —N(R¹¹)C(O)OR¹¹¹, —N(R¹¹)C(O)N(R¹¹)₂, —N(R¹¹)S(O)₂(R¹¹), —S(O)₂R¹¹, —S(O)₂N(R¹¹)₂, —N₃, and —CN; and
R² is selected from halogen, —OR²⁶, —SR²⁶, —N(R²⁶)₂, —C(O)R²⁶, —C(O)OR²⁶, —OC(O)R²⁶, —OC(O)N(R²⁶)₂, —C(O)N(R²⁶)₂, —N(R²⁶)C(O)R²⁶, —N(R²⁶)C(O)OR²⁶, —N(R²⁶)C(O)N(R²⁶)₂, —N(R²⁶)S(O)₂(R²⁶), —N(R²⁶)SO₂N(R²⁶)₂, —N(R²⁶)P(O)(OR²⁶)R²⁶, —S(O)R²⁶, —S(O)₂R²⁶, —S(O)₂N(R²⁶)₂, —NO₂, —CN, and optionally substituted C₁₋₆ alkyl.

12. (canceled)

13. The compound or salt of claim 12, wherein R¹ is selected from —N(R¹³)₂, —C(O)OR¹¹, —C(O)N(R¹¹)₂, —N(R¹¹)C(O)R¹¹, —S(O)₂R¹¹, —N₃, and —CN.

14. (canceled)

15. The compound or salt of claim 11, wherein R² is selected from halogen, —OR²⁶, —SR²⁶, —N(R²⁶)₂, —C(O)R²⁶, —C(O)OR²⁶, —C(O)N(R²⁶)₂, —N(R²⁶)C(O)R²⁶, —N(R²⁶)C(O)OR²⁶, —N(R²⁶)S(O)₂(R²⁶), —S(O)₂R²⁶, —S(O)₂N(R²⁶)₂, —NO₂, —CN, and optionally substituted C₁₋₆ alkyl.

16. The compound or salt of claim 1, wherein R³ and R⁴ are each independently selected at each occurrence from halogen, —OR¹⁴, —N(R¹⁴)₂, —N(R¹⁴)C(O)R¹⁴, —C(O)OR¹⁴, —C(O)N(R¹⁴)₂, —NO₂, and —CN, and optionally substituted C₁₋₄ alkyl;
and R⁵ and R^{5′} taken together are ═O; and R⁶ and R^{6′} taken together are ═O.

17. (canceled)

18. (canceled)

19. (canceled)

20. The compound or salt of claim 1, wherein m is 0 or 1; and n is 0 or 1.

21. The compound or salt of claim 1, wherein
R¹¹ and R¹⁴ are each independently selected at each occurrence from hydrogen and optionally substituted C₁₋₆ alkyl;
R¹² is independently selected at each occurrence from optionally substituted C₁₋₆ alkyl; and
R¹³ is independently selected at each occurrence from hydrogen and optionally substituted C₁₋₆ alkyl.

22. The compound or salt of claim 1, wherein R²¹, R²³, R²⁴, and R²⁶ are each independently selected at each occurrence from hydrogen, optionally substituted C₁₋₆ alkyl, and optionally substituted 3- to 6-membered heterocycle.

23. The compound or salt of claim 1, wherein
R²² and R²⁵ are each independently selected at each occurrence from C₃₋₆ alkyl and C₃₋₆ haloalkyl; and
R³⁰, R³¹, R³³, and R³⁴ are each independently selected at each occurrence from halogen, —N(R⁴¹)₂, —N(R⁴¹)S(O)₂(R⁴¹), —N(R⁴¹)SO₂N(R⁴¹)₂, —N(R⁴¹)P(O)(OR⁴¹)R⁴¹, —S(O)₂R⁴¹, and ═O; and
R³² is independently selected at each occurrence from halogen, —N(R⁴¹)₂, —N(R⁴¹)S(O)₂(R⁴¹), —N(R⁴¹)SO₂N(R⁴¹)₂, —N(R⁴¹)P(O)(OR⁴¹)R⁴¹, —S(O)₂R⁴¹, and ═O; and
R⁴¹ is independently selected at each occurrence from hydrogen, and C₁₋₆ alkyl.

24. (canceled)

25. (canceled)

26. (canceled)

27. (canceled)

28. The compound or salt of claim 1, wherein the compound or salt of Formula (I) is selected from: 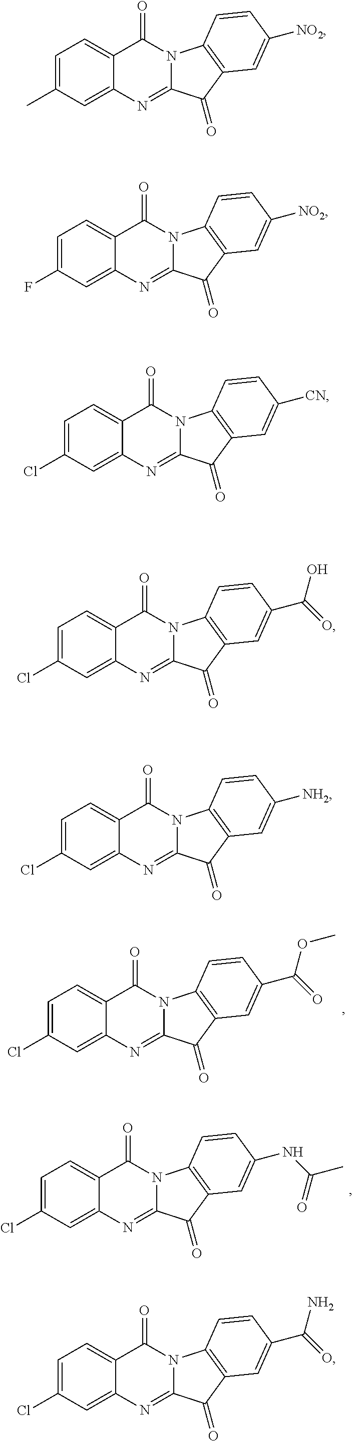 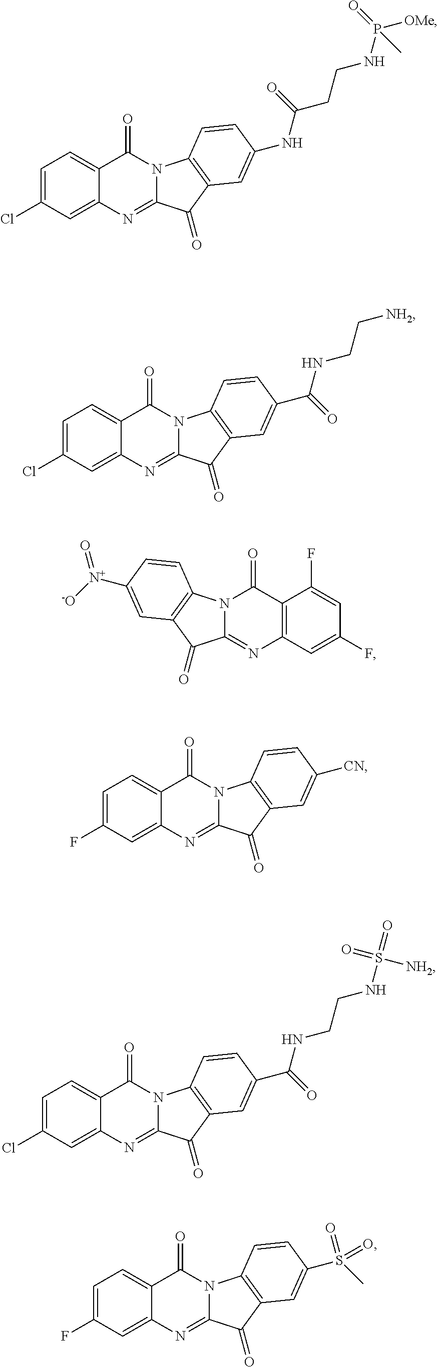 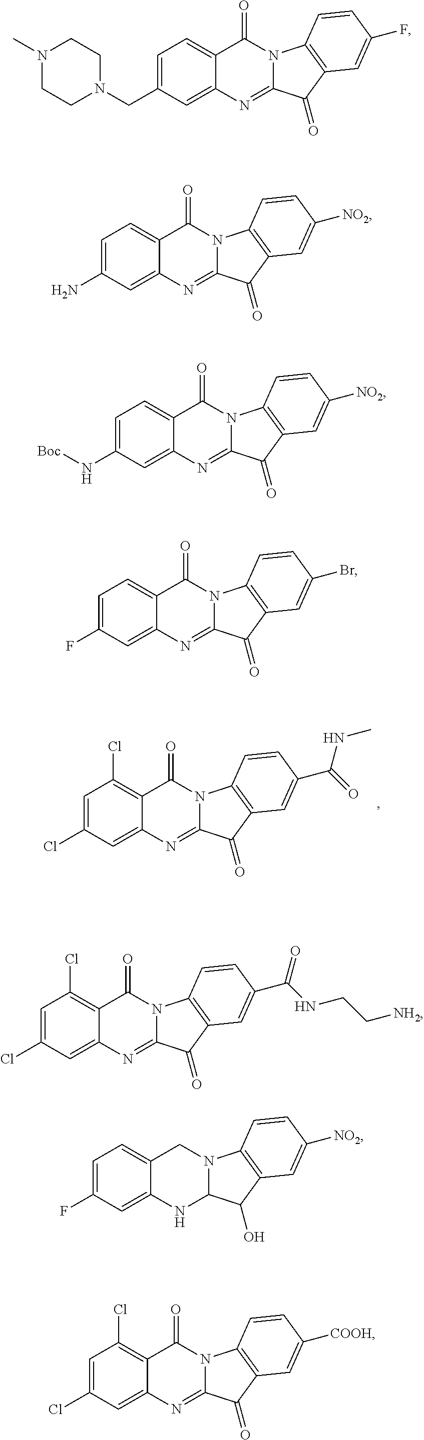 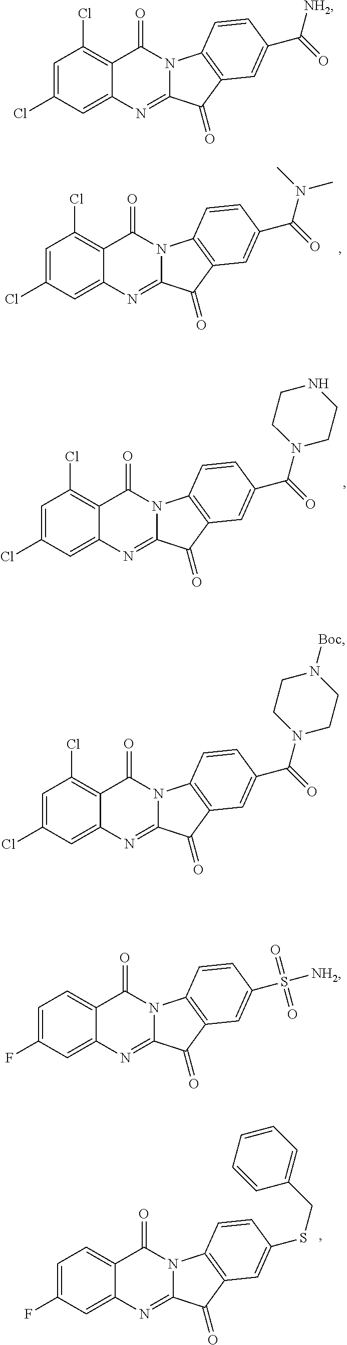 [SMILES: NC(=O)c1ccc2c(c1)C(=O)c1nc3cc(Cl)cc(Cl)c3c(=O)n1-2 ; CN(C)C(=O)c1ccc2c(c1)C(=O)c1nc3cc(Cl)cc(Cl)c3c(=O)n1-2 ; O=C1c2cc(C(=O)N3CCNCC3)ccc2-n2c1nc1cc(Cl)cc(Cl)c1c2=O ; CC(C)(C)OC(=O)N1CCN(C(=O)c2ccc3c(c2)C(=O)c2nc4cc(Cl)cc(Cl)c4c(=O)n2-3)CC1 ; NS(=O)(=O)c1ccc2c(c1)C(=O)c1nc3cc(F)ccc3c(=O)n1-2 ; O=C1c2cc(SCc3ccccc3)ccc2-n2c1nc1cc(F)ccc1c2=O] 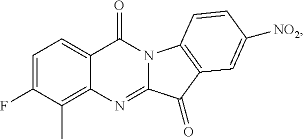 [SMILES: Cc1c(F)ccc2c(=O)n3c(nc12)C(=O)c1cc([N+](=O)[O-])ccc1-3] 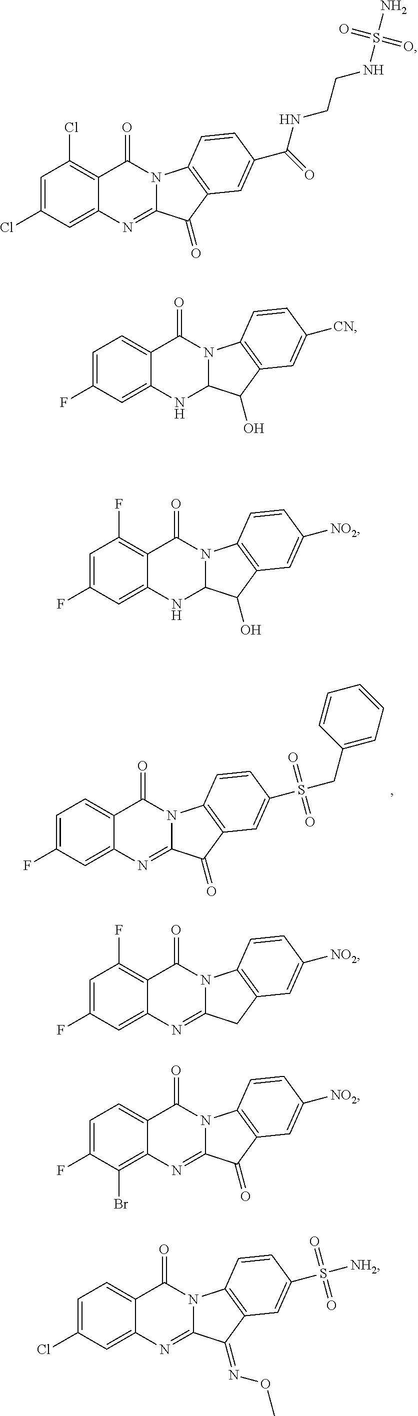 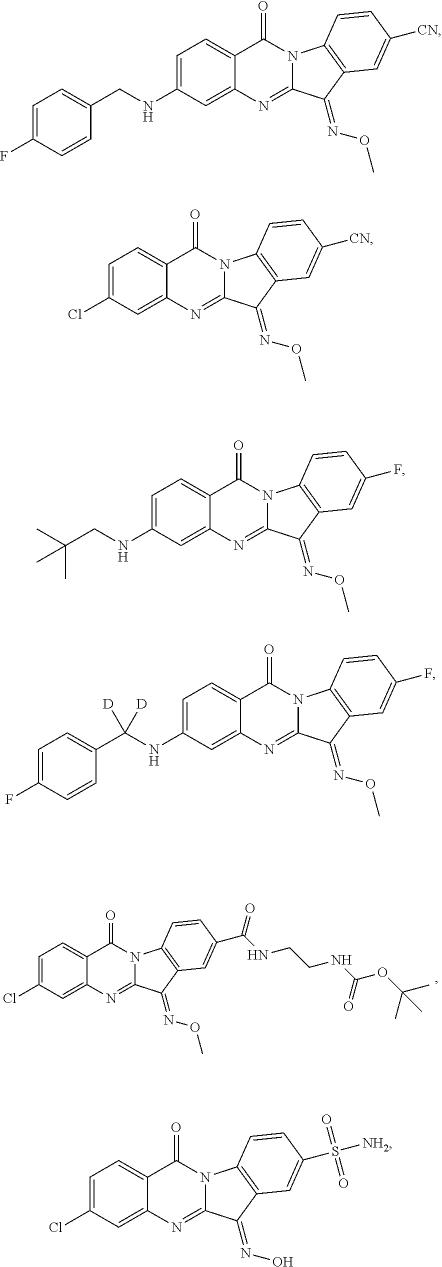 [SMILES: CO/N=C1\c2cc(C#N)ccc2-n2c1nc1cc(NCc3ccc(F)cc3)ccc1c2=O ; CO/N=C1\c2cc(C#N)ccc2-n2c1nc1cc(Cl)ccc1c2=O ; CO/N=C1\c2cc(F)ccc2-n2c1nc1cc(NCC(C)(C)C)ccc1c2=O ; [2H]C([2H])(Nc1ccc2c(=O)n3c(nc2c1)/C(=N/OC)c1cc(F)ccc1-3)c1ccc(F)cc1 ; CO/N=C1\c2cc(C(=O)NCCNC(=O)OC(C)(C)C)ccc2-n2c1nc1cc(Cl)ccc1c2=O ; NS(=O)(=O)c1ccc2c(c1)/C(=N\O)c1nc3cc(Cl)ccc3c(=O)n1-2] 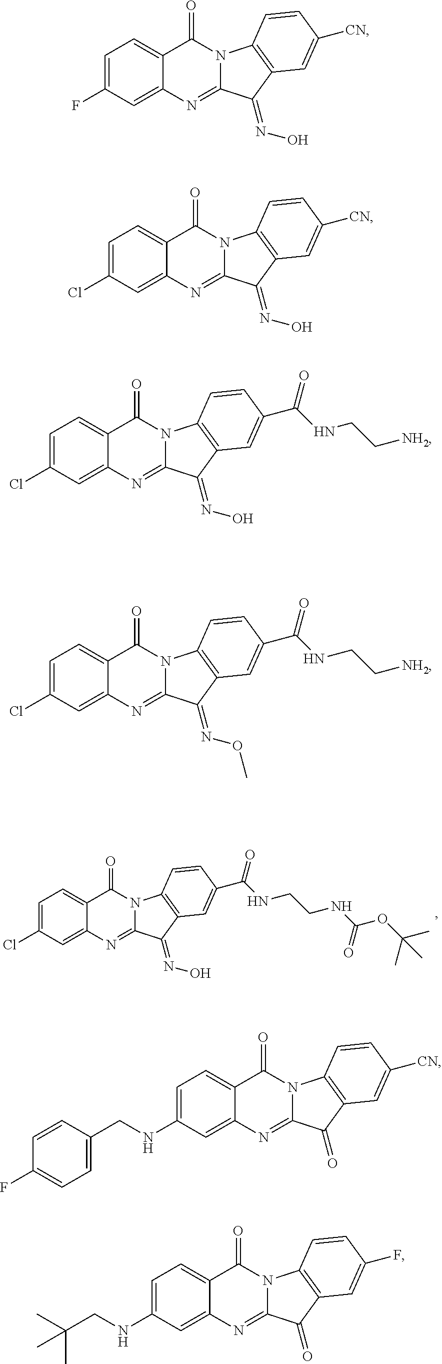 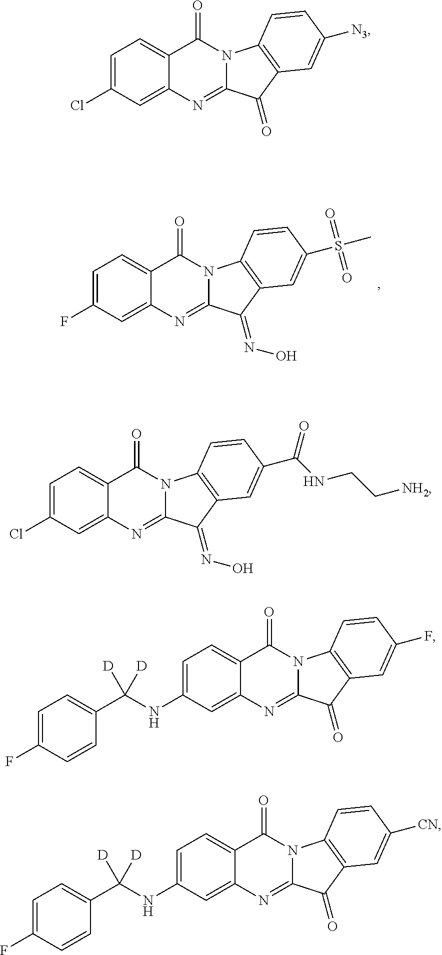 [SMILES: [N-]=[N+]=Nc1ccc2c(c1)C(=O)c1nc3cc(Cl)ccc3c(=O)n1-2 ; CS(=O)(=O)c1ccc2c(c1)/C(=N\O)c1nc3cc(F)ccc3c(=O)n1-2 ; NCCNC(=O)c1ccc2c(c1)/C(=N\O)c1nc3cc(Cl)ccc3c(=O)n1-2 ; [2H]C([2H])(Nc1ccc2c(=O)n3c(nc2c1)C(=O)c1cc(F)ccc1-3)c1ccc(F)cc1 ; [2H]C([2H])(Nc1ccc2c(=O)n3c(nc2c1)C(=O)c1cc(C#N)ccc1-3)c1ccc(F)cc1] 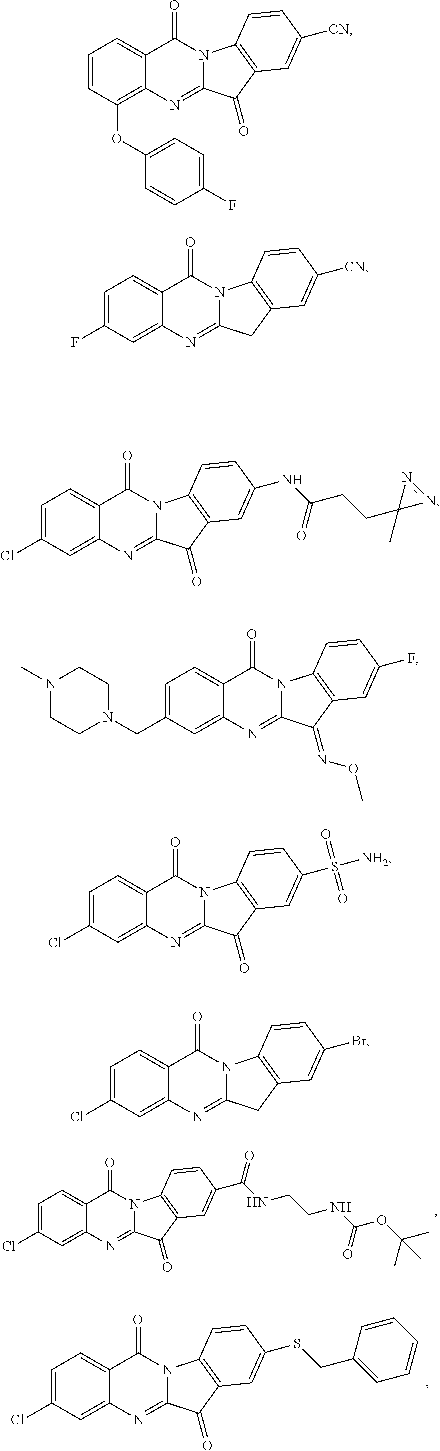 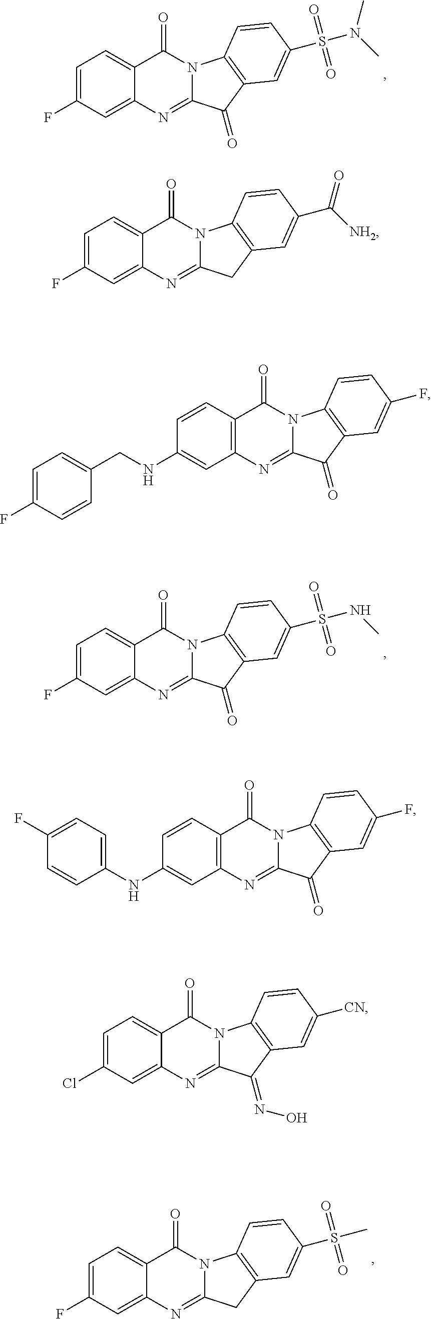 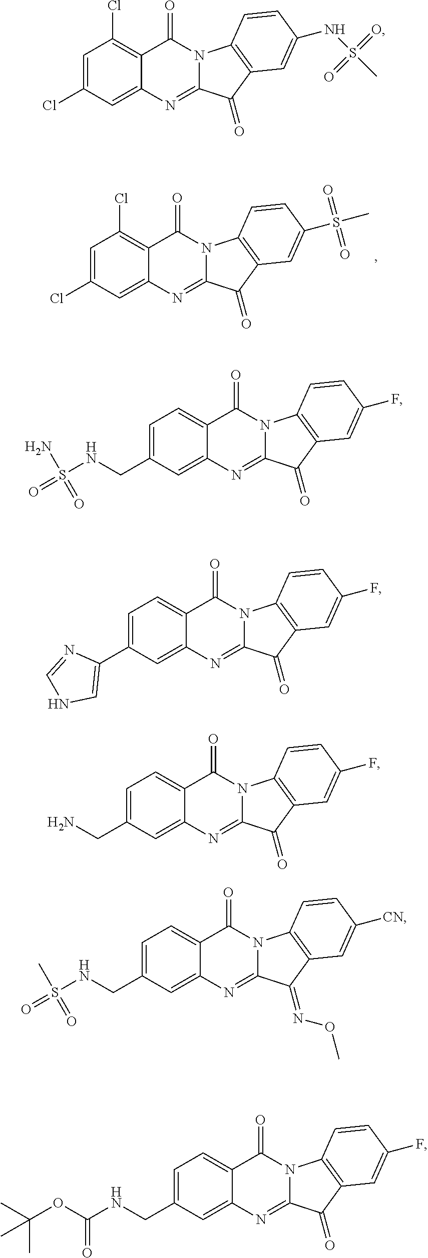 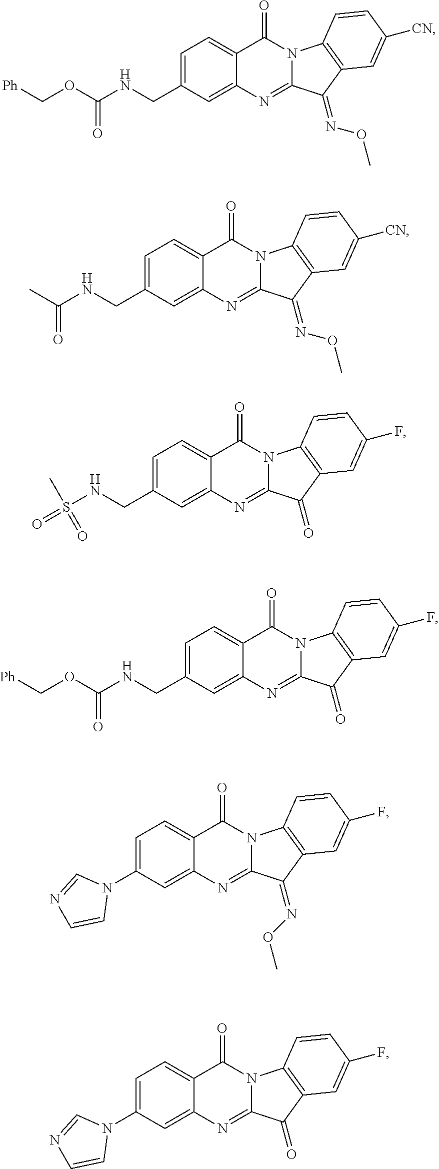 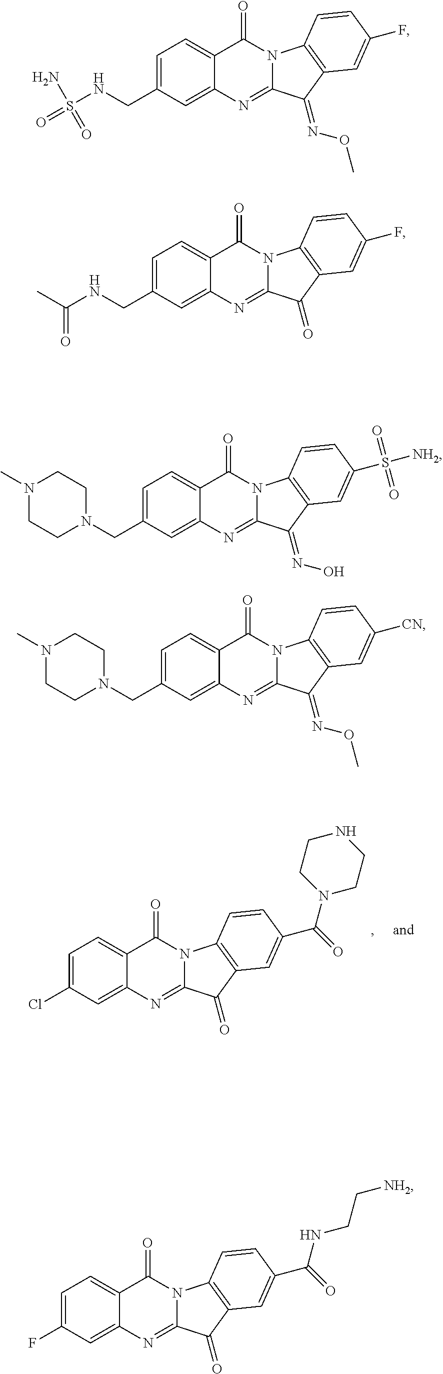
or a pharmaceutically acceptable salt thereof.

29. A compound of Formula (II): 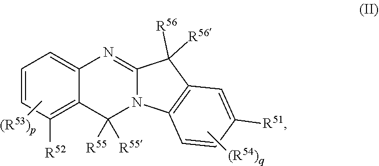
or a pharmaceutically acceptable salt thereof; wherein
R⁵¹ is selected from fluoro, chloro, bromo, iodo, hydroxyl, —C₁₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, —NO₂, and —CN;
when R⁵¹ is fluoro, chloro, or iodo, R⁵² is selected from
bromo, iodo, —N(R⁷¹)₂, —OR⁷¹, —SR⁷¹, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), N(R⁷¹)SO₂N(R⁷¹)₂, —N(R⁷¹)P(O)(OR⁷¹)R⁷¹, —S(O)R⁷¹, —S(O)₂R⁷¹, —S(O)₂N(R⁷¹)₂, —NO₂, and —CN;
C₁ alkyl substituted with one or more substituent independently selected from R⁸⁰, and C₂₋₆ alkyl optionally substituted with one or more substituent independently selected from R⁸⁰; and
C₃₋₆ carbocycle and 3- to 5-membered heterocycle, any of which is optionally substituted with one or more substituent independently selected from R⁸⁰;
when R⁵¹ is C₁ alkyl or —NO₂, R⁵² is selected from
fluoro, iodo, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —N(R⁷²)SO₂N(R⁷²)₂, —N(R⁷²)P(O)(OR⁷²)R⁷², —S(O)R⁷², —S(O)₂R⁷², —S(O)₂N(R⁷²)₂, —NO₂, and —CN;
C₁₋₆ alkyl, optionally substituted with one or more substituent independently selected from R⁸⁰; and
C₃₋₆ carbocycle and 3- to 6-membered heterocycle, any of which is optionally substituted with one or more substituent independently selected from R⁸⁰;
when R⁵¹ is selected from bromo, hydroxyl, —C₂₋₆ alkyl, —C₁₋₆ haloalkyl, —N(R⁶¹)₂, —OR⁶¹, —SR⁶¹, —C(O)R⁶¹, —C(O)OR⁶¹, —OC(O)R⁶¹, —OC(O)N(R⁶¹)₂, —C(O)N(R⁶¹)₂, —N(R⁶¹)C(O)R⁶¹, —N(R⁶¹)C(O)OR⁶¹, —N(R⁶¹)C(O)N(R⁶¹)₂, —N(R⁶¹)S(O)₂(R⁶¹), —S(O)₂R⁶¹, —S(O)₂N(R⁶¹)₂, and —CN, R⁵² is selected from
halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —N(R⁷³)SO₂N(R⁷³)₂, —N(R⁷³)P(O)(OR⁷³)R⁷³, —S(O)R⁷³, —S(O)₂R⁷³, —S(O)₂N(R⁷³)₂, —NO₂, and —CN;
C₁₋₆ alkyl, optionally substituted with one or more substituent independently selected from R⁸⁰; and
C₃₋₆ carbocycle and 3- to 6-membered heterocycle, any of which is optionally substituted with one or more substituent independently selected from R⁸⁰;
R⁵³ and R⁵⁴ are each independently selected at each occurrence from
halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
C₁₋₄ alkyl, optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN; and
C₃₋₄ carbocycle and 3- to 4-membered heterocycle, any of which is optionally substituted with one or more substituents independently selected from halogen, —OR⁶⁴, —SR⁶⁴, —N(R⁶⁴)₂, —N(R⁶⁴)C(O)R⁶⁴, —C(O)R⁶⁴, —C(O)OR⁶⁴, —C(O)N(R⁶⁴)₂, —NO₂, and —CN;
R⁵⁵ and R^{55′} are each independently selected from hydrogen and hydroxyl; or R⁵⁵ and R^{55′} taken together are ═O;
R⁵⁶ and R^{56′} are each independently selected from hydrogen and hydroxyl; or R⁵⁶ and R^{56′} taken together are ═O, ═N—OR⁶⁴, or ═NR²⁶;
p is selected from 0, 1, 2, and 3;
q is selected from 0, 1, 2, and 3;
R⁶¹ and R⁶⁴ are each independently selected at each occurrence from hydrogen;
C₁₋₆ alkyl optionally substituted with one or more substituent independently selected from R⁸¹; and
C₃₋₆ carbocycle and 3- to 6-membered heterocycle, any of which is optionally substituted with one or more substituent independently selected from R⁸¹;
R⁷¹, R⁷², and R⁷³ are each independently selected at each occurrence from hydrogen;
C₁₋₆ alkyl optionally substituted with one or more substituents independently selected from R⁸²; and
C₃₋₆ carbocycle and 3- to 6-membered heterocycle, any of which is optionally substituted with one or more substituent independently selected from C₁₋₄ alkyl, C₁₋₄ haloalkyl, and R⁸²;
R⁸⁰, R⁸¹, and R⁸² are each independently selected at each occurrence from halogen, —OR⁹¹, —SR⁹¹, —N(R⁹¹)₂, —C(O)R⁹¹, —C(O)OR⁹¹, —OC(O)R⁹¹, —OC(O)N(R⁹¹)₂, —C(O)N(R⁹¹)₂, —N(R⁹¹)C(O)R⁹¹, —N(R⁹¹)C(O)OR⁹¹, —N(R⁹¹)C(O)N(R⁹¹)₂, —N(R⁹¹)S(O)₂(R⁹¹), —N(R⁹¹)SO₂N(R⁹¹)₂, —N(R⁹¹)P(O)(OR⁹¹)R⁹¹, —S(O)R⁹¹, —S(O)₂R⁹¹, —S(O)₂N(R⁹¹)₂, —NO₂, ═O, ═S, —CN, C₃₋₆ carbocycle, and 3- to 7-membered heterocycle; and
R⁹¹ is independently selected at each occurrence from hydrogen, C₁₋₆ alkyl, C₁₋₆ haloalkyl, and C₁₋₆ hydroxyalkyl.

30-51. (canceled)

52. A compound of Formula (III): 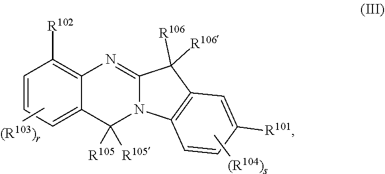
or a pharmaceutically acceptable salt thereof; wherein
R¹⁰¹ is selected from fluoro, chloro, bromo, hydroxyl, —C₁₋₆ alkyl, —C₁₋₆ haloalkyl, —O—C₁₋₆ alkyl, —O—C₁₋₆ haloalkyl, —N(R¹¹¹)₂, —OR¹¹², —SR¹¹¹, —C(O)R¹¹¹, —C(O)OR¹¹¹, —OC(O)R¹¹¹, —OC(O)N(R¹¹¹)₂, —C(O)N(R¹¹¹)₂, —N(R¹¹)C(O)R¹¹¹, —N(R¹¹¹)C(O)OR¹¹¹, —N(R¹¹¹)C(O)N(R¹¹)₂, —N(R¹¹¹)S(O)₂(R¹¹¹), —S(O)₂R¹¹¹, —S(O)₂N(R¹¹¹)₂, —NO₂, and —CN;
when R¹⁰¹ is fluoro, R¹⁰² is selected from
chloro, bromo, iodo, —N(R¹²¹)₂, —OR¹²², —SR¹²¹, —C(O)R¹²¹, —C(O)OR¹²¹, —OC(O)R¹²¹, —OC(O)N(R¹²¹)₂, —C(O)N(R¹²¹)₂, —N(R¹²¹)C(O)R¹²¹, —N(R²¹)C(O)OR¹²¹, —N(R¹²¹)C(O)N(R¹²¹)₂, —N(R¹²¹)S(O)₂(R¹²¹), —N(R¹²¹)SO₂N(R¹²¹)₂, —N(R¹²¹)P(O)(OR¹²¹)R¹²¹, —S(O)R¹²¹, —S(O)₂R¹²¹, —S(O)₂N(R¹²¹)₂, —NO₂, and —CN;
C₁ alkyl substituted with one or more substituent independently selected from R¹²³, and C₂₋₆ alkyl optionally substituted with one or more substituent independently selected from R¹³⁰; and
C₃₋₆ carbocycle and 3- to 6-membered heterocycle, any of which is optionally substituted with one or more substituent independently selected from R¹³⁰;
when R¹⁰¹ is bromo, iodo, hydroxyl, —O—C₁ alkyl, —C(O)OR¹¹¹, —C(O)N(H)(C₂ alkyl), —S(O)₂R¹¹¹, or —S(O)₂N(R¹¹¹)₂, R¹⁰² is selected from
fluoro, —N(R¹²⁴)₂, —OR¹²⁵, —SR¹²⁴, —C(O)R¹²⁴, —C(O)OR¹²⁴, —OC(O)R¹²⁴, —OC(O)N(R¹²⁴)₂, —C(O)N(R¹²⁴)₂, —N(R¹²⁴)C(O)R¹²⁴, —N(R¹²⁴)C(O)OR¹²⁴, —N(R¹²⁴)C(O)N(R¹²⁴)₂, —N(R¹²⁴)S(O)₂(R¹²⁴), —N(R¹²⁴)SO₂N(R¹²⁴)₂, —N(R¹²⁴)P(O)(OR¹²⁴)R¹²⁴, —S(O)R¹²⁴, —S(O)₂R¹²⁴, —S(O)₂N(R¹²⁴)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰;
when R¹⁰¹ is chloro, —C₁₋₆ alkyl, —C₁₋₆ haloalkyl, —O—C₂₋₆ alkyl, —O—C₁₋₆ haloalkyl, —OR¹¹², —SR¹¹¹, —N(R¹¹¹)₂, —C(O)R¹¹¹, —OC(O)R¹¹¹, —C(O)N(R¹¹³)₂, —OC(O)N(R¹¹¹)₂, —N(R¹¹¹)C(O)R¹¹¹, —N(R¹¹¹)C(O)OR¹¹¹, —N(R¹¹¹)C(O)N(R¹¹¹)₂, —N(R¹¹¹)S(O)₂(R¹¹¹), —NO₂, or —CN, R¹⁰² is selected from
halogen, —OR¹²⁶, —SR¹²⁶, —N(R¹²⁶)₂, —C(O)R¹²⁶, —C(O)OR¹²⁶, —OC(O)R¹²⁶, —OC(O)N(R¹²⁶)₂, —C(O)N(R¹²⁶)₂, —N(R¹²⁶)C(O)R¹²⁶, —N(R¹²⁶)C(O)OR¹²⁶, —N(R¹²⁶)C(O)N(R¹²⁶)₂, —N(R¹²⁶)S(O)₂(R¹²⁶), —N(R¹²⁶)SO₂N(R¹²⁶)₂, —N(R¹²⁶)P(O)(OR¹²⁶)R¹²⁶, —S(O)R¹²⁶, —S(O)₂R¹²⁶, —S(O)₂N(R¹²⁶)₂, —NO₂, and —CN;
C₁₋₆ alkyl, optionally substituted with one or more substituent independently selected from R¹³⁰; and
C₃₋₆ carbocycle and 3- to 6-membered heterocycle, any of which is optionally substituted with one or more substituent independently selected from R¹³⁰;
provided that (i) when R¹⁰¹ is NO₂, R¹⁰² cannot be methyl; (ii) when R¹⁰¹ is methyl, R¹⁰² cannot be chloro; and (iii) when R¹⁰¹ is chloro, R¹⁰² cannot be bromo;
R¹⁰³ and R¹⁰⁴ are each independently selected at each occurrence from
halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
C₁₋₄ alkyl, optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN; and
C₃₋₄ carbocycle and 3- to 4-membered heterocycle, any of which is optionally substituted with one or more substituents independently selected from halogen, —OR¹¹⁴, —SR¹¹⁴, —N(R¹¹⁴)₂, —N(R¹¹⁴)C(O)R¹¹⁴, —C(O)R¹¹⁴, —C(O)OR¹¹⁴, —C(O)N(R¹¹⁴)₂, —NO₂, and —CN;
R¹⁰⁵ and R^{105′} are each independently selected from hydrogen and hydroxyl; or R¹⁰⁵ and R^{105′} taken together are ═O;
R¹⁰⁶ and R^{106′} are each independently selected from hydrogen and hydroxyl; or R¹⁰⁶ and R^{106′} taken together are ═O, ═N—OR¹¹⁴, or ═NR¹¹⁴;
r is selected from 0, 1, 2, and 3;
s is selected from 0, 1, 2, and 3;
R¹¹¹ and R¹¹⁴ are each independently selected at each occurrence from hydrogen;
C₁₋₆ alkyl optionally substituted with one or more substituent independently selected from R¹³¹; and
C₃₋₆ carbocycle and 3- to 6-membered heterocycle, any of which is optionally substituted with one or more substituent independently selected from R¹³¹;
R¹¹² is selected at each occurrence from
C₁₋₆ alkyl substituted with one or more substituent independently selected from R¹³²; and
C₃₋₆ carbocycle and 3- to 6-membered heterocycle, any of which is optionally substituted with one or more substituent independently selected from R¹³³;
R¹¹³ is selected at each occurrence from
hydrogen;
C₁ alkyl and C₃₋₆ alkyl optionally substituted with one or more substituent independently selected from R¹³⁴, and C₂ alkyl substituted with one or more substituent independently selected from R¹³⁴; and
C₃₋₅ carbocycle and 3- to 6-membered heterocycle, any of which is optionally substituted with one or more substituent independently selected from R¹³⁴;
R¹²¹, R¹²⁴, and R¹²⁶ are each independently selected at each occurrence from
hydrogen;
C₁₋₆ alkyl optionally substituted with one or more substituents independently selected from R¹³⁴; and
C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²² and R¹²⁵ are each independently selected at each occurrence from
C₂₋₆ alkyl optionally substituted with one or more substituents independently selected from R¹³⁴; and
C₃₋₆ carbocycle and 3- to 6-membered heterocycle, any of which is optionally substituted with one or more substituent independently selected from C₁₋₄ alkyl, C₁₋₄ haloalkyl, and R¹³⁴;
R¹²³ is independently selected at each occurrence from
hydrogen;
C₁₋₆ alkyl optionally substituted with one or more substituents independently selected from R¹³⁴; and
C₃₋₅ carbocycle and 3- to 6-membered heterocycle, any of which is optionally substituted with one or more substituent independently selected from C₁₋₄ alkyl, C₁₋₄ haloalkyl, and R¹³⁴;
R¹³⁰, R¹³¹, R¹³², R¹³³, and R¹³⁴ are each independently selected at each occurrence from halogen, —OR¹⁴¹, —SR¹⁴¹, —N(R¹⁴¹)₂, —C(O)R¹⁴¹, —C(O)OR¹⁴¹, —OC(O)R¹⁴¹, —OC(O)N(R¹⁴¹)₂, —C(O)N(R¹⁴¹)₂, —N(R¹⁴¹)C(O)R¹⁴¹, —N(R¹⁴¹)C(O)OR¹⁴¹, —N(R¹⁴¹)C(O)N(R¹⁴¹)₂, —N(R¹⁴¹)S(O)₂(R¹⁴¹), —N(R¹⁴¹)SO₂N(R¹⁴¹)₂, —N(R¹⁴¹)P(O)(OR¹⁴¹)R¹⁴¹, —S(O)R¹⁴¹, —S(O)₂R¹⁴¹, —S(O)₂N(R¹⁴¹)₂, —NO₂, ═O, ═S, —CN, C₃₋₆ carbocycle, and 3- to 7-membered heterocycle; and
R¹⁴¹ is independently selected at each occurrence from hydrogen, C₁₋₆ alkyl, C₁₋₆ haloalkyl, and C₁₋₆ hydroxyalkyl.

53-68. (canceled)

69. A compound of Formula (IV): 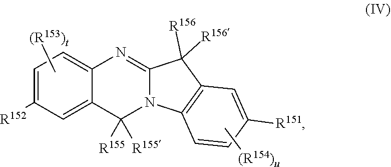
or a pharmaceutically acceptable salt thereof; wherein
R¹⁵¹ is selected from fluoro, bromo, —N(R¹⁶¹)₂, —C(O)R¹⁶¹, —C(O)OR¹⁶¹, —OC(O)R¹⁶¹, —OC(O)N(R¹⁶¹)₂, —C(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), —N(R¹⁶¹)S(O)₂N(R¹⁶¹)₂, —SR¹⁶¹, —S(O)R¹⁶¹, —S(O)₂R¹⁶¹, —S(O)₂N(R¹⁶¹)₂, —CN, —NO₂, —C₁₋₆ haloalkyl, —O—C₂₋₆ alkyl, —O—C₁₋₆ haloalkyl, —OR₃₋₆ carbocycle, and —OR₃₋₆ heterocycle; and 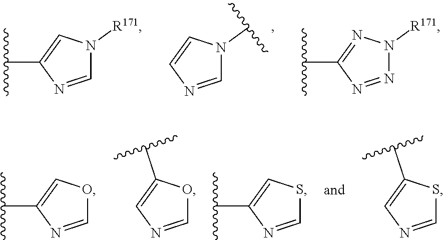
each of which is optionally substituted with one or more substituent independently selected from R¹⁷¹;
when R¹⁵¹ is fluoro, R¹⁵² is selected from
iodo, —NHR¹⁹², —OR¹⁷¹, —SR¹⁷¹, —C(═NR¹⁹⁶)N(R¹⁹⁶)₂, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁹³)C(O)R¹⁷¹, —N(H)C(O)R¹⁹⁴, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, —S(O)₂N(R¹⁷¹)₂, —NR¹⁹⁶(C═NR¹⁹⁶)N(R¹⁹⁶)₂, —N₃, and —CN;
C₁ alkyl substituted with one or more substituent independently selected from R¹⁹⁵, and C₂₋₆ alkyl optionally substituted with one or more substituent independently selected from R¹⁸⁰; and
C₃₋₆ carbocycle, 3-membered heterocycle, 4-membered heterocycle, 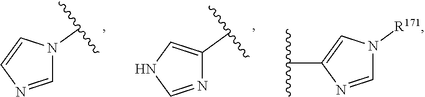
and 6-membered heterocycle, any of which is optionally substituted with one or more substituent independently selected from R¹⁸⁰;
when R¹⁵¹ is 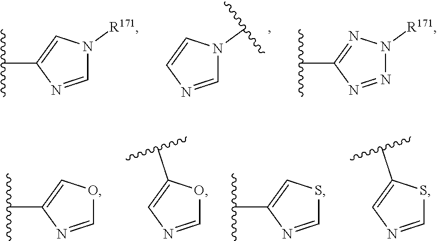
—SR¹⁶¹, —S(O)R¹⁶¹, —S(O)₂R¹⁶¹, —S(O)₂N(R¹⁶¹)₂, or —CN, R¹⁵² is selected from
halogen, —N(R¹⁷⁴)₂, —OR¹⁷⁴, —SR¹⁷⁴, —C(O)R¹⁷⁴, —C(O)OR¹⁷⁴, —OC(O)R¹⁷⁴, —OC(O)N(R¹⁷⁴)₂, —C(O)N(R¹⁷⁴)₂, —N(R¹⁷⁴)C(O)R¹⁷⁴, —N(R¹⁷⁴)C(O)OR¹⁷⁴, —N(R¹⁷⁴)C(O)N(R¹⁷⁴)₂, —N(R¹⁷⁴)S(O)₂(R¹⁷⁴), —N(R¹²⁴)SO₂N(R¹⁷⁴)₂, —N(R¹⁷⁴)P(O)(OR¹⁷⁴)R¹⁷⁴, —S(O)R¹⁷⁴, —S(O)₂R¹⁷⁴, —S(O)₂N(R¹⁷⁴)₂, —NO₂, and —CN;
C₁₋₆ alkyl, optionally substituted with one or more substituent independently selected from R¹⁸⁰; and
C₃₋₆ carbocycle and 3- to 6-membered heterocycle, any of which is optionally substituted with one or more substituent independently selected from R¹⁸⁰;
when R¹⁵¹ is bromo or —NO₂, R¹⁵² is selected from
iodo, —OR¹⁹⁷, —SR¹⁷⁶, —NHR¹⁷⁸, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
C₁ alkyl substituted with one or more substituent independently selected from R¹⁸⁰, C₁ alkyl substituted with one or more substituent independently selected from R¹⁹⁸, and C₂₋₆ alkyl, optionally substituted with one or more substituent independently selected from R¹⁸⁰:
C₃₋₆ carbocycle and 3- to 6-membered heterocycle, any of which is optionally substituted with one or more substituent independently selected from R¹⁸⁰; and
provided that (i) when R¹⁵¹ is NO₂, R¹⁵² cannot be C₁ alkyl substituted with one or more substituent independently selected from R¹⁸⁰; and (ii) when R¹⁵¹ is NO₂, R¹⁵² cannot be ethyl;
when R¹⁵¹ is —C₁₋₆ haloalkyl, —O—C₂₋₆ alkyl, —O—C₁₋₆ haloalkyl, —OR₃₋₆ carbocycle, —OR₃₋₆ heterocycle, —N(R¹⁶¹)₂, —C(O)R¹⁶¹, —OC(O)R¹⁶¹, —C(O)N(R¹⁶¹)₂, —OC(O)N(R¹⁶¹)₂, —N(R¹⁶¹)C(O)R¹⁶¹, —N(R¹⁶¹)C(O)OR¹⁶¹, —N(R¹⁶¹)C(O)N(R¹⁶¹)₂, —N(R¹⁶¹)S(O)₂(R¹⁶¹), or —N(R¹⁶¹)S(O)₂N(R¹⁶¹)₂, R¹⁵² is selected from
halogen, —OR¹⁷⁶, —SR¹⁷⁶, —N(R¹⁷⁶)₂, —C(O)R¹⁷⁶, —C(O)OR¹⁷⁶, —OC(O)R¹⁷⁶, —OC(O)N(R¹⁷⁶)₂, —C(O)N(R¹⁷⁶)₂, —N(R¹⁷⁶)C(O)R¹⁷⁶, —N(R¹⁷⁶)C(O)OR¹⁷⁶, —N(R¹⁷⁶)C(O)N(R¹⁷⁶)₂, —N(R¹⁷⁶)S(O)₂(R¹⁷⁶), —N(R¹⁷⁶)SO₂N(R¹⁷⁶)₂, —N(R¹⁷⁶)P(O)(OR¹⁷⁶)R¹⁷⁶, —S(O)R¹⁷⁶, —S(O)₂R¹⁷⁶, —S(O)₂N(R¹⁷⁶)₂, —NO₂, and —CN;
C₁₋₆ alkyl, optionally substituted with one or more substituent independently selected from R¹⁷⁷; and
C₃₋₆ carbocycle and 3- to 6-membered heterocycle, any of which is optionally substituted with one or more substituent independently selected from R¹⁷⁷;
R¹⁵³ and R¹⁵⁴ are each independently selected at each occurrence from halogen, —OR¹⁶⁴, —NHR¹⁷⁸, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
C₁₋₄ alkyl, optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN; and
C₃₋₆ carbocycle and 3- to 5-membered heterocycle, any of which is optionally substituted with one or more substituents independently selected from halogen, —OR¹⁶⁴, —SR¹⁶⁴, —N(R¹⁶⁴)₂, —N(R¹⁶⁴)C(O)R¹⁶⁴, —C(O)R¹⁶⁴, —C(O)OR¹⁶⁴, —C(O)N(R¹⁶⁴)₂, —NO₂, and —CN;
R¹⁵⁵ and R^{155′} are each independently selected from hydrogen, hydroxyl, and methyl; or R¹⁵⁵ and R^{155′} taken together are ═O;
R¹⁵⁶ and R^{156′} are each independently selected from hydrogen, hydroxyl, and methyl; or R¹⁵⁶ and R^{156′} taken together are ═O, ═N—OR¹⁷¹, or ═NR¹⁷¹;
t is selected from 0, 1, 2, and 3;
u is selected from 0, 1, 2, and 3;
R¹⁶¹ and R¹⁶⁴ are each independently selected at each occurrence from hydrogen;
C₁₋₆ alkyl optionally substituted with one or more substituent independently selected from R¹⁷⁷; and
C₃₋₆ carbocycle and 3- to 6-membered heterocycle, any of which is optionally substituted with one or more substituent independently selected from R¹⁷⁷;
R¹⁷¹, R¹⁷⁴, and R¹⁷⁶ are each independently selected at each occurrence from hydrogen;
C₁₋₆ alkyl optionally substituted with one or more substituents independently selected from R¹⁸⁰; and
C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁷⁷ and R¹⁸⁰ are each independently selected at each occurrence from halogen, —OR¹⁹¹, —SR¹⁹¹, —N(R¹⁹¹)₂, —C(O)R¹⁹¹, —C(O)OR¹⁹¹, —OC(O)R¹⁹¹, —OC(O)N(R¹⁹¹)₂, —C(O)N(R¹⁹¹)₂, —N(R¹⁹¹)C(O)R¹⁹¹, —N(R¹⁹¹)C(O)OR¹⁹¹, —N(R¹⁹¹)C(O)N(R¹⁹¹)₂, —N(R¹⁹¹)S(O)₂(R¹⁹¹), —N(R¹⁹¹)SO₂N(R¹⁹¹)₂, —N(R¹⁹¹)P(O)(OR¹⁹¹)R¹⁹¹, —NR¹⁹⁶(C═NR¹⁹⁶)N(R¹⁹⁶)₂, —S(O)R¹⁹¹, —S(O)₂R¹⁹¹, —S(O)₂N(R¹⁹¹)₂, —NO₂, ═O, ═S, —CN, C₁₋₆ alkyl, C₃₋₆ carbocycle, and 3- to 7-membered heterocycle;
R¹⁷⁸ is selected at each occurrence from
C₁₋₆ alkyl optionally substituted with one or more substituents independently selected from R¹⁸⁰; and
C₃₋₆ carbocycle and 3- to 6-membered heterocycle, any of which is optionally substituted with one or more substituent independently selected from R¹⁸⁰:
R¹⁹¹ is independently selected at each occurrence from hydrogen, C₁₋₆ alkyl, C₁₋₆ haloalkyl, and C₁₋₆ hydroxyalkyl.
R¹⁹² is selected at each occurrence from C₃₋₆ carbocycle and 3- to 6-membered heterocycle, any of which is optionally substituted with one or more substituent independently selected from R¹⁸⁰;
R¹⁹³ is selected at each occurrence from
C₁₋₆ alkyl optionally substituted with one or more substituents independently selected from R¹⁸⁰; and
C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁴ is selected at each occurrence from
C₁₋₆ alkyl substituted with one or more substituents independently selected from R¹⁸⁰;
R¹⁹⁵ is independently selected at each occurrence from halogen, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)S(O)₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
C₁₋₆ alkyl optionally substituted with one or more substituents independently selected from R¹⁸⁰; and
C₅₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁶ is selected at each occurrence from hydrogen, —CN, and OR¹⁷¹;
C₁₋₆ alkyl optionally substituted with one or more substituents independently selected from R¹⁸⁰; and
C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁷ is selected at each occurrence from hydrogen;
C₂₋₆ alkyl optionally substituted with one or more substituents independently selected from R¹⁸⁰; and
C₃₋₆ carbocycle and 3- to 6-membered heterocycle, any of which is optionally substituted with one or more substituent independently selected from C₁₋₄ alkyl, C₁₋₄ haloalkyl, and R¹⁸⁰;
R¹⁹⁸ is independently selected at each occurrence from fluoro, chloro, iodo, —NH₂, —NHR¹⁹², —NR¹⁹⁶(C═NR¹⁹⁶)N(R¹⁹⁶)₂, —OR¹⁷¹, —SR¹⁷¹, —C(O)R¹⁷¹, —C(O)OR¹⁷¹, —OC(O)R¹⁷¹, —OC(O)N(R¹⁷¹)₂, —C(O)N(R¹⁷¹)₂, —N(R¹⁷¹)C(O)R¹⁷¹, —N(R¹⁷¹)C(O)R¹⁷¹, —N(R¹⁹³)C(O)OR¹⁷¹, —N(R¹⁷¹)C(O)N(R¹⁷¹)₂, —N(R¹⁷¹)S(O)₂(R¹⁷¹), —N(R¹⁷¹)SO₂N(R¹⁷¹)₂, —N(R¹⁷¹)P(O)(OR¹⁷¹)R¹⁷¹, —S(O)R¹⁷¹, —S(O)₂R¹⁷¹, and —S(O)₂N(R¹⁷¹)₂;
C₂₋₆ alkyl optionally substituted with one or more substituents independently selected from R¹⁸⁰; and
C₅₋₆ carbocycle and 3- to 6-membered heterocycle, any of which is optionally substituted with one or more substituent independently selected from C₁₋₄ alkyl, C₁₋₄ haloalkyl, and R¹⁸⁰.

70-92. (canceled)

93. A pharmaceutical composition comprising a pharmaceutically acceptable excipient and the compound or salt of claim 1.

94. A method of modulating indoleamine 2,3-dioxygenase 2 (IDO2) in the subject in need thereof, the method comprising administering to the subject the compound or salt of claim 1.

95. The compound or salt of claim 1, wherein the compound or salt of Formula (I) is selected from: 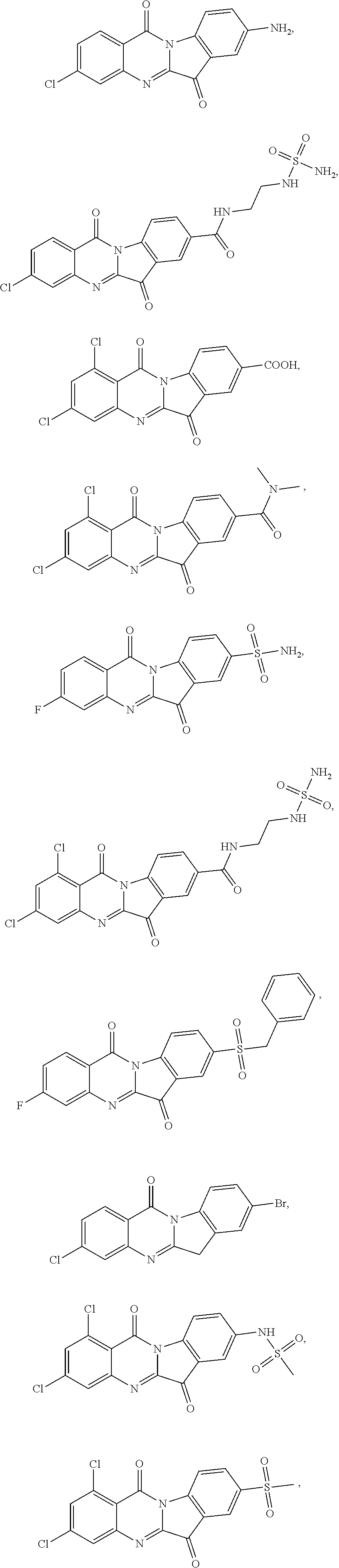
or a salt thereof.
